(19) United States
(12) Patent Application Publication
(10) Pub. No.: US 2023/0116694 A1
(43) Pub. Date: Apr. 13, 2023

(54) INTRAOCULAR LENSES FOR REDUCING PERIPHERAL PSEUDOPHAKIC DYSPHOTOPSIA

(71) Applicant: Brien Holden Vision Institute Limited, Sydney, NSW (AU)

(72) Inventor: Arthur Ho, Mataville, NSW (AU)

(21) Appl. No.: 17/910,243

(22) PCT Filed: Mar. 10, 2021
(86) PCT No.: PCT/IB2021/051990

§ 371 Date: Sep. 8, 2022

Related U.S. Application Data
(60) Provisional application No. 62/988,198, filed on Mar. 11, 2020.

(51) Int. Cl. A61F 2/16

(52) U.S. Cl. CPC A61F 2/1602; A61F 2/1618; A61F 2/1645; A61F 2/1627; A61F 2/1629; A61F 2002/1696

(57) ABSTRACT

An intraocular lens comprising: an optic zone; and a control zone positioned peripherally relative to the optic zone and configured to reduce, minimize, and/or eliminate negative peripheral pseudophakic dysphotopsia (PPD).

### CROSS-REFERENCE TO RELATED APPLICATIONS

This application claims priority to U.S. Provisional Application No. 62/988,198 entitled, Intraocular Lenses for Reducing Peripheral Pseudophakic Dysphotopsia, filed on Mar. 11, 2020. This priority application is incorporated herein by reference in its entirety.

### TECHNICAL FIELD

This disclosure relates to intraocular lenses and more particularly, to intraocular lenses for reducing, minimizing, and/or eliminating peripheral pseudophakic dysphotopsia (e.g., negative and/or positive peripheral pseudophakic dysphotopsia).

### BACKGROUND

Intraocular lens (IOL) implants are lenses implanted into an eye. An IOL may be implanted into an eye to restore, improve, or maintain the vision of the eye. IOLs may be utilized following cataract surgery in which the natural crystalline lens of the eye is removed and replaced with an IOL. An eye that has had its natural lens replaced by an IOL is commonly referred to as “pseudophakic.”

While IOLs are widely used as part of cataract surgery and/or the treatment of near-sighted, far-sighted, and/or astigmatic eyes, IOL implant patients sometimes complain about a phenomenon referred to as peripheral pseudophakic dysphotopsia (PPD). This phenomenon, which can range from being an inconvenience to being visually disturbing, may manifest in two forms—positive and/or negative PPD. Positive PPD is reported by IOL implant patients to be a relatively bright transient light patch in the far peripheral field of a patient's eye. In contrast, negative PPD is perceived as a “dark” or ‘missing’ band or region or patch in the far peripheral field of a patient's eye. In some cases, the PPD may be significant enough that the IOL implant may be removed and replaced by a different IOL (e.g., an IOL with a different design).

Accordingly, there is a need for intraocular lenses for reducing, minimizing, and/or eliminating PPD. Exemplary embodiments may reduce, substantially reduce, minimize, and/or eliminate the effects of PPD (e.g., negative and/or positive PPD) and/or have other advantages as discussed herein. The present disclosure is directed to solving these and other problems disclosed herein. The present disclosure is also directed to pointing out one or more advantages to using exemplary IOL implants described herein.

### SUMMARY

The present disclosure is directed, at least in part, to overcoming and/or ameliorating one or more of the problems described herein.

The present disclosure is directed, at least in part, to an intraocular lens for reducing, minimizing, and/or eliminating peripheral pseudophakic dysphotopsia (e.g., negative and/or positive peripheral pseudophakic dysphotopsia).

The present disclosure is directed, at least in part, to an intraocular lens for reducing, minimizing, and/or eliminating peripheral pseudophakic dysphotopsia (e.g., negative and/or positive peripheral pseudophakic dysphotopsia) by redirecting light rays from peripheral field angles onto retinal locations of the eye that are otherwise void (or substantially void) of peripheral illumination, thus reducing, minimizing, and/or eliminating PPD.

The present disclosure is directed, at least in part, to an intraocular lens comprising: an optic zone; and a control zone positioned peripherally relative to the optic zone and configured to reduce, minimize, and/or eliminate negative peripheral pseudophakic dysphotopsia (PPD).

In some embodiments, the optic zone may comprise a front (anterior) optic surface, a back (posterior) optic surface, a thickness (between front and back optic surfaces which may be constant or vary radially and/or vary circumferentially and/or vary transversely across at least a portion of the optic zone), and a refractive index.

In some embodiments, the control zone may comprise a front (anterior) control surface, a back (posterior) control surface, and an edge. In some embodiments, the control zone may have a thickness that varies radially. For examples, in some embodiments, the thickness may increase towards the periphery or the thickness may decrease towards the periphery.

In some embodiments, the optic zone may comprise a prescribed optical power.

In some embodiments, the optic zone may be configured to deliver an optical power within a large range.

In some embodiments, the optic zone may incorporate any combination of one or more of multifocal optics, which may be refractive and/or diffractive or combinations thereof, for supporting near vision, extended depth of focus optics for supporting near vision, and toric optics for correcting astigmatism.

In some embodiments, the optic zone may be located in a central portion of the IOL and may provide an optical power for supporting vision of the patient.

In some embodiments, the control zone may be positioned towards the periphery of the IOL but may be not extend to the very edge of the IOL.

In some embodiments, the control zone may be positioned towards the periphery of the IOL and extend to the very edge of the IOL.

In some embodiments, the control zone may be configured to control PPD.

In some embodiments, the control zone may be configured to refract light to the dark band region to reduce, significantly reduce, and/or eliminate the occurrence/perception of PPD.

In some embodiments, the control zone may be configured to intercept a portion of oblique light rays (e.g., from light incident on the eye from peripheral field angles) passing through the pupil and redirect and/or distribute the rays to a region on the retina that would otherwise be a dark band.

In some embodiments, the location where the redirected and/or redistributed light hitting the retina may be achieved by appropriate configurations of a back control surface, a front control surface, width of the edge, and/or the thickness or thickness profile of the IOL at the control zone.

In some embodiments, a boundary between the optic zone and the control zone may form an optic-control junction comprising a front optic-control junction that marks the boundary or transition from the front optic surface to the front control surface and a back optic-control junction that marks the boundary or transition from the back optic surface to the front control surface.

In some embodiments, the size (diameter if circular) of the optic zone may be determined by the position of the front optic-control junction and/or the back optic control junction.

In some embodiments, the front optic-control junction may be a point (when viewed as a meridional cross-section) at which the front optic and control surfaces meet.

In some embodiments, the front optic-control junction may be a region (e.g., annulus for a circular IOL) over which the front optic surface transitions (or is blended) to the front control surface.

In some embodiments, the back optic-control junction may be a point (when viewed as a meridional cross-section) at which the back optic and control surfaces meet.

In some embodiments, the back optic-control junction may be a region (e.g., annulus for a circular IOL) over which the back optic surface transitions (or is blended) to the back control surface.

In some embodiments, the position of the front optic-control junction may be set such that the size of the optic zone matches (or closely matches) the size of the patient's pupil.

In some embodiments, the position of the back optic-control junction may be set such that the size of the optic zone matches (or closely matches) the size of the patient's pupil.

In some embodiments, the size of the optic zone may be slightly smaller or larger than the size of the patient's pupil and does not significantly disturb vision.

In some embodiments, the back optic-control junction position may be more peripheral than that of the front optic-control junction.

In some embodiments, the front and/or back control surfaces of the control zone may be configured to have particular surface curvatures and/or profiles to redirect and/or distribute light to otherwise dark band regions of the retina.

In some embodiments, the width of the control zone may be as wide as possible to redirect as much light as possible to redirect light to the otherwise dark band region of the retina without significantly impacting vision.

In some embodiments, the back (posterior) control surface, together with the curvature/surface profile of the front (anterior) control surface may redirect and/or distribute light to a region on the retina that would otherwise be a dark band.

In some embodiments, the back control surface may be convex towards the back of the eye (e.g., concave towards the front of the eye).

In some embodiments, the back control surface may have a steeper curvature (e.g., shorter radius of curvature) than the back optic surface.

In some embodiments, the back control surface profile may vary in curvature (e.g., radius of curvature changes) between back optic-control junction and the edge of the IOL.

In some embodiments, the back control surface profile may be gradually increasing in curvature (e.g., radius of curvature becomes shorter) towards the edge of the IOL.

In some embodiments, the back control surface profile may be gradually decreasing in curvature (e.g., radius of curvature becomes longer) towards the edge of the IOL.

In some embodiments, the back control surface profile may be gradually decreasing and then gradually increasing in curvature (e.g., radius of curvature becomes longer and then shorter) towards the edge of the IOL.

In some embodiments, the back control surface profile may be gradually increasing and then gradually decreasing in curvature (e.g., radius of curvature becomes shorter and then longer) towards the edge of the IOL.

In some embodiments, the back control surface profile may be defined by an aspheric curve; definable by mathematical functions including conics, polynomials, Bezier curves, spline curves, Fourier series, wavelets, or combinations of two or more of such functions.

In some embodiments, a slope of the back control surface proximal to the edge of the IOL may be such that as the back control surface progresses radially outwards (e.g., from axis of the IOL towards the peripheral retina), points on the back control surface become positioned more anteriorly (e.g., towards the iris).

In some embodiments, the absolute value of an angle of a slope relative to a frontal plane of the intraocular lens of the back control surface proximal to the edge of the IOL may be greater than the absolute value of an angle of a slope relative to the frontal plane of the back control surface at the back optic-control junction.

In some embodiments, an angle of a slope of the back control surface relative to a frontal plane of the intraocular lens, at or proximal to the back control-edge junction is more negative in value than an angle of a slope of the back control surface relative to the frontal plane of the intraocular lens at or near to the back optic-control junction.

In some embodiments, a slope of the back control surface proximal to the edge of the IOL and the edge surface may form an angle of less than 90 degrees, about 90 degrees, and/or greater than 90 degrees.

In some embodiments, a slope of the back control surface proximal to the edge of the IOL and the edge surface may form an angle of between 700 and 110°, or between 750 and 105°, or between 800 and 100°.

In some embodiments, the back control surface may be C0-continuous with the back optic surface (e.g., the back control surface meets the back optic surface without a ledge or jump).

In some embodiments, the back control surface may be C1-continuous with the back optic surface (e.g., the back control surface has a common tangent with the back optic surface where they meet).

In some embodiments, the back control surface may be C2-continuous with the back optic surface (e.g., the back control surface has the same instantaneous curvature as the back optic surface at the point where they meet).

In some embodiments, the front control surface may be convex towards the back of the eye (e.g., concave towards the front of the eye).

In some embodiments, the front control surface may have a steeper curvature (e.g., shorter radius of curvature) than the back optic surface.

In some embodiments, the front optic surface may be a positive refracting surface which is convex towards the front of the eye.

In some embodiments, the front control surface profile may vary in curvature (e.g., radius of curvature changes) towards the edge of the IOL.

In some embodiments, the front control surface profile may be gradually increasing in curvature (e.g., radius of curvature becomes shorter) between front optic-control junction and the edge of the IOL.

In some embodiments, the front control surface profile may be gradually decreasing in curvature (e.g., radius of curvature becomes longer) towards the edge of the IOL.

In some embodiments, the front control surface profile may be gradually decreasing and then gradually increasing in curvature (e.g., radius of curvature becomes longer and then shorter) towards the edge of the IOL.

In some embodiments, the front control surface profile may be gradually increasing and then gradually decreasing in curvature (e.g., radius of curvature becomes shorter and then longer) towards the edge of the IOL.

In some embodiments, the front control surface profile may be defined by an aspheric curve; definable by mathematical functions including conics, polynomials, Bezier curves, spline curves, Fourier series, wavelets, or combinations of two or more of such functions.

In some embodiments, a slope of the front control surface proximal to the edge of the IOL may be such that as the front control surface progresses radially outwards (e.g., from axis of the IOL towards the peripheral retina), points on the front control surface become positioned more anteriorly (e.g., towards the iris).

In some embodiments, the absolute value of the angle of a slope relative to a frontal plane of the intraocular lens of the front control surface proximal to the edge of the IOL may be greater than the absolute value of an angle of a slope relative to a frontal plane of the intraocular lens of the front control surface at the front optic-control junction.

In some embodiments, an angle of a slope of the front control surface relative to a frontal plane of the intraocular lens, at or proximal to the front control-edge junction is more negative in value than an angle of a slope of the front control surface relative to the frontal plane of the intraocular lens at or near to the front optic-control junction.

In some embodiments, a slope of the front control surface proximal to the edge of the IOL and the edge surface may form an angle of less than 90 degrees, about 90 degrees, and/or greater than 90 degrees.

In some embodiments, a slope of the front control surface proximal to the edge of the IOL and the edge surface may form an angle of between 700 and 110°, or between 750 and 105°, or between 800 and 100°.

In some embodiments, the front control surface may be C0-continuous with the front optic surface (e.g., the front control surface meets the front optic surface without a ledge or jump).

In some embodiments, the front control surface may be C1-continuous with the front optic surface (e.g., the front control surface has a common tangent with the front optic surface where they meet).

In some embodiments, the front control surface may be C2-continuous with the front optic surface (e.g., the front control surface has the same instantaneous curvature as the front optic surface at the point where they meet).

In some embodiments, the back optic surface and the back control surface may meet to create a gradual transition of ray refraction/deflection angles at the back surface for rays within the optic and control zones in the vicinity of the back optic junction.

In some embodiments, the front optic surface and the front control surface may meet to create a gradual transition of ray refraction/deflection angles at the front surface for rays within the optic and control zones in the vicinity of (e.g., proximal to or near to) the front optic junction.

In some embodiments, the curvature/surface profile of the back control surface and/or the curvature/surface profile of the front control surface may redirect and/or distribute light to a region on the retina that would otherwise be a dark band.

In some embodiments, the edge may be formed by the surface between and joining the front and back control surfaces.

In some embodiments, the edge may be sloped so it faces anteriorly such that a normal to the edge surface and an axis of the IOL may form an angle of less than 45°, 40°, 35°, or 30°.

In some embodiments, the edge may be sloped so it faces anteriorly such that a normal to the edge surface and an axis of the IOL may form an angle of less than about 45°, 40°, 35°, 30°, 25°, 20°, 15°, or 10°.

In some embodiments, the edge may be sloped so it faces anteriorly such that a normal to the edge surface and an axis of the IOL may form an angle of about 40°, 35°, 30°, 25°, 20°, 15°, 10°, 5° or 2.5°.

In some embodiments, the edge may be sloped so it faces anteriorly such that a normal to the edge surface and an axis of the IOL may form an angle of between about 35-45° 35-40°, 25-35°, 25-30°, 15-25°, 10-20°, 10-15°, 5-15°, 0-15°, 5-10°, 0-10°, or 10-40°.

In some embodiments, the edge surface may be sloped so the angle of the slope is substantially the same as a by-pass ray (e.g., the direction of a by-pass ray is substantially parallel to the surface of the edge).

In some embodiments, a width of the edge surface may be about 2.5 mm, 2 mm, 1.5 mm, 1 mm, 0.5 mm, 0.25 mm or 0.1 mm.

In some embodiments, a width of the edge surface may be less than about 2.5 mm, 2 mm, 1.5 mm, 1 mm, 0.5 mm or 0.25 mm.

In some embodiments, the edge surface may be treated to alter its optical characteristics (e.g., one or more of transmission/opacity, scattering/diffusing, spectral transmission, reflectance, etc.).

In some embodiments, the treatment may eliminate or reduce the propagation of light rays that may refract or reflect off the edge either from aqueous to lens (from outside inwards) or from lens to aqueous or vitreous (from inside outwards), or from lens to lens (internal reflection), or from aqueous to aqueous (external reflection).

In some embodiments, the edge surface may be a smooth refracting or reflecting surface, or possesses optical features such as diffraction gratings, meta-surfaces (e.g., nano-optics pillars), frosting (e.g., similar to shower screens to render the surface scattering/diffusing).

In some embodiments, a front control-edge junction may be the location where the front control surface, or a region or zone more peripheral than the front control surface, and the edge of the IOL meet.

In some embodiments, a front control-edge region may be the region on the front surface where the front control surface, or a region or zone more peripheral than the front control surface, joins to the edge of the IOL.

In some embodiments, when regarded as a meridional cross-section, the front control-edge junction may be a sharp corner, a radiused/rounded corner, a chamfered corner, a filleted corner, or a profile that joins the front control surface to the edge.

In some embodiments, a back control-edge junction may be the location where the back control surface, or a region or zone more peripheral than the back control surface, and the edge of the IOL meet.

In some embodiments, a back control-edge region may be the region on the back surface where the back control surface, or a region or zone more peripheral than the back control surface, joins to the edge of the IOL.

In some embodiments, when regarded as a meridional cross-section, the back control-edge junction may be a sharp corner, a radiused/rounded corner, a chamfered corner, a filleted corner, or a profile that joins the back control surface to the edge.

In some embodiments, the intraocular lens may be a supplementary intraocular lens that is implanted to operate in conjunction with (e.g., in combination with, together with) another intraocular lens (e.g., an existing IOL that has been implanted previously). For example, the required prescriptive power is provided by the combination of optical power of the existing IOL and the supplementary intraocular lens, and the supplementary intraocular lens comprises a control zone that is configured to reduce, minimize, and/or eliminate peripheral pseudophakic dysphotopsia.

Other features and advantages of the subject matter described herein will be apparent from the description and drawings, and from the claims.

### BRIEF DESCRIPTION OF THE DRAWINGS

Aspects of the embodiments described herein may be understood from the following detailed description when read with the accompanying figures.

FIG. 1 is a three-dimensional schematic model of an eye with an intraocular lens with ray tracing in accordance with certain embodiments.

FIG. 2 is a ray intercept plot showing the distribution of light rays intercepting the retina for an incident light field angle of about 84 degrees in accordance with certain embodiments.

FIG. 3 is a ray intercept plot showing the distribution of light rays intercepting the retina for an incident light field angle of about 87.5 degrees in accordance with certain embodiments.

FIG. 4 is a ray intercept plot showing the distribution of light rays intercepting the retina for an incident light field angle of about 90 degrees in accordance with certain embodiments.

FIG. 5 is a ray intercept plot showing the distribution of light rays intercepting the retina for an incident light field angle of about 93.5 degrees in accordance with certain embodiments.

FIG. 6 is a ray density plot integrated over a range of field angles showing the intensity of light distribution across the retina in accordance with certain embodiments.

FIG. 7 is an integrated ray density plot (or relative whole field retinal irradiance plot) integrating over a range of field angles and azimuthal angles showing the intensity of light distribution across the retina in accordance with certain embodiments.

FIGS. 8A-F are integrated ray density plots showing the intensity (e.g., relative whole field retinal irradiance, e.g., integrated over a range of field angles and azimuthal angles) of light distribution across the retina for the intraocular lens modelled in FIG. 1 for pupil sizes from 2.5 mm to 5 mm in 0.5 mm steps at an implantation depth of 0 mm in accordance with certain embodiments.

FIGS. 9A-F are integrated ray density plots showing the intensity (e.g., relative whole field retinal irradiance) of light distribution across the retina for the intraocular lens modelled in FIG. 1 for pupil sizes from 2.5 mm to 5 mm in 0.5 mm steps at an implantation depth of 0.1 mm in accordance with certain embodiments.

FIGS. 10A-F are integrated ray density plots showing the intensity (e.g., relative whole field retinal irradiance) of light distribution across the retina for the intraocular lens modelled in FIG. 1 for pupil sizes from 2.5 mm to 5 mm in 0.5 mm steps at an implantation depth of 0.2 mm in accordance with certain embodiments.

FIGS. 11A-F are integrated ray density plots showing the intensity (e.g., relative whole field retinal irradiance) of light distribution across the retina for the intraocular lens modelled in FIG. 1 for pupil sizes from 2.5 mm to 5 mm in 0.5 mm steps at an implantation depth of 0.3 mm in accordance with certain embodiments.

FIGS. 12A-F are integrated ray density plots showing the intensity (e.g., relative whole field retinal irradiance) of light distribution across the retina for the intraocular lens modelled in FIG. 1 for pupil sizes from 2.5 mm to 5 mm in 0.5 mm steps at an implantation depth of 0.4 mm in accordance with certain embodiments.

FIGS. 13A-F are integrated ray density plots showing the intensity (e.g., relative whole field retinal irradiance) of light distribution across the retina for the intraocular lens modelled in FIG. 1 for pupil sizes from 2.5 mm to 5 mm in 0.5 mm steps at an implantation depth of 0.5 mm in accordance with certain embodiments.

FIGS. 14A-F are integrated ray density plots showing the intensity (e.g., relative whole field retinal irradiance) of light distribution across the retina for the intraocular lens modelled in FIG. 1 for pupil sizes from 2.5 mm to 5 mm in 0.5 mm steps at an implantation depth of 0.6 mm in accordance with certain embodiments.

FIGS. 15A-F are integrated ray density plots showing the intensity (e.g., relative whole field retinal irradiance) of light distribution across the retina for the intraocular lens modelled in FIG. 1 for pupil sizes from 2.5 mm to 5 mm in 0.5 mm steps at an implantation depth of 0.7 mm in accordance with certain embodiments.

FIG. 16 is a schematic illustration of a half-meridian section of an intraocular lens for reducing, minimizing, and/or eliminating peripheral pseudophakic dysphotopsia in accordance with certain embodiments.

FIG. 17 is a ray density plot integrated over a range of field angles showing the intensity of light distribution across the retina using an eye modelled in accordance with FIG. 1 and the intraocular lens of FIG. 29 in accordance with certain embodiments.

FIG. 18 is a relative whole field retinal irradiance plot (e.g., integrated ray density plot) integrated over a range of field angles and azimuthal angles showing the intensity of light distribution across the retina for the ray density plot of FIG. 17 in accordance with certain embodiments.

FIGS. 19A-F are integrated ray density plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0 mm in accordance with certain embodiments.

FIGS. 20A-F are integrated ray density plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.1 mm in accordance with certain embodiments.

FIGS. 21A-F are integrated ray density plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.2 mm in accordance with certain embodiments.

FIGS. 22A-F are integrated ray density plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.3 mm in accordance with certain embodiments.

FIGS. 23A-F are integrated ray density plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.4 mm in accordance with certain embodiments.

FIGS. 24A-F are integrated ray density plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.5 mm in accordance with certain embodiments.

FIGS. 25A-F are integrated ray density plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.6 mm in accordance with certain embodiments.

FIGS. 26A-F are integrated ray density plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.7 mm in accordance with certain embodiments.

FIG. 27 is a schematic illustration of a half-meridian section of an intraocular lens for reducing, minimizing, and/or eliminating peripheral pseudophakic dysphotopsia in accordance with certain embodiments.

FIG. 28 is an exemplary embodiment of an implementation of Eq. 1 for defining a control surface profile of an intraocular lens in accordance with certain embodiments.

FIG. 29 is a schematic illustration of a half-meridian section of an intraocular lens for reducing, minimizing, and/or eliminating peripheral pseudophakic dysphotopsia in accordance with certain embodiments.

FIG. 30 is an exemplary embodiment of an implementation of Eq. 2 for defining a control surface profile of an intraocular lens in accordance with certain embodiments.

FIG. 31 is a schematic illustration of a half-meridian section of an intraocular lens for reducing, minimizing, and/or eliminating peripheral pseudophakic dysphotopsia in accordance with certain embodiments.

FIG. 32 is a schematic illustration of a half-meridian section of an intraocular lens for reducing, minimizing, and/or eliminating peripheral pseudophakic dysphotopsia in accordance with certain embodiments.

FIG. 33 is a schematic illustration of a half-meridian section of an intraocular lens for reducing, minimizing, and/or eliminating peripheral pseudophakic dysphotopsia in accordance with certain embodiments.

FIG. 34 is a schematic illustration of a half-meridian section of an intraocular lens for reducing, minimizing, and/or eliminating peripheral pseudophakic dysphotopsia in accordance with certain embodiments.

FIG. 35 is a schematic illustration of a half-meridian section of an intraocular lens for reducing, minimizing, and/or eliminating peripheral pseudophakic dysphotopsia in accordance with certain embodiments.

FIG. 36 is a schematic illustration of a half-meridian section of an intraocular lens for reducing, minimizing, and/or eliminating peripheral pseudophakic dysphotopsia in accordance with certain embodiments.

FIG. 37 is a schematic illustration of a half-meridian section of an intraocular lens for reducing, minimizing, and/or eliminating peripheral pseudophakic dysphotopsia in accordance with certain embodiments.

FIG. 38 is a schematic illustration of a half-meridian section of an intraocular lens for reducing, minimizing, and/or eliminating peripheral pseudophakic dysphotopsia in accordance with certain embodiments.

FIG. 39 is a schematic illustration of a half-meridian section of an intraocular lens for reducing, minimizing, and/or eliminating peripheral pseudophakic dysphotopsia in accordance with certain embodiments.

FIG. 40 is a schematic illustration of a half-meridian section of an intraocular lens for reducing, minimizing, and/or eliminating peripheral pseudophakic dysphotopsia in accordance with certain embodiments.

FIGS. 41A-F are integrated ray density plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0 mm in accordance with certain embodiments.

FIGS. 42A-F are integrated ray density plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.1 mm in accordance with certain embodiments.

FIGS. 43A-F are integrated ray density plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.2 mm in accordance with certain embodiments.

FIGS. 44A-F are integrated ray density plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.3 mm in accordance with certain embodiments.

FIGS. 45A-F are integrated ray density plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.4 mm in accordance with certain embodiments.

FIGS. 46A-F are integrated ray density plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.5 mm in accordance with certain embodiments.

FIGS. 47A-F are integrated ray density plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.6 mm in accordance with certain embodiments.

FIGS. 48A-F are integrated ray density plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.7 mm in accordance with certain embodiments.

FIGS. 49A-F are integrated ray density plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0 mm in accordance with certain embodiments.

FIGS. 50A-F are integrated ray density plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.1 mm in accordance with certain embodiments.

FIGS. 51A-F are integrated ray density plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.2 mm in accordance with certain embodiments.

FIGS. 52A-F are integrated ray density plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.3 mm in accordance with certain embodiments.

FIGS. 53A-F are integrated ray density plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.4 mm in accordance with certain embodiments.

FIGS. 54A-F are integrated ray density plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.5 mm in accordance with certain embodiments.

FIGS. 55A-F are integrated ray density plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.6 mm in accordance with certain embodiments.

FIGS. 56A-F are integrated ray density plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.7 mm in accordance with certain embodiments.

FIGS. 57A-F are integrated ray density plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0 mm in accordance with certain embodiments.

FIGS. 58A-F are integrated ray density plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.1 mm in accordance with certain embodiments.

FIGS. 59A-F are integrated ray density plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.2 mm in accordance with certain embodiments.

FIGS. 60A-F are integrated ray density plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.3 mm in accordance with certain embodiments.

FIGS. 61A-F are integrated ray density plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.4 mm in accordance with certain embodiments.

FIGS. 62A-F are integrated ray density plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.5 mm in accordance with certain embodiments.

FIGS. 63A-F are integrated ray density plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.6 mm in accordance with certain embodiments.

FIGS. 64A-F are integrated ray density plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.7 mm in accordance with certain embodiments.

FIGS. 65A-F are integrated ray density plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0 mm in accordance with certain embodiments.

FIGS. 66A-F are integrated ray density plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.1 mm in accordance with certain embodiments.

FIGS. 67A-F are integrated ray density plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.2 mm in accordance with certain embodiments.

FIGS. 68A-F are integrated ray density plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.3 mm in accordance with certain embodiments.

FIGS. 69A-F are integrated ray density plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.4 mm in accordance with certain embodiments.

FIGS. 70A-F are integrated ray density plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.5 mm in accordance with certain embodiments.

FIGS. 71A-F are integrated ray density plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.6 mm in accordance with certain embodiments.

FIGS. 72A-F are integrated ray density plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.7 mm in accordance with certain embodiments.

FIGS. 73A-F are integrated ray density plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0 mm in accordance with certain embodiments.

FIGS. 74A-F are integrated ray density plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.1 mm in accordance with certain embodiments.

FIGS. 75A-F are integrated ray density plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.2 mm in accordance with certain embodiments.

FIGS. 76A-F are integrated ray density plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.3 mm in accordance with certain embodiments.

FIGS. 77A-F are integrated ray density plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.4 mm in accordance with certain embodiments.

FIGS. 78A-F are integrated ray density plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.5 mm in accordance with certain embodiments.

FIGS. 79A-F are integrated ray density plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.6 mm in accordance with certain embodiments.

FIGS. 80A-F are integrated ray density plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.7 mm in accordance with certain embodiments.

FIG. 81 is a schematic illustration of a half-meridian section of an intraocular lens for reducing, minimizing, and/or eliminating peripheral pseudophakic dysphotopsia in accordance with certain embodiments.

FIG. 82 is a schematic illustration of a half-meridian section of an intraocular lens for reducing, minimizing, and/or eliminating peripheral pseudophakic dysphotopsia in accordance with certain embodiments.

FIG. 83 is a schematic illustration of a half-meridian section of an intraocular lens for reducing, minimizing, and/or eliminating peripheral pseudophakic dysphotopsia in accordance with certain embodiments.

FIG. 84 is a schematic illustration of a half-meridian section of an intraocular lens for reducing, minimizing, and/or eliminating peripheral pseudophakic dysphotopsia in accordance with certain embodiments.

FIGS. 85A-F are integrated ray density plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0 mm in accordance with certain embodiments.

FIGS. 86A-F are integrated ray density plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.1 mm in accordance with certain embodiments.

FIGS. 87A-F are integrated ray density plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.2 mm in accordance with certain embodiments.

FIGS. 88A-F are integrated ray density plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.3 mm in accordance with certain embodiments.

FIGS. 89A-F are integrated ray density plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.4 mm in accordance with certain embodiments.

FIGS. 90A-F are integrated ray density plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.5 mm in accordance with certain embodiments.

FIGS. 91A-F are integrated ray density plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.6 mm in accordance with certain embodiments.

FIGS. 92A-F are integrated ray density plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.7 mm in accordance with certain embodiments.

FIGS. 93A-F are integrated ray density plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0 mm in accordance with certain embodiments.

FIGS. 94A-F are integrated ray density plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.1 mm in accordance with certain embodiments.

FIGS. 95A-F are integrated ray density plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.2 mm in accordance with certain embodiments.

FIGS. 96A-F are integrated ray density plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.3 mm in accordance with certain embodiments.

FIGS. 97A-F are integrated ray density plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.4 mm in accordance with certain embodiments.

FIGS. 98A-F are integrated ray density plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.5 mm in accordance with certain embodiments.

FIGS. 99A-F are integrated ray density plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.6 mm in accordance with certain embodiments.

FIGS. 100A-F are integrated ray density plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.7 mm in accordance with certain embodiments.

FIG. 101 is a schematic illustration of a half-meridian section of an intraocular lens that functions as a supplementary intraocular lens for reducing, minimizing, and/or eliminating peripheral pseudophakic dysphotopsia in accordance with certain embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

The subject headings used in the detailed description are included for the ease of reference of the reader and should not be used to limit the subject matter found throughout the disclosure or the claims. The subject headings should not be used in construing the scope of the claims or the claim limitations.

The terms “about” as used in this disclosure is to be understood to be interchangeable with the term approximate or approximately. In some instances, the term “about” may be understood to be interchangeable with the term approximal or approximally.

The term “comprise” and its derivatives (e.g., comprises, comprising) as used in this specification is to be taken to be inclusive of features to which it refers, and is not meant to exclude the presence of additional features unless otherwise stated or implied.

The term “intraocular lens (“IOL”) as used herein is any lens implanted in the eye for restoration, partial restoration, correction, and/or improvement of vision. The IOL may be used following cataract surgery in which the natural crystalline lens is removed and replaced by an IOL. An eye that has had its natural lens replaced by an IOL is referred to as “pseudophakic.”

Also as used herein, intraocular lens may refer to a singular (e.g., stand-alone, or monolithic) intraocular lens that is implanted alone for pseudophakia, or may refer to one or more of a system of intraocular lenses that are implanted to operate in combination (e.g., conjunction, or together) to provide the required vision correction, or may refer to a supplementary intraocular lens that is implanted to operate in combination with (e.g., in conjunction with, or together with, or in unison with) an existing (e.g., implanted earlier) intraocular lens to provide a ‘supplementary’ function such as reduction, minimization and/or elimination of peripheral pseudophakic dysphotopsia.

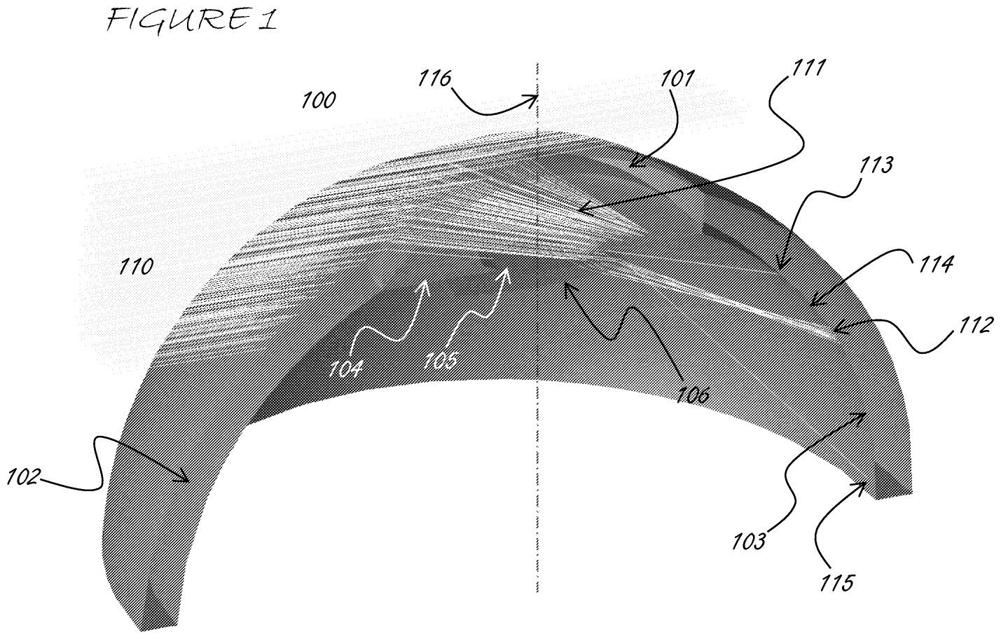

FIG. 1 is a three-dimensional schematic model of an eye with an intraocular lens with ray tracing in accordance with certain embodiments. As illustrated, FIG. 1 shows an eye 100 implanted with an intraocular lens (IOL) 106. This model in FIG. 1 was computer generated using Zemax Opticstudio (version 18) ray-tracing in non-sequential ray-tracing mode and the dimensions of the eye 100 are based approximally on the Arizona Eye Model. The IOL 106 is an equi-convex (e.g., bi-convex with the same front and back surface radii of curvature) design with an optic zone diameter of 6 mm. The eye 100 comprises a cornea 101, a sclera 102, a retinal surface 103 at the inner surface of the sclera 102, an iris 104 and a pupil 105. The IOL 106 is located below (e.g., more posterior to) the iris 104 and pupil 105 as may be typical of IOL implantation following surgical removal of cataract. While in practice some components of the natural human eye are slightly tilted and decentered relative to each other, for general visual optical modelling, the eye may be (and have generally been) treated as approximally rotationally symmetric about an axis of the eye 116, and an axis of the IOL may be treated as approximally coincident with an axis of the eye.

As illustrated, a beam of light rays 110 is incident onto the eye at about an 89.3 degree field angle (e.g., 89.3 degrees from the axis 116 of the eye). Anterior chamber rays 111 are light rays that are refracted by the cornea 101 into the anterior chamber. In some embodiments, some anterior chamber rays may be blocked by the iris 104. Refracted rays (e.g., arriving at retinal position 112) are light rays that after refraction by the cornea 101 and traversal of the anterior chamber, passes through the opening of the pupil 105, are refracted by the IOL 106 and ultimately reaches a portion of the retina 103. By-pass rays (e.g., arriving at retinal position 113) are light rays that after refraction by the cornea 101 and traversal of the anterior chamber and passing through the pupil 105, miss the IOL 106 and directly reach a portion of the retina 103. Due to the obliquity of the rays, the diameter of the pupil, the diameter of the IOL and/or the implantation depth of the IOL, the by-pass rays miss the IOL and are therefore not refracted by the IOL—that is, they by-pass the IOL to directly reach the retina.

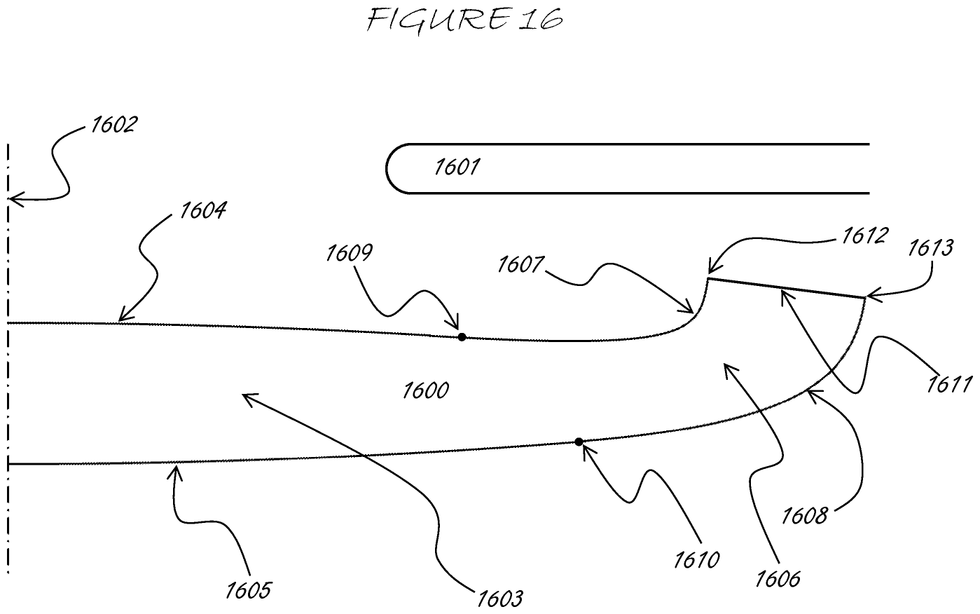

As used herein, the “implantation depth” of an IOL is the distance between the iris/pupil of the eye and the IOL. The implantation depth may vary according to the anatomy or geometry of the eye, the design of the IOL (e.g., the design of the haptic for fixing, aligning or centering the IOL in the eye), and/or the surgical procedure used to implant the IOL. The IOL of the present invention may employ any of a number of haptic designs for fixing, aligning or centering the implanted IOL in the eye such as J-loops, C-loops, plate-shaped haptics, etc. In certain embodiments, the haptic or fixation devices may be attached wholly or in part to the IOL at its lens edge (for example, 1611 in FIG. 16) or the back control surface (for example, 1608 in FIG. 16) or combinations thereof.

The “dark band” region 114 is a region on the retinal surface 103 between the portion of the retina intercepting the refracted rays 112 and the portion of the retina intercepting the by-pass rays 113. In some instances, with certain field angles, eye geometry, IOL geometry and/or optical properties, and/or implantation depth, there may be a region on the retina where no light (e.g., no light, substantially no light, minimal light, etc.) reaches regardless of field angle. This dark band region 114 may be considered to be void of photic retinal stimulus and may be perceived by the patient as a dark region in their visual field. In some embodiments, this dark region may be the basis of negative peripheral pseudophakic dysphotopsia (PPD).

In the computer model of FIG. 1, the edge of the IOL 106 was assumed to be transparent with a flat, square edge and sharp corner. With this assumption, at certain field angles, a small amount of light, following refraction through the front surface of the IOL, may undergo refraction or reflection at the edge surface of the IOL. Such rays (e.g., edge rays) are deflected to reach a different retina position 115. In practice, IOL edges are typically curved or profiled (e.g., rounded, radiused, chamfered, beveled, filleted) and would spread widely light that reaches the lens edge thereby rendering such rays of negligible consequence to the vision of the patient.

As previously mentioned, the model in FIG. 1 was computer generated using Zemax Opticstudio (version 18) ray-tracing in non-sequential mode and the dimensions of the eye 100 are approximally based on the Arizona Eye Model. Based on the Arizona Eye Model, and to facilitate modeling of retinal irradiance, the eye 100 may have the following characteristics: an iris thickness of 0.25 mm; a scleral internal radius of 12 mm; a retinal radius of 11.995 mm (e.g., the retinal radius may be set about 5 m less than the internal scleral radius to facilitate computation of ray intercept to the retina during ray-tracing analyses; ensuring traced rays intercept the retina surface before the sclera). This minute reduction (5 μm) in effective internal scleral radius does not substantially alter the results in terms of irradiance and distribution of light rays on the retina. In the model of FIG. 1, the IOL may have the following characteristics: a lens optic diameter of 6.0 mm; front radius=+22.54 mm; back radius=−22.54 mm (in the sign convention adopted herein, a negative value in radius for a back surface indicates a convex surface); center thickness=0.55 mm; and lens material refractive index=1.55 resulting in an optical power of about +19 D (diopters). Ray-tracing analyses of the model may be run over a field angle range of about 550 to 100° (e.g., in 0.5° step); and/or for pupil diameter range of 2.5 mm-5.0 mm (e.g., in 0.5 mm step). In some embodiments, the modelled implantation depth of the IOL may be 0 mm to 0.7 mm (e.g., in 0.1 mm step).

In the particular model in FIG. 1, the diameter of the pupil 105 formed by the aperture in the iris 104 was 3 mm and the IOL implantation depth (distance from the pupil/iris plane to the front of the IOL) is 0.3 mm. The field angle (angle between direction of incident light 110 and axis of the eye 116 (where 0° is light approaching the eye from directly in front) was about 89.3°. For clarity, only 500 rays have been included in the rendering of the model in FIG. 1.

A beam of light incident on the eye 100 from the field angle of about 89.3° after refraction by the cornea 101, traverses through and across the anterior chamber. A portion of the anterior chamber rays 111 may be obstructed from further propagation by anatomical features such as the iris 104. Other rays may pass through the pupil 105. Of the rays that pass through the pupil 105, a proportion of rays will be refracted by the optics of the IOL 106. Such refracted rays are directed to the retinal surface 103 at retinal position 112. For a given geometry of an eye and optical properties of the IOL (e.g., that of FIG. 1), depending on the incident field angle, the position of irradiance on the retina 112 on the retina surface 103 may vary—more posteriorly or more anteriorly. Of the rays that pass through the pupil 105, and depending on incident field angle, another proportion of rays may by-pass the IOL 106 and reach the retina 103 without refraction by the IOL 106. Such by-pass rays may be directed to a more anterior position on the retina at 113. Depending on the incident field angle, the position of irradiance on the retina 113 on the retina surface 103 may vary—more posteriorly or more anteriorly. However, the position is independent of the refracting power of the IOL. Thus, there is a ‘gap’ (or “dark band” 114) between the two irradiated positions 113 and 112. As discussed above, the region 114 may be devoid of (or at least substantially devoid of) light.

For certain combinations of properties of the eye (including combinations of one or more of corneal shape and power, anterior chamber depth, and/or pupil diameter) and IOL properties (including combinations of one or more of power and power profile, lens form or shape, diameter and/or thickness and refractive index of the IOL) as well as physiological and surgical factors such as implantation depth, a dark band region on the retina may exist regardless of the field angle or meridian or azimuth, or azimuthal angle, where a meridian is a plane passing through the eye's axis. For example, incident light arriving from a position on the horizontal plane may be considered to be in the horizontal meridian and a light source lying in a plane at 450 from the horizontal plane may be described by an azimuthal or meridional angle of 45°.

The occurrence of the dark band 114 may explain the phenomenon referred to as negative PPD in which patients (following IOL implantation) report a band, or patch or region, in their visual field which is void of light—that is, a ‘dark band’ across their vision.

Concomitantly, the position of rays that reach the retina after refraction by the IOL at position 112 and rays that reach the more anterior position 113 by-passing the IOL, do depend on the field angle of the incident light 110. As the patient rotates their eye or head to view towards different directions, a bright light source (e.g., similar to a lit streetlamp at night) may appear transiently in that retinal position. This may create the impression of a ‘jump’ or a ‘flash of light’ consistent with reports of positive PPD. For example, if a spot of light is moving towards the patient's peripheral field of vision, from a lower field angle (closer to front-on to the eye's direction of gaze) towards a greater field angle (more peripheral), its rays may initially be refracted by the IOL to a position such as 112 on the retina. As the spot of light moves more peripherally in the patient's field of vision, the light spot on the retina will move more anteriorly. However, at certain more peripheral field angles, rays from the spot of light will by-pass the IOL and appear to ‘jump’ to the more anterior retina position 113, then finally extinguish as the increasing peripheral field angle exceeds the total visual field of the eye. This discontinuity in the perceived light spot movement may give the impression of a ‘flash’ to the patient.

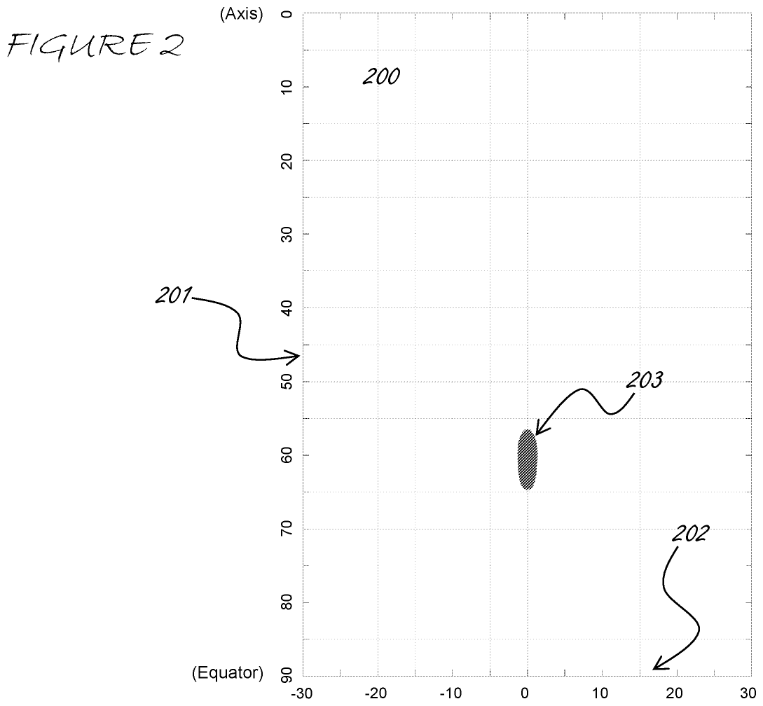

FIGS. 2-15 show plots from computation ray-tracing analysis of the eye and IOL model of FIG. 1 with the parameter values for the eye and IOL as detailed above.

FIGS. 2-5 are ray intercept plots showing the distribution of light rays intercepting the retina for various incident light field angles. The non-sequential ray-tracing model shown in FIG. 1 was used to compute (using Zemax Opticstudio) the propagation of about 100,000 light rays incident at a range of field angles from 55° to 110° in 0.5° steps for an equi-convex IOL with a square edge profile. The ray-intercept plots show the distribution of light rays intercepting the retina for a given incident light field angle (or just incident angle or just field angle). That is, each ray intercepting the retina is plotted as a single point representing the point of intersection of that ray with the retina surface. By plotting the rays that reached the retina, where rays cluster and/or are concentrated, a cluster of points reveal retina light spots or brighter light patches. The light source for the incident light beam with a beam width of about 3 mm was set to model a point source at infinity (i.e., a light source position a long way from the eye) producing a beam of parallel light rays incident on the eye.

In the ray-intercept plots, the horizontal axis (e.g., 202 in FIG. 2) represents a transverse (or meridional or azimuthal) retinal position. Zero degree along this axis represents the antero-posterior positions along the retina that are in the same meridional plane as the incident light source. The sclera/retina of the eye is assumed to be spherical. In the ray-tracing model, only the anterior hemisphere of the sclera and retina was modelled since PPD is considered to be a visual phenomenon relating to the periphery to far periphery of the visual field. Light rays from the peripheral to far peripheral field, following refraction by the eye and IOL, typically intercept the retina at the peripheral to far peripheral retina, which is the portion of the retina anterior to the equator of the sphere representing the retina. The equator is the locus of positions around the retina where the retina surface intersects a frontal or coronal plane that bisects the eye into an anterior hemisphere and a posterior hemisphere.

The vertical axis (e.g., 201 in FIG. 2) in the ray-intercept plots represents the antero-posterior position of the anterior hemisphere of the retina surface. The scaling is in degrees relative to the center of the sphere representing the sclera/retina. Thus, 0° (towards the top of the plots) represents the direction along the axis of the eye (i.e., facing directly forward, or in the direction the eye is looking), and 90° (at the bottom of the plots) represents a position at the equator of the retina. Note the scaling of this axis is such that ‘straight ahead’ (0°) for the patient's vision is upwards. Thus, higher positions along the vertical axis (i.e., with lower angular degrees values) represent more peripheral to far peripheral positions on the retina.

FIGS. 2-5 show the irradiance distribution of light on the peripheral to far peripheral retina for select exemplary angles of incidence as the angle of incident of a light source gradually increases (e.g., becomes more peripheral in position in the visual field). The pupil size modelled was 3 mm and the implantation depth 0.3 mm.

FIG. 2 is a ray intercept plot 200 showing the distribution of light rays intercepting the retina for an incident light field angle of about 84 degrees in accordance with certain embodiments. When a beam of light from infinity is incident on the eye at an angle of about 84°, effectively all (e.g., substantially all) light rays that pass through the pupil are incident on the retina at a single ray cluster position creating a light spot 203 after being refracted by the IOL. These correspond to the refracted rays at retinal position 112 as discussed in FIG. 1. The non-circular shape of the retinal light spot 203 may be due, at least in part, to combinations of one or more of the peripheral aberrations of the eye such as (radial or oblique) astigmatism and coma as well as peripheral refractive defocus in which the light rays may not be focused on the retina but in front of (more anterior, e.g., in the vitreous or posterior chamber of the eye) or behind (more posterior) the retina.

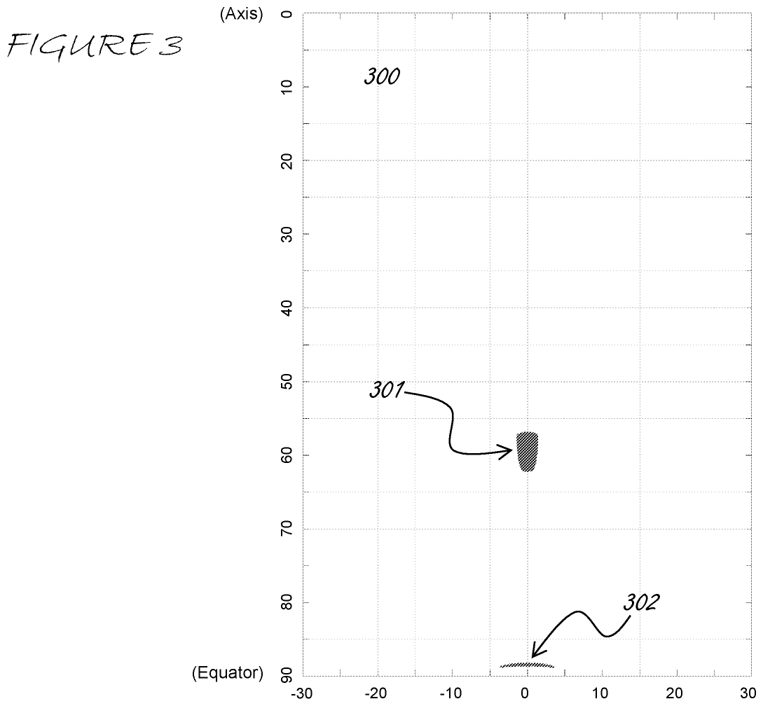

FIG. 3 is a ray intercept plot 300 showing the distribution of light rays intercepting the retina for an incident light field angle of about 87.5 degrees in accordance with certain embodiments. When a beam of light from infinity is incident on the eye at a more peripheral angle of 87.5°, the majority of light rays that pass through the pupil are incident on the retina at the refracted light spot 301 after being refracted by the IOL. A proportion of the light rays, after refraction by the front surface of the IOL, is refracted by the flat/square edge surface of the modelled IOL in FIG. 1. This directs the ‘edge’ rays more posteriorly, to an ‘edge’ ray spot 302. The square edge of the IOL in FIG. 1 may be a simplification for ray-tracing purpose. For example, in some IOLs, the edge shapes may be rounded, radiused, chamfered, beveled or filleted and would spread light across the retina substantially eliminating the existence of an ‘edge’ ray spot 302 or rendering its visual impact negligible.

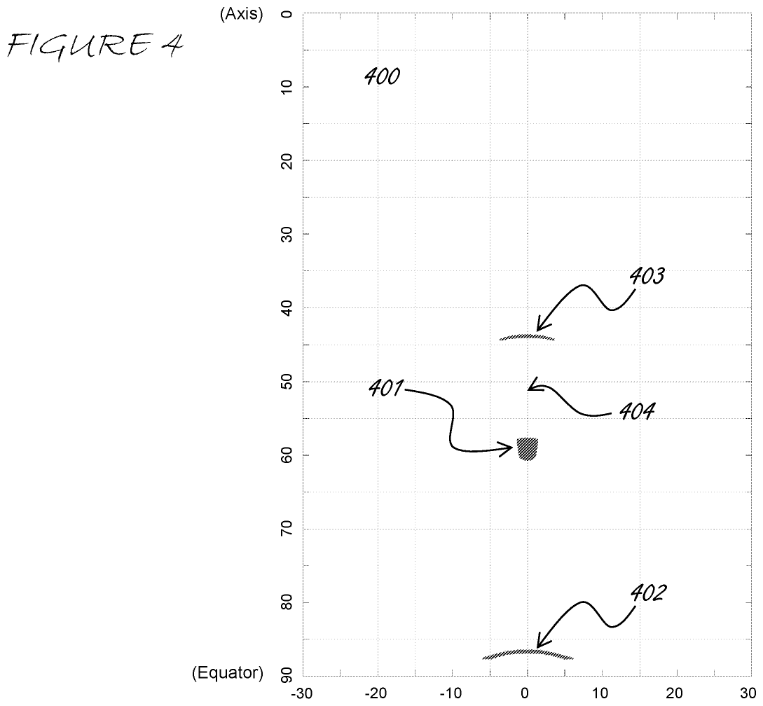

FIG. 4 is a ray intercept plot 400 showing the distribution of light rays intercepting the retina for an incident light field angle of about 90 degrees in accordance with certain embodiments. When a beam of light from infinity is incident on the eye at an even more peripheral angle of about 90°, the majority of light rays that pass through the pupil are incident on the retina at the refracted light spot 401 after being refracted by the IOL. A proportion of the light rays, after refraction by the front surface of the IOL, is refracted by the flat/square edge surface of the IOL of FIG. 1. This directs the ‘edge’ rays more posteriorly, to an ‘edge’ ray spot 402. The square edge of the IOL in FIG. 1 may be a simplification for ray-tracing purposes. For example, in some IOLs, the edge shapes may be rounded, radiused, chamfered, beveled or filleted and would spread light across the retina substantially eliminating the existence of an ‘edge’ ray spot 402. At this field angle, some rays also by-pass the IOL traversing the space in the posterior chamber between the back of the iris and the front of the IOL. These rays arrive at the retina to produce the by-pass ray spot 403 located more anteriorly on the retina surface than the refracted light spot 401. The gap 404 between refracted rays retinal light spot 401 and by-pass rays retinal light spot 403 is a region void (e.g., substantially void) of light. As is described herein, with certain combinations of eye properties, IOL properties and surgical factors, such a gap 404 may persist regardless of incident light field angle producing a dark band which may explain the phenomenon of negative PPD.

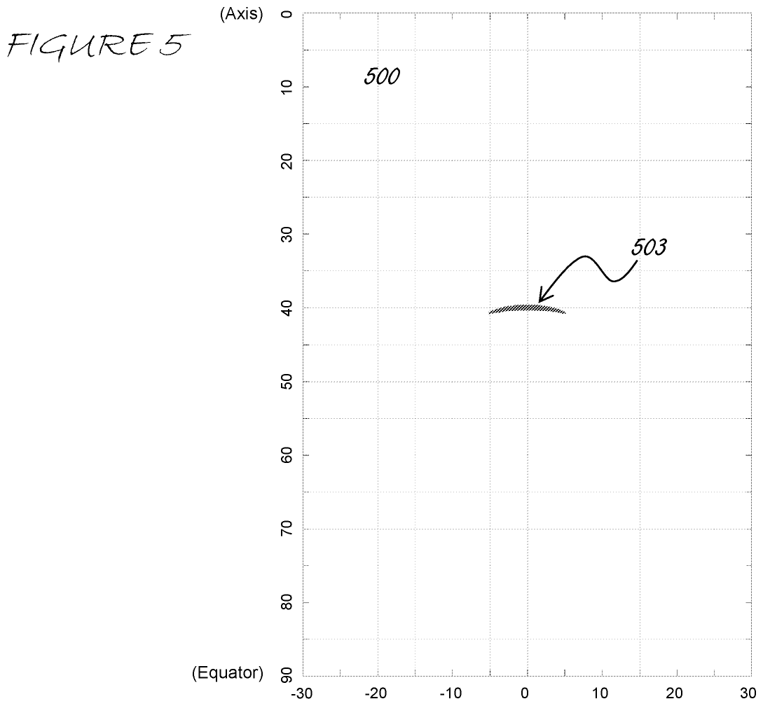

FIG. 5 is a ray intercept plot 500 showing the distribution of light rays intercepting the retina for an incident light field angle of about 93.5 degrees in accordance with certain embodiments. When a beam of light from infinity is incident on the eye at an even greater peripheral angle of about 93.5°, effectively all light rays that pass through the pupil by-pass the IOL traversing the space in the posterior chamber between the back of the iris and the front of the IOL. These rays arrive at the retina to produce the by-pass ray spot 503. In some instances, beyond a particular peripheral field angle, such as this peripheral angle of 93.5°, no refracted light spot (i.e., intended retina image) is formed and the patient perceives only a single source of light, which is the projection of the by-pass rays retinal light spot back into visual space.

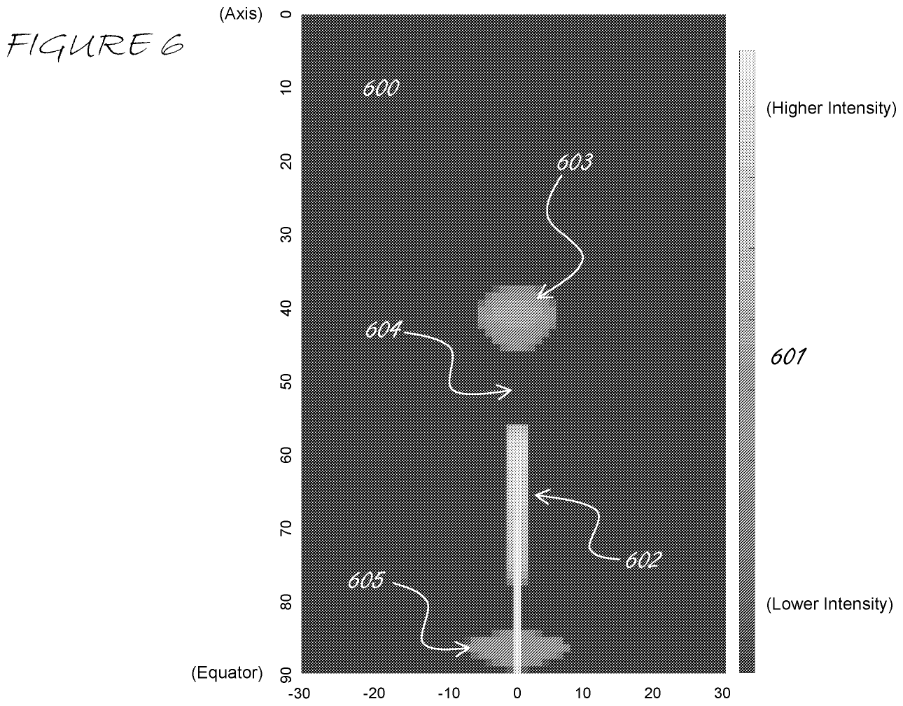

FIG. 6 is a ray density plot 600 integrated over a range of field angles showing the intensity of light distribution across the retina in accordance with certain embodiments. FIG. 6 shows a ray-density plot 600 integrating over a range of field angles along a single meridian (e.g., a single azimuthal angle). The horizontal axis and the vertical axis of the plot are the same as those used for FIGS. 2-5. The horizontal axis represents transverse positions on the retina as azimuthal (or meridional) angles; and the vertical axis represents antero-posterior retinal positions as angles relative to the axis of the eye and subtended at the geometrical center of a sphere modelling the sclera/retina.

This ray-density plot was computed using the model detailed in FIG. 1 for a 3 mm pupil diameter and 0.3 mm implantation depth with an equi-convex IOL whose front and back optic surfaces extend across the entire optic diameter of about 6 mm. The integration over field angles is from 550 to 1000 in 0.5° steps (i.e., a total of 91 field angles). At each field angle, 100,000 rays are traced non-sequentially to model the effect of a point light source at infinity along a single meridian (azimuthal angle) of the eye. For each of the field angles, a ray-intercept plot is generated (similar to FIGS. 2-5). The light rays from the ray-intercept plots, over all the field angles, are then integrated (i.e., summated) and their relative retinal density (or relative retinal irradiance, or relative light intensity, calculated as the number of rays per unit area on the retina) is computed. The resultant intensity at a particular retinal position (azimuth angle by antero-posterior retinal position) is plotted as a grey-scale value for which a bright shading indicates a relatively high intensity and a dark shading indicates a relatively low intensity. The shading used for the relative retinal intensity is indicated by the grey-scale 601 (the illustrated scale is logarithmic with a full-range of 4.5 log-units (base 10)).

On the ray density plot 600 integrating over field angles, the region of the retina irradiated by refracted light rays can be seen as the refracted light region 602. In the modelling, a minor amount of this intensity is contributed to by ‘edge’ rays and is seen as a low intensity cluster 605. More anteriorly, a smaller region representing the positions of the retina irradiated by by-pass rays is seen in the by-pass region 603.

As illustrated, there is a region 604 between the refracted rays retinal region 602 and the by-pass rays retinal region 603 which is void (e.g., substantially void) of light. This is the dark-band region 604 towards which no or minimal light arrives regardless of incident light field angle. This dark-band may provide an explanation for the negative PPD phenomena.

To facilitate understanding and interpretation of FIG. 6, if zero azimuth angle represents the horizontal meridian of a patient's eye looking to a point on the horizontal plane (e.g., the horizon), then the range of light incident (from 55° to 100°) can be imagined as an extended light source which is uniformly bright and provide a continuous line source of light along the horizon stretching from 55° from the patient's direction of gaze, around to 100° from the patient's direction of gaze. While the line of light of this extended light source is continuous and uniformly bright, from the retinal ray-density integrated over field angles, the patient would perceive an interval of light which is interrupted or missing at the visual field direction associated with the dark band retinal region of 604. Slightly more peripheral to this dark-band, the patient would ‘see’ or perceive a ‘stub’ of light produced by the by-pass rays. The dark-band region exists regardless of the field angle. That is, even if the patient were to turn her eye or head to change the direction of gaze, there would still be an interruption to the line of light—the dark-band remaining at the same angle to the patient's direction of gaze regardless of where the patient may be looking. This is consistent with reports from patients experiencing negative PPD.

The refracted light region 602 may be understood as the retinal region produced by the summation of refracted light spots (e.g., spots 401 from FIG. 4) over field angles 55° to 100° in 0.5° increments. In some embodiments, there may also be some contributions from ‘edge’ ray spots 605 (e.g., contributed to by spots 402 in FIG. 4). The region 603 may be understood as retinal region produced by the summation of by-pass light spots (e.g., contributed to by spots 403 in FIG. 4) over field angles 55° to 100° in 0.5° increments.

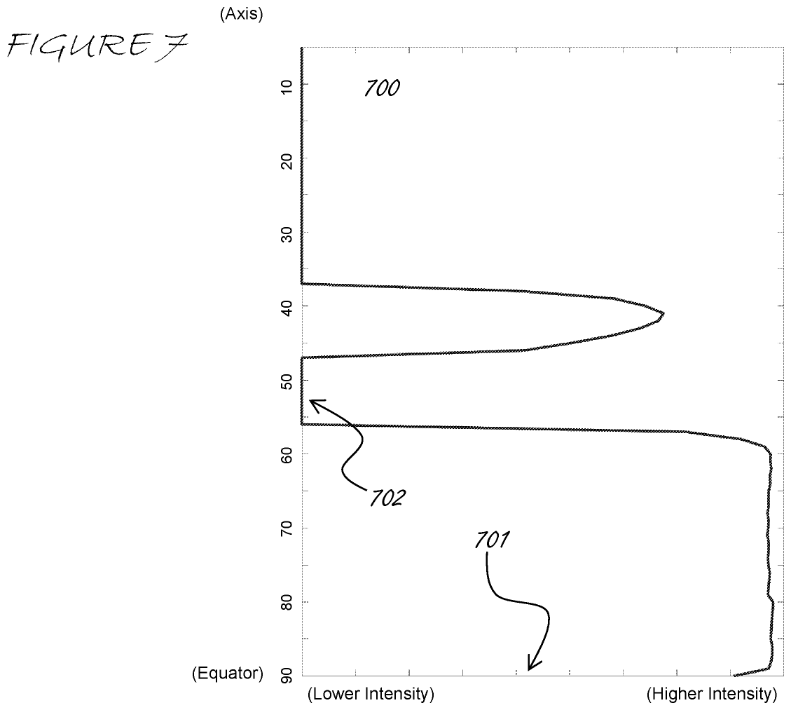

FIG. 7 is an integrated ray density (or relative retinal irradiance, or relative whole field retinal irradiance) plot 700 integrated over a range of field angles and azimuthal angles showing the intensity of light distribution across the retina in accordance with certain embodiments.

The ray-density plot 600 of FIG. 6, integrating ray-intercept (retinal intensity) over field angles, illustrates the appearance to the patient of a continuous line light source lying along a single meridian. To portray the appearance of a uniformly bright visual field, such as a wide clear sky, or snow-scape, or a large blank wall, the retinal ray-density (e.g., FIG. 6) is integrated over (or around) the meridians (or azimuthal angles) of the eye (or visual field of the eye). Mathematically, this may be considered as a convolution of the ray-density plot (e.g., the plot in FIG. 6) over a plurality of azimuthal angles. FIG. 7 illustrates a relative ‘whole field’ retinal intensity plot 700. As used herein, the term “whole field” refers to integrating light source from multiple field angles (e.g., between 55° and 100°) and convolving around all multiple azimuthal angles (e.g., a total of 360°, that is, all meridians of the whole visual field). In this example, since the light source is assumed to have a constant brightness regardless of field angle or azimuthal angle, a convolution around azimuthal angles is mathematically equivalent to a sum of the ray-density plot values at each retinal angular position around the axis of the eye. The resultant relative whole field retinal irradiance is plotted according to the horizontal axis 701 for which lesser amount of retina irradiance or intensity lies to the left and greater amount of retina irradiance or intensity lies to the right (the scale is logarithmic (base 10) with a full range of about 4.5 log units).

From the plot 700, which are results for the IOL modelled in FIG. 1 and modelling a 3 mm pupil diameter and 0.3 mm implantation depth, refracted ray regions of the retina provides relatively constant irradiance of the retina antero-posteriorly from 90° to approximally 56°. Between approximally the 56° and 47° retinal positions, a dark band region 702 occurs. This dark band exists regardless of the direction (field angle or meridian) of the light source. In certain reports of patients with negative PPD, one complaint is the disturbance of a dark band when the visual field is uniformly bright (such as clear sky, or snow-scape). The results in FIG. 7 appear to be consistent with this type of complaint.

Reports of negative PPD suggest that the phenomenon is inconsistent and varies between patients and IOL designs. The eye/IOL non-sequential ray-tracing modelling described herein provide an explanation for the apparent inconsistency in reports of negative PPD because the existence of a dark-band appears to vary as a combination of pupil diameter and/or implantation depth of the IOL.

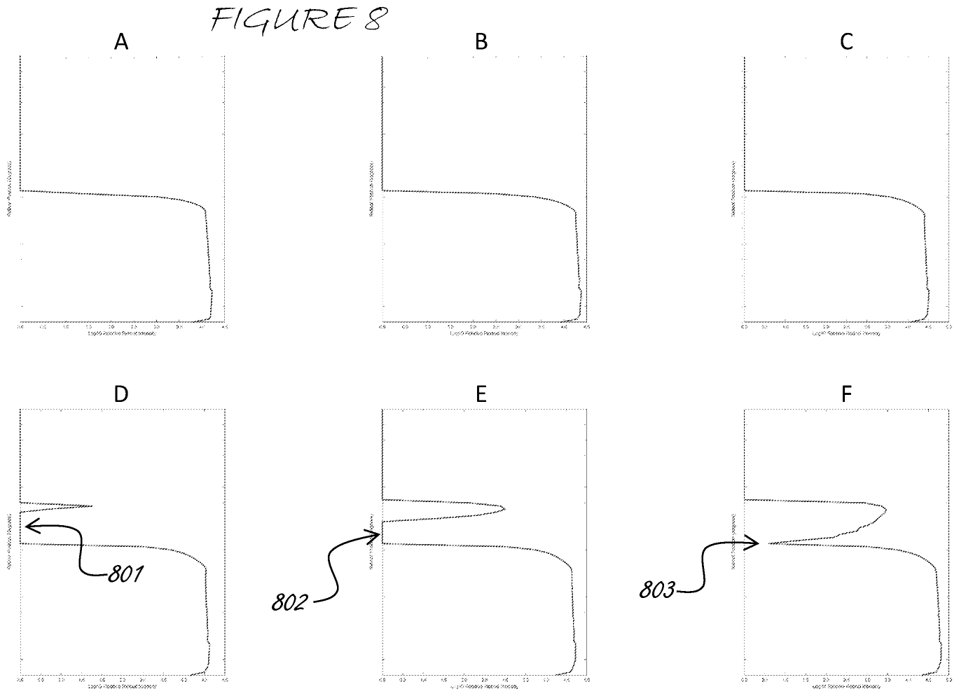

FIGS. 8-15 illustrate the relative whole-field retinal irradiance or intensity for the same IOL as modelled in FIG. 1 while varying pupil size from 2.5 mm to 5 mm diameter in 0.5 mm steps, and implantation depth from 0 mm to 0.7 mm in 0.1 mm steps. As described herein, the IOL modelled is an equi-convex design with front and back surfaces extending across the entire optic diameter of 6 mm. In all cases, about 100,000 rays are traced non-sequentially through the eye model for each field angle and a ray-density plot is generated integrating over the field angles (e.g., the 91 field angles). Then, the ray-density results are convolved around azimuthal angles to produce an integrated ray density plot, or relative whole-field retinal irradiance or intensity plot. The plot axes and scales, and intensity grey-scale, of the plots shown in FIGS. 8-15 are the same as those used in FIG. 7.

FIGS. 8A-F are relative retinal irradiance plots showing the intensity (e.g., relative whole field retinal intensity) of light distribution across the retina for the intraocular lens modelled in FIG. 1 for pupil sizes from 2.5 mm to 5 mm in 0.5 mm steps at an implantation depth of 0 mm in accordance with certain embodiments. FIG. 8A illustrates the relative whole-field retinal irradiance for 0 mm IOL implantation depth and a 2.5 mm pupil diameter. FIG. 8B illustrates the relative whole-field retinal irradiance for 0 mm IOL implantation depth and a 3 mm pupil diameter. FIG. 8C illustrates the relative whole-field retinal irradiance for 0 mm IOL implantation depth and a 3.5 mm pupil diameter. FIG. 8D illustrates the relative whole-field retinal irradiance for 0 mm IOL implantation depth and a 4 mm pupil diameter. FIG. 8E illustrates the relative whole-field retinal irradiance for 0 mm IOL implantation depth and a 4.5 mm pupil diameter. FIG. 8F illustrates the relative whole-field retinal irradiance for 0 mm IOL implantation depth and a 5 mm pupil diameter. As illustrated, at this implantation depth, dark band retinal regions occur for the 4 mm and 4.5 mm pupil sizes at positions 801 and 802 respectively. A relative dark band, within which the irradiance is not zero but is many orders of magnitude (in this example, about four base-10 log units) in intensity below that for the refracted rays regions and hence would be perceived to be a dark band by the individual, occurs at the 5 mm pupil size at position 803.

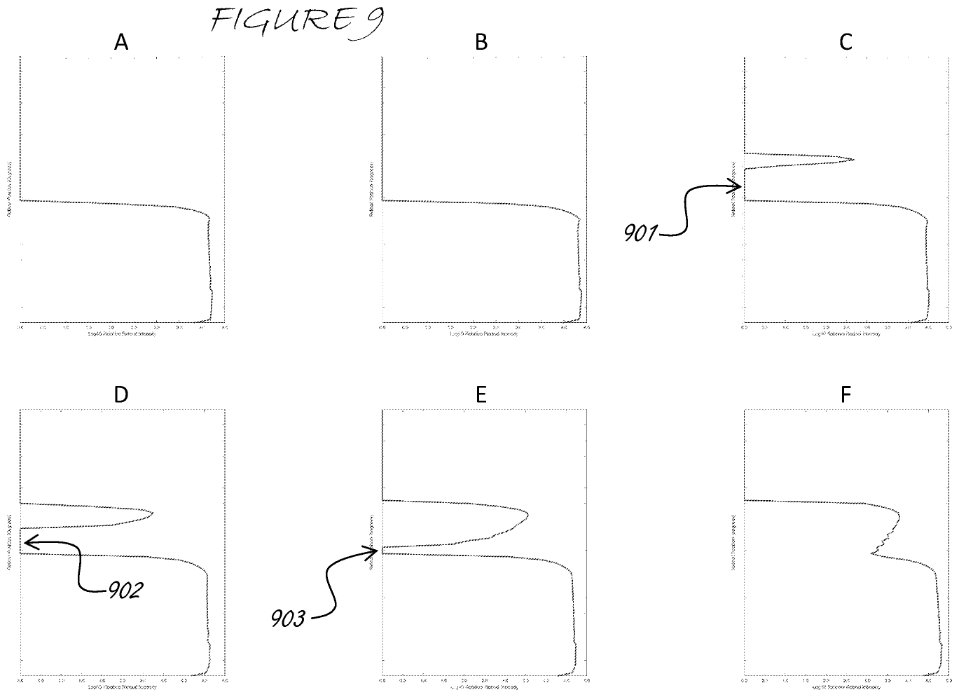

FIGS. 9A-F are relative retinal irradiance plots showing the intensity (e.g., relative whole field retinal intensity) of light distribution across the retina for the intraocular lens modelled in FIG. 1 for pupil sizes from 2.5 mm to 5 mm in 0.5 mm steps at an implantation depth of 0.1 mm in accordance with certain embodiments. FIG. 9A illustrates the relative whole-field retinal irradiance for 0.1 mm IOL implantation depth and a 2.5 mm pupil diameter. FIG. 9B illustrates the relative whole-field retinal irradiance for 0.1 mm IOL implantation depth and a 3 mm pupil diameter. FIG. 9C illustrates the relative whole-field retinal irradiance for 0.1 mm IOL implantation depth and a 3.5 mm pupil diameter. FIG. 9D illustrates the relative whole-field retinal irradiance for 0.1 mm IOL implantation depth and a 4 mm pupil diameter. FIG. 9E illustrates the relative whole-field retinal irradiance for 0.1 mm IOL implantation depth and a 4.5 mm pupil diameter. FIG. 9F illustrates the relative whole-field retinal irradiance for 0.1 mm IOL implantation depth and a 5 mm pupil diameter. As illustrated, at this implantation depth, dark band retinal regions 901, 902 and 903 occur for the 3.5 mm, 4 mm and 4.5 mm pupil sizes respectively.

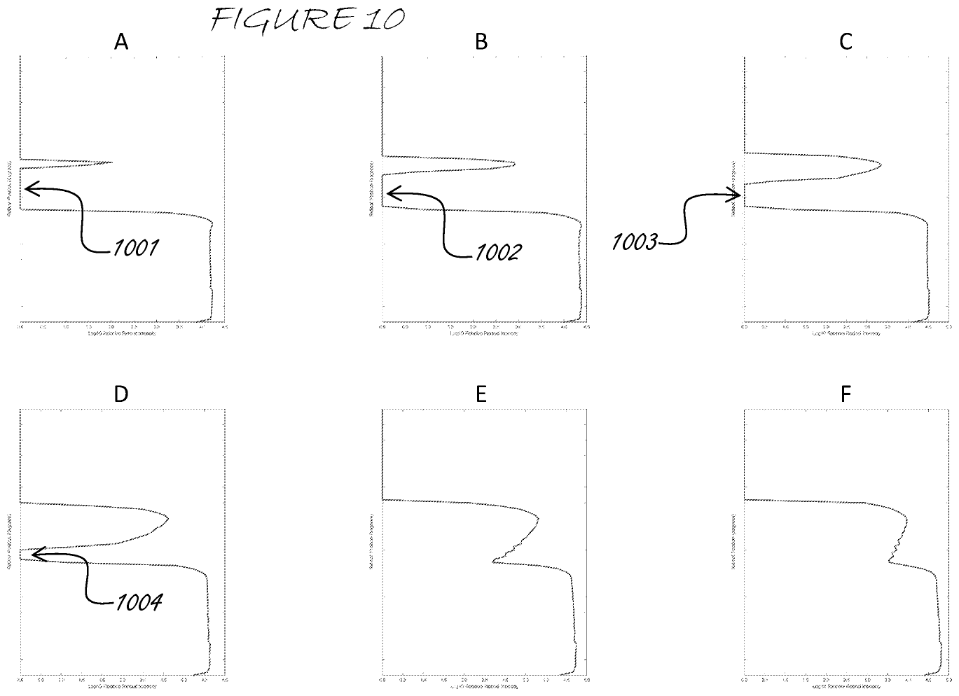

FIGS. 10A-F are relative retina irradiance plots showing the intensity (e.g., relative whole field retinal intensity) of light distribution across the retina for the intraocular lens modelled in FIG. 1 for pupil sizes from 2.5 mm to 5 mm in 0.5 mm steps at an implantation depth of 0.2 mm in accordance with certain embodiments. FIG. 10A illustrates the relative whole-field retinal irradiance for 0.2 mm IOL implantation depth and a 2.5 mm pupil diameter. FIG. 10B illustrates the relative whole-field retinal irradiance for 0.2 mm IOL implantation depth and a 3 mm pupil diameter. FIG. 10C illustrates the relative whole-field retinal irradiance for 0.2 mm IOL implantation depth and a 3.5 mm pupil diameter. FIG. 10D illustrates the relative whole-field retinal irradiance for 0.2 mm IOL implantation depth and a 4 mm pupil diameter. FIG. 10E illustrates the relative whole-field retinal irradiance for 0.2 mm IOL implantation depth and a 4.5 mm pupil diameter. FIG. 10F illustrates the relative whole-field retinal irradiance for 0.2 mm IOL implantation depth and a 5 mm pupil diameter. As illustrated, at this implantation depth, dark band retinal regions 1001, 1002, 1003 and 1004 occur for the 2.5 mm, 3 mm, 3.5 mm and 4 mm pupil sizes respectively.

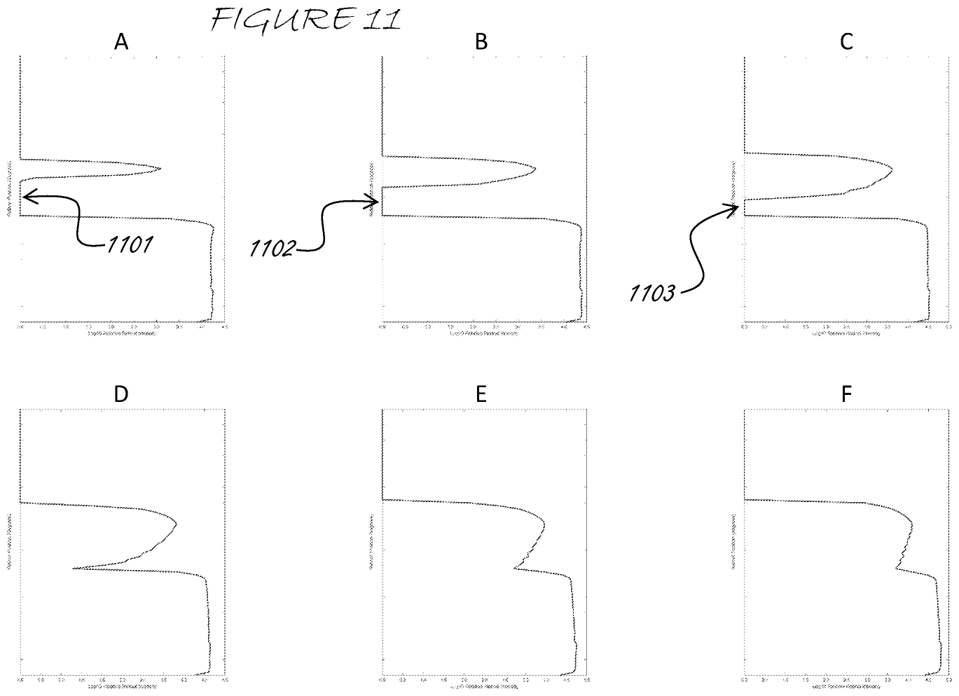

FIGS. 11A-F are relative whole field retinal irradiance plots showing the intensity (e.g., relative retina intensity) of light distribution across the retina for the intraocular lens modelled in FIG. 1 for pupil sizes from 2.5 mm to 5 mm in 0.5 mm steps at an implantation depth of 0.3 mm in accordance with certain embodiments. FIG. 11A illustrates the relative whole-field retinal irradiance for 0.3 mm IOL implantation depth and a 2.5 mm pupil diameter. FIG. 11B illustrates the relative whole-field retinal irradiance for 0.3 mm IOL implantation depth and a 3 mm pupil diameter. FIG. 11C illustrates the relative whole-field retinal irradiance for 0.3 mm IOL implantation depth and a 3.5 mm pupil diameter. FIG. 11D illustrates the relative whole-field retinal irradiance for 0.3 mm IOL implantation depth and a 4 mm pupil diameter. FIG. 11E illustrates the relative whole-field retinal irradiance for 0.3 mm IOL implantation depth and a 4.5 mm pupil diameter. FIG. 11F illustrates the relative whole-field retinal irradiance for 0.3 mm IOL implantation depth and a 5 mm pupil diameter. As illustrated, at this implantation depth, dark band retinal regions 1101, 1102, and 1103 occur for the 2.5 mm, 3 mm, and 3.5 mm pupil sizes.

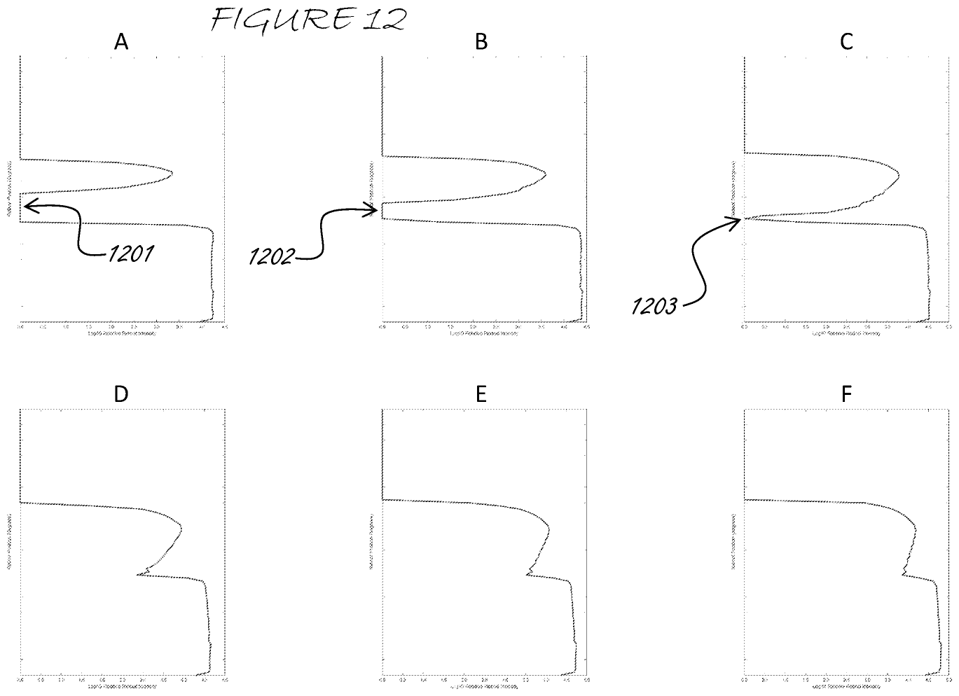

FIGS. 12A-F are relative retinal intensity plots showing the intensity (e.g., relative whole field retinal irradiance) of light distribution across the retina for the intraocular lens modelled in FIG. 1 for pupil sizes from 2.5 mm to 5 mm in 0.5 mm steps at an implantation depth of 0.4 mm in accordance with certain embodiments. FIG. 12A illustrates the relative whole-field retinal irradiance for 0.4 mm IOL implantation depth and a 2.5 mm pupil diameter. FIG. 12B illustrates the relative whole-field retinal irradiance for 0.4 mm IOL implantation depth and a 3 mm pupil diameter. FIG. 12C illustrates the relative whole-field retinal irradiance for 0.4 mm IOL implantation depth and a 3.5 mm pupil diameter. FIG. 12D illustrates the relative whole-field retinal irradiance for 0.4 mm IOL implantation depth and a 4 mm pupil diameter. FIG. 12E illustrates the relative whole-field retinal irradiance for 0.4 mm IOL implantation depth and a 4.5 mm pupil diameter. FIG. 12F illustrates the relative whole-field retinal irradiance for 0.4 mm IOL implantation depth and a 5 mm pupil diameter. As illustrated, at this implantation depth, dark band retinal regions 1201, 1202 and 1203 occur for the 2.5 mm, 3 mm and 3.5 mm pupil sizes respectively.

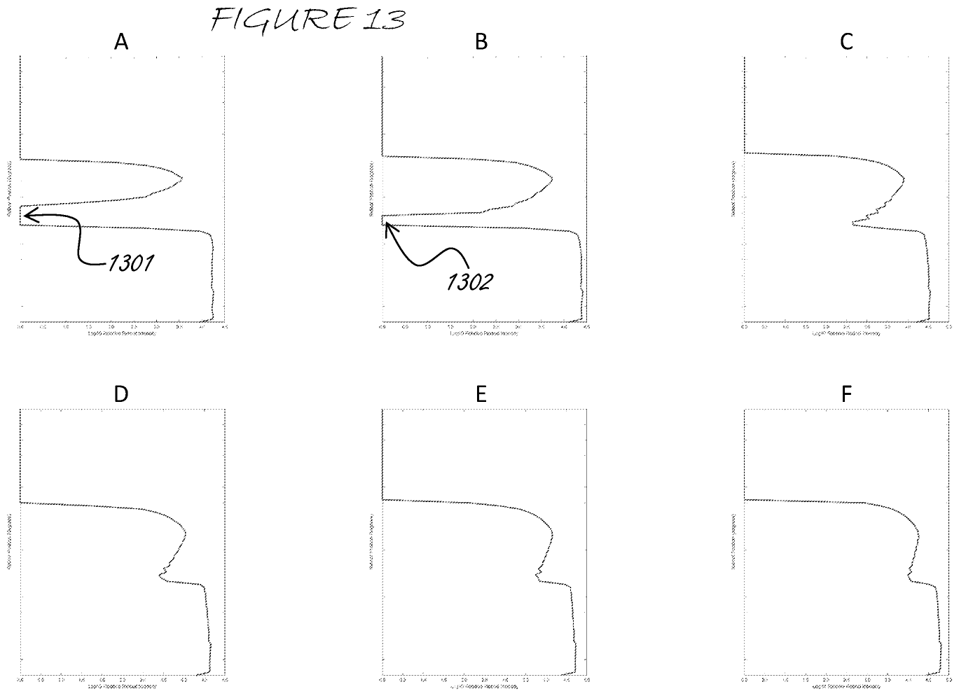

FIGS. 13A and F are relative retinal irradiance plots showing the intensity (e.g., relative whole field retinal irradiance) of light distribution across the retina for the intraocular lens modelled in FIG. 1 for pupil sizes from 2.5 mm to 5 mm in 0.5 mm steps at an implantation depth of 0.5 mm in accordance with certain embodiments. FIG. 13A illustrates the relative whole-field retinal irradiance for 0.5 mm IOL implantation depth and a 2.5 mm pupil diameter. FIG. 13B illustrates the relative whole-field retinal irradiance for 0.5 mm IOL implantation depth and a 3 mm pupil diameter. FIG. 13C illustrates the relative whole-field retinal irradiance for 0.5 mm IOL implantation depth and a 3.5 mm pupil diameter. FIG. 13D illustrates the relative whole-field retinal irradiance for 0.5 mm IOL implantation depth and a 4 mm pupil diameter. FIG. 13E illustrates the relative whole-field retinal irradiance for 0.5 mm IOL implantation depth and a 4.5 mm pupil diameter. FIG. 13F illustrates the relative whole-field retinal irradiance for 0.5 mm IOL implantation depth and a 5 mm pupil diameter. As illustrated, at this implantation depth, dark band retinal regions 1301 and 1302 occurs for the 2.5 mm and 3 mm pupil sizes respectively.

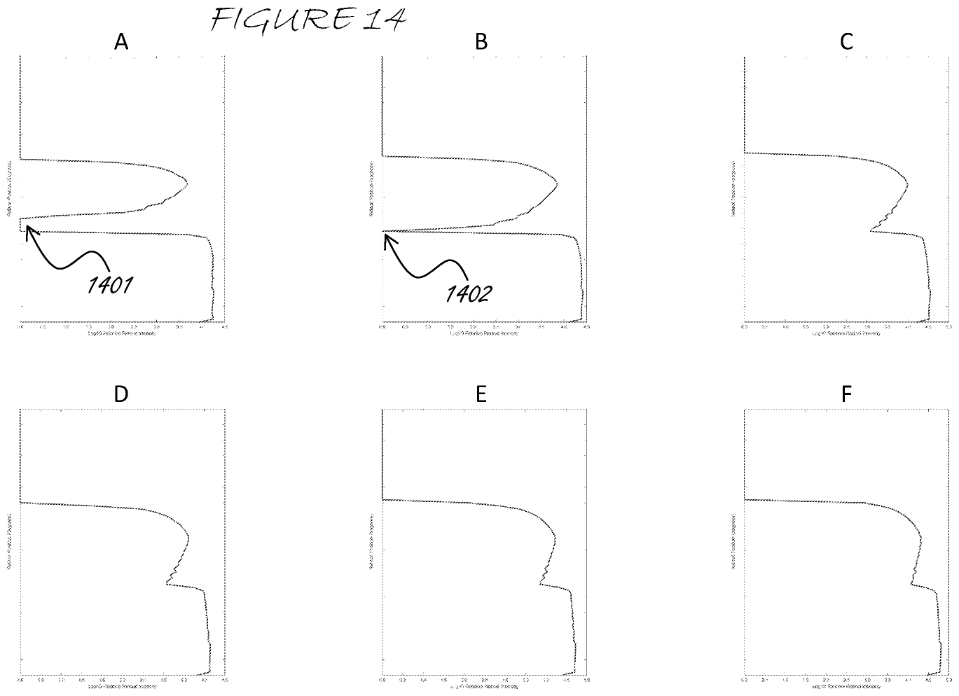

FIGS. 14A-F are relative retinal irradiance plots showing the intensity (e.g., relative whole field retinal irradiance) of light distribution across the retina for the intraocular lens modelled in FIG. 1 for pupil sizes from 2.5 mm to 5 mm in 0.5 mm steps at an implantation depth of 0.6 mm in accordance with certain embodiments. FIG. 14A illustrates the relative whole-field retinal irradiance for 0.6 mm IOL implantation depth and a 2.5 mm pupil diameter. FIG. 14B illustrates the relative whole-field retinal irradiance for 0.6 mm IOL implantation depth and a 3 mm pupil diameter. FIG. 14C illustrates the relative whole-field retinal irradiance for 0.6 mm IOL implantation depth and a 3.5 mm pupil diameter. FIG. 14D illustrates the relative whole-field retinal irradiance for 0.6 mm IOL implantation depth and a 4 mm pupil diameter. FIG. 14E illustrates the relative whole-field retinal irradiance for 0.6 mm IOL implantation depth and a 4.5 mm pupil diameter. FIG. 14F illustrates the relative whole-field retinal irradiance for 0.6 mm IOL implantation depth and a 5 mm pupil diameter. As illustrated, at this implantation depth, dark band retinal regions 1401 and 1402 occur for the 2.5 mm and 3 mm pupil sizes respectively.

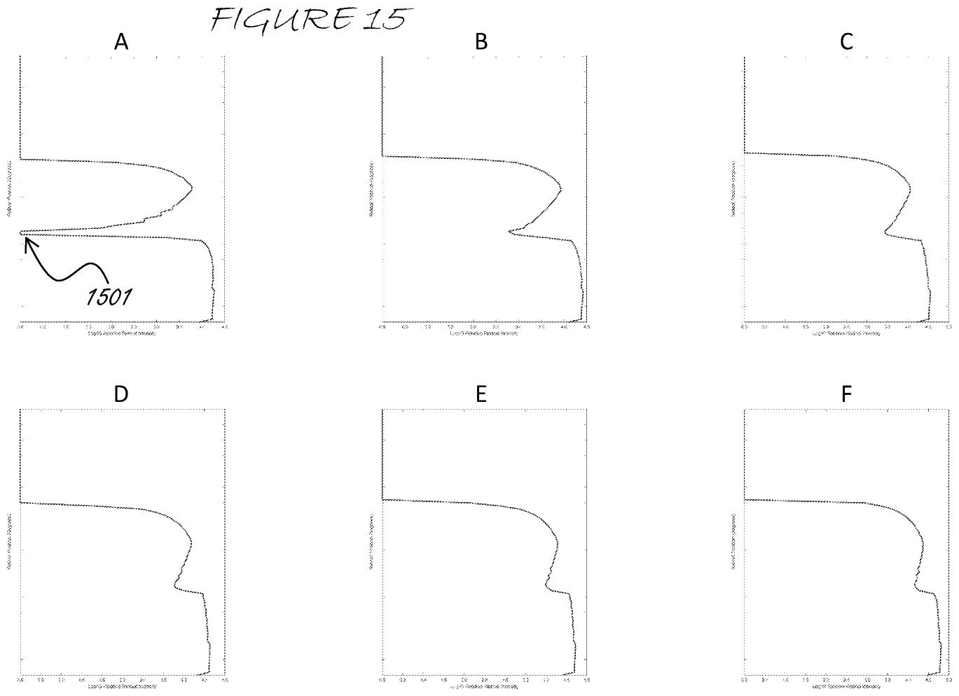

FIGS. 15A-F are relative retinal irradiance plots showing the intensity (e.g., relative whole field retinal irradiance) of light distribution across the retina for the intraocular lens modelled in FIG. 1 for pupil sizes from 2.5 mm to 5 mm in 0.5 mm steps at an implantation depth of 0.7 mm in accordance with certain embodiments. FIG. 15A illustrates the relative whole-field retinal irradiance for 0.7 mm IOL implantation depth and a 2.5 mm pupil diameter. FIG. 15B illustrates the relative whole-field retinal irradiance for 0.7 mm IOL implantation depth and a 3 mm pupil diameter. FIG. 15C illustrates the relative whole-field retinal irradiance for 0.7 mm IOL implantation depth and a 3.5 mm pupil diameter. FIG. 15D illustrates the relative whole-field retinal irradiance for 0.7 mm IOL implantation depth and a 4 mm pupil diameter. FIG. 15E illustrates the relative whole-field retinal irradiance for 0.7 mm IOL implantation depth and a 4.5 mm pupil diameter. FIG. 15F illustrates the relative whole-field retinal irradiance for 0.7 mm IOL implantation depth and a 5 mm pupil diameter. As illustrated, at this implantation depth, a dark band retinal region 1501 occurs for the 2.5 mm pupil size.

FIG. 16 is a schematic illustration of a half-meridian section of an intraocular lens reducing, minimizing, and/or eliminating peripheral pseudophakic dysphotopsia in accordance with certain embodiments. For orientation, in FIG. 16, upwards in the figure is towards the anterior (front) of the eye/IOL (e.g., towards the incoming light to the eye) and downwards is towards the posterior (back) of the eye/IOL (e.g., towards the retina and fovea of the eye). An axis of the eye/IOL 1602 is positioned at the left of FIG. 16. Thus, towards the right of the figure is a direction radially outwards from the center/axis of the eye/IOL. (It is understood by vision scientists, designers of IOL and eye-care practitioners that while the eye may not be exactly rotationally symmetrical, it may be reasonably approximal to be rotationally symmetrical about an axis.) As illustrated, FIG. 16 shows a half-meridional cross section of an IOL 1600 about the axis of the optical system 1602. The cross section of the IOL 1600 is illustrated as being implanted posteriorly to the iris 1601 of and eye. In some embodiments, the relative positions of the IOL 1600 and the iris 1601 may vary depending on e.g., pupil size, iris topography and/or implantation depth.

The meridional cross section of FIG. 16 is for purpose of illustration only and may not be isometric (e.g., anisometric); that is, the distance (or dimensions or scaling) in the horizontal direction may not be the same as that in the vertical direction.

The IOL 1600 comprises an optic zone 1603 and a control zone 1606. In some embodiments, prescribed optical power of the IOL 1600 may be provided by the optic zone 1603.

The optic zone of the IOL may be characterized by any combination of one or more of a front (anterior) optic surface 1604, a back (posterior) optic surface 1605, a thickness (between front and back optic surfaces e.g., along axis 1602), and a refractive index of the IOL material.

In some embodiments, the control zone 1606 may be configured to control PPD (e.g., negative and/or positive PPD). As illustrated, the control zone 1606 may comprise a front (anterior) control surface 1607, a back (posterior) control surface 1608 and an edge 1611. A boundary between the optic zone 1603 and the control zone 1606 forms an optic-control junction. The front optic-control junction 1609 marks the boundary or transition from the front optic surface 1604 to the front control surface 1607. The back optic-control junction 1610 marks the boundary or transition from the back optic surface 1605 to the back control surface 1608.

As the control zone 1606 is positioned towards the periphery of the IOL 1600, the optic zone 1603 (which is more centrally located) of the IOL 1600 may, in some embodiments, function in the same way as conventional IOLs. For example, the optic zone 1603 may be configured to deliver an optical power within a large range. The optic zone 1603 may incorporate any combination of one or more of a range of conventional IOL optics including multifocal optics or extended depth of focus optics for supporting near vision, diffractive optics, toric optics for correcting astigmatism, etc.

In some embodiments, the intersection of the control surfaces 1607, 1608 and the edge 1611 may form junctions 1612, 1613 between the control surfaces and the edge. For example, the front control surface 1607 may meet the edge 1611 at the front control-edge junction 1612 and the back control surface 1608 may meet the edge 1611 at the back control-edge junction 1613.

In some embodiments, the front and/or back control surfaces 1607, 1608 of control zone 1606 may be configured to have particular surface curvatures and/or profiles to redirect and/or distribute light to otherwise dark band regions of the retina. By configuring the control zone 1606 to fill-in (e.g., refract light to) the dark band region, the IOL 1600 may reduce, significantly reduce, and/or eliminate the occurrence/perception of PPD.

In some embodiments, the eye (either with natural crystalline lens, or with IOL) may be approximal to a rotationally symmetric optical system and an axis 1602 may be used to reference directions and radial or transverse distances.

In some embodiments, the optic zone 1603 may be located in the central portion of the IOL and provide the optical power for supporting vision of the patient. Optical characteristics (e.g., power, aberrations, depth of focus, etc.) of the optic zone may be determined by the curvature or profile of the front and back optic surfaces 1604, 1605, the IOL thickness, as well as the refractive index of the IOL material. The optic zone on either or both of the front and back optic zone surfaces 1604, 1605 may be circular or polygonal (e.g., hexagonal) or a freeform shape according to the visual/optic purpose of the optic zone. The size of the optic zone may be a few millimeters (e.g., about 1.5 mm, or about 2 mm, or about 3 mm, or about 4 mm, or 5 about mm or about 6 mm). The thickness of the IOL 1600 may be selected according to the optical requirements or purpose of the optic zone, for example, greater thickness to provide a sufficiently large size optic zone for a higher power IOL (that requires greater surface curvatures or shorter radii of curvature), or lower thickness to enable the IOL to be rolled or folded to facilitate implantation through small corneal incisions. The IOL material may be selected from a range of suitable ophthalmic materials (e.g., conventional ophthalmic materials) including hydrogel, hydrophilic materials, hydrophobic materials, silicone materials, acrylic or acrylate type materials, or more advanced materials such as gradient index (GRIN) or photosensitive materials (e.g., light adjustable), etc. Such IOL materials may have refractive indices ranging from about 1.4 to about 1.6. The surface profile of the front and/or back optic surface 1604, 1605 may be a combination of one or more of spherical (e.g., circle cross section), conic section, polynomials, Zernikes, superconics, Bezier, spline, Fourier, wavelets, kinoform, echelettes, phase steps, annuli, lenslets, lenslet arrays, etc.

In some embodiments, the IOL may utilize optic zone designs and configurations including single-vision at any of a wide range of powers, multifocal or extended depth of focus for facilitating near vision, toric surfaces or power for correcting astigmatism. The optic zone may utilize optical approaches including refractive or diffractive surfaces, or advanced optical surfaces such as meta-surfaces or nanostructures. In some embodiments, the size (diameter if circular) of the optic zone may be determined by either one or both of the position of the optic-control junctions 1609, 1610.

The control zone 1606 may be configured to control negative PPD. In some embodiments, the control zone may be configured to intercept a portion of oblique light rays (e.g., from light incident on the eye from peripheral field angles) passing through the pupil and redirect and/or distribute the rays to a region on the retina that would otherwise be a dark band. In some embodiments, this may be achieved by appropriate configurations of a back control surface 1608, a front control surface 1607, the thickness or thickness profile of the IOL at the control zone 1606 and/or the width (or length, e.g., distance between the control-edge junction points 1612, 1613) of the edge 1611.

In some embodiments, the control zone 1606 may be positioned towards the periphery of the IOL but may not necessarily extend to the very edge of the IOL. In some embodiments, the control zone 1606 may extend to the edge of the IOL. In some embodiments, the width of the control zone 1606 may be as wide as possible to redirect as much light as possible to “fill in” the otherwise dark band region of the retina but without significantly impacting vision.

In some embodiments, the back (posterior) control surface 1608, together with the curvature/surface profile of the front (anterior) control surface 1607, and/or the thickness or thickness profile of the IOL at the control zone 1606, and/or the width (or length, e.g., distance between the control-edge junction points 1612 and 1613) of the edge 1611, may redirect and/or distribute light to a region on the retina that would otherwise be a dark band. In some embodiments, the control zone 1606 may have a thickness that varies radially. For examples, in some embodiments, the thickness may increase towards the periphery or the thickness may decrease towards the periphery.

In some embodiments, the back control surface 1608 may be convex (e.g., substantially convex, or generally convex, i.e., convex when considered across the expanse of the back control surface 1608) towards the back of the eye (e.g., concave towards the front of the eye) as illustrated in FIG. 16. In some embodiments, the back control surface 1608 may have a steeper curvature (e.g., shorter radius of curvature) than the back optic surface 1605. In some embodiments, an absolute value for the radius of curvature of the back control surface 1608 may be smaller (e.g., lesser in value) than an absolute value of the radius of curvature of the back optic surface 1605. For example, the back optic surface 1605 may be a positive refracting surface which is convex and the absolute value of curvature for the back optic surface may be lower (e.g., has a greater absolute radius of curvature) than that for the back control surface 1608 which has greater curvature (e.g., has a lesser absolute radius of curvature) along the meridional cross section. The curvature of a plano (e.g., flat, plane, with zero optical power) surface (e.g., optic surface, control surface) is considered to be zero (e.g., lowest absolute value for curvature) and the radius of curvature of a plano surface is considered to be infinite (e.g., highest absolute value for radius of curvature).

In some embodiments, the back control surface 1608 may have a curvature opposite in sign to the curvature of the back optic surface 1605. For example, the back optic surface 1605 may be a negative refracting surface which is convex towards the front of the eye while the back control surface 1608 may be concave towards the front of the eye; that is, the two surfaces have opposite signs in curvature. When considering the sign of a radius of curvature value, the radius of curvature of a surface may be measured from the surface to the center of curvature. When the direction from the surface to the center of curvature is in the same direction as the direction of travel of incoming light (e.g., from the anterior/front of the eye to the posterior/back of the eye), the radius of curvature is positive in value (i.e., has a positive sign). If the direction from the surface to center of curvature is in the opposite direction to the direction of travel of incoming light, the radius of curvature is negative in value (i.e., has a negative sign). For example, a surface that is convex towards the front of the eye has a positive radius of curvature. The curvature of the surface is determined as the reciprocal of the radius of curvature and its sign matches that of the associated radius of curvature. That is, a surface with a positive radius of curvature has a positive curvature value and conversely, a surface with a negative radius of curvature has a negative curvature value. For example, a surface that is concave towards the front of the eye has a negative radius of curvature and its curvature is also negative in value.

In some embodiments, the back control surface 1608 may vary in curvature (e.g., local curvature or instantaneous curvature) along its profile.

In some embodiments, the back control surface 1608 profile may be increasing in curvature (e.g., radius of curvature becomes shorter) towards the edge 1611 of the IOL 1600. In some embodiments, the back control surface 1608 profile may be decreasing in curvature (e.g., radius of curvature becomes longer) towards the edge of the IOL 1600. In some embodiments, the back control surface 1608 profile may decrease in curvature (e.g., radius of curvature becomes longer) then increase in curvature (e.g., radius of curvature becomes shorter) towards the edge 1611 of the IOL 1600. In some embodiments, the back control surface 1608 profile may increase in curvature (e.g., radius of curvature becomes shorter) then decrease in curvature (e.g., radius of curvature becomes longer) towards the edge 1611 of the IOL 1600.

In some embodiments, the slope relative to (e.g., referenced to, or measured from) a frontal plane along the back control surface 1608 near to the back control-edge junction 1613 is such that as the back control surface 1608 progresses radially outwards (e.g., from axis of the IOL towards the peripheral retina), points on the back control surface 1608 become positioned more anteriorly (e.g., towards the iris). A frontal plane is a plane that is parallel to the plane of the iris (whose aperture forms the pupil) of the eye and is perpendicular to an axis 1602 of the eye.

In some embodiments, the absolute value of the angle of a slope relative to a frontal plane of the back control surface 1608 at or near to the back control-edge junction 1613 is greater than the absolute value of the angle of a slope relative to a frontal plane of the back control surface 1608 at, or near to, the back optic-control junction 1610.

In some embodiments, a slope of the back control surface 1608, relative to (e.g., referenced to, or measured from) a frontal plane, along the back control surface 1608 in a point or region not coincident with (e.g. not on, not co-located), but is proximal (e.g. near to, in the vicinity of) the back control-edge junction 1613, is such that as the back control surface 1608 progresses radially outwards (e.g., in a direction from axis of the IOL towards the peripheral retina), points on the back control surface 1608 close to (e.g., near to or at) the back control-edge junction 1613 become positioned more anteriorly (e.g., towards the cornea of the eye), and the absolute value of an angle of a slope, relative to a frontal plane, of the back control surface 1608 at the back control-edge junction 1613, is greater than the absolute value of a slope, relative to a frontal plane, of a point or region of the back control surface 1608 not on (e.g. not coincident with, not co-located with) but near to (e.g. proximal to) the back control-edge junction 1613. The distance from the back control-edge junction 1613 to a point or region on the back control surface 1608 that is not on (e.g., not co-located, not coincident with) but proximal to (e.g., near to) the back control-edge junction 1613, may be less than 2 mm, less than 1.5 mm, less than 1 mm, less than 0.5 mm or less than 0.25 mm.

In some embodiments, an angle of a slope of the back control surface 1608, relative to a frontal plane, at (e.g., co-located with, coincident with), or near to (e.g., proximal to, in the vicinity of), the back control-edge junction 1613 is more negative in value than an angle of a slope of the back control surface 1608, relative to a frontal plane at, or near to, the back optic-control junction 1610. For the angle of a slope of an IOL surface (e.g. optic surface, control surface, edge) relative to a frontal plane, the sign of the angle is considered to be positive when a point on a tangent to the surface of the slope becomes more posterior in position (e.g., nearer the back of the eye, or towards the fovea or retina) as the point progresses radially outwards (e.g., away from an axis of the IOL) along a tangent to the surface of the slope. Conversely, the sign of the angle of a slope relative to a frontal plane is considered to be negative when a point on the surface of the slope becomes more anterior in position (i.e., nearer the front of the eye, or towards the cornea or the incoming light source) as the point progresses radially outwards (e.g., away from an axis of the IOL) along a tangent of the surface of the slope. Note that the value of one or both of the angles may be positive or negative in sign in this comparison of such some embodiments. For example, the angle of the slope of the back control surface 1608 at the back optic-junction point 1610 as illustrated in FIG. 16 is (slightly) negative in value, while the angle of the slope on the back control surface 1608 near to the control-edge junction 1613 as illustrated in FIG. 16 is more negative in value.

In some embodiments, the back control surface 1608 profile may be defined by an aspheric curve: definable by mathematical functions including conics, polynomials, Bezier curves, spline curves, Fourier series, wavelets, or combinations of two or more of such functions.

In some embodiments, the back control surface 1608 may be C0-continuous with the back optic surface 1605. For example, the back control surface 1608 may meet the back optic surface 1605 without a ledge or ‘jump’. In some embodiments, the back control surface 1608 may be C1-continuous with the back optic surface 1605. For example, the back control surface 1608 may have a common tangent with the back optic surface 1605 where they meet.

In some embodiments, the back control surface 1608 may be C2-continuous with the back optic surface 1605. For example, the back control surface may have the same instantaneous curvature as the back optic surface at the point where they meet. In some embodiments, this may help ensure a gradual transition of ray refraction/deflection angles or image formation at the back surface for rays within the optic and control zones in the vicinity of the back optic junction 1610.

The front (anterior) control surface 1607 is the surface on the front surface of the IOL 1600 that lies within the control zone 1606. Together with the curvature/surface profile of the back control surface 1608, the thickness or thickness profile of the IOL at the control zone 1606 and/or the width (or length, e.g., distance between the control-edge junction points 1612, 1613) of the edge 1611, the curvature/surface profile of the front control surface 1607 may redirect and/or distribute light to a region on the retina that would otherwise be a dark band.

In some embodiments, the front control surface 1607 may be convex (e.g., substantially convex, or generally convex, e.g., convex when considered across the expanse of the front control surface 1607) towards the back of the eye (e.g., concave towards the front of the eye) as illustrated in FIG. 16. In some embodiments, the front control surface 1607 may have a steeper curvature (e.g., shorter radius of curvature) than the front optic surface 1604. In some embodiments, an absolute value for the radius of curvature of the front control surface 1607 may be smaller (e.g., lesser in value) than an absolute value of the radius of curvature of the front optic surface 1604. For example, the front optic surface 1604 may be a positive refracting surface which is convex and the absolute value of curvature for the front optic surface is lower (i.e., has a greater absolute radius of curvature) than that for the front control surface 1607 which has greater curvature (i.e., has a lesser absolute radius of curvature) along the meridional cross section.

In some embodiments, the front control surface 1607 may have a curvature opposite in sign to the curvature of the front optic surface 1604. For example, the front optic surface 1604 may be a positive refracting surface which is convex towards the front of the eye while the front control surface 1607 may be concave towards the front of the eye; that is, the two surfaces may have opposite signs in curvature.

In some embodiments, the front control surface 1607 may vary in curvature (e.g., local curvature or instantaneous curvature) along its profile.

In some embodiments, the front control surface 1607 profile may be increasing in curvature (e.g., the radius of curvature becomes shorter) towards the edge 1611 of the IOL 1600.

In some embodiments, the front control surface 1607 profile may be decreasing in curvature (e.g., radius of curvature becomes longer) towards the edge of the IOL 1600. In some embodiments, the front control surface 1607 profile may decrease in curvature (e.g., radius of curvature becomes longer) then increase in curvature (e.g., radius of curvature becomes shorter) towards the edge 1611 of the IOL 1600. In some embodiments, the front control surface 1607 profile may increase in curvature (e.g., radius of curvature becomes shorter) then decrease in curvature (e.g., radius of curvature becomes longer) towards the edge 1611 of the IOL 1600.

In some embodiments, the slope relative to (e.g., referenced to, or measured from) a frontal plane along the front control surface 1607 near to (e.g., proximal to, or in the vicinity of) the front control-edge junction 1612 is such that as the front control surface 1607 progresses radially outwards (e.g., from axis of the IOL towards the peripheral retina), points on the front control surface 1607 become positioned more anteriorly (e.g., towards the iris). A frontal plane is a plane that is parallel to the plane of the iris of the eye and is perpendicular to an axis 1602 of the eye/IOL.

In some embodiments, the absolute value of a slope relative to a frontal plane of the front control surface 1607 at or near to the front control-edge junction 1612 is greater than the absolute value of the angle of a slope relative to a frontal plane of the front control surface 1607 at the front optic-control junction 1609.

In some embodiments, a slope of the front control surface 1607, relative to (e.g., referenced to, or measured from) a frontal plane, along the front control surface 1607 in a point or region not coincident with (e.g. not co-located, not on), but is proximal to (e.g. near to, in the vicinity of) the front control-edge junction 1612, is such that as the front control surface 1607 progresses radially outwards (e.g., in a direction from axis of the IOL towards the peripheral retina), points on the front control surface 1607 close to (e.g., near to or at) the front control-edge junction 1612 become positioned more anteriorly (e.g., towards the iris), and the absolute value of an angle of a slope, relative to a frontal plane, of the front control surface 1607 at the front control-edge junction 1612, is greater than the absolute value of a slope, relative to a frontal plane, of a point or region of the front control surface 1607 not on (e.g. not coincident with, not co-located with) but proximal to (e.g. near to) the front optic-control junction 1609. The distance from the front control-edge junction 1612 to a point or region on the front control surface 1607 that is not on (e.g., not co-located, not coincident with) but proximal to (e.g., near to) the front control-edge junction 1612, may be less than 2 mm, less than 1.5 mm, less than 1 mm, less than 0.5 mm or less than 0.25 mm.

In some embodiments, an angle of a slope of the front control surface 1607, relative to a frontal plane, at (e.g., co-located with, coincident with), or near to (e.g., proximal to, in the vicinity of), the front control-edge junction 1612 is more negative in value than an angle of a slope of the front control surface 1607, relative to a frontal plane at, or near to, the front optic-control junction 1609.

In some embodiments, the front control surface 1607 profile may be defined by an aspheric curve, definable by mathematical functions including conics, polynomials, Bezier curves, spline curves, Fourier series, wavelets, or combinations of two or more of such functions. In some embodiments, the front control surface 1607 may be C0-continuous with the front optic surface 1604. For example, the front control surface 1607 may meet the front optic surface at a common point, without a ledge or jump.

In some embodiments, the front control surface 1607 may be C1-continuous with the front optic surface 1604. For example, the front control surface 1607 may have a common tangent with the front optic surface 1604 where they meet. In some embodiments, the front control surface 1607 may be C2-continuous with the front optic surface 1604. For example, the front control surface 1607 may have the same instantaneous curvature as the front optic surface 1604 at the point where they meet. In some embodiments, this may help ensure a gradual transition of ray refraction/deflection angles or image formation at the front surface for rays within the optic and control zones in the vicinity of the front optic junction 1609.

The front optic-control junction 1609, also referred to as the front optic-control boundary, is the location or region on the front surface of the IOL 1600 where the front optic surface 1604 meets the front control surface 1607. In some embodiments, the radial/transverse position of the front optic-control junction 1609 may impose a limit on the size of the optic zone 1603. In some embodiments, the front optic-control junction 1609 may be easily definable as an individual point and in some embodiments, the front optic control junction may be a less definable region between the optic zone 1603 and the control zone 1606. In some embodiments, the front optic-control junction 1609 may be a ‘point’ (when viewed as a meridional cross-section) at which the front optic 1604 and control 1607 surfaces directly meet, or may be a region (e.g., annulus for a circular IOL) over which the front optic surface 1604 transitions (or is blended) to the front control surface 1607.

In some embodiments, the position of the front optic-control junction 1609 may be set such that the size of the optic zone 1603 matches (or closely matches) the size of the patient's pupil. In some embodiments, (e.g., due to the Stiles-Crawford Effect), light-rays passing the periphery of the pupil may produce a lesser response by the photo-receptors (e.g., rods and cones) of the retina. Accordingly, matching (or substantially matching) the size of the patient's pupil may not require the front optic-control junction 1609 to be positioned such that size of the optic zone 1603 is the same as the pupil size, but that it can be smaller (or larger) and still not significantly disturb vision.

The back optic-control junction 1610, also referred to as the back optic-control boundary, is the location or region on the back surface where the back optic surface 1605 meets the back control surface 1608. In some embodiments, the radial/transverse position of the back optic-control junction may impose a limit on the size of the optic zone 1603. In some embodiments, the back optic-control junction 1610 may be easily definable as an individual point and in some embodiments, the back optic control junction 1610 may be a less definable region between the optic zone 1603 and the control zone 1606. In some embodiments, the back optic-control junction may be a ‘point’ (when viewed as a meridional cross-section) at which the back optic 1605 and control 1608 surfaces directly meet, or may be a region (e.g., annulus for a circular IOL) over which the back optic surface 1605 transitions (or is blended) to the back control surface 1608.

In some embodiments, the position of the back optic-control junction may be set such that the size of the optic zone 1603 matches (or closely matches) the size of the patient's pupil. In some embodiments (e.g., due to the Stiles-Crawford Effect), light-rays passing the periphery of the pupil may produce a lesser response by the photo-receptors (e.g., rods and cones) of the retina. Accordingly, matching the size of the patient's pupil may not require the back optic-control junction 1610 to be the same as the pupil size, but that may be smaller (or larger) and still not significantly disturb vision. In some embodiments, the back optic-control junction 1610 position may be more peripheral (e.g., further from the axis, closer to the edge) than that of the front optic-control junction 1609.

The edge 1611 of the IOL 1600 is defined as a surface (e.g., substantially cylindrical or conical if the IOL is circular) between and joining the front 1607 and back 1608 surface of the IOL 1600. In some embodiments the edge 1611 may be substantially straight, at least partially curved, and/or undulating or otherwise varying between the front surface 1607 and the back (also more peripheral) surface 1608. In some embodiments, where the control zone 1606 extends to the limit of the lens size, the edge may be formed by the surface between and joining the front and back control surfaces 1607 and 1608 respectively. In some embodiments, the edge 1611 may be sloped so it faces anteriorly such that a normal to the edge surface 1611 and an axis 1602 of the IOL form an angle of less than 40°, 35°, 30°, or 200 (where 0° means the edge 1611 surface is facing directly forward (i.e., the normal to the edge surface 1611 is parallel to the axis 1602 and the edge surface lies in a frontal plane and faces anteriorly towards the iris), and 900 means the edge surface faces directly outwards, parallel to a meridional plane of the eye). For an angle between a normal of an edge and an axis of an IOL, the sign convention is such that a positive angle indicates a normal to an edge such that, at the edge, points that are more anterior along the normal of the edge (e.g., towards the front of the eye) are positioned further radially (e.g., towards the periphery) from the axis. In some embodiments, the angle between a normal of the edge and an axis of the IOL may be about 45°, 40°, 35°, 30°, 25°, 20°, 15°, 10°, or 5°. In some embodiments, the angle may be less than about 45°, 40°, 35°, 30°, 25°, 20°, 15°, or 10°. In some embodiments, the angle may be between about 35-45°, 35-40°, 25-35°, 25-30°, 15-25°, 10-20°, 10-15°, 5-10°, 0-15°, 0-20°, 0-30°, 0-40° or 10-40°.

In some embodiments, the edge surface 1611 may be sloped so the angle of the slope is substantially the same as a by-pass ray. That is, the direction of a by-pass ray is substantially parallel to the surface of the edge 1611.

In some embodiments, a slope of the back control surface 1608 at or near the back control-edge junction 1613 forms an angle of about 90° (e.g., is perpendicular to) with a slope of the edge surface 1611 at or near the back control-edge junction 1613.

In some embodiments, a slope of the back control surface 1608 at or near the back control-edge junction 1613 forms an angle equal to or less than about 90° with a slope of the edge surface 1611 at or near the back control-edge junction 1613 where the angle is subtended within the material of the lens (e.g., according to FIG. 16, the angle is formed clockwise from the back control surface 1608 to the edge surface 1611).

In some embodiments, a slope of the back control surface 1608 at or near the back control-edge junction 1613 forms an angle equal to or greater than about 90° with a slope of the edge surface 1611 at or near the back control-edge junction 1613 where the angle is subtended within the material of the lens (e.g., according to FIG. 16, the angle is formed clockwise from the back control surface 1608 to the edge surface 1611).

In some embodiments, a slope of the front control surface 1607 at or near the front control-edge junction 1612 forms an angle of about 90° (e.g., is perpendicular to) with a slope of the edge surface 1611 at or near the front control-edge junction 1612.

In some embodiments, a slope of the front control surface 1607 at or near the front control-edge junction 1612 forms an angle equal to or less than about 90° with a slope of the edge surface 1611 at or near the front control-edge junction 1612 where the angle is subtended within the material of the lens (e.g., according to FIG. 16, the angle is formed anti-clockwise from the front control surface 1607 to the edge surface 1611).

In some embodiments, a slope of the front control surface 1607 at or near the front control-edge junction 1612 forms an angle equal to or greater than about 90° with a slope of the edge surface 1611 at or near the front control-edge junction 1612 where the angle is subtended within the material of the lens (e.g., according to FIG. 16, the angle is formed anti-clockwise from the front control surface 1607 to the edge surface 1611).

In some embodiments, the width of the edge surface 1611 may be about 2.5 mm, 2 mm, 1.5 mm, 1 mm, 0.5 mm or 0.25 mm. In some embodiments, the width of the edge surface 1611 may be measured in a radial direction (e.g., along a horizontal direction according to FIG. 16). In some embodiments, the width of the edge surface 1611 may be measured along (e.g., in a direction parallel to) the edge surface 1611.

In some embodiments, the width of the edge surface 1611 may be less than about 2.5 mm, 2 mm, 1.5 mm, 1 mm, 0.5 mm or 0.25 mm.

In some embodiments, the front control-edge junction 1612 and the back control-edge junction 1613 may be coincident (e.g., substantially coincident, in very close proximity) so the edge surface 1611 may be very narrow in width, or substantially a ‘knife edge’ (e.g., a wedge shape, a taper).

In some embodiments, the edge surface may be treated to alter its optical characteristics such as one or more of transmission/opacity, scattering/diffusing, spectral transmission, reflectance, etc. The treatment may eliminate or reduce the propagation of light rays (e.g., ‘edge’ rays) that may refract or reflect off the edge either from aqueous to lens (from outside inwards), or from lens to aqueous/vitreous (from inside outwards), or from lens to lens (internal reflection), or from aqueous/vitreous to aqueous/vitreous (external reflection).

In some embodiments, the edge surface 1611 may be a smooth refracting or reflecting surface, or may possess optical features such as diffraction gratings, meta-surfaces (e.g., nano-optics pillars), frosting (e.g., similar to shower screens to render the surface scattering/diffusing), etc.

The front control-edge junction 1612 is the location where the front control surface 1607, or a region or zone more peripheral than the front control surface, and the edge 1611 of the IOL meet. When regarded as a meridional cross-section, the front control-edge junction 1612 may be a sharp corner, a radiused/rounded corner, a chamfered corner, a beveled corner, a filleted corner, or a profile that joins the front control surface 1607 to the edge 1611.

In certain embodiments, the front control curve 1607 may be separated from the front control-edge junction 1612 or the edge 1611 in which case, the front control surface 1607 may appear as a ring or annulus shape that does not continue to the lens edge 1611 when seen from front-on to the IOL 1600.

The back control-edge junction 1613 is the location where the back control surface 1608, or a region or zone more peripheral than the front control surface, and the edge 1611 of the IOL meet. When regarded as a meridional cross-section, the back control-edge junction 1613 may be a sharp corner, a radiused/rounded corner, a chamfered corner, a beveled corner, a filleted corner, or a profile that joins the back control surface 1608 to the edge 1611.

In certain embodiments, the back control curve 1608 may be separated from the back control-edge junction 1613 or the IOL edge 1611 in which case, the back control surface 1608 may be seen as a ring or annulus shape that does not continue to the lens edge 1611 as seen from front-on to the IOL 1600.

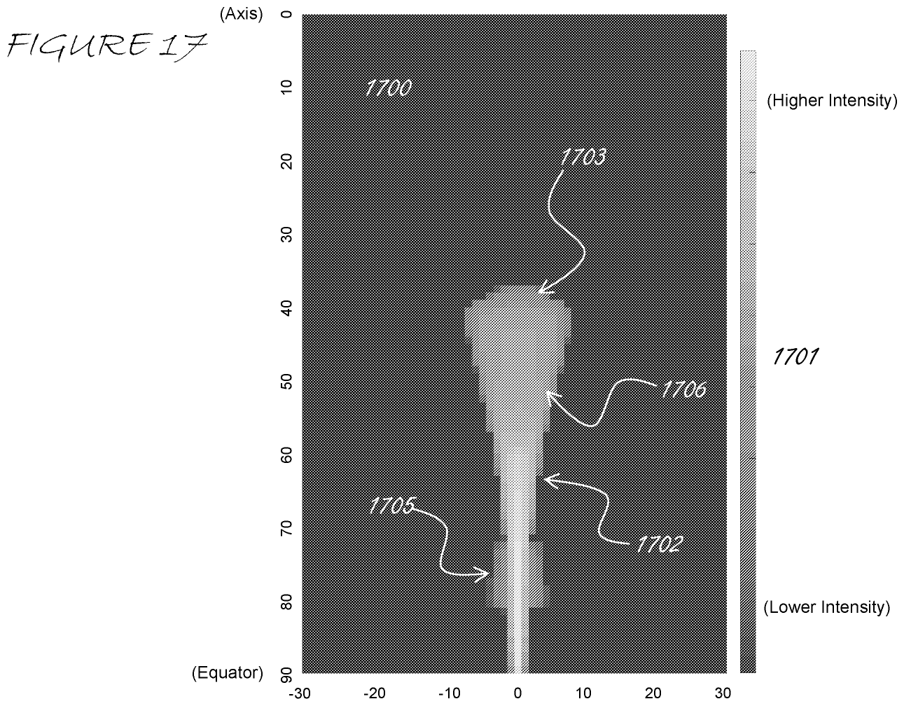

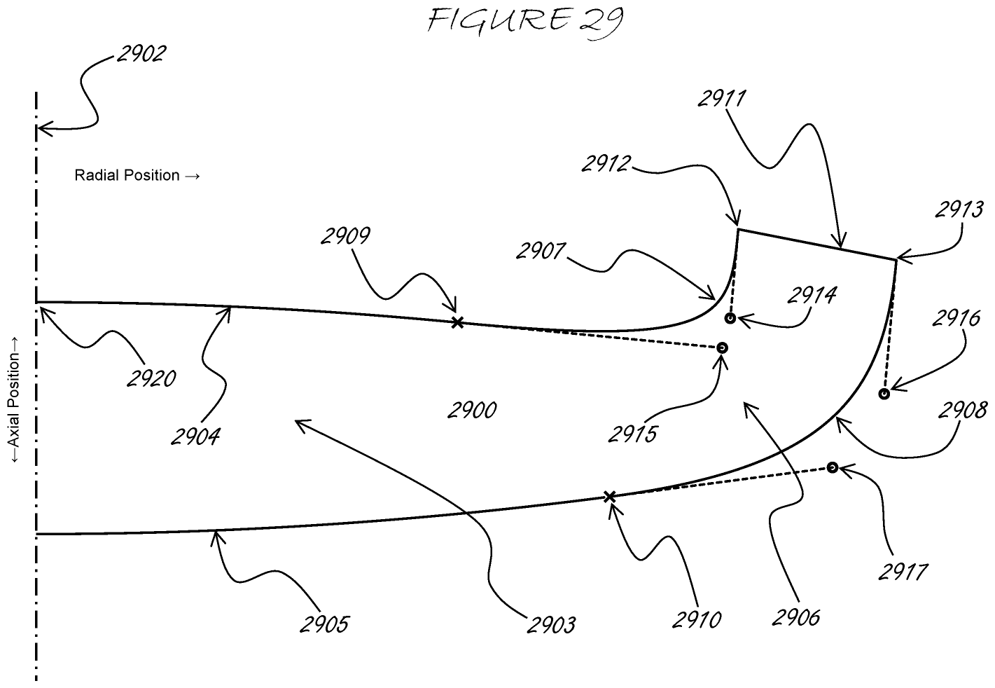

FIG. 17 is a ray density plot integrated over a range of field angles showing the intensity of light distribution across the retina using an eye modelled in accordance with FIG. 1 and the intraocular lens of FIG. 29 in accordance with certain embodiments. FIG. 17 shows a ray-density plot 1700 integrated over a range of field angles along a single meridian (e.g., a single azimuthal angle). The horizontal axis and the vertical axis of the plot are the same as those used for FIG. 6. The horizontal axis represents transverse positions on the retina as azimuthal (or meridional) angles and the vertical axis represents antero-posterior retinal positions as angles relative to the axis of the eye and subtended at the geometrical center of a sphere modelling the sclera/retina.

This ray-density plot was computed using the model detailed in FIG. 1 for a 3 mm pupil diameter and 0.3 mm implantation depth but with an IOL (see, FIG. 29) that incorporates a control zone for distributing light to the otherwise dark band region on the retina. The integration over field angles is from 55° to 100° in 0.5° steps (i.e., a total of 91 field angles). At each field angle, about 100,000 rays are traced non-sequentially to model the effect of a point light source at infinity along a single meridian (azimuthal angle) of the eye. For each field angle, a ray-intercept plot is generated (similar to those shown in FIGS. 2-5). All light rays from the ray-intercept plots, over all field angles, are then integrated and their relative retinal density (or relative light intensity, or relative irradiance, calculated as the number of rays per unit area) is computed. The parameters used in computation of this ray-density plot 1700, are the same as those used for the ray-density plot 600 of FIG. 6 with the exception of the replacement of the equi-convex IOL (FIG. 6) with a conventional edge with an IOL having a control zone for controlling negative PPD (see FIG. 16 and previous explanations). The resultant intensity at retinal positions (azimuth angle by antero-posterior retinal position) is plotted as a grey-scale for which a bright shading indicates a high intensity and a dark shading indicates a low intensity. The shading used for the relative retinal intensity is indicated by the grey-scale 1701. On the ray density plot 1700 integrating field-angles, the region of the retina irradiated by refracted light rays can be seen as the refracted light region 1702. In the modelling, a minor amount of this intensity 1705 is contributed to by edge rays (e.g., similar to 402 in FIG. 4). As mentioned, such edge rays may be controlled by suitable choice of surface features of the IOL edge such as frosting, opacifying, etc. More anteriorly in plot 1700, a smaller region representing the positions of the retina irradiated by by-pass rays (e.g., similar to 403 in FIG. 4) is seen in the by-pass region 1703. The region 1706 between the refracted rays retinal region 1702 and the by-pass rays retinal region 1703 is the region on the retina that exhibited a dark band region (e.g., dark band region 604 in FIG. 6) for conventional IOLs. This region 1706 now shows a continuity of retinal irradiance from the refracted rays region 1702 to the by-pass rays region 1703. Accordingly, the control zone of the IOL effectively ‘fills’ the potential dark band on the retina. In some embodiments, this control zone may reduce, eliminate, substantially eliminate or significantly reduce the occurrence of negative PPD.

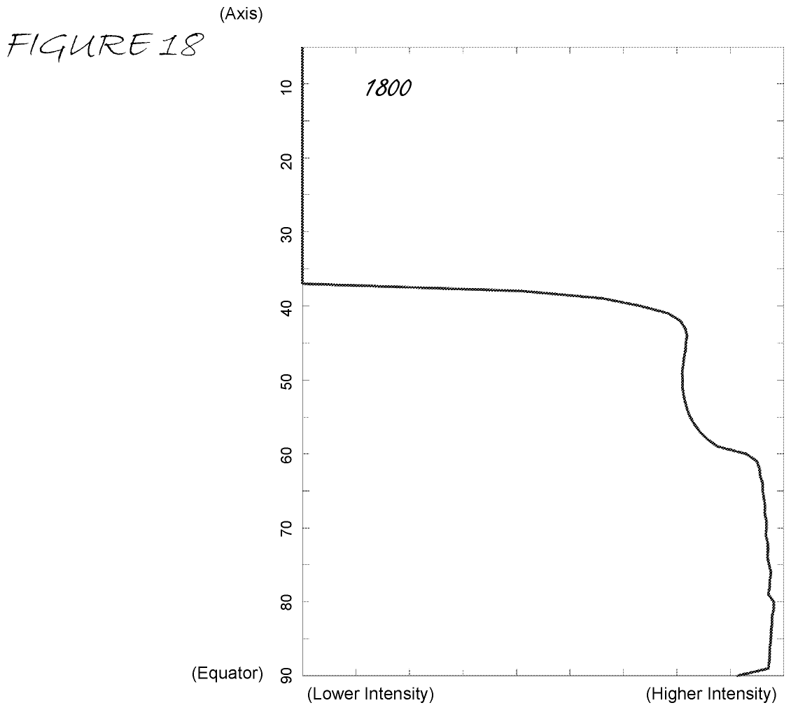

FIG. 18 is a relative whole field retinal irradiance plot (e.g., integrated ray density plot) integrated over a range of field angles and azimuthal angles showing the intensity of light distribution across the retina for the ray density plot of FIG. 17 in accordance with certain embodiments. FIG. 18 show a relative whole field retinal irradiance (or relative retinal intensity, or integrated ray density plot) plot 1800 for the ray-density plot of FIG. 17 convolved around azimuthal angles. From the plot 1800, which are results for the scenario with 3 mm pupil diameter and 0.3 mm implantation depth, it can be seen that the dark band region of the retina, for a conventional IOL, between approximally 47° and 56° retinal position (e.g., dark band 702 in FIG. 7) has been eliminated (or at least reduced or significantly reduced), resulting in a more continuous retinal irradiance across the field angles.

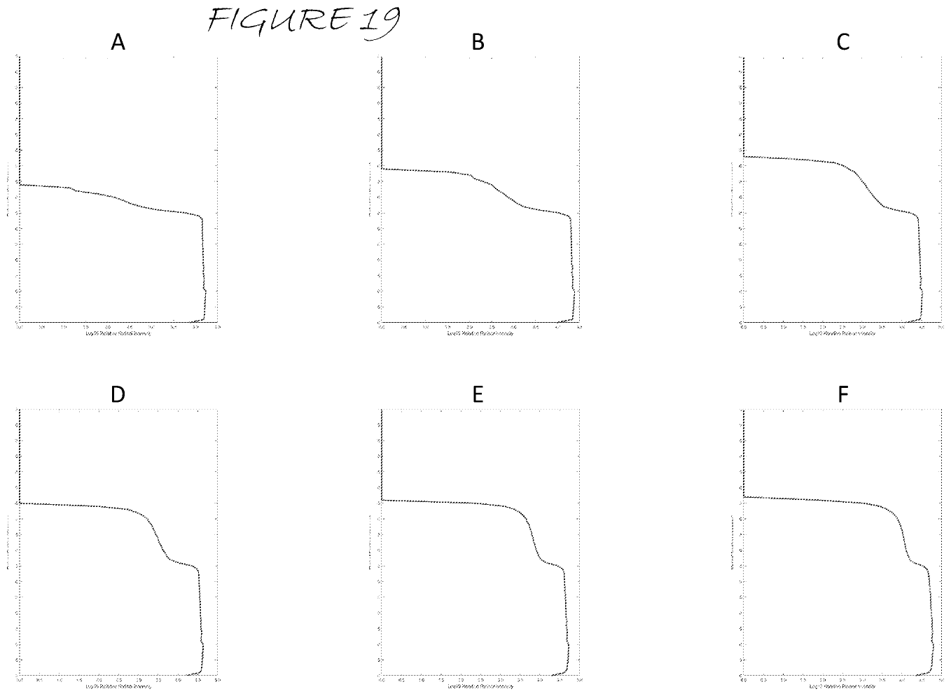

As illustrated, FIG. 18 demonstrates the effectiveness of an IOL with a control zone for reducing/eliminating the dark band region (e.g., for the scenario of 3 mm pupil diameter and implantation depth of 0.3 mm). As FIGS. 8-15 illustrated the occurrence of dark band regions for various combinations of pupil size and implantation depth, FIGS. 19-26 illustrate the relative whole-field retinal intensity while varying pupil size from 2.5 mm to 5 mm diameter in 0.5 mm steps and implantation depth from 0 mm to 0.7 mm in 0.1 mm steps. The IOL modelled is an example of an IOL (see FIG. 29) with a control zone for distributing light rays to irradiate the otherwise dark band on the retina. In other words, FIGS. 19-26 are analogous to the results presented respectively in FIGS. 8-15 with the exception that the convention equi-convex IOL has been replaced by an exemplary IOL with a control zone as described herein. In all cases, about 100,000 rays are traced non-sequentially through the eye model for each field angle. A ray-density plot is generated integrating over the (e.g., 91) field angles. Then, the ray-density results are convolved around azimuthal angles to produce a relative whole-field retinal irradiance plot. The plot axes and scales, and intensity grey-scale, of all plots shown in FIGS. 19-26 are the same as those used in FIGS. 7-15.

FIGS. 19A-F are relative retina irradiance plots showing the intensity (e.g., relative whole field retinal intensity or integrated ray density plots) of light distribution across the retina for the intraocular lens modelled in FIG. 29 for pupil sizes from 2.5 mm to 5 mm in 0.5 mm steps at an implantation depth of 0 mm in accordance with certain embodiments. FIG. 19A illustrates the relative whole-field retinal irradiance for 0 mm IOL implantation depth and a 2.5 mm pupil diameter. FIG. 19B illustrates the relative whole-field retinal irradiance for 0 mm IOL implantation depth and a 3 mm pupil diameter. FIG. 19C illustrates the relative whole-field retinal irradiance for 0 mm IOL implantation depth and a 3.5 mm pupil diameter. FIG. 19D illustrates the relative whole-field retinal irradiance for 0 mm IOL implantation depth and a 4 mm pupil diameter. FIG. 19E illustrates the relative whole-field retinal irradiance for 0 mm IOL implantation depth and a 4.5 mm pupil diameter. FIG. 19F illustrates the relative whole-field retinal irradiance for 0 mm IOL implantation depth and a 5 mm pupil diameter. As illustrated, at this implantation depth, no dark band retinal region occurs for the illustrated pupil sizes.

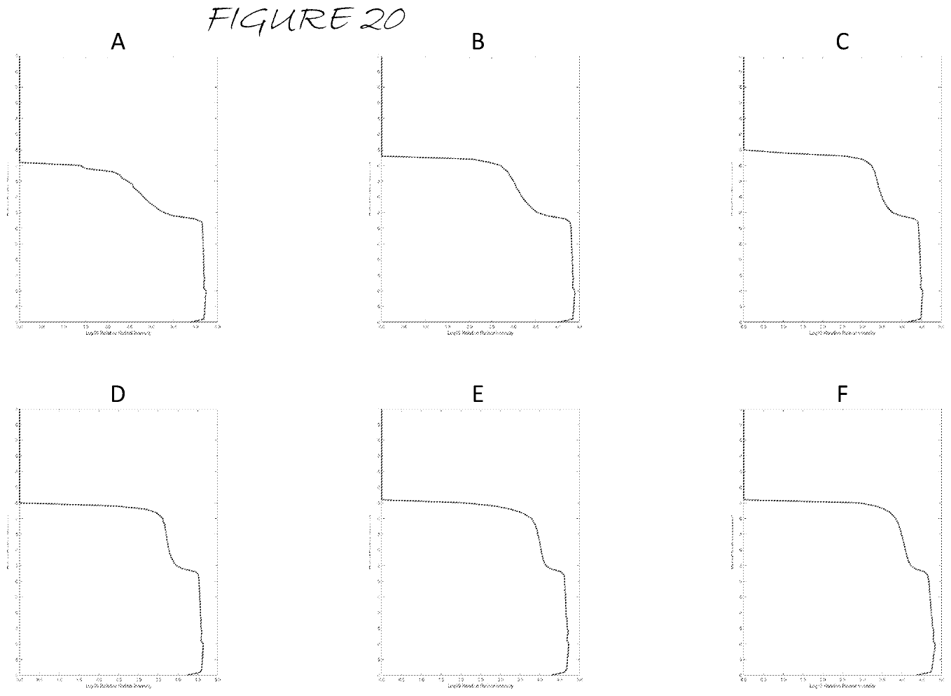

FIGS. 20A-F are relative retina irradiance plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.1 mm in accordance with certain embodiments. FIG. 20A illustrates the relative whole-field retinal irradiance for 0.1 mm IOL implantation depth and a 2.5 mm pupil diameter. FIG. 20B illustrates the relative whole-field retinal irradiance for 0.1 mm IOL implantation depth and a 3 mm pupil diameter. FIG. 20C illustrates the relative whole-field retinal irradiance for 0.1 mm IOL implantation depth and a 3.5 mm pupil diameter. FIG. 20D illustrates the relative whole-field retinal irradiance for 0.1 mm IOL implantation depth and a 4 mm pupil diameter. FIG. 20E illustrates the relative whole-field retinal irradiance for 0.1 mm IOL implantation depth and a 4.5 mm pupil diameter. FIG. 20F illustrates the relative whole-field retinal irradiance for 0.1 mm IOL implantation depth and a 5 mm pupil diameter. As illustrated, at this implantation depth, no dark band retinal region occurs for the illustrated pupil sizes.

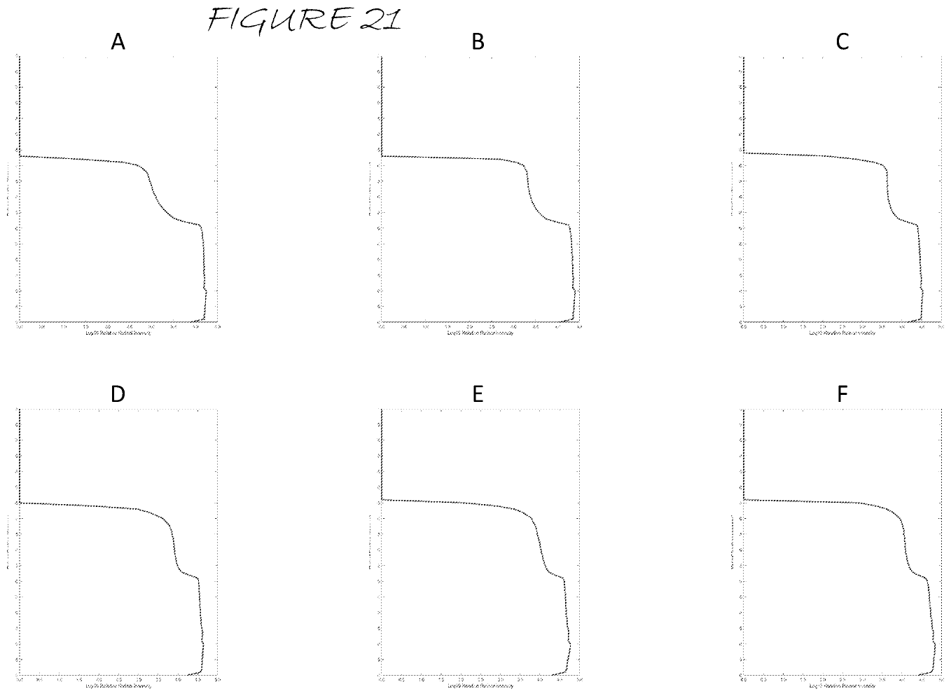

FIGS. 21A-F are relative retina irradiance plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.2 mm in accordance with certain embodiments. FIG. 21A illustrates the relative whole-field retinal irradiance for 0.2 mm IOL implantation depth and a 2.5 mm pupil diameter. FIG. 21B illustrates the relative whole-field retinal irradiance for 0.2 mm IOL implantation depth and a 3 mm pupil diameter. FIG. 21C illustrates the relative whole-field retinal irradiance for 0.2 mm IOL implantation depth and a 3.5 mm pupil diameter. FIG. 21D illustrates the relative whole-field retinal irradiance for 0.2 mm IOL implantation depth and a 4 mm pupil diameter. FIG. 21E illustrates the relative whole-field retinal irradiance for 0.2 mm IOL implantation depth and a 4.5 mm pupil diameter. FIG. 21F illustrates the relative whole-field retinal irradiance for 0.2 mm IOL implantation depth and a 5 mm pupil diameter. As illustrated, at this implantation depth, no dark band retinal region occurs for the illustrated pupil sizes.

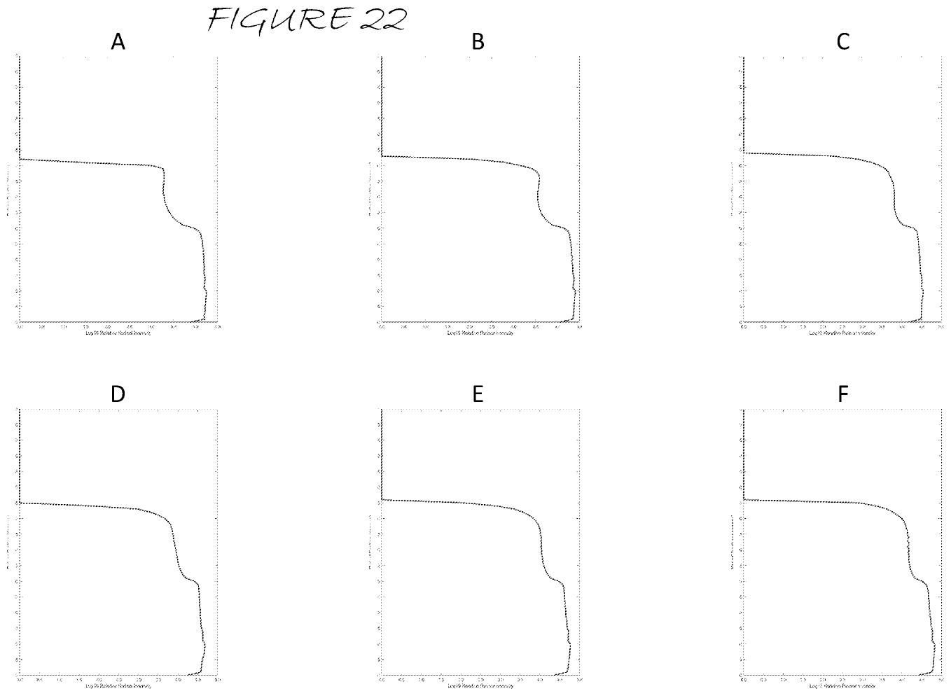

FIGS. 22A-F are relative retina irradiance plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.3 mm in accordance with certain embodiments. FIG. 22A illustrates the relative whole-field retinal irradiance for 0.3 mm IOL implantation depth and a 2.5 mm pupil diameter. FIG. 22B illustrates the relative whole-field retinal irradiance for 0.3 mm IOL implantation depth and a 3 mm pupil diameter. FIG. 22C illustrates the relative whole-field retinal irradiance for 0.3 mm IOL implantation depth and a 3.5 mm pupil diameter. FIG. 22D illustrates the relative whole-field retinal irradiance for 0.3 mm IOL implantation depth and a 4 mm pupil diameter. FIG. 22E illustrates the relative whole-field retinal irradiance for 0.3 mm IOL implantation depth and a 4.5 mm pupil diameter. FIG. 22F illustrates the relative whole-field retinal irradiance for 0.3 mm IOL implantation depth and a 5 mm pupil diameter. As illustrated, at this implantation depth, no dark band retinal region occurs for the illustrated pupil sizes.

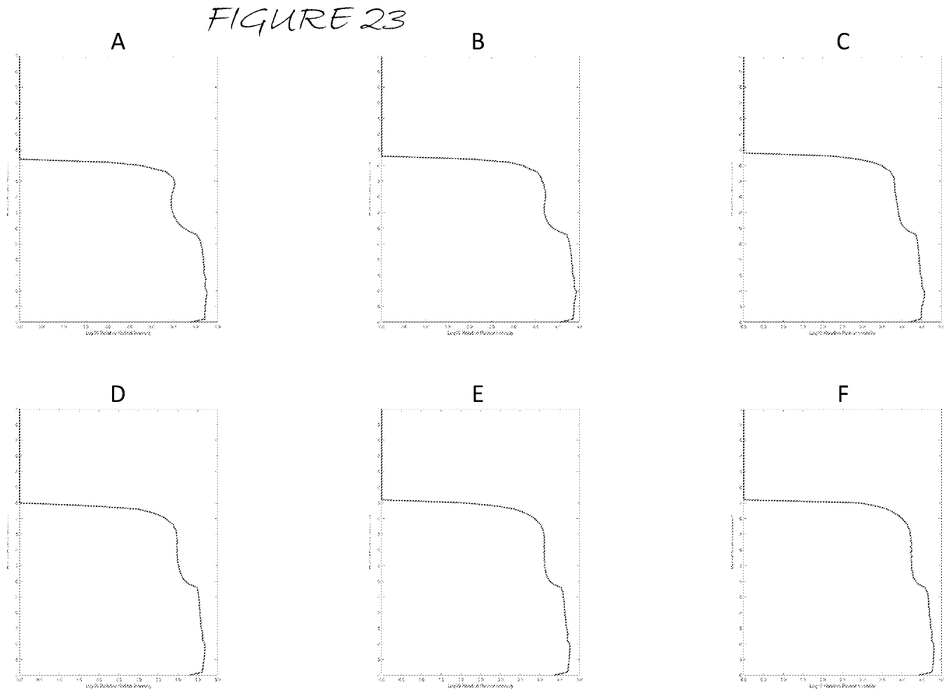

FIGS. 23A-F are relative retina irradiance plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.4 mm in accordance with certain embodiments. FIG. 23A illustrates the relative whole-field retinal irradiance for 0.4 mm IOL implantation depth and a 2.5 mm pupil diameter. FIG. 23B illustrates the relative whole-field retinal irradiance for 0.4 mm IOL implantation depth and a 3 mm pupil diameter. FIG. 23C illustrates the relative whole-field retinal irradiance for 0.4 mm IOL implantation depth and a 3.5 mm pupil diameter. FIG. 23D illustrates the relative whole-field retinal irradiance for 0.4 mm IOL implantation depth and a 4 mm pupil diameter. FIG. 23E illustrates the relative whole-field retinal irradiance for 0.4 mm IOL implantation depth and a 4.5 mm pupil diameter. FIG. 23F illustrates the relative whole-field retinal irradiance for 0.4 mm IOL implantation depth and a 5 mm pupil diameter. As illustrated, at this implantation depth, no dark band retinal region occurs for the illustrated pupil sizes.

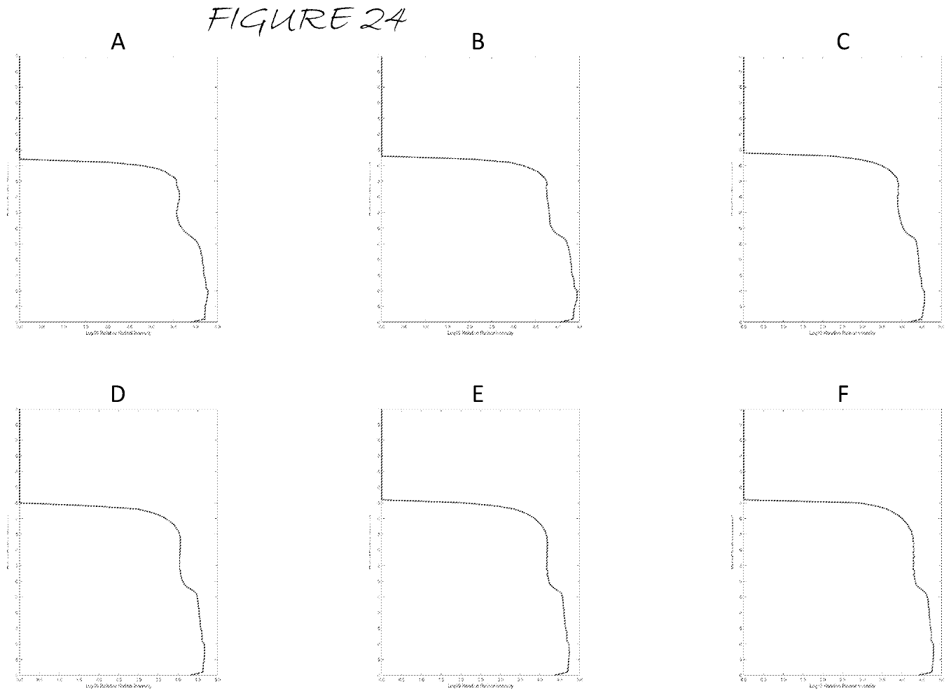

FIGS. 24A-F are relative retina irradiance plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.5 mm in accordance with certain embodiments. FIG. 24A illustrates the relative whole-field retinal irradiance for 0.5 mm IOL implantation depth and a 2.5 mm pupil diameter. FIG. 24B illustrates the relative whole-field retinal irradiance for 0.5 mm IOL implantation depth and a 3 mm pupil diameter. FIG. 24C illustrates the relative whole-field retinal irradiance for 0.5 mm IOL implantation depth and a 3.5 mm pupil diameter. FIG. 24D illustrates the relative whole-field retinal irradiance for 0.5 mm IOL implantation depth and a 4 mm pupil diameter. FIG. 24E illustrates the relative whole-field retinal irradiance for 0.5 mm IOL implantation depth and a 4.5 mm pupil diameter. FIG. 24F illustrates the relative whole-field retinal irradiance for 0.5 mm IOL implantation depth and a 5 mm pupil diameter. As illustrated, at this implantation depth, no dark band retinal region occurs for the illustrated pupil sizes.

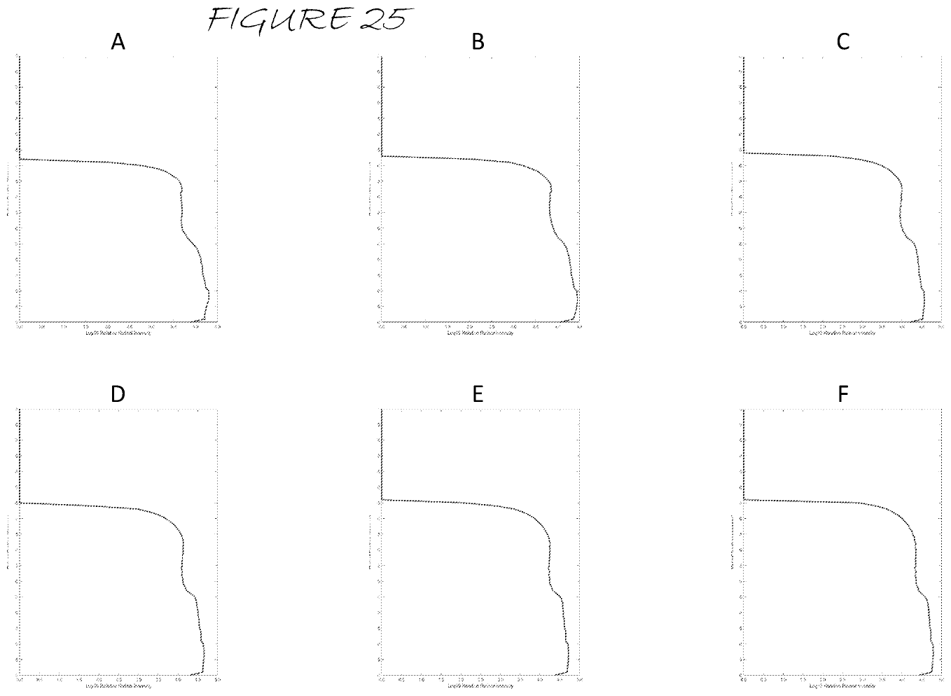

FIGS. 25A-F are relative retina irradiance plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.6 mm in accordance with certain embodiments. FIG. 25A illustrates the relative whole-field retinal irradiance for 0.6 mm IOL implantation depth and a 2.5 mm pupil diameter. FIG. 25B illustrates the relative whole-field retinal irradiance for 0.6 mm IOL implantation depth and a 3 mm pupil diameter. FIG. 25C illustrates the relative whole-field retinal irradiance for 0.6 mm IOL implantation depth and a 3.5 mm pupil diameter. FIG. 25D illustrates the relative whole-field retinal irradiance for 0.6 mm IOL implantation depth and a 4 mm pupil diameter. FIG. 25E illustrates the relative whole-field retinal irradiance for 0.6 mm IOL implantation depth and a 4.5 mm pupil diameter. FIG. 25F illustrates the relative whole-field retinal irradiance for 0.6 mm IOL implantation depth and a 5 mm pupil diameter. As illustrated, at this implantation depth, no dark band retinal region occurs for the illustrated pupil sizes.

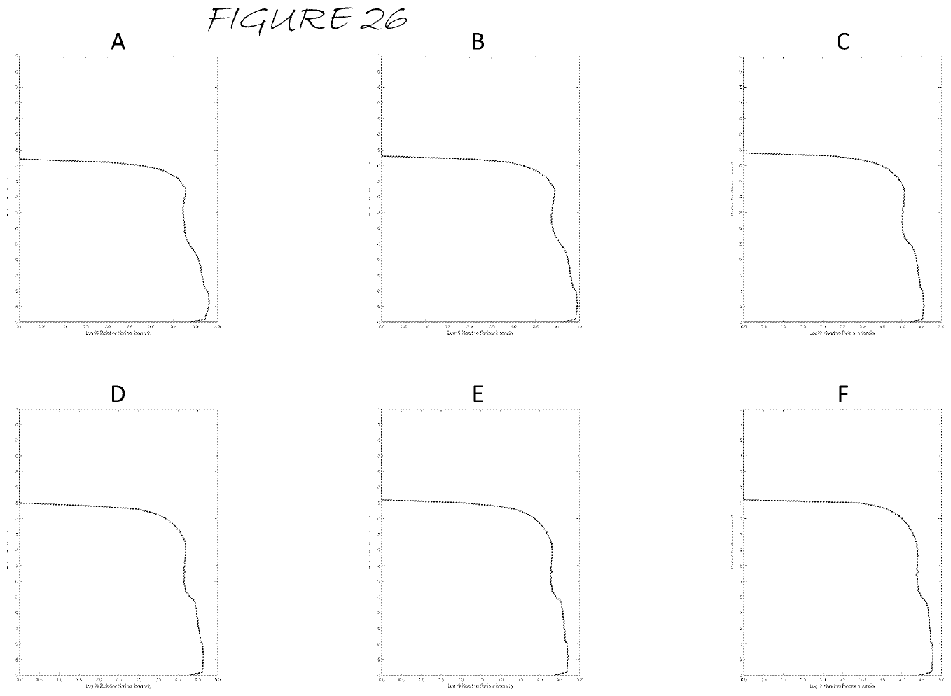

FIGS. 26A-F are relative retina irradiance plots showing the intensity (e.g., relative whole field retinal irradiance) of light distribution across the retina for the intraocular lens modelled in FIG. 29 for pupil sizes from 2.5 mm to 5 mm in 0.5 mm steps at an implantation depth of 0.7 mm in accordance with certain embodiments. FIG. 26A illustrates the relative whole-field retinal irradiance for 0.7 mm IOL implantation depth and a 2.5 mm pupil diameter. FIG. 26B illustrates the relative whole-field retinal irradiance for 0.7 mm IOL implantation depth and a 3 mm pupil diameter. FIG. 26C illustrates the relative whole-field retinal irradiance for 0.7 mm IOL implantation depth and a 3.5 mm pupil diameter. FIG. 26D illustrates the relative whole-field retinal irradiance for 0.7 mm IOL implantation depth and a 4 mm pupil diameter. FIG. 26E illustrates the relative whole-field retinal irradiance for 0.7 mm IOL implantation depth and a 4.5 mm pupil diameter. FIG. 26F illustrates the relative whole-field retinal irradiance for 0.7 mm IOL implantation depth and a 5 mm pupil diameter. As illustrated, at this implantation depth, no dark band retinal region occurs for the illustrated pupil sizes.

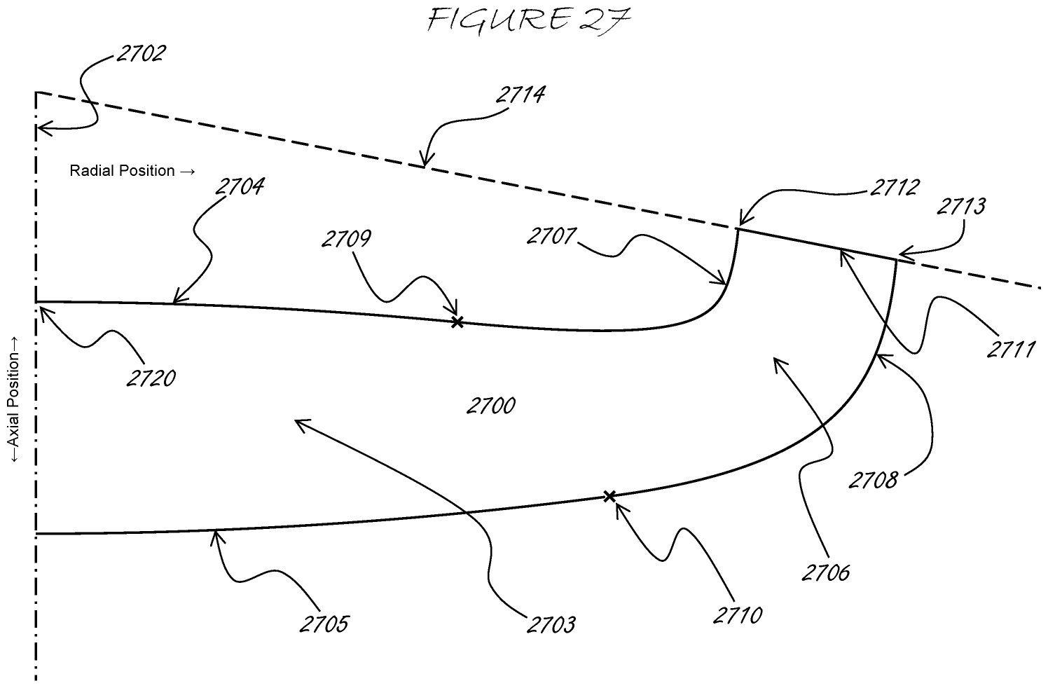

FIG. 27 is a schematic illustration of a half-meridian section of an intraocular lens for reducing, minimizing, and/or eliminating peripheral pseudophakic dysphotopsia in accordance with certain embodiments. The prescription for the exemplary IOL illustrated in FIG. 27 is provided in Table 1 below.

TABLE 1

| | | | |
|---|---|---|---|
| Feature/Parameter | Unit | Value | Comment |

OPTIC ZONE

| | | | |
|---|---|---|---|
| refractive index | | 1.55 | |
| nominal power of IOL | (D) | +19 | when immersed in water |
| front optic radius of curvature | (mm) | 22.541 | |
| central thickness | (mm) | 0.551 | |
| back optic radius of curvature | (mm) | −22.541 | negative value means convex |
| | | | towards the back |

CONTROL ZONE

| | | | |
|---|---|---|---|
| front optic-control junction radial | (mm) | 1.469 | radial distance from axis of IOL |
| position | | | |
| front optic-control junction axial | (mm) | 0.048 | axial distance from vertex plane |
| position | | | of IOL |
| back optic-control junction radial | (mm) | 2.000 | radial distance from axis of IOL |
| position | | | |
| back optic-control junction axial | (mm) | 0.462 | axial distance from vertex plane |
| position | | | of IOL |
| angle of edge | (°) | 7.595 | relative to frontal plane |
| front edge point radial position | (mm) | 2.448 | radial distance from axis of IOL |
| front edge point axial position | (mm) | −0.174 | relative to apex of IOL (negative |
| | | | value means in front of IOL apex) |
| back edge point radial position | (mm) | 3.000 | radial distance from axis of IOL |
| back edge point axial position | (mm) | −0.100 | relative to apex of IOL |
| front control surface profile | | | |
| polynomial order | | 6 | apex of polynomial is at front |
| | | | edge point |
| 2nd order coefficient | | −9.783E−01 | |
| 6th order coefficient | | −4.555E+02 | |
| front polynomial tilt angle | (°) | 7.595 | tilt of axis of polynomial relative |
| | | | to frontal plane |
| back control surface profile | | | |
| polynomial order | | 4 | apex of polynomial is at back |
| | | | edge point |
| 2nd order coefficient | | −6.365E−01 | |
| 4th order coefficient | | −2.721E+00 | |
| back polynomial tilt angle | (°) | 7.595 | tilt of axis of polynomial relative |
| | | | to frontal plane |

The meridional cross section of FIG. 27 is for purpose of illustration only and may not be isometric (e.g., anisometric); that is, the distance (or dimensions or scaling) in the horizontal (or radial) direction may not be the same as that in the vertical (or axial) direction.

In the exemplary embodiment of FIG. 27, the IOL 2700 is made of material with a refractive index of 1.55 and provides a refractive power of about +19 D through the optic zone 2703. The optic is equiconvex with an anterior surface 2704 radius of curvature equal to 22.541 mm. The convex back optic zone surface 2705 has a radius of −22.541 mm. As would be readily understood by a person of ordinary skill in the art, a sign convention is used herein whereby distances are considered positive when measured in the antero-posterior direction of travel of light in the IOL or eye. In this sign convention, a radius is measured from the surface to the center of the radius. Thus, a convex back surface (i.e. convex in the posterior direction) has a negative value for its radius of curvature. The central thickness of the IOL 2700 is 0.551 mm.

A control zone 2706 for controlling, reducing and/or eliminating peripheral pseudophakic dysphotopsia (PPD) is provided as part of IOL 2700. The control zone 2706 comprises a front control surface 2707 and a back control surface 2708. The front (anterior) control surface 2707 extends from the junction 2709 (sometimes referred to as the “front optic-control junction”) between the front optic zone 2704 and the front control surface 2707 to a front edge point 2712 (sometimes referred to as the “front control-edge junction”). The back (posterior) control surface 2708 extends from the junction 2710 (sometimes referred to as the “back optic-control junction”) between the back optic zone 2705 and the back control surface 2708 to a back edge point 2713 (sometimes referred to as the “back control-edge junction”). The front edge point 2712 and back edge point 2713 are joined by the edge 2711 of the IOL.

The radial and axial positions of the front optic-control junction 2709, back optic-control junction 2710, front edge point 2712 and back edge point 2713 for this exemplary IOL are given in Table 1 above. As would be readily understood by persons of ordinary skill in the art, radial distances are measured from the axis 2702 of the IOL and in a direction perpendicular to the axis 2702 of the IOL. Axial distances are measured from a frontal plane containing the front apex 2720 of the IOL and in a direction perpendicular to the frontal plane. A frontal plane is a plane that is parallel to the plane of the iris (whose aperture forms the pupil) of the eye and is perpendicular to an axis 2702 of the eye and IOL. (It is understood by vision scientists, designers of IOL and eye-care practitioners that while the eye may not be exactly rotationally symmetrical, it may be reasonably approximal to be rotationally symmetrical. Similarly, the axis of an IOL may be reasonably approximal to be coincident with the axis of the eye.)

Using the same sign convention, axial positions that are in front of (that is, in a direction from the apex that is against the direction of travel of light in the eye) the apex 2720 of the IOL has a negative axial distance while axial positions that are behind or more posterior than apex 2720 are considered positive.

In this exemplary IOL (with prescription listed in Table 1 above), the profiles of the front control surface 2707 and back control surface 2708 are defined using polynomial equations which are then translated by select radial and axial distances and tilted by a select angle to provide continuity between a control surface (2707 for front, 2708 for back) and the profile of the optic zone (2704 for front, 2705 for back) at their respective junction (2709 for front, 2710 for back).

In some embodiments, the polynomial equation may be of a form described by below:

r=a₂z²+aₖ|z|^{k} Eq. (1)

where r and z are respectively the local radial and axial coordinates for points on the control surface profile and k is an exponent greater than 2, and a₂ and aₖ are coefficients associated with the second-order and k^{th}-order component of the polynomial. Coordinates r and z of Eq. 1 may be local to (e.g., specific to) the polynomial and may be distinct from the radial and axial coordinates with respect to the IOL and/or the eye. The exponent k need not be restricted to integers but may have value drawn from the set of real numbers (e.g. a decimal number such as 2.573).

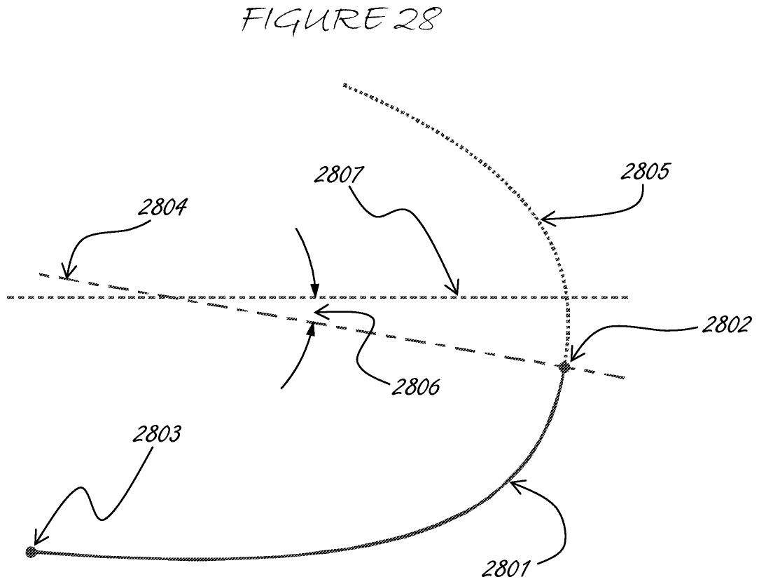

FIG. 28 is an exemplary embodiment of an implementation of Eq. 1 for defining a control surface profile of an intraocular lens in accordance with certain embodiments. In FIG. 28, a polynomial of the form described by Eq. 1 is used to define the control surface 2801 joining an optic-control junction 2803 and an edge point 2802.

As illustrated, the prescribed polynomial is translated to place its apex at the edge point 2802. The polynomial exhibits symmetry along an axis 2804. The polynomial is tilted by an angle 2806 relative to a frontal plane 2807 of the eye and IOL, and that its axis of symmetry 2804 is coincident with the edge of the IOL (e.g., 2711 in FIG. 27).

Eq. 1 describes a polynomial curve symmetric about its axis 2804 which therefore possesses an upper arm 2805 and a lower arm 2801, in this case, the lower arm 2801 (with the greater positive z or axial value) is used for defining the profile of the control surface.

Table 1 lists the values for the order k, 2^{nd}-order coefficient, k^{th}-order coefficient and the tilt angle for the front control surface profile and the back control surface profile. For both control surface profiles, the polynomial is translated to place its apex on their respective edge point.

The edge of this exemplary IOL is at an angle of about 7.6° relative to a frontal plane of the IOL or eye. That is, a normal to the lens edge is at angle of about 7.6° to an axis of the IOL. For this exemplary IOL, the angle between the lens edge and the front control surface as the front control surface approaches the front edge point (that is, the angle of a tangent to the front control surface at or near to the front edge point) is about 90°. This angle is considered as the “internal” angle of the front edge point of the IOL. The internal angle is the angle subtended within the bulk (or material) of the IOL at the front edge point. In FIG. 27, the angle is that between the tangent to the front control curve 2707 at the front edge point 2712 and the lens edge 2711 between front edge point 2712 and back edge point 2713 (choosing the value for the angle that is less than 180°). The angle between the lens edge and the back control surface as the back control surface approaches the back edge point (that is, the angle of a tangent to the back control surface at or near to the back edge point) is about 90°. Similarly to the angle at the front edge point, this angle between the lens edge and the back control surface is measured as that between the tangent to the back control curve 2708 at the back edge point 2713 and the lens edge 2711 between back edge point 2713 and front edge point 2712 (choosing the value for the angle that is less than 180°).

In some embodiments, control surface profiles may be described using other mathematical functions such as one or more of splines curves, Fourier series, etc. For example, control surface profiles for an IOL with substantially the same optic zone prescription as that of FIG. 27 may be defined using Bezier segments. The prescription for such an exemplary IOL with control surface profile for controlling PPD is described below with respect to e.g., FIG. 29 and Table 2.

FIG. 29 is a schematic illustration of a half-meridian of an intraocular lens for reducing, minimizing, and/or eliminating peripheral pseudophakic dysphotopsia in accordance with certain embodiments. This exemplary embodiment of an IOL is the design used to obtain the results described with respect to FIGS. 17-26. The prescription for such an exemplary IOL with control surface profile for controlling PPD is given in Table 2 below.

TABLE 2

| | | | |
|---|---|---|---|
| Feature/Parameter | Unit | Value | Comment |

OPTIC ZONE

| | | | |
|---|---|---|---|
| refractive index | | 1.55 | |
| nominal power of IOL | (D) | +19 | when immersed in water |
| front optic radius of curvature | (mm) | 22.541 | |
| central thickness | (mm) | 0.551 | |
| back optic radius of curvature | (mm) | −22.541 | negative value means convex |
| | | | towards the back |

CONTROL ZONE

| | | | |
|---|---|---|---|
| front optic-control junction radial | (mm) | 1.469 | radial distance from axis of IOL |
| position | | | |
| front optic-control junction axial | (mm) | 0.048 | axial distance from vertex plane |
| position | | | of IOL |
| back optic-control junction radial | (mm) | 2.000 | radial distance from axis of IOL |
| position | | | |
| back optic-control junction axial | (mm) | 0.462 | axial distance from vertex plane |
| position | | | of IOL |
| angle of edge | (°) | 7.595 | relative to frontal plane |
| front edge point radial position | (mm) | 2.448 | radial distance from axis of IOL |
| front edge point axial position | (mm) | −0.174 | relative to apex of IOL (negative |
| | | | value means in front of IOL apex) |
| back edge point radial position | (mm) | 3.000 | radial distance from axis of IOL |
| back edge point axial position | (mm) | −0.100 | relative to apex of IOL |
| front control surface profile | | | |
| front edge Bezier | (mm) | 2.420 | radial distance from axis of IOL |
| tangent point radial position | | | |
| front edge Bezier | (mm) | 0.038 | axial distance from vertex plane |
| tangent point axial position | | | of IOL |
| front junction Bezier | (mm) | 2.393 | radial distance from axis of IOL |
| tangent point radial position | | | |
| front junction Bezier | (mm) | 0.108 | axial distance from vertex plane |
| tangent point axial position | | | of IOL |
| back control surface profile | | | |
| back edge Bezier | (mm) | 2.958 | radial distance from axis of IOL |
| tangent point radial position | | | |
| back edge Bezier | (mm) | 0.218 | axial distance from vertex plane |
| tangent point axial position | | | of IOL |
| back junction Bezier | (mm) | 2.778 | radial distance from axis of IOL |
| tangent point radial position | | | |
| back junction Bezier | (mm) | 0.393 | axial distance from vertex plane |
| tangent point axial position | | | of IOL |

As described, the IOL 2900 is made of material with a refractive index of 1.55 and provides a refractive power of about +19 D through the optic zone 2903. The optic is equiconvex with anterior surface 2904 radius of curvature 22.541 mm. The convex back optic zone surface 2905 has a radius of −22.541 mm (using the same sign convention described above). Central thickness of the IOL 2900 is 0.551 mm.

A control zone 2906 for controlling, reducing and/or eliminating PPD is provided in IOL 2900. The control zone 2906 comprises a front control surface 2907 and a back control surface 2908. The front (anterior) control surface 2907 extends from the front optic-control junction 2909 to a front edge point 2912. The back (posterior) control surface 2908 extends from the back optic-control junction 2910 to a back edge point 2913. The front edge point 2912 and back edge point 2913 is joined by the edge 2911 of the IOL.

The radial and axial positions of the front optic-control junction 2909, back optic-control junction 2910, front edge point 2912 and back edge point 2913 for this exemplary IOL are given in Table 2 according to the same sign convention used above with respect to Table 1.

In this exemplary IOL (with prescription listed in Table 2), the profiles of the front control surface 2907 and back control surface 2908 are defined using Bezier curve segments to provide continuity between control surface (2907 for front, 2908 for back) and the profile of the optic zone (2904 for front, 2905 for back) at their respective junction (2909 for front, 2910 for back).

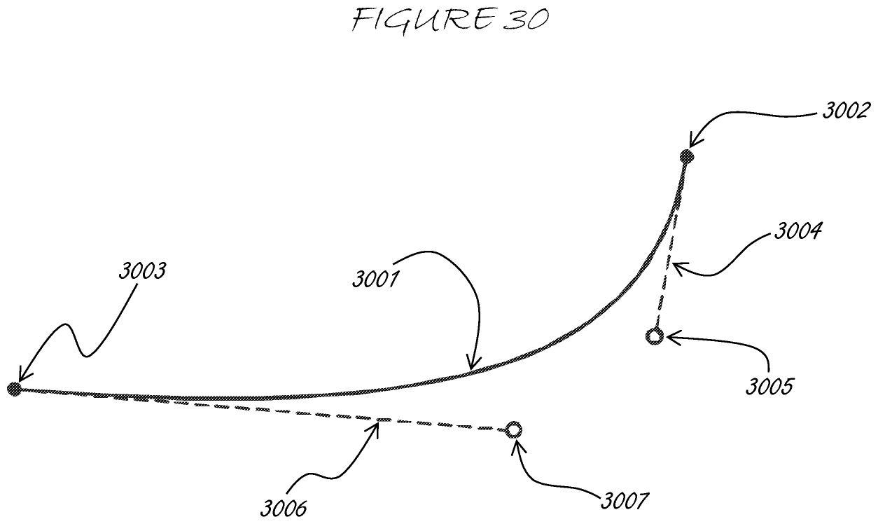

FIG. 30 is a schematic illustration of a portion of the half-meridian of an intraocular lens described in FIG. 29 in accordance with certain embodiments. As illustrated in FIG. 30, the cubic Bezier segment 3001 has four cardinal points that determine the shape of the Bezier segment. These are the start point 3002 and end point 3003 of the Bezier curve as well as two points 3005 and 3007 (referred to as “tangent points”). It should be noted that while the start point and end points represent physical points that lie on the control surface at the ends of the Bezier segment profile, the tangent points are not physical points but are mathematical constructs for defining the profile of the Bezier curve segment.

The equation for a cubic Bezier segment is:

$$[ \begin{matrix}r \\ z\end{matrix} ] = {( 1 - t )}^{3} [ \begin{matrix}{r}_{0} \\ {z}_{0}\end{matrix} ] + 3 {t ( 1 - t )}^{2} [ \begin{matrix}{r}_{1} \\ {z}_{1}\end{matrix} ] + 3 {t}^{2} ( 1 - t ) [ \begin{matrix}{r}_{2} \\ {z}_{2}\end{matrix} ] + {t}^{3} [ \begin{matrix}{r}_{3} \\ {z}_{3}\end{matrix} ] \qquad Eq . ( 2 )$$

where [r, z] are the radial and axial coordinates for points along the Bezier segment between start point 3002 with coordinates [r₀, z₀] and end point 3003 with coordinates [r₃, z₃] evaluated parametrically with parameter t with values between 0 and 1 inclusive, whereby t=0 represents the start point 3002, and t=1 represents the end point 3003. The start tangent point 3005 (i.e. the tangent point associated with the start point) has coordinates [r₁, z₁], and the end tangent point 3007 has coordinates [r₂, z₂].

FIG. 29 illustrates the implementation of Bezier segment in the exemplary IOL. For the front control surface 2907, the Bezier segment has start point (corresponding to point 3002 in FIG. 30) at the front edge point 2912, end point (corresponding to point 3003 in FIG. 30) at the front optic-control junction 2909. The positions of the front start tangent point 2914 and front end tangent point 2915 (corresponding to points 3005 and 3007 in FIG. 30 respectively) are also shown.

For the back control surface 2908, the Bezier segment has start point (corresponding to point 3002 in FIG. 30) at the back edge point 2913, end point (corresponding to point 3003 in FIG. 30) at the back optic-control junction 2910. The positions of the back start tangent point

2916 and back end tangent point 2917 (corresponding to points 3005 and 3007 in FIG. 30 respectively) are also shown.

Table 2 gives the radial and axial coordinates (positions) of the relevant points.

The edge of this exemplary IOL is at an angle of about 7.6° relative to a frontal plane of the IOL or eye. That is, a normal to the lens edge is at angle of about 7.6° to an axis of the IOL. For this exemplary IOL, the angle between the lens edge and the front control surface as the front control surface approaches the front edge point (that is, the angle of a tangent to the front control surface at or near to the front edge point) is about 90°. This angle is considered as the “internal” angle of the front edge point of the IOL. The internal angle is the angle subtended within the bulk (or material) of the IOL at the front edge point. In FIG. 29, the angle is that between the tangent to the front control curve 2907 at the front edge point 2912 and the lens edge 2911 between front edge point 2912 and back edge point 2913 (choosing the value for the angle that is less than 180°). The angle between the lens edge and the back control surface as the back control surface approaches the back edge point (that is, the angle of a tangent to the back control surface at or near to the back edge point) is about 90°. Similarly to the angle at the front edge point, this angle between the lens edge and the back control surface is measured as that between the tangent to the back control curve 2908 at the back edge point 2913 and the lens edge 2911 between back edge point 2913 and front edge point 2912 (choosing the value for the angle that is less than 180°).

For the purpose of optical ray-tracing analyses of PPD (e.g., FIGS. 17 to 26), an IOL that utilizes a Bezier segment for defining a control curve may be modelled using the Part Designer function of Zemax Opticstudio. The Sketch tab within Zemax Part Designer may be used to render the Bezier segment curves for the front and back control curves as well as the IOL edge. The Sketch may then be combined with the optic zone of the IOL which may also be rendered in Part Designer. The fully rendered IOL, complete with optic and control regions, may then be exported as a computer-aided design (CAD) file using a format such as the Initial Graphics Exchange Specification (IGES). The IGES file may then be loaded into the Zemax Opticstudio model (including IOL and eye) as a CAD Part: STEP/IGES/SAT surface type.

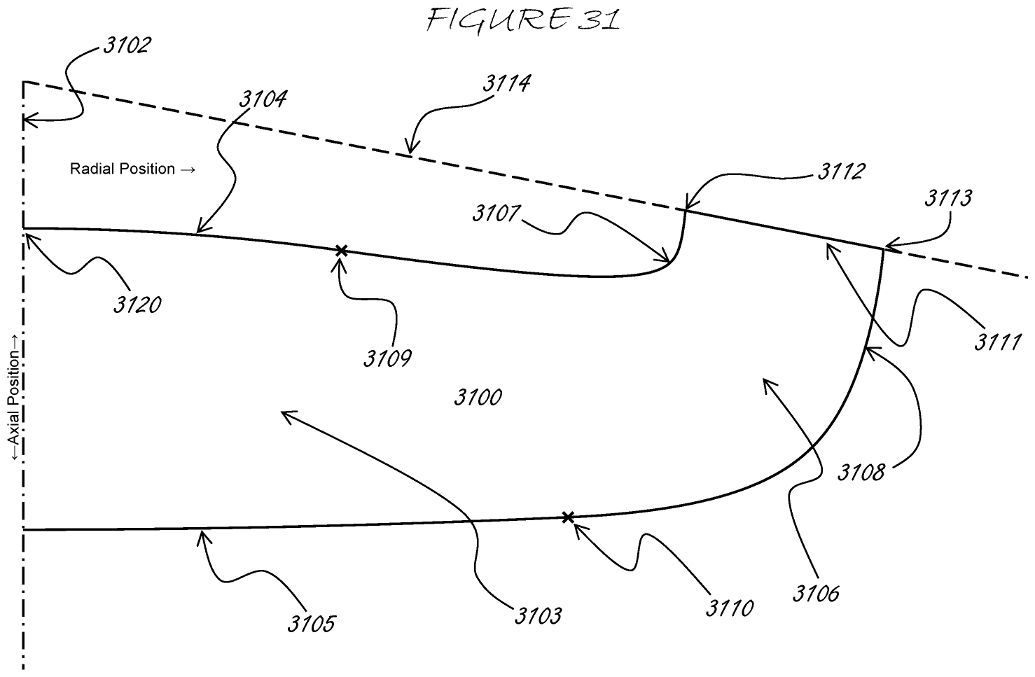

FIG. 31 is a schematic illustration of a half-meridian of an intraocular lens for reducing, minimizing, and/or eliminating peripheral pseudophakic dysphotopsia in accordance with certain embodiments. The prescription for this exemplary IOL for controlling PPD is given in Table 3 below.

TABLE 3

| | | | |
|---|---|---|---|
| Feature/Parameter | Unit | Value | Comment |

OPTIC ZONE

| | | | |
|---|---|---|---|
| refractive index | | 1.55 | |
| nominal power of IOL | (D) | +22 | when immersed in water |
| front optic radius of curvature | (mm) | 11.633 | |
| central thickness | (mm) | 0.717 | |
| back optic radius of curvature | (mm) | −61.536 | negative value means convex |
| | | | towards the back |

CONTROL ZONE

| | | | |
|---|---|---|---|
| front optic-control junction radial | (mm) | 1.109 | radial distance from axis of IOL |
| position | | | |
| front optic-control junction axial | (mm) | 0.053 | axial distance from vertex plane |
| position | | | of IOL |
| back optic-control junction radial | (mm) | 1.900 | radial distance from axis of IOL |
| position | | | |
| back optic-control junction axial | (mm) | 0.688 | axial distance from vertex plane |
| position | | | of IOL |
| angle of edge | (°) | 7.595 | relative to frontal plane |
| front edge point radial position | (mm) | 2.309 | radial distance from axis of IOL |
| front edge point axial position | (mm) | −0.042 | relative to apex of IOL |
| | | | (negative value means in front |
| | | | of IOL apex) |
| back edge point radial position | (mm) | 3.000 | radial distance from axis of IOL |
| back edge point axial position | (mm) | 0.050 | relative to apex of IOL |
| front control surface profile | | | |
| polynomial order | | 6 | apex of polynomial is at front |
| | | | edge point |
| 2nd order coefficient | | −9.261E−01 | |
| 6th order coefficient | | −4.275E+03 | |
| front polynomial tilt angle | (°) | 7.595 | tilt of axis of polynomial |
| | | | relative to frontal plane |
| back control surface profile | | | |
| polynomial order | | 6 | apex of polynomial is at back |
| | | | edge point |
| 2nd order coefficient | | −5.468E−01 | |
| 6th order coefficient | | −3.060E+00 | |
| back polynomial tilt angle | (°) | 7.595 | tilt of axis of polynomial |
| | | | relative to frontal plane |

The IOL 3100 is made of material with a refractive index of 1.55 and provides a refractive power of about +22 D through the optic zone 3103. The optic zone has an anterior surface 3104 radius of curvature of 11.633 mm. The convex back optic zone surface 3105 has a radius of curvature of −61.536 mm. Central thickness of the IOL 3100 is 0.717 mm.

The radial and axial positions of the front optic-control junction 3109, back optic-control junction 3110, front edge point 3112 and back edge point 3113 for this exemplary IOL are given in Table 3.

In this exemplary IOL, the profiles of the front control surface 3107 and back control surface 3108 are defined using polynomial equations which are then translated by select radial and axial distances and tilted by a select angle to provide continuity between a control surface (3107 for front, 3108 for back) and the profile of the optic zone (3104 for front, 3105 for back) at their respective junction (3109 for front, 3110 for back).

The form of the polynomial equation has previously been described with respect to Eq. 1. The prescribed polynomial is translated to place its apex at the edge point (3112 for front and 3113 for back) corresponding to point 2802 in FIG. 28. The polynomial is tilted by an angle (corresponding to 2806 in FIG. 28) relative to a frontal plane of the eye and IOL, and that its axis of symmetry 3114 (corresponding to 2804 in FIG. 28) is coincident with the edge 3111 of the IOL.

Table 3 lists the values for the order k, 2^{nd}-order coefficient, k^{th}-order coefficient and the tilt angle for the front control surface profile and the back control surface profile. For both control surface profiles, the polynomial is translated to place its apex on their respective edge point and tilted so their axes are tilted to an angle 3114.

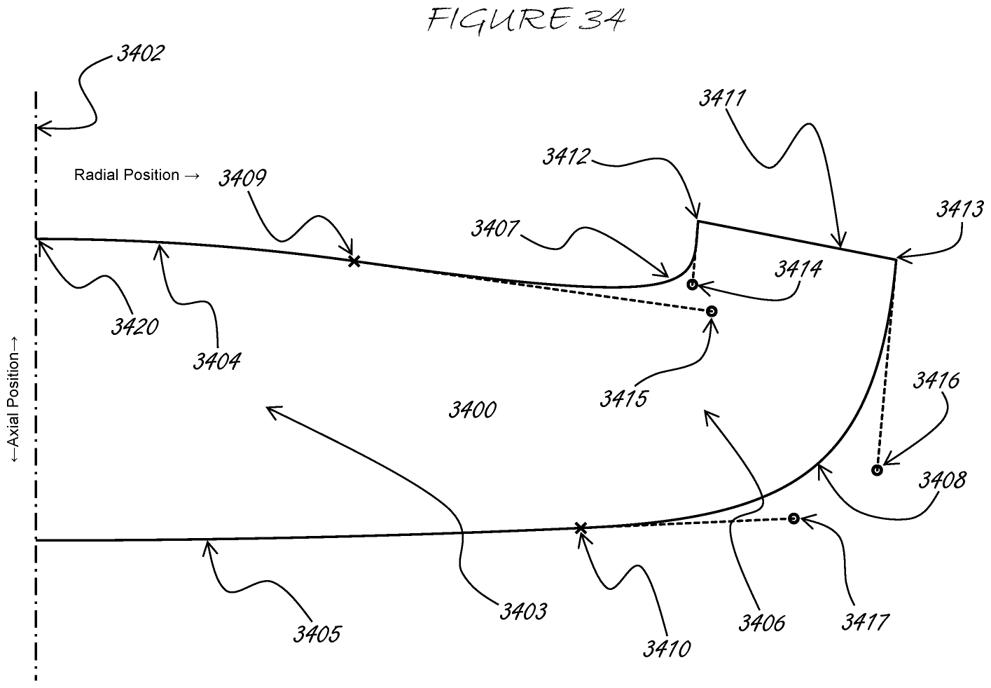

FIG. 34 is a schematic illustration of a half-meridian of an intraocular lens for reducing, minimizing, and/or eliminating peripheral pseudophakic dysphotopsia in accordance with certain embodiments. The prescription for this exemplary IOL with the same optic zone parameters as those described above with respect to FIG. 31 but with control surface profile defined using Bezier segments and given in Table 4 below.

TABLE 4

| | | | |
|---|---|---|---|
| Feature/Parameter | Unit | Value | Comment |

OPTIC ZONE

| | | | |
|---|---|---|---|
| refractive index | | 1.55 | |
| nominal power of IOL | (D) | +22 | when immersed in water |
| front optic radius of | (mm) | 11.633 | |
| curvature | | | |
| central thickness | (mm) | 0.717 | |
| back optic radius of | (mm) | −61.536 | negative value means |
| curvature | | | convex towards the back |

CONTROL ZONE

| | | | |
|---|---|---|---|
| front optic-control junction | (mm) | 1.109 | radial distance from axis |
| radial position | | | of IOL |
| front optic-control junction | (mm) | 0.053 | axial distance from vertex |
| axial position | | | plane of IOL |
| back optic-control junction | (mm) | 1.900 | radial distance from axis |
| radial position | | | of IOL |
| back optic-control junction | (mm) | 0.688 | axial distance from vertex |
| axial position | | | plane of IOL |
| angle of edge | (°) | 7.595 | relative to frontal plane |
| front edge point radial | (mm) | 2.309 | radial distance from axis |
| position | | | of IOL |
| front edge point axial | (mm) | −0.042 | relative to apex of IOL |
| position | | | (negative value means in |
| | | | front of IOL apex) |
| back edge point radial | (mm) | 3.000 | radial distance from axis |
| position | | | of IOL |
| back edge point axial | (mm) | 0.050 | relative to apex of IOL |
| position | | | |
| front control surface profile | | | |
| front edge Bezier | (mm) | 2.289 | radial distance from axis |
| tangent point radial position | | | of IOL |
| front edge Bezier | (mm) | 0.109 | axial distance from vertex |
| tangent point axial position | | | plane of IOL |
| front junction Bezier | (mm) | 2.357 | radial distance from axis |
| tangent point radial position | | | of IOL |
| front junction Bezier | (mm) | 0.173 | axial distance from vertex |
| tangent point axial position | | | plane of IOL |
| back control surface profile | | | |
| back edge Bezier | (mm) | 2.933 | radial distance from axis |
| tangent point radial position | | | of IOL |
| back edge Bezier | (mm) | 0.550 | axial distance from vertex |
| tangent point axial position | | | plane of IOL |
| back junction Bezier | (mm) | 2.643 | radial distance from axis |
| tangent point radial position | | | of IOL |
| back junction Bezier | (mm) | 0.665 | axial distance from vertex |
| tangent point axial position | | | plane of IOL |

The IOL 3400 is made of material with a refractive index of 1.55 and provides a refractive power of about +22 D through the optic zone 3403. The optic zone has an anterior surface 3404 radius of curvature of 11.633 mm. The convex back optic zone surface 3405 has a radius of −61.536 mm. Central thickness of the IOL 3400 is 0.717 mm.

A control zone 3406 for controlling, reducing and/or eliminating PPD is provided in IOL 3400. The control zone 3406 comprises a front control surface 3407 and a back control surface 3408. The front/anterior control surface 3407 extends from the front optic-control junction 3409 to a front edge point 3412. The back/posterior control surface 3408 extends from the back optic-control junction 3410 to a back edge point 3413. The front edge point 3412 and back edge point 3413 is joined by the edge 3411 of the IOL.

The radial and axial positions of the front optic-control junction 3409, back optic-control junction 3410, front edge point 3412 and back edge point 3413 for this exemplary IOL are given in Table 4.

In this exemplary IOL the profiles of the front control surface 3407 and back control surface 3408 are defined using Bezier segments between control surface (3407 for front, 3408 for back) and the profile of the optic zone (3404 for front, 3405 for back) at their respective junction (3409 for front, 3410 for back). The tangent points for the front control curve are located at 3414 (front edge start tangent point) and 3415 (front optic-control junction end tangent point), and for the back control curve are located at 3416 (back edge start tangent point) and 3417 (back optic-control junction end tangent point). The form of the equation for the cubic Bezier segment has been defined in Eq. 2 above.

Table 4 gives the radial and axial coordinates (positions) of the relevant points.

The exemplary IOLs of FIG. 31 and FIG. 34, whose prescriptions are detailed in Table 3 and Table 4 respectively, are C0-continuous and C1-continuous at both their front optic-control junction and back optic-control junction. That is, at those points, their front and back optic surfaces meet (e.g., their optic and control surfaces are continuous or join with each other, without an abrupt change such as a ledge) and share common tangents with their respective front and back control surfaces.

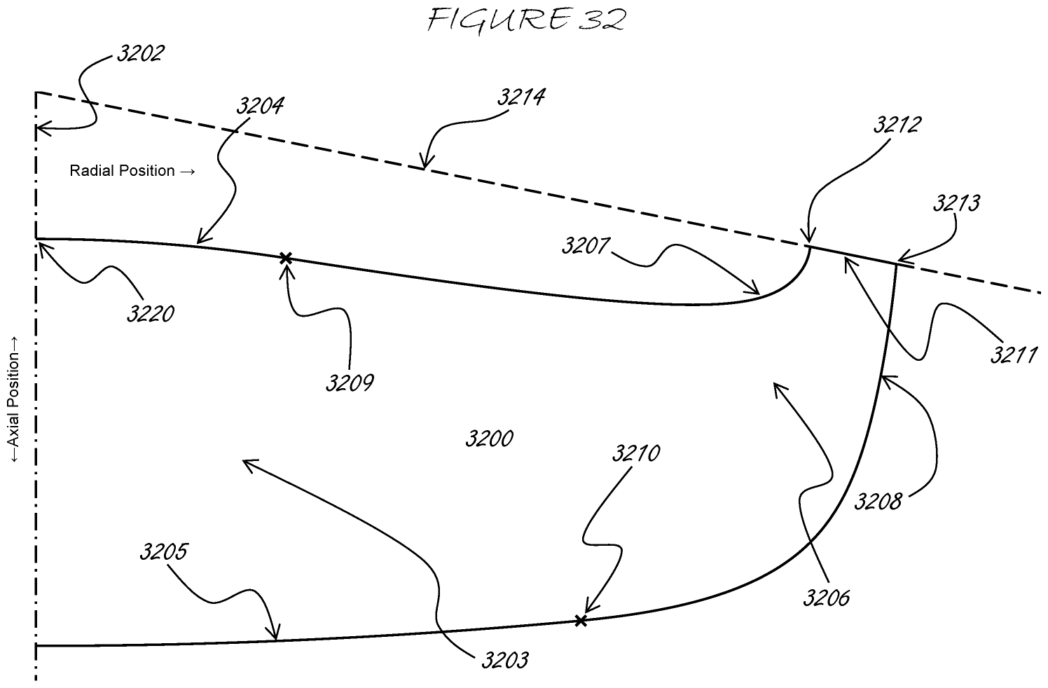

FIG. 32 is a schematic illustration of a half-meridian of an intraocular lens for reducing, minimizing, and/or eliminating peripheral pseudophakic dysphotopsia in accordance with certain embodiments. The prescription for this exemplary IOL for controlling PPD is given in Table 5 below.

TABLE 5

| | | | |
|---|---|---|---|
| Feature/Parameter | Unit | Value | Comment |

OPTIC ZONE

| | | | |
|---|---|---|---|
| refractive index | | 1.53 | |
| nominal power of IOL | (D) | +30 | when immersed in water |
| front optic radius of curvature | (mm) | 8.287 | |
| central thickness | (mm) | 0.968 | |
| back optic radius of curvature | (mm) | −29.927 | negative value means convex |
| | | | towards the back |

CONTROL ZONE

| | | | |
|---|---|---|---|
| front optic-control junction radial | (mm) | 0.871 | radial distance from axis of IOL |
| position | | | |
| front optic-control junction axial | (mm) | 0.046 | axial distance from vertex plane of |
| position | | | IOL |
| back optic-control junction radial | (mm) | 1.900 | radial distance from axis of IOL |
| position | | | |
| back optic-control junction axial | (mm) | 0.908 | axial distance from vertex plane of |
| position | | | IOL |
| angle of edge | (°) | 7.782 | relative to frontal plane |
| front edge point radial position | (mm) | 2.699 | radial distance from axis of IOL |
| front edge point axial position | (mm) | 0.019 | relative to apex of IOL (negative |
| | | | value means in front of IOL apex) |
| back edge point radial position | (mm) | 3.000 | radial distance from axis of IOL |
| back edge point axial position | (mm) | 0.060 | relative to apex of IOL |
| front control surface profile | | | |
| polynomial order | | 6 | apex of polynomial is at front |
| | | | edge point |
| 2nd order coefficient | | −6.218E+00 | |
| 6th order coefficient | | −3.144E+03 | |
| front polynomial tilt angle | (°) | 7.782 | tilt of axis of polynomial relative |
| | | | to frontal plane |
| back control surface profile | | | |
| polynomial order | | 6 | apex of polynomial is at back edge |
| | | | point |
| 2nd order coefficient | | −2.446E−01 | |
| 6th order coefficient | | −7.875E−01 | |
| back polynomial tilt angle | (°) | 7.782 | tilt of axis of polynomial relative |
| | | | to frontal plane |

The IOL 3200 is made of material with a refractive index of 1.53 and provides a refractive power of about +30 D through the optic zone 3203. The optic zone has an anterior surface 3204 radius of curvature of 8.287 mm. The convex back optic zone surface 3205 has a radius of −29.927 mm. Central thickness of the IOL 3200 is 0.968 mm.

The radial and axial positions of the front optic-control junction 3209, back optic-control junction 3210, front edge point 3212 and back edge point 3213 for this exemplary IOL are given in Table 5.

In this exemplary IOL, the profiles of the front control surface 3207 and back control surface 3208 are defined using polynomial equations which are then translated by select radial and axial distances and tilted by a select angle to provide continuity between a control surface (3207 for front, 3208 for back) and the profile of the optic zone (3204 for front, 3205 for back) at their respective junction (3209 for front, 3210 for back).

The form of the polynomial equation has been described with respect to Eq. 1 above. The prescribed polynomial is translated to place its apex at the edge point (3212 for front and 3213 for back) corresponding to point 2802 in FIG. 28. The polynomial is tilted by an angle relative to a frontal plane of the eye and IOL, and its axis of symmetry 3214 is coincident with the edge 3211 of the IOL.

Table 5 lists the values for the order k, 2ⁿd-order coefficient, k^{th}-order coefficient and the tilt angle for the front control surface profile and the back control surface profile. For both control surface profiles, the polynomial is translated to place its apex on their respective edge point and tilted so their axes 3214 are tilted to a select angle specified in Table 5.

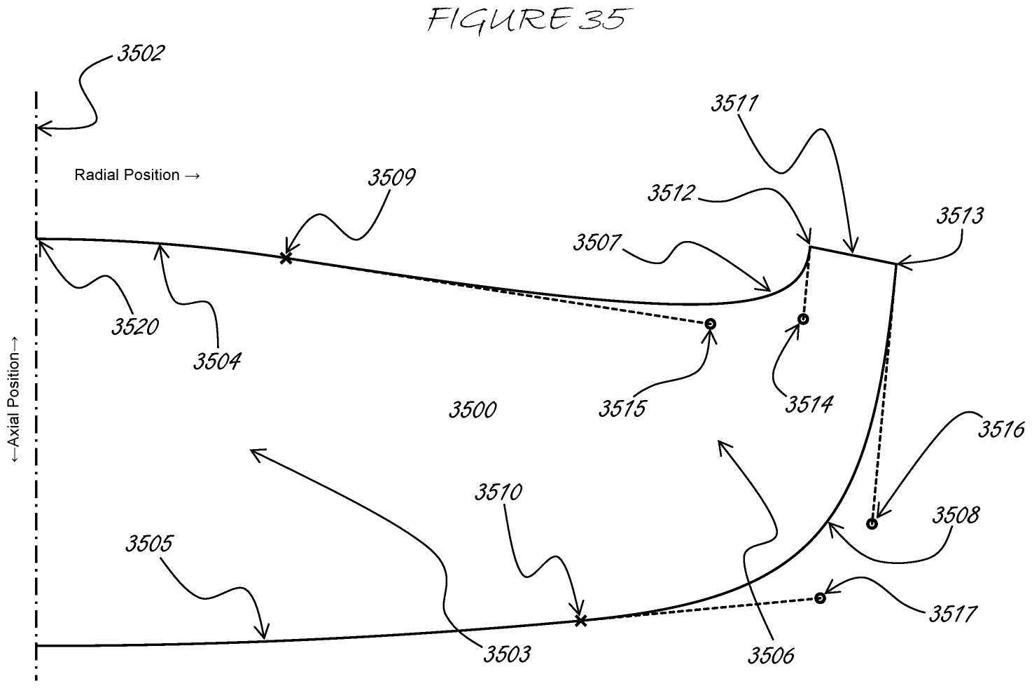

FIG. 35 is a schematic illustration of a half-meridian of an intraocular lens for reducing, minimizing, and/or eliminating peripheral pseudophakic dysphotopsia in accordance with certain embodiments. The prescription for this exemplary IOL with the same optic zone parameters as those for FIG. 32 but with control surface profile defined using Bezier segments is given in Table 6 below.

TABLE 6

| | | | |
|---|---|---|---|
| Feature/Parameter | Unit | Value | Comment |

OPTIC ZONE

| | | | |
|---|---|---|---|
| refractive index | | 1.53 | |
| nominal power of IOL | (D) | +30 | when immersed in water |
| front optic radius of curvature | (mm) | 8.287 | |
| central thickness | (mm) | 0.968 | |
| back optic radius of curvature | (mm) | −29.927 | negative value means convex |
| | | | towards the back |

CONTROL ZONE

| | | | |
|---|---|---|---|
| front optic-control junction radial | (mm) | 0.871 | radial distance from axis of IOL |
| position | | | |
| front optic-control junction axial | (mm) | 0.046 | axial distance from vertex plane of |
| position | | | IOL |
| back optic-control junction radial | (mm) | 1.900 | radial distance from axis of IOL |
| position | | | |
| back optic-control junction axial | (mm) | 0.908 | axial distance from vertex plane of |
| position | | | IOL |
| angle of edge | (°) | 7.782 | relative to frontal plane |
| front edge point radial position | (mm) | 2.699 | radial distance from axis of IOL |
| front edge point axial position | (mm) | 0.019 | relative to apex of IOL (negative |
| | | | value means in front of IOL apex) |
| back edge point radial position | (mm) | 3.000 | radial distance from axis of IOL |
| back edge point axial position | (mm) | 0.060 | relative to apex of IOL |
| front control surface profile | | | |
| front edge Bezier | (mm) | 2.676 | radial distance from axis of IOL |
| tangent point radial position | | | |
| front edge Bezier | (mm) | 0.191 | axial distance from vertex plane of |
| tangent point axial position | | | IOL |
| front junction Bezier | (mm) | 2.352 | radial distance from axis of IOL |
| tangent point radial position | | | |
| front junction Bezier | (mm) | 0.202 | axial distance from vertex plane of |
| tangent point axial position | | | IOL |
| back control surface profile | | | |
| back edge Bezier | (mm) | 2.916 | radial distance from axis of IOL |
| tangent point radial position | | | |
| back edge Bezier | (mm) | 0.678 | axial distance from vertex plane of |
| tangent point axial position | | | IOL |
| back junction Bezier | (mm) | 2.735 | radial distance from axis of IOL |
| tangent point radial position | | | |
| back junction Bezier | (mm) | 0.855 | axial distance from vertex plane of |
| tangent point axial position | | | IOL |

The IOL 3500 is made of material with a refractive index of 1.53 and provides a refractive power of about +30 D through the optic zone 3503. The optic zone has an anterior surface 3504 radius of curvature of 8.287 mm. The convex back optic zone surface 3505 has a radius of −29.927 mm. Central thickness of the IOL 3500 is 0.968 mm.

A control zone 3506 for controlling, reducing and/or eliminating PPD is provided in IOL 3500. The control zone 3506 comprises a front control surface 3507 and a back control surface 3508. The front/anterior control surface 3507 extends from the front optic-control junction 3509 to a front edge point 3512. The back/posterior control surface 3508 extends from the back optic-control junction 3510 to a back edge point 3513. The front edge point 3512 and back edge point 3513 is joined by the edge 3511 of the IOL.

The radial and axial positions of the front optic-control junction 3509, back optic-control junction 3510, front edge point 3512 and back edge point 3513 for this exemplary IOL are given in Table 6.

In this exemplary IOL the profiles of the front control surface 3507 and back control surface 3508 are defined using Bezier segments between control surface (3507 for front, 3508 for back) and the profile of the optic zone (3504 for front, 3505 for back) at their respective junction (3509 for front, 3510 for back). The tangent points for the front control curve are located at 3514 (front edge start tangent point) and 3515 (front optic-control junction end tangent point), and for the back control curve are located at 3516 (back edge start tangent point) and 3517 (back optic-control junction end tangent point). The form of the equation for the cubic Bezier segment is defined in Eq. 2 above.

Table 6 gives the radial and axial coordinates (positions) of the relevant points.

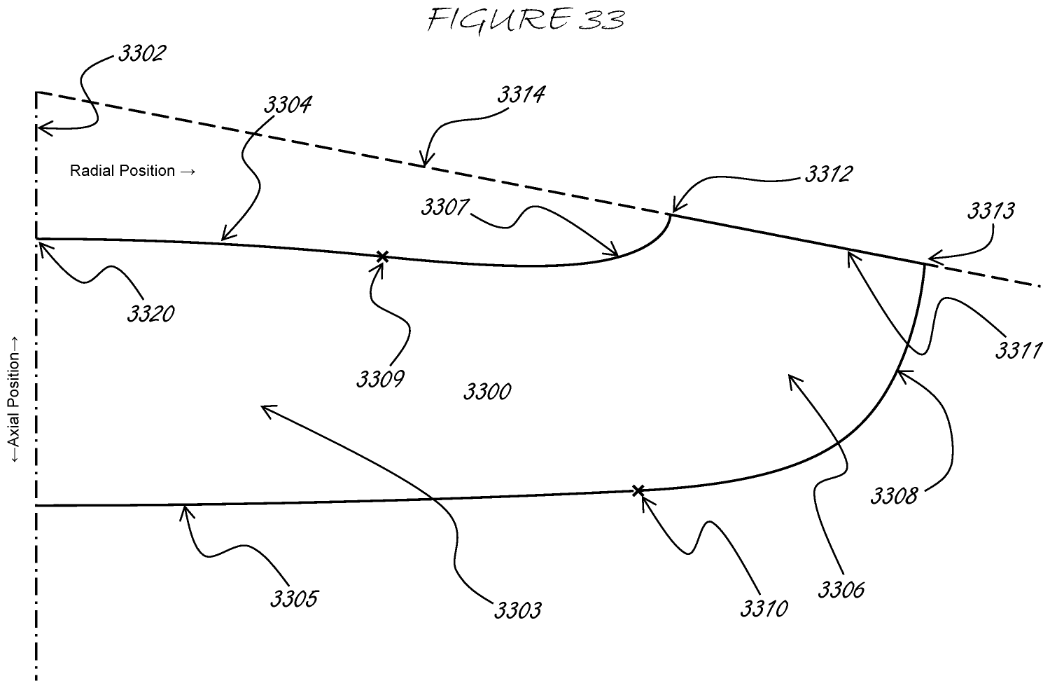

FIG. 33 is a schematic illustration of a half-meridian of an intraocular lens for reducing, minimizing, and/or eliminating peripheral pseudophakic dysphotopsia in accordance with certain embodiments. The prescription for this exemplary IOL for controlling PPD is given in Table 7 below.

TABLE 7

| | | | |
|---|---|---|---|
| Feature/Parameter | Unit | Value | Comment |

OPTIC ZONE

| | | | |
|---|---|---|---|
| refractive index | | 1.47 | |
| nominal power of IOL | (D) | +10 | when immersed in water |
| front optic radius of curvature | (mm) | 17.302 | |
| central thickness | (mm) | 0.634 | |
| back optic radius of curvature | (mm) | −62.350 | negative value means convex |
| | | | towards the back |

CONTROL ZONE

| | | | |
|---|---|---|---|
| front optic-control junction radial | (mm) | 1.208 | radial distance from axis of IOL |
| position | | | |
| front optic-control junction axial | (mm) | 0.042 | axial distance from vertex plane |
| position | | | of IOL |
| back optic-control junction radial | (mm) | 2.100 | radial distance from axis of IOL |
| position | | | |
| back optic-control junction axial | (mm) | 0.599 | axial distance from vertex plane |
| position | | | of IOL |
| angle of edge | (°) | 7.534 | relative to frontal plane |
| front edge point radial position | (mm) | 2.213 | radial distance from axis of IOL |
| front edge point axial position | (mm) | −0.057 | relative to apex of IOL (negative |
| | | | value means in front of IOL |
| | | | apex) |
| back edge point radial position | (mm) | 3.100 | radial distance from axis of IOL |
| back edge point axial position | (mm) | 0.060 | relative to apex of IOL |
| front control surface profile | | | |
| polynomial order | | 6 | apex of polynomial is at front |
| | | | edge point |
| 2nd order coefficient | | −1.019E+01 | |
| 4th order coefficient | | −2.947E+03 | |
| front polynomial tilt angle | (°) | 7.534 | tilt of axis of polynomial relative |
| | | | to frontal plane |
| back control surface profile | | | |
| polynomial order | | 6 | apex of polynomial is at back |
| | | | edge point |
| 2nd order coefficient | | −8.674E−01 | |
| 6th order coefficient | | −6.205E+00 | |
| back polynomial tilt angle | (°) | 7.534 | tilt of axis of polynomial relative |
| | | | to frontal plane |

The IOL 3300 is made of material with a refractive index of 1.47 and provides a refractive power of about +10 D through the optic zone 3303. The optic zone has an anterior surface 3304 radius of curvature of 17.302 mm. The convex back optic zone surface 3305 has a radius of −62.350 mm. Central thickness of the IOL 3300 is 0.634 mm.

The radial and axial positions of the front optic-control junction 3309, back optic-control junction 3310, front edge point 3312 and back edge point 3313 for this exemplary IOL are given in Table 7.

In this exemplary IOL, the profiles of the front control surface 3307 and back control surface 3308 are defined using polynomial equations which are then translated by select radial and axial distances and tilted by a select angle to provide continuity between a control surface (3307 for front, 3308 for back) and the profile of the optic zone (3304 for front, 3305 for back) at their respective junction (3309 for front, 3310 for back).

The form of the polynomial equation has been described above with respect to Eq. 1. The prescribed polynomial is translated to place its apex at the edge point (3312 for front and 3313 for back) corresponding to point 2802 in FIG. 28. The polynomial is tilted by an angle relative to a frontal plane of the eye and IOL, and its axis of symmetry 3314 is coincident with the edge 3311 of the IOL.

Table 7 lists the values for the order k, 2^{nd}-order coefficient, k^{th}-order coefficient and the tilt angle for the front control surface profile and the back control surface profile. For both control surface profiles, the polynomial is translated to place its apex on their respective edge point and tilted so their axes 3314 are tilted to a select angle specified in Table 7.

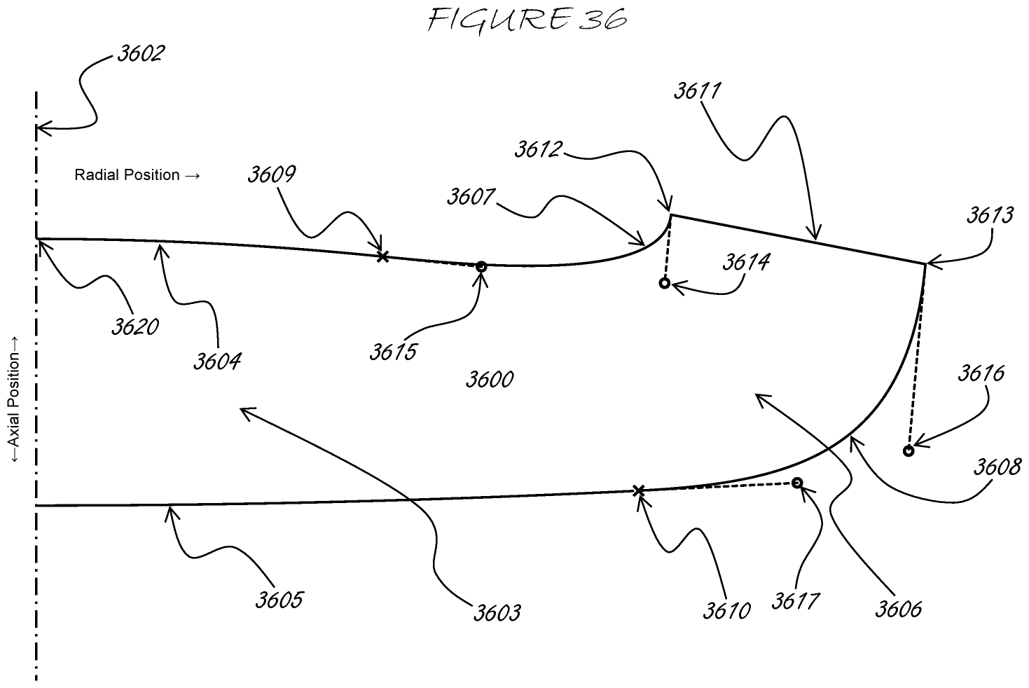

FIG. 36 is a schematic illustration of a half-meridian of an intraocular lens for reducing, minimizing, and/or eliminating peripheral pseudophakic dysphotopsia in accordance with certain embodiments. The prescription for this exemplary IOL with the same optic zone parameters as those for FIG. 33 but with control surface profile defined using Bezier segments is given in Table 8 below.

TABLE 8

| | | | |
|---|---|---|---|
| Feature/Parameter | Unit | Value | Comment |

OPTIC ZONE

| | | | |
|---|---|---|---|
| refractive index | | 1.47 | |
| nominal power of IOL | (D) | +10 | when immersed in water |
| front optic radius of curvature | (mm) | 17.302 | |
| central thickness | (mm) | 0.634 | |
| back optic radius of curvature | (mm) | −62.350 | negative value means convex |
| | | | towards the back |

CONTROL ZONE

| | | | |
|---|---|---|---|
| front optic-control junction radial | (mm) | 1.208 | radial distance from axis of IOL |
| position | | | |
| front optic-control junction axial | (mm) | 0.042 | axial distance from vertex plane of |
| position | | | IOL |
| back optic-control junction radial | (mm) | 2.100 | radial distance from axis of IOL |
| position | | | |
| back optic-control junction axial | (mm) | 0.599 | axial distance from vertex plane of |
| position | | | IOL |
| angle of edge | (°) | 7.534 | relative to frontal plane |
| front edge point radial position | (mm) | 2.213 | radial distance from axis of IOL |
| front edge point axial position | (mm) | −0.057 | relative to apex of IOL (negative |
| | | | value means in front of IOL apex) |
| back edge point radial position | (mm) | 3.100 | radial distance from axis of IOL |
| back edge point axial position | (mm) | 0.060 | relative to apex of IOL |
| front control surface profile | | | |
| front edge Bezier | (mm) | 2.191 | radial distance from axis of IOL |
| tangent point radial position | | | |
| front edge Bezier | (mm) | 0.106 | axial distance from vertex plane of |
| tangent point axial position | | | IOL |
| front junction Bezier | (mm) | 1.552 | radial distance from axis of IOL |
| tangent point radial position | | | |
| front junction Bezier | (mm) | 0.066 | axial distance from vertex plane of |
| tangent point axial position | | | IOL |
| back control surface profile | | | |
| back edge Bezier | (mm) | 3.041 | radial distance from axis of IOL |
| tangent point radial position | | | |
| back edge Bezier | (mm) | 0.505 | axial distance from vertex plane of |
| tangent point axial position | | | IOL |
| back junction Bezier | (mm) | 2.653 | radial distance from axis of IOL |
| tangent point radial position | | | |
| back junction Bezier | (mm) | 0.580 | axial distance from vertex plane of |
| tangent point axial position | | | IOL |

The IOL 3600 is made of material with a refractive index of 1.47 and provides a refractive power of about +10 D through the optic zone 3603. The optic zone has an anterior surface 3604 radius of curvature of 17.302 mm. The convex back optic zone surface 3605 has a radius of −62.350 mm. Central thickness of the IOL 3600 is 0.634 mm.

A control zone 3606 for controlling, reducing and/or eliminating PPD is provided in IOL 3600. The control zone 3606 comprises a front control surface 3607 and a back control surface 3608. The front/anterior control surface 3607 extends from the front optic-control junction 3609 to a front edge point 3612. The back/posterior control surface 3608 extends from the back optic-control junction 3610 to a back edge point 3613. The front edge point 3612 and back edge point 3613 is joined by the edge 3611 of the IOL.

The radial and axial positions of the front optic-control junction 3609, back optic-control junction 3610, front edge point 3612 and back edge point 3613 for this exemplary IOL are given in Table 8.

In this exemplary IOL the profiles of the front control surface 3607 and back control surface 3608 are defined using Bezier segments between control surface (3607 for front, 3608 for back) and the profile of the optic zone (3604 for front, 3605 for back) at their respective junction (3609 for front, 3610 for back). The tangent points for the front control curve are located at 3614 (front edge start tangent point) and 3615 (front optic-control junction end tangent point), and for the back control curve are located at 3616 (back edge start tangent point) and 3617 (back optic-control junction end tangent point). The form of the equation for the cubic Bezier segment is defined in Eq. 2 above.

Table 8 gives the radial and axial coordinates (positions) of the relevant points.

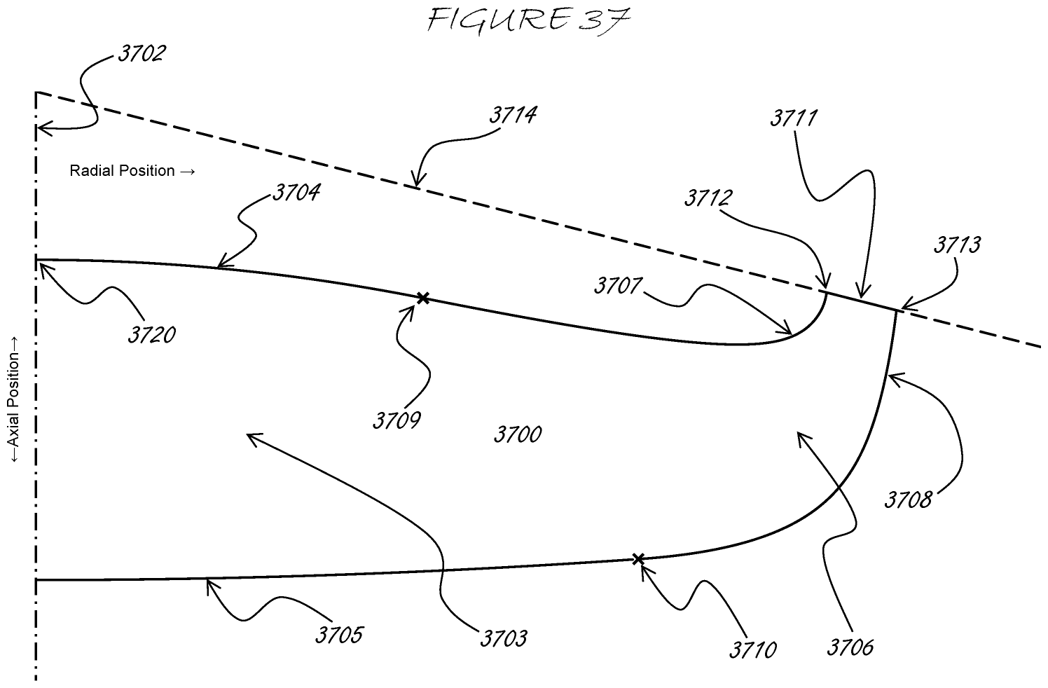

FIG. 37 is a schematic illustration of a half-meridian of an intraocular lens for reducing, minimizing, and/or eliminating peripheral pseudophakic dysphotopsia in accordance with certain embodiments. The prescription for this further exemplary IOL for controlling peripheral pseudophakic dysphotopsia is given in Table 9 below.

TABLE 9

| | | | |
|---|---|---|---|
| Feature/Parameter | Unit | Value | Comment |

OPTIC ZONE

| | | | |
|---|---|---|---|
| refractive index | | 1.54 | |
| nominal power of IOL | (D) | +25 | when immersed in water |
| front optic radius of curvature | (mm) | 10.058 | |
| central thickness | (mm) | 0.761 | |
| back optic radius of curvature | (mm) | −44.569 | negative value means convex |
| | | | towards the back |

CONTROL ZONE

| | | | |
|---|---|---|---|
| front optic-control junction radial | (mm) | 1.350 | radial distance from axis of IOL |
| position | | | |
| front optic-control junction axial | (mm) | 0.091 | axial distance from vertex plane |
| position | | | of IOL |
| back optic-control junction radial | (mm) | 2.100 | radial distance from axis of IOL |
| position | | | |
| back optic-control junction axial | (mm) | 0.711 | axial distance from vertex plane |
| position | | | of IOL |
| angle of edge | (°) | 9.834 | relative to frontal plane |
| front edge radial position | (mm) | 2.756 | radial distance from axis of IOL |
| front edge axial position | (mm) | 0.078 | relative to apex of IOL (negative |
| | | | value means in front of IOL |
| | | | apex) |
| back edge radial position | (mm) | 3.000 | radial distance from axis of IOL |
| back edge axial position | (mm) | 0.120 | relative to apex of IOL |
| front control surface profile | | | |
| polynomial order | | 5 | apex of polynomial is at front |
| | | | edge point |
| 2nd order coefficient | | −5.720E+00 | |
| 6th order coefficient | | −3.854E+03 | |
| front polynomial tilt angle | (°) | 9.834 | tilt of axis of polynomial relative |
| | | | to frontal plane |
| back control surface profile | | | |
| polynomial order | | 5 | apex of polynomial is at back |
| | | | edge point |
| 2nd order coefficient | | −3.785E−01 | |
| 5th order coefficient | | −2.678E+00 | |
| back polynomial tilt angle | (°) | 9.834 | tilt of axis of polynomial relative |
| | | | to frontal plane |

The IOL 3700 is made of material with a refractive index of 1.54 and provides a refractive power of about +25 D through the optic zone 3703. The optic zone has an anterior surface 3704 radius of curvature of 10.058 mm. The convex back optic zone surface 3705 has a radius of −44.569 mm. Central thickness of the IOL 3700 is 0.761 mm.

The radial and axial positions of the front optic-control junction 3709, back optic-control junction 3710, front edge point 3712 and back edge point 3713 for this exemplary IOL are given in Table 9.

In this exemplary IOL, the profiles of the front control surface 3707 and back control surface 3708 are defined using polynomial equations which are then translated by select radial and axial distances and tilted by a select angle to provide continuity between a control surface (3707 for front, 3708 for back) and the profile of the optic zone (3704 for front, 3705 for back) at their respective junction (3709 for front, 3710 for back).

The form of the polynomial equation has been described above with respect to Eq. 1. The prescribed polynomial is translated to place its apex at the edge point (3712 for front and 3713 for back) corresponding to point 2802 in FIG. 28. The polynomial is tilted by an angle relative to a frontal plane of the eye and IOL, and its axis of symmetry 3714 is coincident with the edge 3711 of the IOL.

Table 9 lists the values for the order k, 2ⁿd-order coefficient, k^{th}-order coefficient and the tilt angle for the front control surface profile and the back control surface profile. For both control surface profiles, the polynomial is translated to place its apex on their respective edge point and tilted so their axes 3714 are tilted to a select angle specified in Table 9.

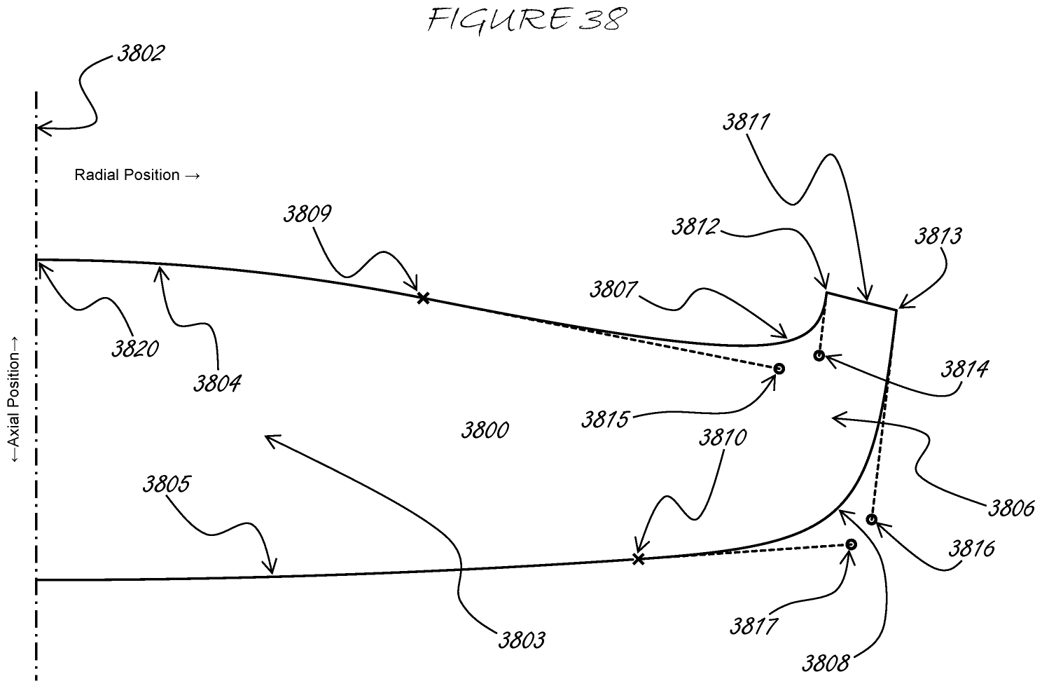

FIG. 38 is a schematic illustration of a half-meridian of an intraocular lens for reducing, minimizing, and/or eliminating peripheral pseudophakic dysphotopsia in accordance with certain embodiments. The prescription for this exemplary IOL with the same optic zone parameters as those for FIG. 37 but with control surface profile defined using Bezier segments is given in Table 10 below.

TABLE 10

| | | | |
|---|---|---|---|
| Feature/Parameter | Unit | Value | Comment |

OPTIC ZONE

| | | | |
|---|---|---|---|
| refractive index | | 1.54 | |
| nominal power of IOL | (D) | +25 | when immersed in water |
| front optic radius of | (mm) | 10.058 | |
| curvature | | | |
| central thickness | (mm) | 0.761 | |
| back optic radius of | (mm) | −44.569 | negative value means |
| curvature | | | convex towards the back |

CONTROL ZONE

| | | | |
|---|---|---|---|
| front optic-control junction | (mm) | 1.350 | radial distance from axis |
| radial position | | | of IOL |
| front optic-control junction | (mm) | 0.091 | axial distance from vertex |
| axial position | | | plane of IOL |
| back optic-control junction | (mm) | 2.100 | radial distance from axis |
| radial position | | | of IOL |
| back optic-control junction | (mm) | 0.711 | axial distance from vertex |
| axial position | | | plane of IOL |
| angle of edge | (°) | 9.834 | relative to frontal plane |
| front edge radial position | (mm) | 2.756 | radial distance from axis |
| | | | of IOL |
| front edge axial position | (mm) | 0.078 | relative to apex of IOL |
| | | | (negative value means in |
| | | | front of IOL apex) |
| back edge radial position | (mm) | 3.000 | radial distance from axis |
| | | | of IOL |
| back edge axial position | (mm) | 0.120 | relative to apex of IOL |
| front control surface profile | | | |
| front edge Bezier | (mm) | 2.730 | radial distance from axis |
| tangent point radial position | | | of IOL |
| front edge Bezier | (mm) | 0.228 | axial distance from vertex |
| tangent point axial position | | | plane of IOL |
| front junction Bezier | (mm) | 2.591 | radial distance from axis |
| tangent point radial position | | | of IOL |
| front junction Bezier | (mm) | 0.259 | axial distance from vertex |
| tangent point axial position | | | plane of IOL |
| back control surface profile | | | |
| back edge Bezier | (mm) | 2.914 | radial distance from axis |
| tangent point radial position | | | of IOL |
| back edge Bezier | (mm) | 0.617 | axial distance from vertex |
| tangent point axial position | | | plane of IOL |
| back junction Bezier | (mm) | 2.843 | radial distance from axis |
| tangent point radial position | | | of IOL |
| back junction Bezier | (mm) | 0.676 | axial distance from vertex |
| tangent point axial position | | | plane of IOL |

The IOL 3800 is made of material with a refractive index of 1.54 and provides a refractive power of about +25 D through the optic zone 3803. The optic zone has an anterior surface 3804 radius of curvature of 10.058 mm. The convex back optic zone surface 3805 has a radius of 44.569 mm. Central thickness of the IOL 3800 is 0.761 mm.

A control zone 3806 for controlling, reducing and/or eliminating PPD is provided in IOL 3800. The control zone 3806 comprises a front control surface 3807 and a back control surface 3808. The front/anterior control surface 3807 extends from the front optic-control junction 3809 to a front edge point 3812. The back/posterior control surface 3808 extends from the back optic-control junction 3810 to a back edge point 3813. The front edge point 3812 and back edge point 3813 is joined by the edge 3811 of the IOL.

The radial and axial positions of the front optic-control junction 3809, back optic-control junction 3810, front edge point 3812 and back edge point 3813 for this exemplary IOL are given in Table 10.

In this exemplary IOL the profiles of the front control surface 3807 and back control surface 3808 are defined using Bezier segments between control surface (3807 for front, 3808 for back) and the profile of the optic zone (3804 for front, 3805 for back) at their respective junction (3809 for front, 3810 for back). The tangent points for the front control curve are located at 3814 (front edge start tangent point) and 3815 (front optic-control junction end tangent point), and for the back control curve are located at 3816 (back edge start tangent point) and 3817 (back optic-control junction end tangent point). The form of the equation for the cubic Bezier segment is defined in Eq. 2.

Table 10 gives the radial and axial coordinates (positions) of the relevant points.

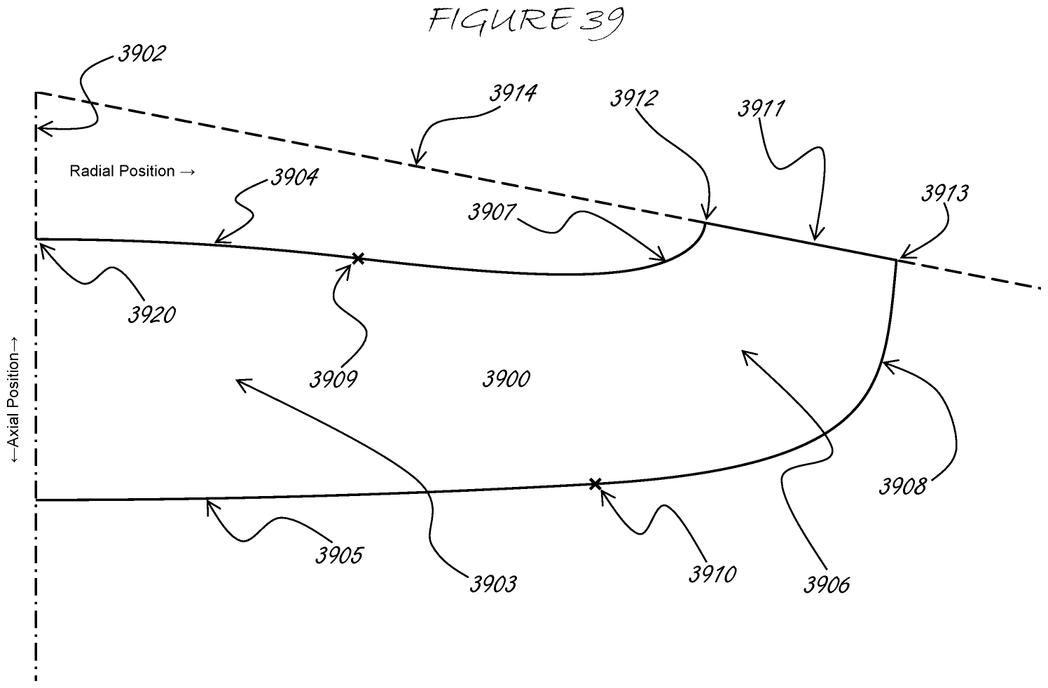

FIG. 39 is a schematic illustration of a half-meridian of an intraocular lens for reducing, minimizing, and/or eliminating peripheral pseudophakic dysphotopsia in accordance with certain embodiments. The prescription for this exemplary IOL for controlling PPD is given in Table 11 below.

TABLE 11

| | | | |
|---|---|---|---|
| Feature/Parameter | Unit | Value | Comment |

OPTIC ZONE

| | | | |
|---|---|---|---|
| refractive index | | 1.47 | |
| nominal power of IOL | (D) | +12.5 | when immersed in water |
| front optic radius of curvature | (mm) | 13.830 | |
| central thickness | (mm) | 0.620 | |
| back optic radius of curvature | (mm) | −50.000 | negative value means convex |
| | | | towards the back |

CONTROL ZONE

| | | | |
|---|---|---|---|
| front optic-control junction radial | (mm) | 1.123 | radial distance from axis of IOL |
| position | | | |
| front optic-control junction axial | (mm) | 0.046 | axial distance from vertex plane |
| position | | | of IOL |
| back optic-control junction radial | (mm) | 1.950 | radial distance from axis of IOL |
| position | | | |
| back optic-control junction axial | (mm) | 0.582 | axial distance from vertex plane |
| position | | | of IOL |
| angle of edge | (°) | 7.595 | relative to frontal plane |
| front edge point radial position | (mm) | 2.333 | radial distance from axis of IOL |
| front edge point axial position | (mm) | −0.039 | relative to apex of IOL (negative |
| | | | value means in front of IOL |
| | | | apex) |
| back edge point radial position | (mm) | 3.000 | radial distance from axis of IOL |
| back edge point axial position | (mm) | 0.050 | relative to apex of IOL |
| front control surface profile | | | |
| polynomial order | | 4 | apex of polynomial is at front |
| | | | edge point |
| 2nd order coefficient | | −1.000E+01 | |
| 6th order coefficient | | −2.829E+03 | |
| front polynomial tilt angle | (°) | 7.595 | tilt of axis of polynomial relative |
| | | | to frontal plane |
| back control surface profile | | | |
| polynomial order | | 4 | apex of polynomial is at back |
| | | | edge point |
| 2nd order coefficient | | −4.251E−02 | |
| 4th order coefficient | | −4.835E+00 | |
| back polynomial tilt angle | (°) | 7.595 | tilt of axis of polynomial relative |
| | | | to frontal plane |

The IOL 3900 is made of material with a refractive index of 1.47 and provides a refractive power of +12.5 D through the optic zone 3903. The optic zone has an anterior surface 3904 radius of curvature of 13.830 mm. The convex back optic zone surface 3905 has a radius of −50.0 mm. Central thickness of the IOL 3900 is 0.62 mm.

The radial and axial positions of the front optic-control junction 3909, back optic-control junction 3910, front edge point 3912 and back edge point 3913 for this exemplary IOL are given in Table 11.

In this exemplary IOL, the profiles of the front control surface 3907 and back control surface 3908 are defined using polynomial equations which are then translated by select radial and axial distances and tilted by a select angle to provide continuity between a control surface (3907 for front, 3908 for back) and the profile of the optic zone (3904 for front, 3905 for back) at their respective junction (3909 for front, 3910 for back).

The form of the polynomial equation has been described above with respect to Eq. 1. The prescribed polynomial is translated to place its apex at the edge point (3912 for front and 3913 for back) corresponding to point 2802 in FIG. 28. The polynomial is tilted by an angle relative to a frontal plane of the eye and IOL, and its axis of symmetry 3914 is coincident with the edge 3911 of the IOL.

Table 11 lists the values for the order k, 2nd-order coefficient, k^{th}-order coefficient and the tilt angle for the front control surface profile and the back control surface profile. For both control surface profiles, the polynomial is translated to place its apex on their respective edge point and tilted so their axes 3914 are tilted to a select angle specified in Table 11.

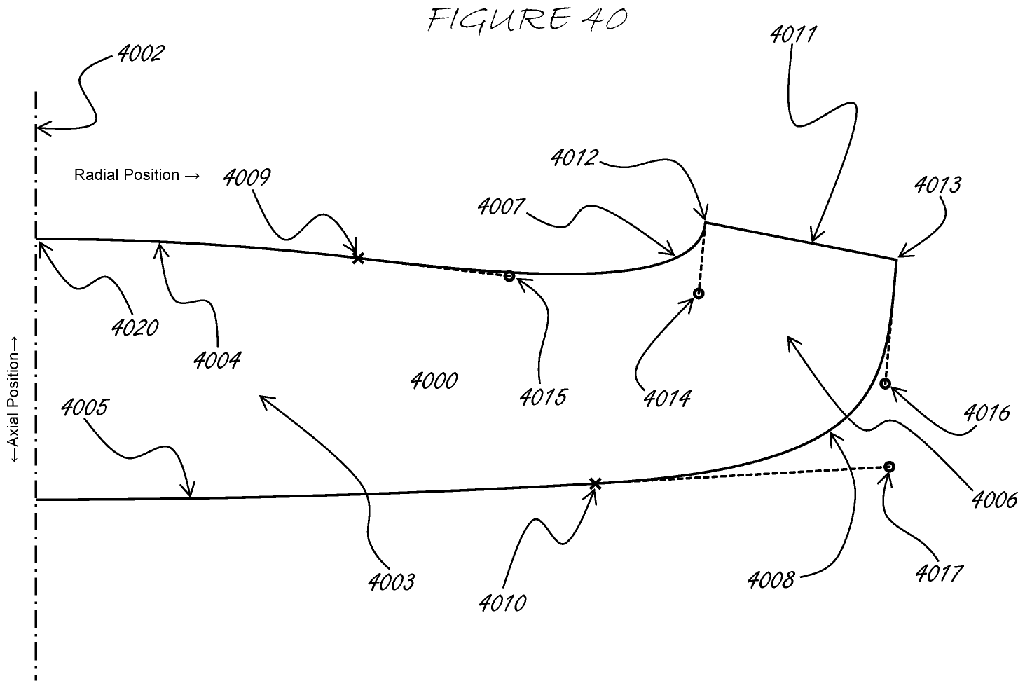

FIG. 40 is a schematic illustration of a half-meridian of an intraocular lens for reducing, minimizing, and/or eliminating peripheral pseudophakic dysphotopsia in accordance with certain embodiments. The prescription for this exemplary IOL with the same optic zone parameters as those for FIG. 39 but with control surface profile defined using Bezier segments is given in Table 12 below.

TABLE 12

| | | | |
|---|---|---|---|
| Feature/Parameter | Unit | Value | Comment |

OPTIC ZONE

| | | | |
|---|---|---|---|
| refractive index | | 1.47 | |
| nominal power of IOL | (D) | +12.5 | when immersed in water |
| front optic radius of curvature | (mm) | 13.830 | |
| central thickness | (mm) | 0.620 | |
| back optic radius of curvature | (mm) | −50.000 | negative value means convex |
| | | | towards the back |

CONTROL ZONE

| | | | |
|---|---|---|---|
| front optic-control junction radial | (mm) | 1.123 | radial distance from axis of IOL |
| position | | | |
| front optic-control junction axial | (mm) | 0.046 | axial distance from vertex plane of |
| position | | | IOL |
| back optic-control junction radial | (mm) | 1.950 | radial distance from axis of IOL |
| position | | | |
| back optic-control junction axial | (mm) | 0.582 | axial distance from vertex plane of |
| position | | | IOL |
| angle of edge | (°) | 7.595 | relative to frontal plane |
| front edge point radial position | (mm) | 2.333 | radial distance from axis of IOL |
| front edge point axial position | (mm) | −0.039 | relative to apex of IOL (negative |
| | | | value means in front of IOL apex) |
| back edge point radial position | (mm) | 3.000 | radial distance from axis of IOL |
| back edge point axial position | (mm) | 0.050 | relative to apex of IOL |
| front control surface profile | | | |
| front edge Bezier | (mm) | 2.310 | radial distance from axis of IOL |
| tangent point radial position | | | |
| front edge Bezier | (mm) | 0.130 | axial distance from vertex plane of |
| tangent point axial position | | | IOL |
| front junction Bezier | (mm) | 1.650 | radial distance from axis of IOL |
| tangent point radial position | | | |
| front junction Bezier | (mm) | 0.089 | axial distance from vertex plane of |
| tangent point axial position | | | IOL |
| back control surface profile | | | |
| back edge Bezier | (mm) | 2.961 | radial distance from axis of IOL |
| tangent point radial position | | | |
| back edge Bezier | (mm) | 0.344 | axial distance from vertex plane of |
| tangent point axial position | | | IOL |
| back junction Bezier | (mm) | 2.975 | radial distance from axis of IOL |
| tangent point radial position | | | |
| back junction Bezier | (mm) | 0.542 | axial distance from vertex plane of |
| tangent point axial position | | | IOL |

The IOL 4000 is made of material with a refractive index of 1.47 and provides a refractive power of about +12.5 D through the optic zone 4003. The optic zone has an anterior surface 4004 radius of curvature of 13.830 mm. The convex back optic zone surface 4005 has a radius of −50.0 mm. Central thickness of the IOL 4000 is 0.62 mm.

A control zone 4006 for controlling, reducing and/or eliminating PPD is provided in IOL 4000. The control zone 4006 comprises a front control surface 4007 and a back control surface 4008. The front/anterior control surface 4007 extends from the front optic-control junction 4009 to a front edge point 4012. The back/posterior control surface 4008 extends from the back optic-control junction 4010 to a back edge point 4013. The front edge point 4012 and back edge point 4013 is joined by the edge 4011 of the IOL.

The radial and axial positions of the front optic-control junction 4009, back optic-control junction 4010, front edge point 4012 and back edge point 4013 for this exemplary IOL are given in Table 12.

In this exemplary IOL the profiles of the front control surface 4007 and back control surface 4008 are defined using Bezier segments between control surface (4007 for front, 4008 for back) and the profile of the optic zone (4004 for front, 4005 for back) at their respective junction (4009 for front, 4010 for back). The tangent points for the front control curve are located at 4014 (front edge start tangent point) and 4015 (front optic-control junction end tangent point), and for the back control curve are located at 4016 (back edge start tangent point) and 4017 (back optic-control junction end tangent point). The form of the equation for the cubic Bezier segment is defined in Eq. 2 above.

Table 12 gives the radial and axial coordinates (positions) of the relevant points.

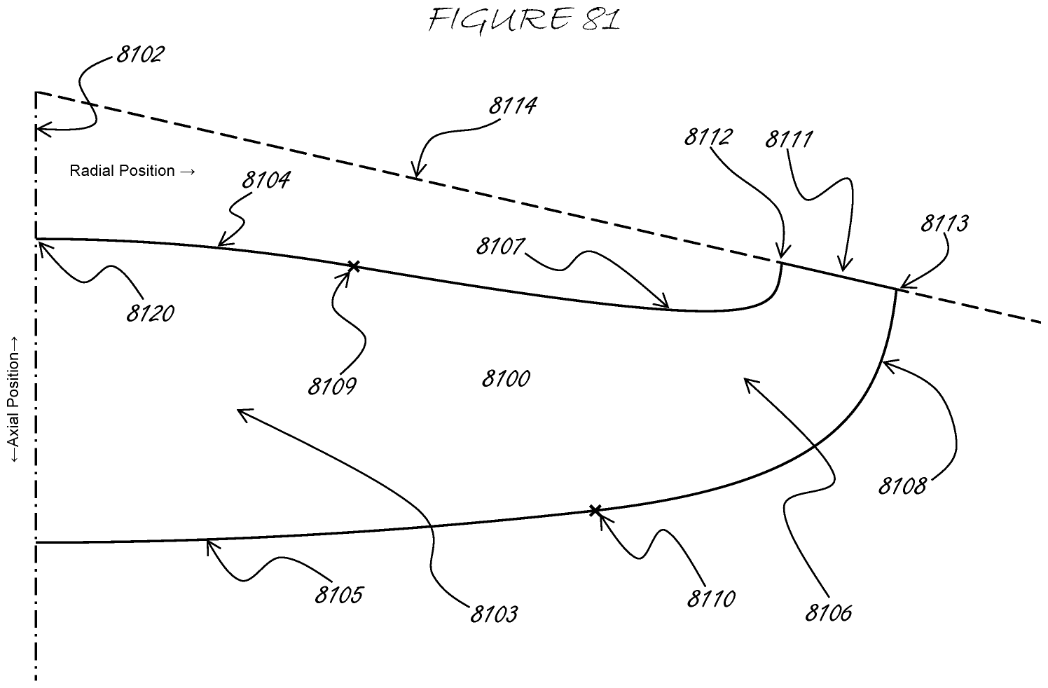

FIG. 81 is a schematic illustration of a half-meridian of an intraocular lens for reducing, minimizing, and/or eliminating peripheral pseudophakic dysphotopsia in accordance with certain embodiments. The prescription for this exemplary IOL for controlling PPD is given in Table 13 below.

TABLE 13

| | | | |
|---|---|---|---|
| Feature/Parameter | Unit | Value | Comment |

OPTIC ZONE

| | | | |
|---|---|---|---|
| refractive index | | 1.47 | |
| nominal power of IOL | (D) | +20.00 | when immersed in water |
| front optic central | (mm) | 9.330 | |
| radius of curvature | | | |
| front optic conic constant | | −2.950 | |
| central thickness | (mm) | 0.722 | |
| back optic central | (mm) | −24.590 | negative value means convex |
| radius of curvature | | | towards the back |
| back optic conic constant | | −11.220 | |

CONTROL ZONE

| | | | |
|---|---|---|---|
| front optic-control junction radial | (mm) | 1.107 | radial distance from axis of IOL |
| position | | | |
| front optic-control junction axial | (mm) | 0.065 | axial distance from vertex plane |
| position | | | of IOL |
| back optic-control junction radial | (mm) | 1.950 | radial distance from axis of IOL |
| position | | | |
| back optic-control junction axial | (mm) | 0.646 | axial distance from vertex plane |
| position | | | of IOL |
| angle of edge | (°) | 8.904 | relative to frontal plane |
| front edge radial position | (mm) | 2.600 | radial distance from axis of IOL |
| front edge axial position | (mm) | 0.057 | relative to apex of IOL (negative |
| | | | value means in front of IOL |
| | | | apex) |
| back edge radial position | (mm) | 3.000 | radial distance from axis of IOL |
| back edge axial position | (mm) | 0.120 | relative to apex of IOL |
| front control surface profile | | | |
| polynomial order | | 4.25 | apex of polynomial is at front |
| | | | edge point |
| 2-nd order coefficient | | −8.777E−01 | |
| 4.25-th order coefficient | | −6.253E+02 | |
| front polynomial tilt angle | (°) | 8.904 | tilt of axis of polynomial relative |
| | | | to frontal plane |
| back control surface profile | | | |
| polynomial order | | 3.5 | apex of polynomial is at back |
| | | | edge point |
| 2nd order coefficient | | −6.598E−01 | |
| 3.50-th order coefficient | | −2.477E+00 | |
| back polynomial tilt angle | (°) | 8.904 | tilt of axis of polynomial relative |
| | | | to frontal plane |

The IOL 8100 is made of material with a refractive index of 1.47 and provides a refractive power of about +20 D through the optic zone 8103. The optic zone has an anterior surface 8104 central radius of curvature of 9.33 mm and is aspheric with a conic constant of −2.95. The convex back optic zone surface 8105 is aspheric with a central radius of −24.59 mm and a conic constant of −11.22. Central thickness of the IOL 8100 is 0.722 mm.

In the aspheric front and back surfaces of IOL 8100, a central radius of curvature is an instantaneous radius at the apex (or vertex or at the central, axial point) of the aspheric surface. A conic constant is a dimensionless value describing the asphericity of a surface.

In some embodiment, an aspheric surface with a central radius and a conic constant may be described by below:

$$Z = \frac{c {R}^{2}}{1 + \sqrt{1 - ( 1 + q ) {c}^{2} {R}^{2}}} \qquad Eq . ( 3 )$$

where R and Z are the radial and axial coordinates for points on the aspheric surface with the vertex (or apex, or central/axial point) at the origin (i.e., R=0, Z=0), and c is the central curvature (reciprocal of central radius of curvature) and q is the conic constant. When c=0, the surface is flat (or plane or plano). When q=0, the surface section is a part of a circle (or sphere). When q<0 and q>−1, the surface section is a part of a prolate ellipse (i.e., an ellipse which local radius of curvature increases towards the periphery). For q=−1, the surface section is a part of a parabola. When q<−1, the surface section is a part of a hyperbola. Aspheric surface with an oblate ellipse section has positive values for q.

The radial and axial positions of the front optic-control junction 8109, back optic-control junction 8110, front edge point 8112 and back edge point 8113 for this exemplary IOL are given in Table 13.

In this exemplary IOL, the profiles of the front control surface 8107 and back control surface 8108 are defined using polynomial equations which are then translated by select radial and axial distances and tilted by a select angle to provide continuity between a control surface (8107 for front, 8108 for back) and the profile of the optic zone (8104 for front, 8105 for back) at their respective junction (8109 for front, 8110 for back).

The form of the polynomial equation has been described above with respect to Eq. 1. The prescribed polynomial is translated to place its apex at the edge point (8112 for front and 8113 for back) corresponding to point 2802 in FIG. 28. The polynomial is tilted by an angle relative to a frontal plane of the eye and IOL, and its axis of symmetry 8114 is coincident with the edge 8111 of the IOL.

Table 13 lists the values for the order k, 2nd-order coefficient, k^{th}-order coefficient and the tilt angle for the front control surface profile and the back control surface profile. For both control surface profiles, the polynomial is translated to place its apex on their respective edge point and tilted so their axes 8114 are tilted to a select angle specified in Table 13. The value for order k need not be integer (or whole) values as in this exemplary IOL, the front surface polynomial order k is 4.25 and that for the back surface polynomial order k is 3.5 (see Table 13).

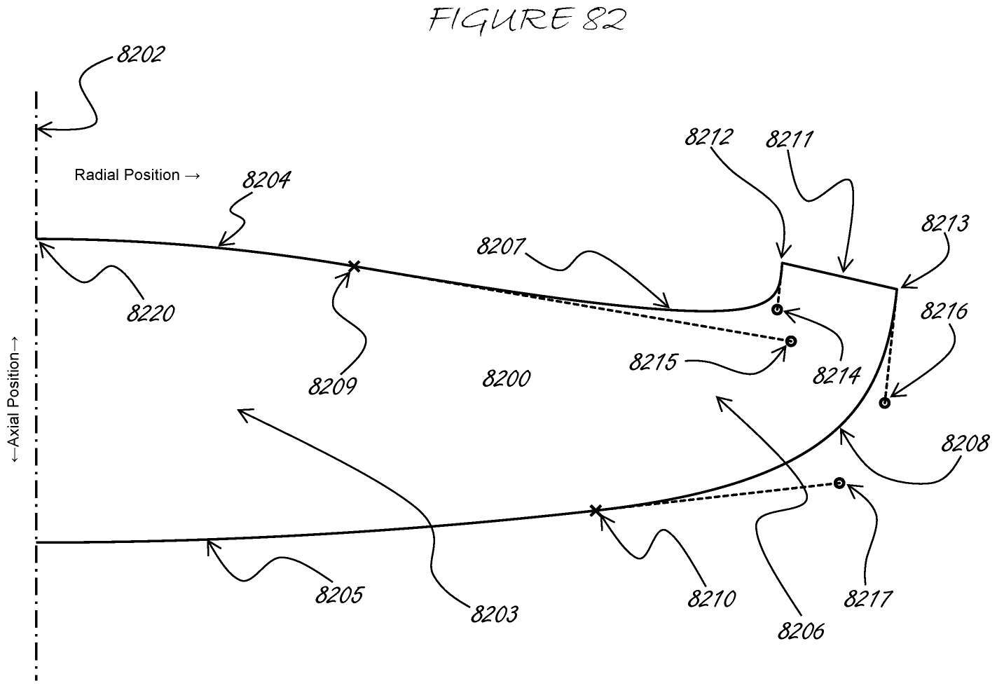

FIG. 82 is a schematic illustration of a half-meridian of an intraocular lens for reducing, minimizing, and/or eliminating peripheral pseudophakic dysphotopsia in accordance with certain embodiments. The prescription for this exemplary IOL with the same optic zone parameters as those for FIG. 81 but with control surface profile defined using Bezier segments is given in Table 14 below.

TABLE 14

| | | | |
|---|---|---|---|
| Feature/Parameter | Unit | Value | Comment |

OPTIC ZONE

| | | | |
|---|---|---|---|
| refractive index | | 1.47 | |
| nominal power of IOL | (D) | +20.00 | when immersed in water |
| front optic central | (mm) | 9.330 | |
| radius of curvature | | | |
| front optic conic constant | | −2.950 | |
| central thickness | (mm) | 0.722 | |
| back optic central | (mm) | −24.590 | negative value means convex |
| radius of curvature | | | towards the back |
| back optic conic constant | | −11.220 | |

CONTROL ZONE

| | | | |
|---|---|---|---|
| front optic-control junction radial | (mm) | 1.107 | radial distance from axis of IOL |
| position | | | |
| front optic-control junction axial | (mm) | 0.065 | axial distance from vertex plane of |
| position | | | IOL |
| back optic-control junction radial | (mm) | 1.950 | radial distance from axis of IOL |
| position | | | |
| back optic-control junction axial | (mm) | 0.646 | axial distance from vertex plane of |
| position | | | IOL |
| angle of edge | (°) | 8.904 | relative to frontal plane |
| front edge radial position | (mm) | 2.600 | radial distance from axis of IOL |
| front edge axial position | (mm) | 0.057 | relative to apex of IOL (negative |
| | | | value means in front of IOL apex) |
| back edge radial position | (mm) | 3.000 | radial distance from axis of IOL |
| back edge axial position | (mm) | 0.120 | relative to apex of IOL |
| front control surface profile | | | |
| front edge Bezier | (mm) | 2.583 | radial distance from axis of IOL |
| tangent point radial position | | | |
| front edge Bezier | (mm) | 0.168 | axial distance from vertex plane of |
| tangent point axial position | | | IOL |
| front junction Bezier | (mm) | 2.631 | radial distance from axis of IOL |
| tangent point radial position | | | |
| front junction Bezier | (mm) | 0.244 | axial distance from vertex plane of |
| tangent point axial position | | | IOL |
| back control surface profile | | | |
| back edge Bezier | (mm) | 2.958 | radial distance from axis of IOL |
| tangent point radial position | | | |
| back edge Bezier | (mm) | 0.390 | axial distance from vertex plane of |
| tangent point axial position | | | IOL |
| back junction Bezier | (mm) | 2.800 | radial distance from axis of IOL |
| tangent point radial position | | | |
| back junction Bezier | (mm) | 0.581 | axial distance from vertex plane of |
| tangent point axial position | | | IOL |

The IOL 8200 is made of material with a refractive index of 1.47 and provides a refractive power of about +20 D through the optic zone 8203. The optic zone has an aspheric anterior surface 8204 with central radius of curvature of 9.33 mm and conic constant of −2.95. The convex aspheric back optic zone surface 8205 has a central radius of −24.59 mm and conic constant value −11.22. Central thickness of the IOL 8200 is 0.722 mm.

A control zone 8206 for controlling, reducing and/or eliminating PPD is provided in IOL 8200. The control zone 8206 comprises a front control surface 8207 and a back control surface 8208. The front/anterior control surface 8207 extends from the front optic-control junction 8209 to a front edge point 8212. The back/posterior control surface 8208 extends from the back optic-control junction 8210 to a back edge point 8213. The front edge point 8212 and back edge point 8213 is joined by the edge 8211 of the IOL.

The radial and axial positions of the front optic-control junction 8209, back optic-control junction 8210, front edge point 8212 and back edge point 8213 for this exemplary IOL are given in Table 14.

In this exemplary IOL the profiles of the front control surface 8207 and back control surface 8208 are defined using Bezier segments between control surface (8207 for front, 8208 for back) and the profile of the optic zone (8204 for front, 8205 for back) at their respective junction (8209 for front, 8210 for back). The tangent points for the front control curve are located at 8214 (front edge start tangent point) and 8215 (front optic-control junction end tangent point), and for the back control curve are located at 8216 (back edge start tangent point) and 8217 (back optic-control junction end tangent point). The form of the equation for the cubic Bezier segment is defined in Eq. 2 above.

Table 14 gives the radial and axial coordinates (positions) of the relevant points.

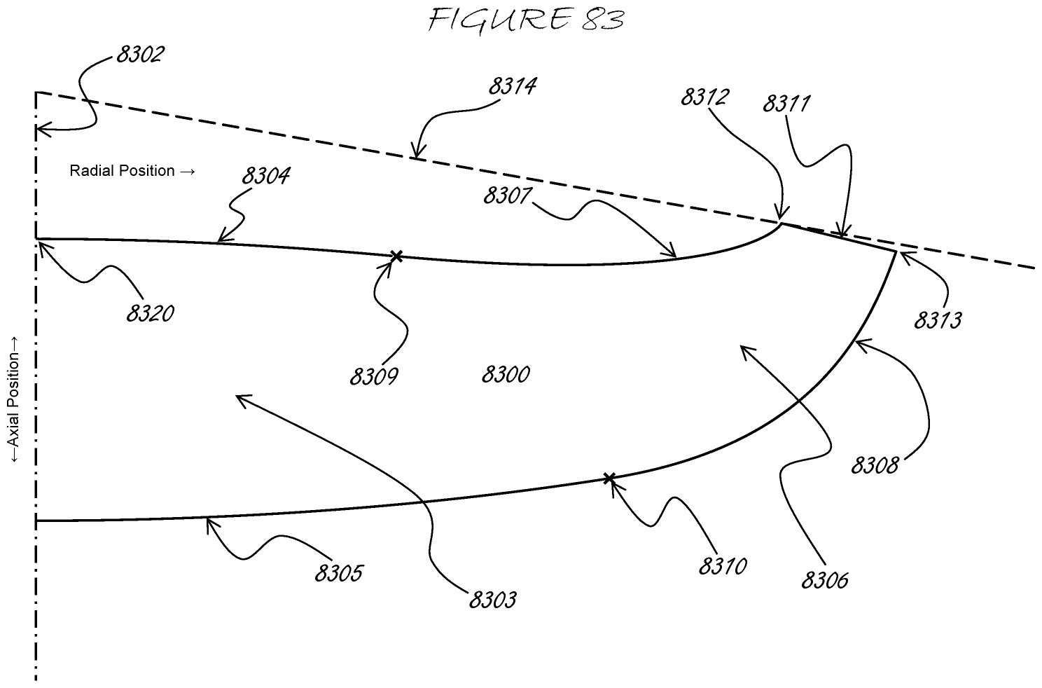

FIG. 83 is a schematic illustration of a half-meridian of an intraocular lens for reducing, minimizing, and/or eliminating peripheral pseudophakic dysphotopsia in accordance with certain embodiments. The prescription for this exemplary IOL for controlling PPD is given in Table 15 below.

TABLE 15

| | | | |
|---|---|---|---|
| Feature/Parameter | Unit | Value | Comment |

OPTIC ZONE

| | | | |
|---|---|---|---|
| refractive index | | 1.53 | |
| nominal power of IOL | (D) | +20.0 | when immersed in water |
| front optic central | (mm) | 18.120 | |
| radius of curvature | | | |
| front optic conic constant | | −45.830 | |
| central thickness | (mm) | 0.670 | |
| back optic central | (mm) | −21.130 | negative value means |
| radius of curvature | | | convex towards the back |
| back optic conic constant | | 23.120 | |

CONTROL ZONE

| | | | |
|---|---|---|---|
| front optic-control | (mm) | 1.257 | radial distance from axis |
| junction radial position | | | of IOL |
| front optic-control | (mm) | 0.041 | axial distance from vertex |
| junction axial position | | | plane of IOL |
| back optic-control | (mm) | 2.000 | radial distance from axis |
| junction radial position | | | of IOL |
| back optic-control | (mm) | 0.570 | axial distance from vertex |
| junction axial position | | | plane of IOL |
| angle of edge | (°) | 9.464 | relative to frontal plane |
| front edge radial position | (mm) | 2.600 | radial distance from axis |
| | | | of IOL |
| front edge axial position | (mm) | −0.037 | relative to apex of IOL |
| | | | (negative value means in |
| | | | front of IOL apex) |
| back edge radial position | (mm) | 3.000 | radial distance from axis |
| | | | of IOL |
| back edge axial position | (mm) | 0.030 | relative to apex of IOL |
| front control surface profile | | | |
| order/exponent of power | | 1.353 | local coordinates: origin/ |
| curve | | | apex of edge point |
| amplitude of power curve | | −1.972 | power curve is located at |
| | | | front |
| front power curve tilt angle | (°) | 26.871 | angle of normal of front |
| | | | control surface power |
| | | | curve at front edge point |
| | | | relative to frontal plane |
| back control surface profile | | | |
| order/exponent of power | | 2.220 | local coordinates: origin/ |
| curve | | | apex of edge point |
| amplitude of power curve | | −0.755 | power curve is located at |
| | | | back |
| back power curve tilt angle | (°) | 26.871 | angle of normal of back |
| | | | control surface power |
| | | | curve at back edge point |
| | | | relative to frontal plane |

The IOL 8300 is made of material with a refractive index of 1.53 and provides a refractive power of about +20 D through the optic zone 8303. The optic zone has an aspheric anterior surface 8304 central radius of curvature of 18.12 mm with a conic constant of −45.83. The aspheric convex back optic zone surface 8305 has a central radius of −21.13 mm and conic constant of 23.12. Central thickness of the IOL 8300 is 0.67 mm.

The radial and axial positions of the front optic-control junction 8309, back optic-control junction 8310, front edge point 8312 and back edge point 8313 for this exemplary IOL are given in Table 15.

In this exemplary IOL, the profiles of the front control surface 8307 and back control surface 8308 may be described using a power function which may be of a form described by below:

y=Bx^{p} Eq. (4)

where x and y are respectively the local radial and axial coordinates for points on the control surface profile and p is an exponent of the power function and B is an amplitude of the power function. Coordinates x and y of Eq. 4 may be local to (i.e., specific to the coordinate system of) the power function and may be distinct from the radial and axial coordinate system with respect to the IOL and/or the eye. The exponent p need not be restricted to integers and may take on a value drawn from the set of real numbers including positive and negative values. The amplitude B may also take on values drawn from the set of real numbers including positive and negative values.

To render the control curve, the power function curve (as defined in its local coordinates according to Eq. 4) is translated so that its apex (x=0, y=0 in the local coordinate system) is placed on the edge point (8312 for front and 8313 for back) of the IOL.

The translated power function is then rotated about its apex (now at the edge point) so that its y-axis (in its local coordinate system) is tilted at an angle with respect to (e.g., relative to) the frontal plane of the IOL and/or eye.

Table 15 lists the values for various parameters for the exemplary IOL of FIG. 83. The power curve for the front control surface profile has amplitude B of −1.972 and exponent p of 1.353 while the back control surface power curve is described with an amplitude B of −0.755 and an exponent p of 2.22. The local y-axes of the power curves describing the front and the back control surfaces are tilted by 26.8710 relative to a frontal plane of the IOL.

The edge of this exemplary IOL is at an angle of about 9.5° relative to a frontal plane of the IOL or eye. That is, a normal to the lens edge is at angle of about 9.5° to an axis of the IOL. For this exemplary IOL, the angle between the lens edge and the front control surface as the front control surface approaches the front edge point (that is, the angle of a tangent to the front control surface at or near to the front edge point) is about 107.4°. This angle is considered as the “internal” angle of the front edge point of the IOL. The internal angle is the angle subtended within the bulk (or material) of the IOL at the front edge point. In FIG. 83, the angle is that between the tangent to the front control curve 8307 at the front edge point 8312 and the lens edge 8311 between front edge point 8312 and back edge point 8313 (choosing the value for the angle that is less than 180°). The angle between the lens edge and the back control surface as the back control surface approaches the back edge point (that is, the angle of a tangent to the back control surface at or near to the back edge point) is about 72.6°. Similarly to the angle at the front edge point, this angle between the lens edge and the back control surface is measured as that between the tangent to the back control curve 8308 at the back edge point 8313 and the lens edge 8311 between back edge point 8313 and front edge point 8312 (choosing the value for the angle that is less than 180°).

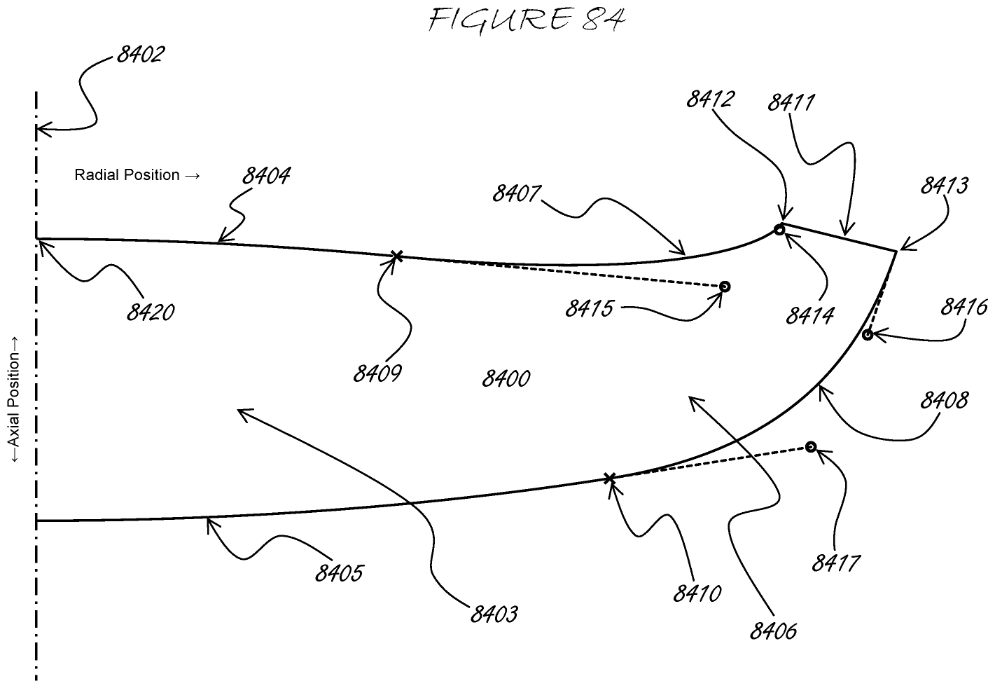

FIG. 84 is a schematic illustration of a half-meridian of an intraocular lens for reducing, minimizing, and/or eliminating peripheral pseudophakic dysphotopsia in accordance with certain embodiments. The prescription for this exemplary IOL with the same optic zone parameters as those for FIG. 83 but with control surface profile defined using Bezier segments is given in Table 16 below.

TABLE 16

| | | | |
|---|---|---|---|
| Feature/Parameter | Unit | Value | Comment |

OPTIC ZONE

| | | | |
|---|---|---|---|
| refractive index | | 1.53 | |
| nominal power of IOL | (D) | +20.0 | when immersed in water |
| front optic central | (mm) | 18.120 | |
| radius of curvature | | | |
| front optic conic constant | | −45.830 | |
| central thickness | (mm) | 0.670 | |
| back optic central | (mm) | −21.130 | negative value means convex |
| radius of curvature | | | towards the back |
| back optic conic constant | | 23.120 | |

CONTROL ZONE

| | | | |
|---|---|---|---|
| front optic-control junction radial | (mm) | 1.257 | radial distance from axis of IOL |
| position | | | |
| front optic-control junction axial | (mm) | 0.041 | axial distance from vertex plane of |
| position | | | IOL |
| back optic-control junction radial | (mm) | 2.000 | radial distance from axis of IOL |
| position | | | |
| back optic-control junction axial | (mm) | 0.570 | axial distance from vertex plane of |
| position | | | IOL |
| angle of edge | (°) | 9.464 | relative to frontal plane |
| front edge radial position | (mm) | 2.600 | radial distance from axis of IOL |
| front edge axial position | (mm) | −0.037 | relative to apex of IOL (negative |
| | | | value means in front of IOL apex) |
| back edge radial position | (mm) | 3.000 | radial distance from axis of IOL |
| back edge axial position | (mm) | 0.030 | relative to apex of IOL |
| front control surface profile | | | |
| front edge Bezier | (mm) | 2.592 | radial distance from axis of IOL |
| tangent point radial position | | | |
| front edge Bezier | (mm) | −0.021 | axial distance from vertex plane of |
| tangent point axial position | | | IOL |
| front junction Bezier | (mm) | 2.402 | radial distance from axis of IOL |
| tangent point radial position | | | |
| front junction Bezier | (mm) | 0.113 | axial distance from vertex plane of |
| tangent point axial position | | | IOL |
| back control surface profile | | | |
| back edge Bezier | (mm) | 2.900 | radial distance from axis of IOL |
| tangent point radial position | | | |
| back edge Bezier | (mm) | 0.228 | axial distance from vertex plane of |
| tangent point axial position | | | IOL |
| back junction Bezier | (mm) | 2.702 | radial distance from axis of IOL |
| tangent point radial position | | | |
| back junction Bezier | (mm) | 0.495 | axial distance from vertex plane of |
| tangent point axial position | | | IOL |

The IOL 8400 is made of material with a refractive index of 1.53 and provides a refractive power of about +20 D through the optic zone 8403. The optic zone is aspheric with an anterior surface 8404 central radius of curvature of 18.12 mm and a conic constant of −45.83. The convex back optic zone aspheric surface 8405 has a central radius of −21.13 mm. Central thickness of the IOL 8400 is 0.67 mm.

A control zone 8406 for controlling, reducing and/or eliminating PPD is provided in IOL 8400. The control zone 8406 comprises a front control surface 8407 and a back control surface 8408. The front/anterior control surface 8407 extends from the front optic-control junction 8409 to a front edge point 8412. The back/posterior control surface 8408 extends from the back optic-control junction 8410 to a back edge point 8413. The front edge point 8412 and back edge point 8413 is joined by the edge 8411 of the IOL.

The radial and axial positions of the front optic-control junction 8409, back optic-control junction 8410, front edge point 8412 and back edge point 8413 for this exemplary IOL are given in Table 16.

In this exemplary IOL the profiles of the front control surface 8407 and back control surface 8408 are defined using Bezier segments between control surface (4007 for front, 8408 for back) and the profile of the optic zone (4004 for front, 8405 for back) at their respective junction (4009 for front, 8410 for back). The tangent points for the front control curve are located at 8414 (front edge start tangent point) and 8415 (front optic-control junction end tangent point), and for the back control curve are located at 8416 (back edge start tangent point) and 8417 (back optic-control junction end tangent point). The form of the equation for the cubic Bezier segment is defined in Eq. 2 above.

Table 16 gives the radial and axial coordinates (positions) of the relevant points.

The edge of this exemplary IOL is at an angle of about 9.5° relative to a frontal plane of the IOL or eye. That is, a normal to the lens edge is at angle of about 9.5° to an axis of the IOL. For this exemplary IOL, the angle between the lens edge and the front control surface as the front control surface approaches the front edge point (that is, the angle of a tangent to the front control surface at or near to the front edge point) is about 107.4°. This angle is considered as the “internal” angle of the front edge point of the IOL. The internal angle is the angle subtended within the bulk (or material) of the IOL at the front edge point. In FIG. 84, the angle is that between the tangent to the front control curve 8407 at the front edge point 8412 and the lens edge 8411 between front edge point 8412 and back edge point 8413 (choosing the value for the angle that is less than 180°). The angle between the lens edge and the back control surface as the back control surface approaches the back edge point (that is, the angle of a tangent to the back control surface at or near to the back edge point) is about 72.6°. Similarly to the angle at the front edge point, this angle between the lens edge and the back control surface is measured as that between the tangent to the back control curve 8408 at the back edge point 8413 and the lens edge 8411 between back edge point 8413 and front edge point 8412 (choosing the value for the angle that is less than 180°).

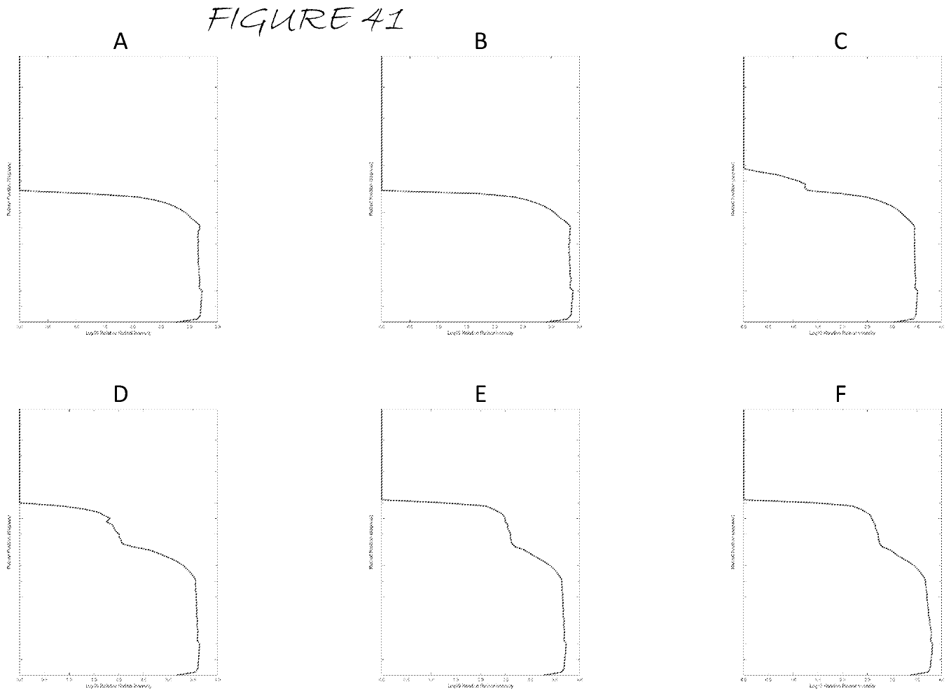

FIGS. 41-48 illustrate the relative whole-field retinal intensity of another exemplary IOL while varying pupil size from 2.5 mm to 5 mm diameter in 0.5 mm steps and an implantation depth from 0 mm to 0.7 mm in 0.1 mm steps. The IOL modelled is another example of an IOL (see FIG. 34 and Table 4) with a control zone for distributing light rays to irradiate the otherwise dark band on the retina. For FIGS. 41-48, about 10,000 rays are traced non-sequentially through the eye model for each field angle. A ray-density plot is generated integrating over the field angles (e.g., 58° to 98° in 0.50 steps). Then, the ray-density results are convolved around azimuthal angles to produce a relative whole-field retinal irradiance plot. The plot axes and scales, and intensity grey-scale, of all plots shown in FIGS. 41-48 are the same as those used in FIGS. 8-15 and FIGS. 19-26 with the exception that the horizontal axis indicating relative whole-field retinal irradiance values, while still using a logarithmic scale (base 10) has a full-scale range of 4 log units (versus the 4.5 log units range for FIGS. 8-15 and FIGS. 19-26). This is due to the fewer rays (10,000 versus 100,000) traced for the model, resulting in lower number of rays intercepting the retina.

FIGS. 41A-F are relative retina irradiance plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0 mm in accordance with certain embodiments. FIG. 41A illustrates the relative whole-field retinal irradiance for 0 mm IOL implantation depth and a 2.5 mm pupil diameter. FIG. 41B illustrates the relative whole-field retinal irradiance for 0 mm IOL implantation depth and a 3 mm pupil diameter. FIG. 41C illustrates the relative whole-field retinal irradiance for 0 mm IOL implantation depth and a 3.5 mm pupil diameter. FIG. 41D illustrates the relative whole-field retinal irradiance for 0 mm IOL implantation depth and a 4 mm pupil diameter. FIG. 41E illustrates the relative whole-field retinal irradiance for 0 mm IOL implantation depth and a 4.5 mm pupil diameter. FIG. 41F illustrates the relative whole-field retinal irradiance for 0 mm IOL implantation depth and a 5 mm pupil diameter. As illustrated, at this implantation depth, no dark band retinal region occurs for the illustrated pupil sizes.

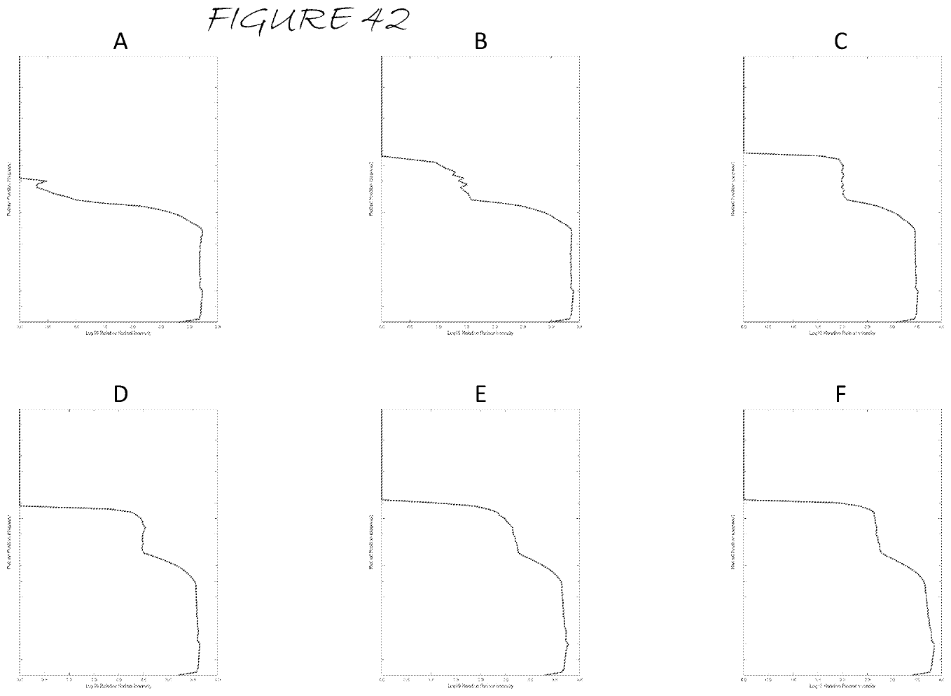

FIGS. 42A-F are relative retina irradiance plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.1 mm in accordance with certain embodiments. FIG. 42A illustrates the relative whole-field retinal irradiance for 0.1 mm IOL implantation depth and a 2.5 mm pupil diameter. FIG. 42B illustrates the relative whole-field retinal irradiance for 0.1 mm IOL implantation depth and a 3 mm pupil diameter. FIG. 42C illustrates the relative whole-field retinal irradiance for 0.1 mm IOL implantation depth and a 3.5 mm pupil diameter. FIG. 42D illustrates the relative whole-field retinal irradiance for 0.1 mm IOL implantation depth and a 4 mm pupil diameter. FIG. 42E illustrates the relative whole-field retinal irradiance for 0.1 mm IOL implantation depth and a 4.5 mm pupil diameter. FIG. 42F illustrates the relative whole-field retinal irradiance for 0.1 mm IOL implantation depth and a 5 mm pupil diameter. As illustrated, at this implantation depth, no dark band retinal region occurs for the illustrated pupil sizes.

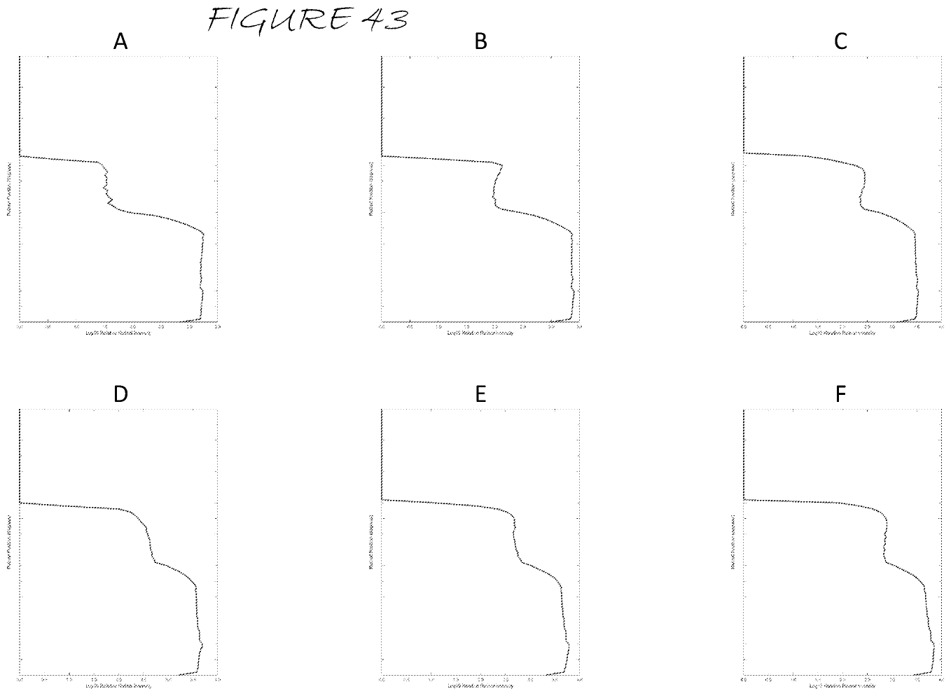

FIGS. 43A-F are relative retina irradiance plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.2 mm in accordance with certain embodiments. FIG. 43A illustrates the relative whole-field retinal irradiance for 0.2 mm IOL implantation depth and a 2.5 mm pupil diameter. FIG. 43B illustrates the relative whole-field retinal irradiance for 0.2 mm IOL implantation depth and a 3 mm pupil diameter. FIG. 43C illustrates the relative whole-field retinal irradiance for 0.2 mm IOL implantation depth and a 3.5 mm pupil diameter. FIG. 43D illustrates the relative whole-field retinal irradiance for 0.2 mm IOL implantation depth and a 4 mm pupil diameter. FIG. 43E illustrates the relative whole-field retinal irradiance for 0.2 mm IOL implantation depth and a 4.5 mm pupil diameter. FIG. 43F illustrates the relative whole-field retinal irradiance for 0.2 mm IOL implantation depth and a 5 mm pupil diameter. As illustrated, at this implantation depth, no dark band retinal region occurs for the illustrated pupil sizes.

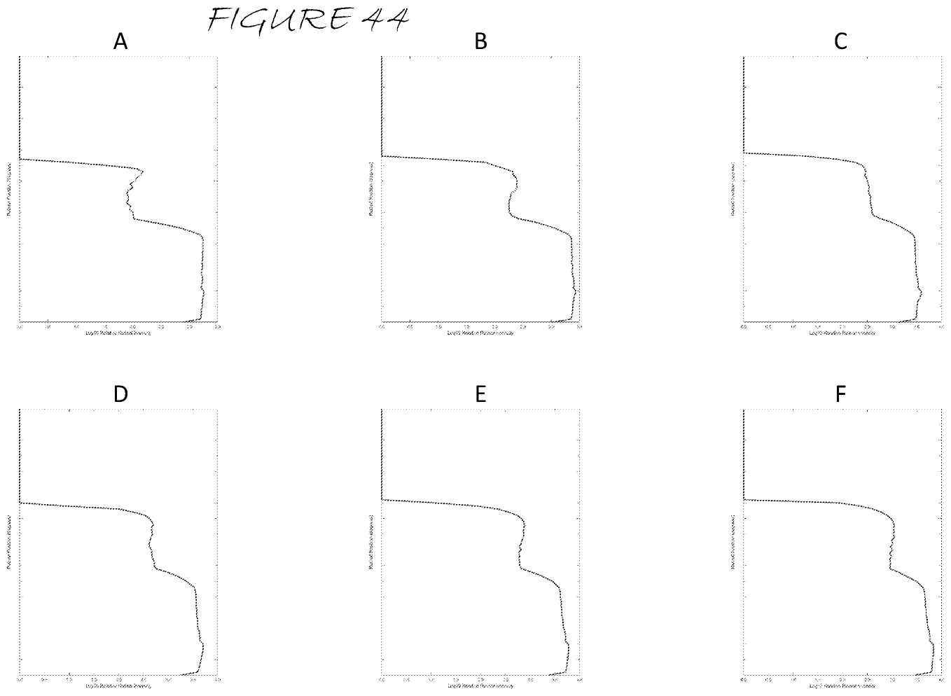

FIGS. 44A-F are relative retina irradiance plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.3 mm in accordance with certain embodiments. FIG. 44A illustrates the relative whole-field retinal irradiance for 0.3 mm IOL implantation depth and a 2.5 mm pupil diameter. FIG. 44B illustrates the relative whole-field retinal irradiance for 0.3 mm IOL implantation depth and a 3 mm pupil diameter. FIG. 44C illustrates the relative whole-field retinal irradiance for 0.3 mm IOL implantation depth and a 3.5 mm pupil diameter. FIG. 44D illustrates the relative whole-field retinal irradiance for 0.3 mm IOL implantation depth and a 4 mm pupil diameter. FIG. 44E illustrates the relative whole-field retinal irradiance for 0.3 mm IOL implantation depth and a 4.5 mm pupil diameter. FIG. 44F illustrates the relative whole-field retinal irradiance for 0.3 mm IOL implantation depth and a 5 mm pupil diameter. As illustrated, at this implantation depth, no dark band retinal region occurs for the illustrated pupil sizes.

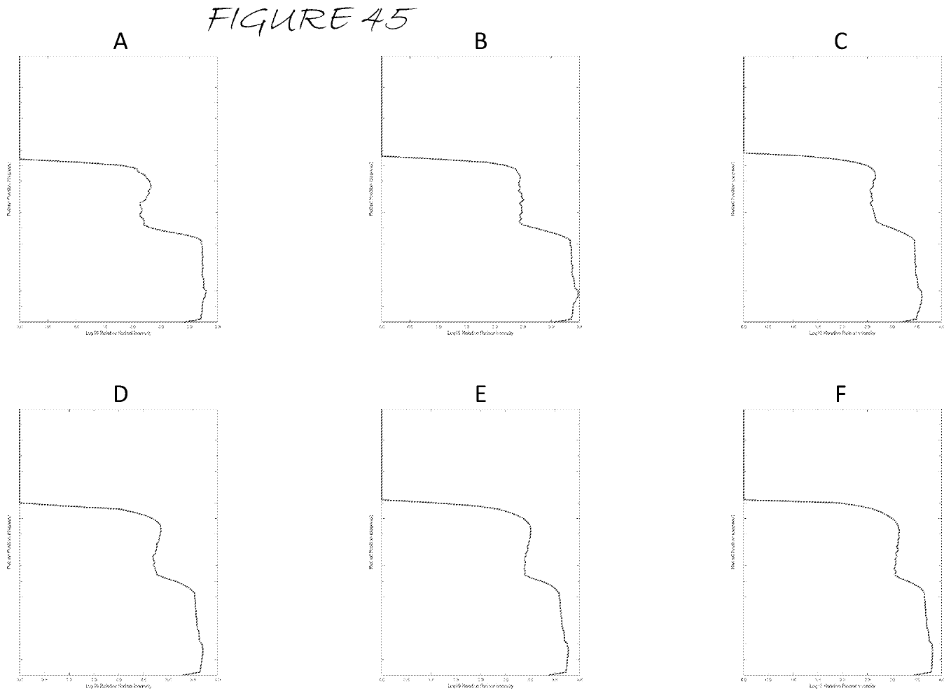

FIGS. 45A-F are relative retina irradiance plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.4 mm in accordance with certain embodiments. FIG. 45A illustrates the relative whole-field retinal irradiance for 0.4 mm IOL implantation depth and a 2.5 mm pupil diameter. FIG. 45B illustrates the relative whole-field retinal irradiance for 0.4 mm IOL implantation depth and a 3 mm pupil diameter. FIG. 45C illustrates the relative whole-field retinal irradiance for 0.4 mm IOL implantation depth and a 3.5 mm pupil diameter. FIG. 45D illustrates the relative whole-field retinal irradiance for 0.4 mm IOL implantation depth and a 4 mm pupil diameter. FIG. 45E illustrates the relative whole-field retinal irradiance for 0.4 mm IOL implantation depth and a 4.5 mm pupil diameter. FIG. 45F illustrates the relative whole-field retinal irradiance for 0.4 mm IOL implantation depth and a 5 mm pupil diameter. As illustrated, at this implantation depth, no dark band retinal region occurs for the illustrated pupil sizes.

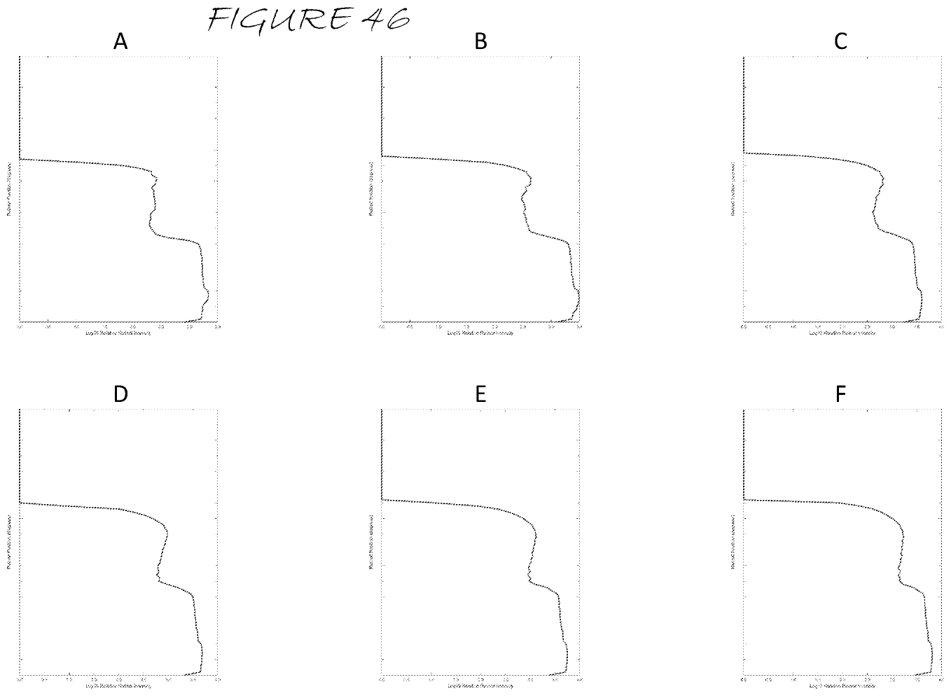

FIGS. 46A-F are relative retina irradiance plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.5 mm in accordance with certain embodiments. FIG. 46A illustrates the relative whole-field retinal irradiance for 0.5 mm IOL implantation depth and a 2.5 mm pupil diameter. FIG. 46B illustrates the relative whole-field retinal irradiance for 0.5 mm IOL implantation depth and a 3 mm pupil diameter. FIG. 46C illustrates the relative whole-field retinal irradiance for 0.5 mm IOL implantation depth and a 3.5 mm pupil diameter. FIG. 46D illustrates the relative whole-field retinal irradiance for 0.5 mm IOL implantation depth and a 4 mm pupil diameter. FIG. 46E illustrates the relative whole-field retinal irradiance for 0.5 mm IOL implantation depth and a 4.5 mm pupil diameter. FIG. 46F illustrates the relative whole-field retinal irradiance for 0.5 mm IOL implantation depth and a 5 mm pupil diameter. As illustrated, at this implantation depth, no dark band retinal region occurs for the illustrated pupil sizes.

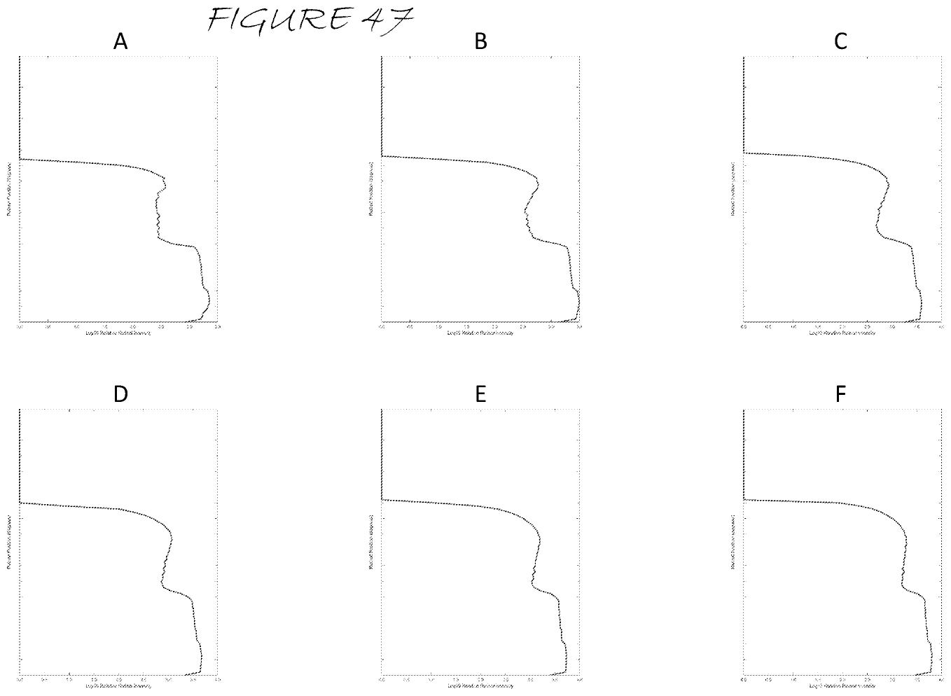

FIGS. 47A-F are relative retina irradiance plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.6 mm in accordance with certain embodiments. FIG. 47A illustrates the relative whole-field retinal irradiance for 0.6 mm IOL implantation depth and a 2.5 mm pupil diameter. FIG. 47B illustrates the relative whole-field retinal irradiance for 0.6 mm IOL implantation depth and a 3 mm pupil diameter. FIG. 47C illustrates the relative whole-field retinal irradiance for 0.6 mm IOL implantation depth and a 3.5 mm pupil diameter. FIG. 47D illustrates the relative whole-field retinal irradiance for 0.6 mm IOL implantation depth and a 4 mm pupil diameter. FIG. 47E illustrates the relative whole-field retinal irradiance for 0.6 mm IOL implantation depth and a 4.5 mm pupil diameter. FIG. 47F illustrates the relative whole-field retinal irradiance for 0.6 mm IOL implantation depth and a 5 mm pupil diameter. As illustrated, at this implantation depth, no dark band retinal region occurs for the illustrated pupil sizes.

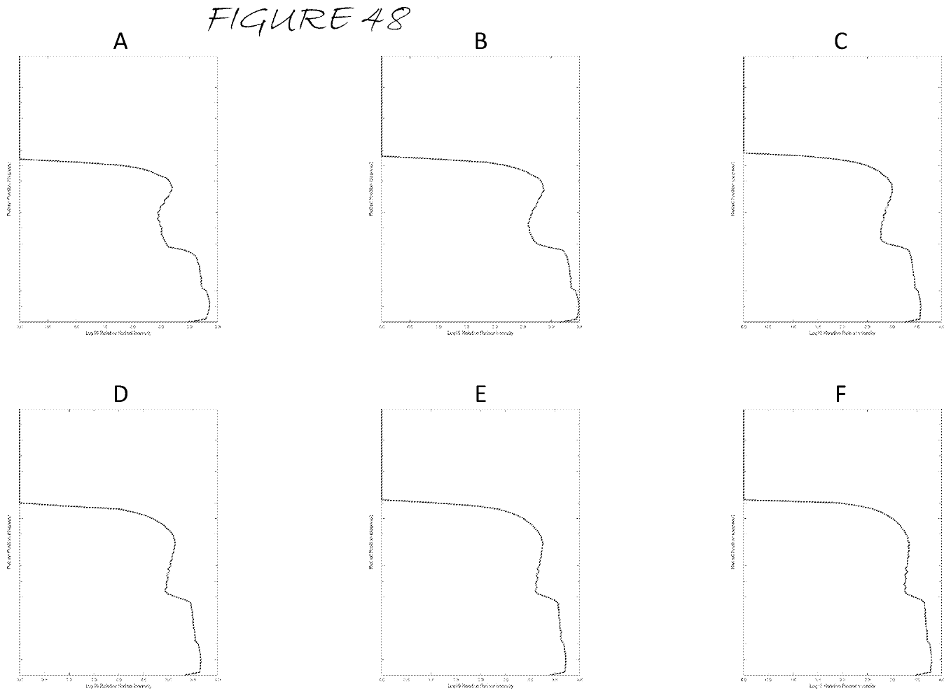

FIGS. 48A-F are relative retina irradiance plots showing the intensity (e.g., relative whole field retinal irradiance) of light distribution across the retina for the intraocular lens modelled in FIG. 34 for pupil sizes from 2.5 mm to 5 mm in 0.5 mm steps at an implantation depth of 0.7 mm in accordance with certain embodiments. FIG. 48A illustrates the relative whole-field retinal irradiance for 0.7 mm IOL implantation depth and a 2.5 mm pupil diameter. FIG. 48B illustrates the relative whole-field retinal irradiance for 0.7 mm IOL implantation depth and a 3 mm pupil diameter. FIG. 48C illustrates the relative whole-field retinal irradiance for 0.7 mm IOL implantation depth and a 3.5 mm pupil diameter. FIG. 48D illustrates the relative whole-field retinal irradiance for 0.7 mm IOL implantation depth and a 4 mm pupil diameter. FIG. 48E illustrates the relative whole-field retinal irradiance for 0.7 mm IOL implantation depth and a 4.5 mm pupil diameter. FIG. 48F illustrates the relative whole-field retinal irradiance for 0.7 mm IOL implantation depth and a 5 mm pupil diameter. As illustrated, at this implantation depth, no dark band retinal region occurs for the illustrated pupil sizes.

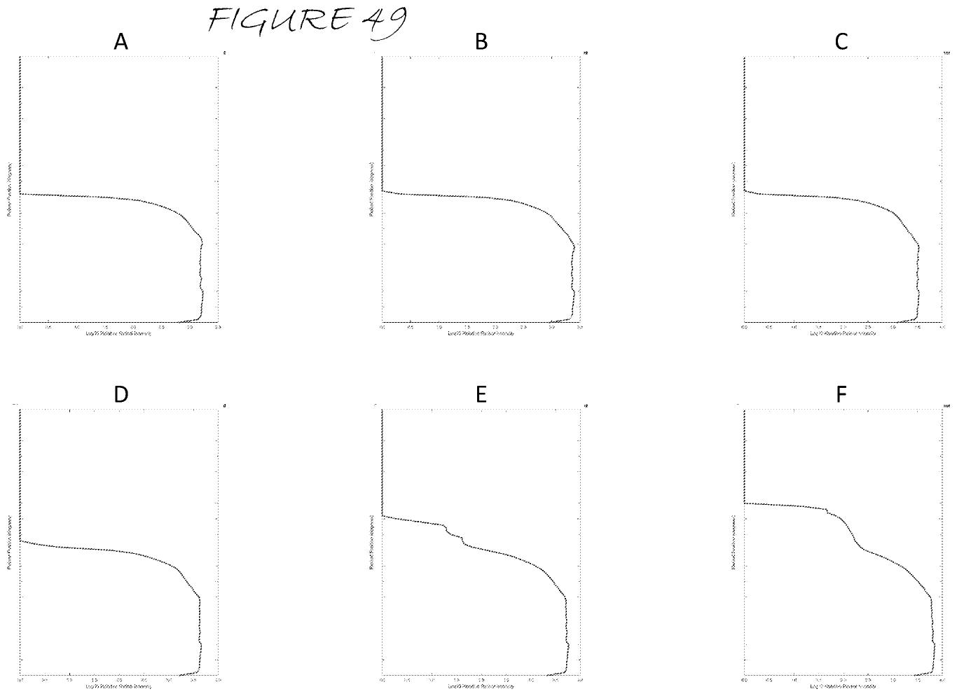

FIGS. 49-56 illustrate the relative whole-field retinal intensity of another exemplary IOL while varying pupil size from 2.5 mm to 5 mm diameter in 0.5 mm steps and an implantation depth from 0 mm to 0.7 mm in 0.1 mm steps. The IOL modelled is another example of an IOL (see FIG. 35 and Table 6) with a control zone for distributing light rays to irradiate the otherwise dark band on the retina. For FIGS. 49-56, about 10,000 rays are traced non-sequentially through the eye model for each field angle. A ray-density plot is generated integrating over the field angles (e.g., 58° to 98° in 0.5° steps). Then, the ray-density results are convolved around azimuthal angles to produce a relative whole-field retinal irradiance plot. The plot axes and scales, and intensity grey-scale, of all plots shown in FIGS. 49-56 are the same as those used in FIGS. 8-15 and FIGS. 19-26 with the exception that the horizontal axis indicating relative whole-field retinal irradiance values, while still using a logarithmic scale (base 10) has a full-scale range of 4 log units (versus the 4.5 log units range for FIGS. 8-15 and FIGS. 19-26). This is due to the fewer rays (10,000 versus 100,000) traced for the model, resulting in lower values in relative retinal irradiance.

FIGS. 49A-F are relative retina irradiance plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0 mm in accordance with certain embodiments. FIG. 49A illustrates the relative whole-field retinal irradiance for 0 mm IOL implantation depth and a 2.5 mm pupil diameter. FIG. 49B illustrates the relative whole-field retinal irradiance for 0 mm IOL implantation depth and a 3 mm pupil diameter. FIG. 49C illustrates the relative whole-field retinal irradiance for 0 mm IOL implantation depth and a 3.5 mm pupil diameter. FIG. 49D illustrates the relative whole-field retinal irradiance for 0 mm IOL implantation depth and a 4 mm pupil diameter. FIG. 49E illustrates the relative whole-field retinal irradiance for 0 mm IOL implantation depth and a 4.5 mm pupil diameter. FIG. 49F illustrates the relative whole-field retinal irradiance for 0 mm IOL implantation depth and a 5 mm pupil diameter. As illustrated, at this implantation depth, no dark band retinal region occurs for the illustrated pupil sizes.

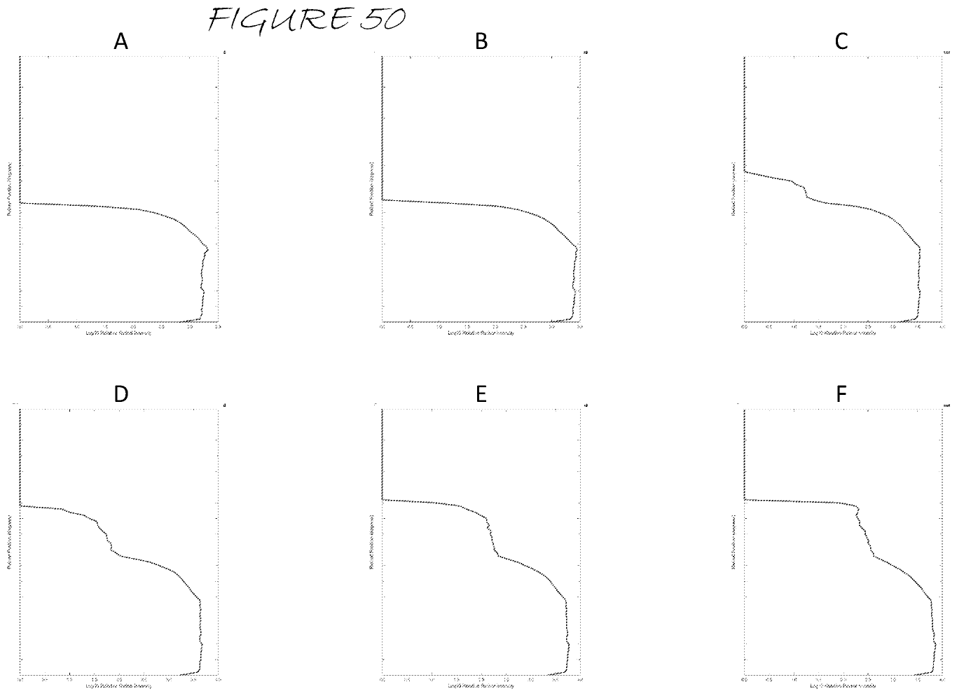

FIGS. 50A-F are relative retina irradiance plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.1 mm in accordance with certain embodiments. FIG. 50A illustrates the relative whole-field retinal irradiance for 0.1 mm IOL implantation depth and a 2.5 mm pupil diameter. FIG. 50B illustrates the relative whole-field retinal irradiance for 0.1 mm IOL implantation depth and a 3 mm pupil diameter. FIG. 50C illustrates the relative whole-field retinal irradiance for 0.1 mm IOL implantation depth and a 3.5 mm pupil diameter. FIG. 50D illustrates the relative whole-field retinal irradiance for 0.1 mm IOL implantation depth and a 4 mm pupil diameter. FIG. 50E illustrates the relative whole-field retinal irradiance for 0.1 mm IOL implantation depth and a 4.5 mm pupil diameter. FIG. 50F illustrates the relative whole-field retinal irradiance for 0.1 mm IOL implantation depth and a 5 mm pupil diameter. As illustrated, at this implantation depth, no dark band retinal region occurs for the illustrated pupil sizes.

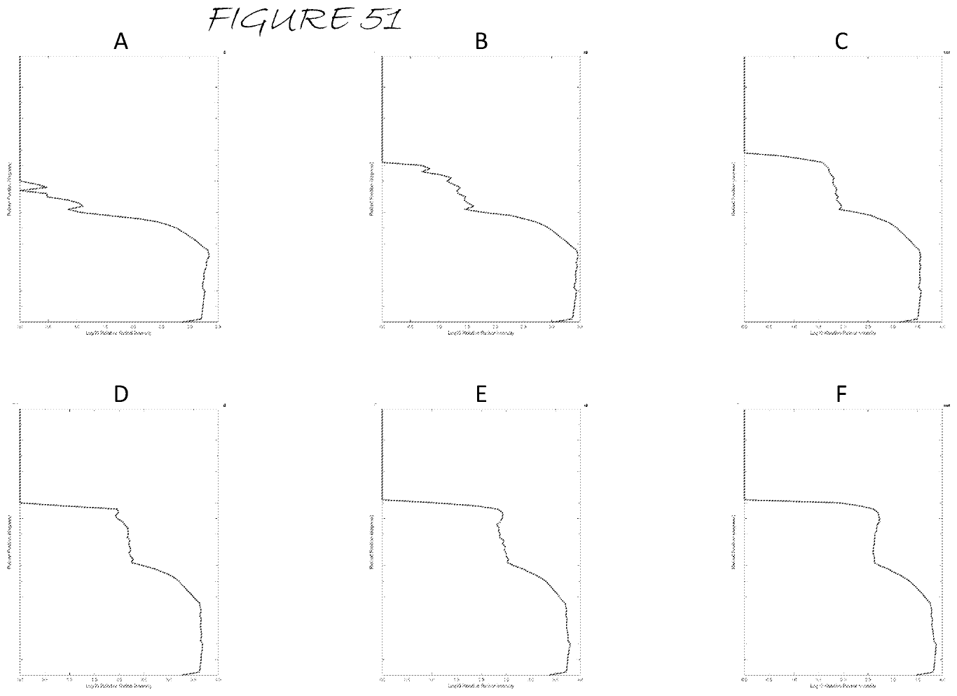

FIGS. 51A-F are relative retina irradiance plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.2 mm in accordance with certain embodiments. FIG. 51A illustrates the relative whole-field retinal irradiance for 0.2 mm IOL implantation depth and a 2.5 mm pupil diameter. FIG. 51B illustrates the relative whole-field retinal irradiance for 0.2 mm IOL implantation depth and a 3 mm pupil diameter. FIG. 51C illustrates the relative whole-field retinal irradiance for 0.2 mm IOL implantation depth and a 3.5 mm pupil diameter. FIG. 51D illustrates the relative whole-field retinal irradiance for 0.2 mm IOL implantation depth and a 4 mm pupil diameter. FIG. 51E illustrates the relative whole-field retinal irradiance for 0.2 mm IOL implantation depth and a 4.5 mm pupil diameter. FIG. 51F illustrates the relative whole-field retinal irradiance for 0.2 mm IOL implantation depth and a 5 mm pupil diameter. As illustrated, at this implantation depth, no dark band retinal region occurs for the illustrated pupil sizes.

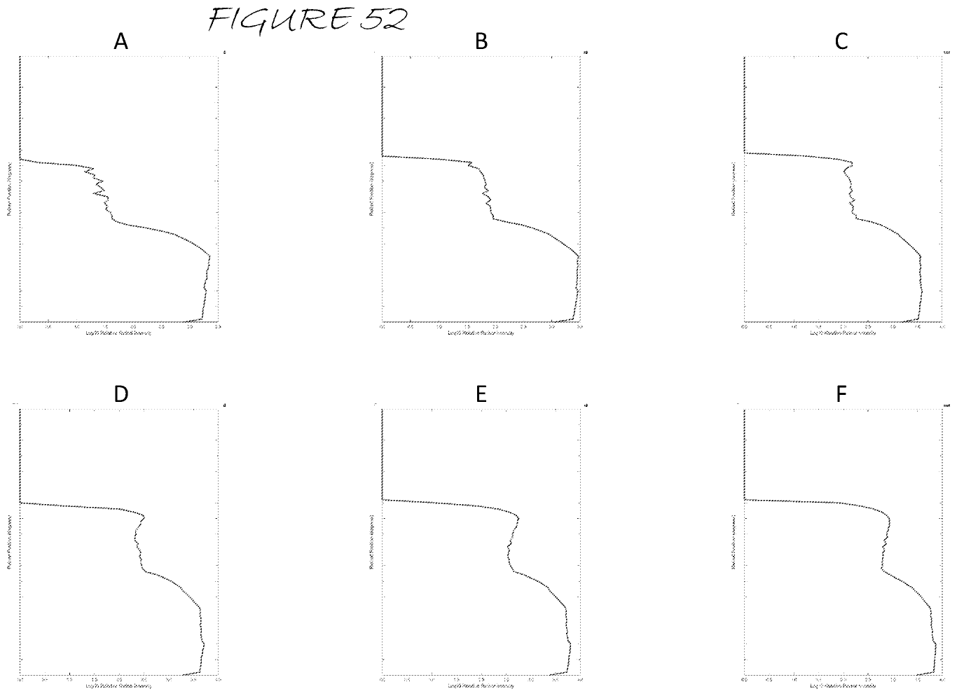

FIGS. 52A-F are relative retina irradiance plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.3 mm in accordance with certain embodiments. FIG. 52A illustrates the relative whole-field retinal irradiance for 0.3 mm IOL implantation depth and a 2.5 mm pupil diameter. FIG. 52B illustrates the relative whole-field retinal irradiance for 0.3 mm IOL implantation depth and a 3 mm pupil diameter. FIG. 52C illustrates the relative whole-field retinal irradiance for 0.3 mm IOL implantation depth and a 3.5 mm pupil diameter. FIG. 52D illustrates the relative whole-field retinal irradiance for 0.3 mm IOL implantation depth and a 4 mm pupil diameter. FIG. 52E illustrates the relative whole-field retinal irradiance for 0.3 mm IOL implantation depth and a 4.5 mm pupil diameter. FIG. 52F illustrates the relative whole-field retinal irradiance for 0.3 mm IOL implantation depth and a 5 mm pupil diameter. As illustrated, at this implantation depth, no dark band retinal region occurs for the illustrated pupil sizes.

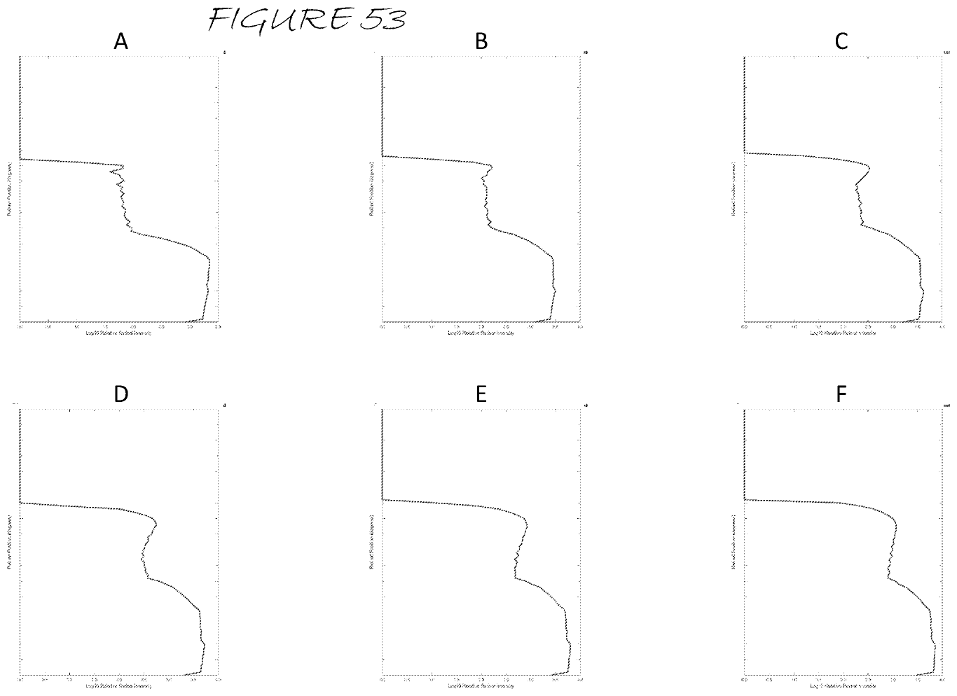

FIGS. 53A-F are relative retina irradiance plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.4 mm in accordance with certain embodiments. FIG. 53A illustrates the relative whole-field retinal irradiance for 0.4 mm IOL implantation depth and a 2.5 mm pupil diameter. FIG. 53B illustrates the relative whole-field retinal irradiance for 0.4 mm IOL implantation depth and a 3 mm pupil diameter. FIG. 53C illustrates the relative whole-field retinal irradiance for 0.4 mm IOL implantation depth and a 3.5 mm pupil diameter. FIG. 53D illustrates the relative whole-field retinal irradiance for 0.4 mm IOL implantation depth and a 4 mm pupil diameter. FIG. 53E illustrates the relative whole-field retinal irradiance for 0.4 mm IOL implantation depth and a 4.5 mm pupil diameter. FIG. 53F illustrates the relative whole-field retinal irradiance for 0.4 mm IOL implantation depth and a 5 mm pupil diameter. As illustrated, at this implantation depth, no dark band retinal region occurs for the illustrated pupil sizes.

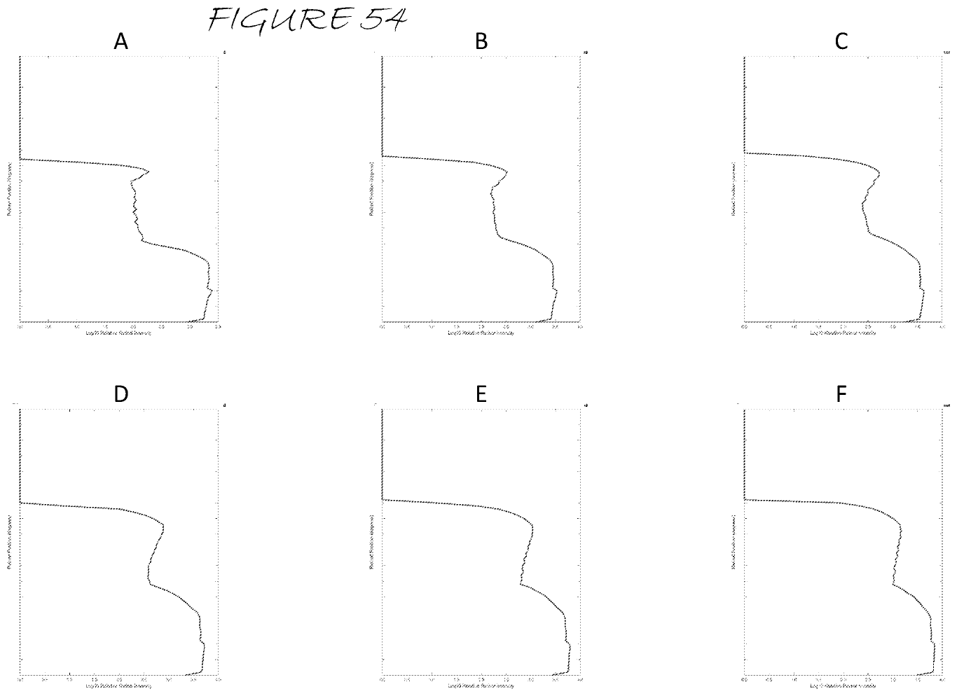

FIGS. 54A-F are relative retina irradiance plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.5 mm in accordance with certain embodiments. FIG. 54A illustrates the relative whole-field retinal irradiance for 0.5 mm IOL implantation depth and a 2.5 mm pupil diameter. FIG. 54B illustrates the relative whole-field retinal irradiance for 0.5 mm IOL implantation depth and a 3 mm pupil diameter. FIG. 54C illustrates the relative whole-field retinal irradiance for 0.5 mm IOL implantation depth and a 3.5 mm pupil diameter. FIG. 54D illustrates the relative whole-field retinal irradiance for 0.5 mm IOL implantation depth and a 4 mm pupil diameter. FIG. 54E illustrates the relative whole-field retinal irradiance for 0.5 mm IOL implantation depth and a 4.5 mm pupil diameter. FIG. 54F illustrates the relative whole-field retinal irradiance for 0.5 mm IOL implantation depth and a 5 mm pupil diameter. As illustrated, at this implantation depth, no dark band retinal region occurs for the illustrated pupil sizes.

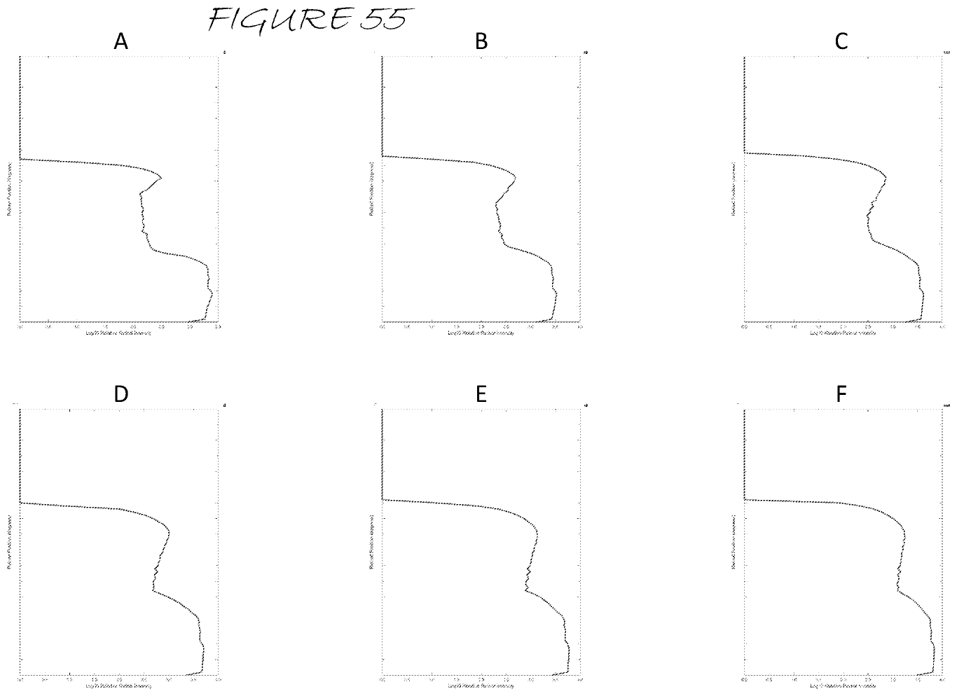

FIGS. 55A-F are relative retina irradiance plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.6 mm in accordance with certain embodiments. FIG. 55A illustrates the relative whole-field retinal irradiance for 0.6 mm IOL implantation depth and a 2.5 mm pupil diameter. FIG. 55B illustrates the relative whole-field retinal irradiance for 0.6 mm IOL implantation depth and a 3 mm pupil diameter. FIG. 55C illustrates the relative whole-field retinal irradiance for 0.6 mm IOL implantation depth and a 3.5 mm pupil diameter. FIG. 55D illustrates the relative whole-field retinal irradiance for 0.6 mm IOL implantation depth and a 4 mm pupil diameter. FIG. 55E illustrates the relative whole-field retinal irradiance for 0.6 mm IOL implantation depth and a 4.5 mm pupil diameter. FIG. 55F illustrates the relative whole-field retinal irradiance for 0.6 mm IOL implantation depth and a 5 mm pupil diameter. As illustrated, at this implantation depth, no dark band retinal region occurs for the illustrated pupil sizes.

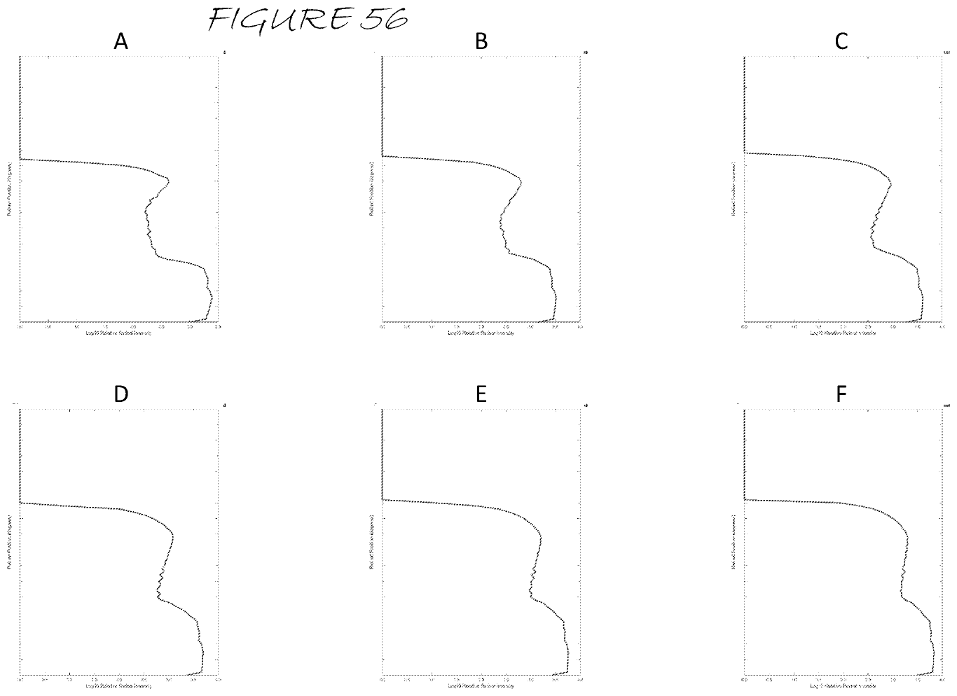

FIGS. 56A-F are relative retina irradiance plots showing the intensity (e.g., relative whole field retinal irradiance) of light distribution across the retina for the intraocular lens modelled in FIG. 35 for pupil sizes from 2.5 mm to 5 mm in 0.5 mm steps at an implantation depth of 0.7 mm in accordance with certain embodiments. FIG. 56A illustrates the relative whole-field retinal irradiance for 0.7 mm IOL implantation depth and a 2.5 mm pupil diameter. FIG. 56B illustrates the relative whole-field retinal irradiance for 0.7 mm IOL implantation depth and a 3 mm pupil diameter. FIG. 56C illustrates the relative whole-field retinal irradiance for 0.7 mm IOL implantation depth and a 3.5 mm pupil diameter. FIG. 56D illustrates the relative whole-field retinal irradiance for 0.7 mm IOL implantation depth and a 4 mm pupil diameter. FIG. 56E illustrates the relative whole-field retinal irradiance for 0.7 mm IOL implantation depth and a 4.5 mm pupil diameter. FIG. 56F illustrates the relative whole-field retinal irradiance for 0.7 mm IOL implantation depth and a 5 mm pupil diameter. As illustrated, at this implantation depth, no dark band retinal region occurs for the illustrated pupil sizes.

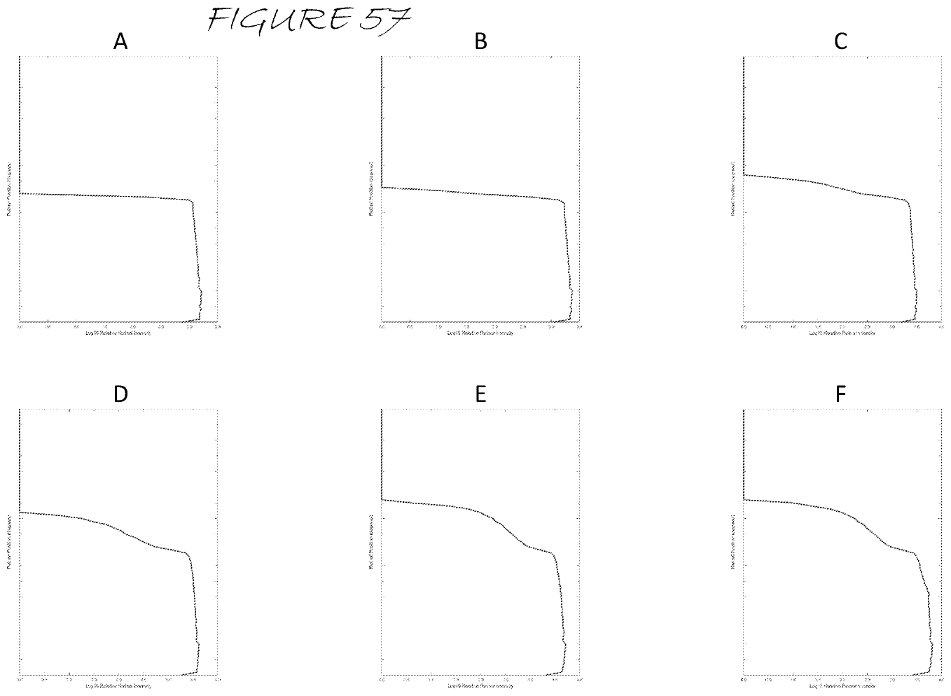

FIGS. 57-64 illustrate the relative whole-field retinal intensity of another exemplary IOL while varying pupil size from 2.5 mm to 5 mm diameter in 0.5 mm steps and an implantation depth from 0 mm to 0.7 mm in 0.1 mm steps. The IOL modelled is another example of an IOL (see FIG. 36 and Table 8) with a control zone for distributing light rays to irradiate the otherwise dark band on the retina. For FIGS. 57-64, about 10,000 rays are traced non-sequentially through the eye model for each field angle. A ray-density plot is generated integrating over the field angles (e.g., 580 to 980 in 0.50 steps). Then, the ray-density results are convolved around azimuthal angles to produce a relative whole-field retinal irradiance plot. The plot axes and scales, and intensity grey-scale, of all plots shown in FIGS. 57-64 are the same as those used in FIGS. 8-15 and FIGS. 19-26 with the exception that the horizontal axis indicating relative whole-field retinal irradiance values, while still using a logarithmic scale (base 10) has a full-scale range of 4 log units (versus the 4.5 log units range for FIGS. 8-15 and FIGS. 19-26). This is due to the fewer rays (10,000 versus 100,000) traced for the model, resulting in lower values in relative retinal irradiance.

FIGS. 57A-F are relative retina irradiance plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0 mm in accordance with certain embodiments. FIG. 57A illustrates the relative whole-field retinal irradiance for 0 mm IOL implantation depth and a 2.5 mm pupil diameter. FIG. 57B illustrates the relative whole-field retinal irradiance for 0 mm IOL implantation depth and a 3 mm pupil diameter. FIG. 57C illustrates the relative whole-field retinal irradiance for 0 mm IOL implantation depth and a 3.5 mm pupil diameter. FIG. 57D illustrates the relative whole-field retinal irradiance for 0 mm IOL implantation depth and a 4 mm pupil diameter. FIG. 57E illustrates the relative whole-field retinal irradiance for 0 mm IOL implantation depth and a 4.5 mm pupil diameter. FIG. 57F illustrates the relative whole-field retinal irradiance for 0 mm IOL implantation depth and a 5 mm pupil diameter. As illustrated, at this implantation depth, no dark band retinal region occurs for the illustrated pupil sizes.

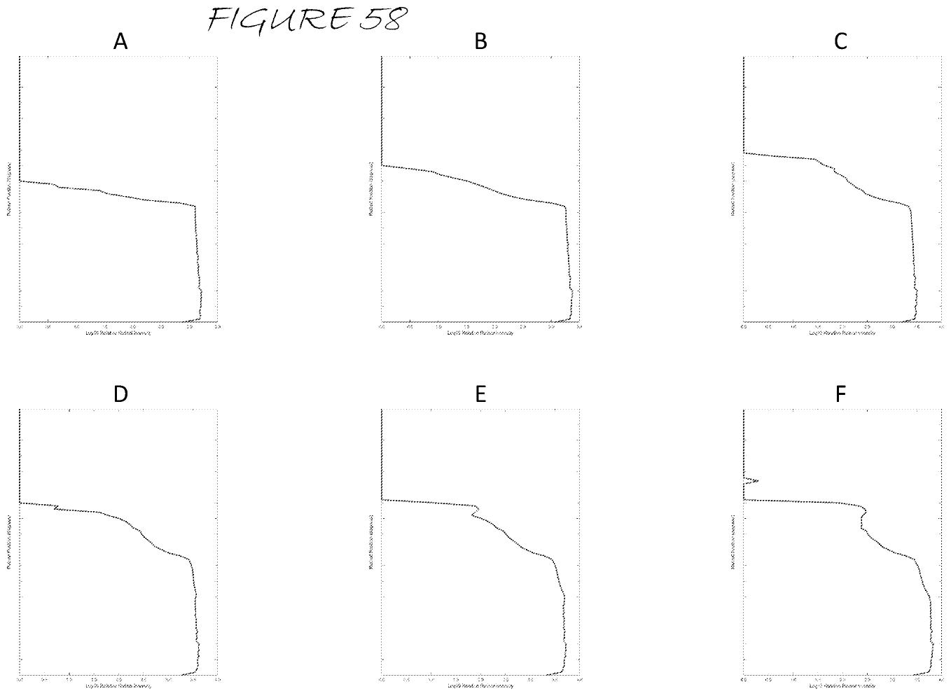

FIGS. 58A-F are relative retina irradiance plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.1 mm in accordance with certain embodiments. FIG. 58A illustrates the relative whole-field retinal irradiance for 0.1 mm IOL implantation depth and a 2.5 mm pupil diameter. FIG. 58B illustrates the relative whole-field retinal irradiance for 0.1 mm IOL implantation depth and a 3 mm pupil diameter. FIG. 58C illustrates the relative whole-field retinal irradiance for 0.1 mm IOL implantation depth and a 3.5 mm pupil diameter. FIG. 58D illustrates the relative whole-field retinal irradiance for 0.1 mm IOL implantation depth and a 4 mm pupil diameter. FIG. 58E illustrates the relative whole-field retinal irradiance for 0.1 mm IOL implantation depth and a 4.5 mm pupil diameter. FIG. 58F illustrates the relative whole-field retinal irradiance for 0.1 mm IOL implantation depth and a 5 mm pupil diameter. As illustrated, at this implantation depth, no dark band retinal region occurs for the illustrated pupil sizes.

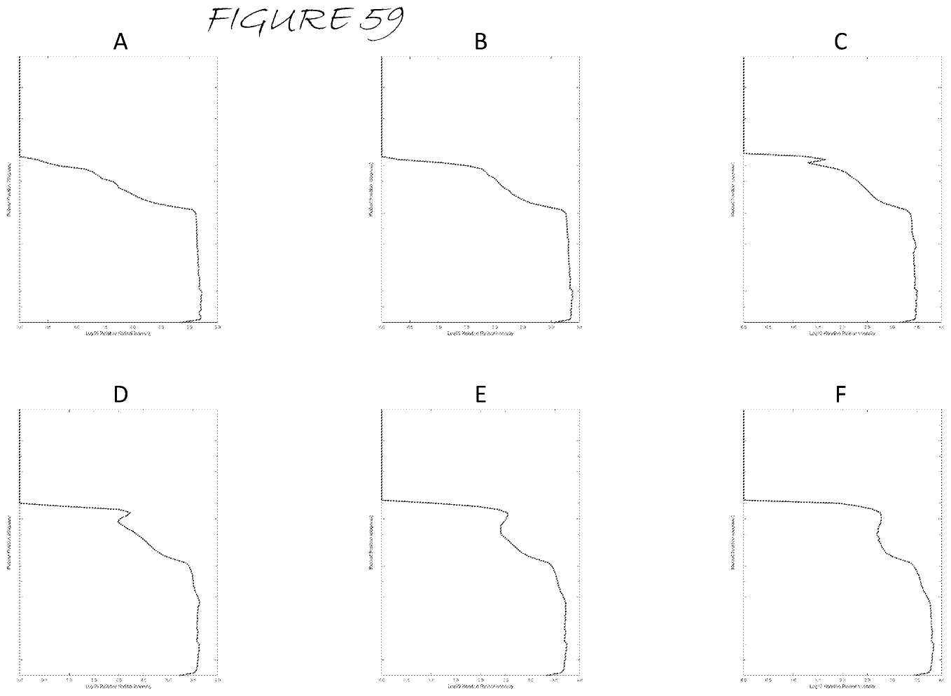

FIGS. 59A-F are relative retina irradiance plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.2 mm in accordance with certain embodiments. FIG. 59A illustrates the relative whole-field retinal irradiance for 0.2 mm IOL implantation depth and a 2.5 mm pupil diameter. FIG. 59B illustrates the relative whole-field retinal irradiance for 0.2 mm IOL implantation depth and a 3 mm pupil diameter. FIG. 59C illustrates the relative whole-field retinal irradiance for 0.2 mm IOL implantation depth and a 3.5 mm pupil diameter. FIG. 59D illustrates the relative whole-field retinal irradiance for 0.2 mm IOL implantation depth and a 4 mm pupil diameter. FIG. 59E illustrates the relative whole-field retinal irradiance for 0.2 mm IOL implantation depth and a 4.5 mm pupil diameter. FIG. 59F illustrates the relative whole-field retinal irradiance for 0.2 mm IOL implantation depth and a 5 mm pupil diameter. As illustrated, at this implantation depth, no dark band retinal region occurs for the illustrated pupil sizes.

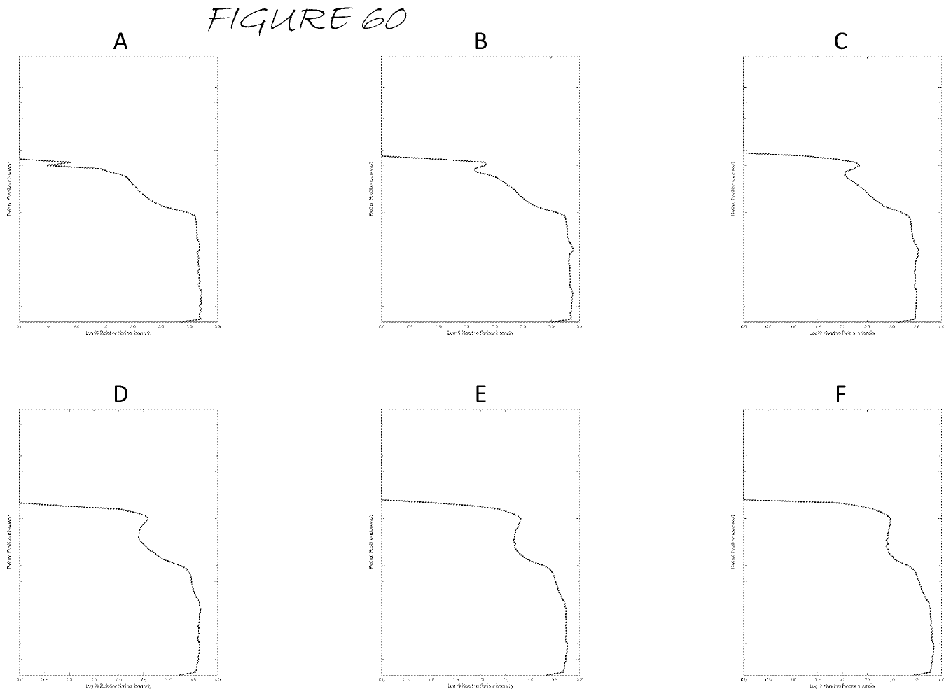

FIGS. 60A-F are relative retina irradiance plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.3 mm in accordance with certain embodiments. FIG. 60A illustrates the relative whole-field retinal irradiance for 0.3 mm IOL implantation depth and a 2.5 mm pupil diameter. FIG. 60B illustrates the relative whole-field retinal irradiance for 0.3 mm IOL implantation depth and a 3 mm pupil diameter. FIG. 60C illustrates the relative whole-field retinal irradiance for 0.3 mm IOL implantation depth and a 3.5 mm pupil diameter. FIG. 60D illustrates the relative whole-field retinal irradiance for 0.3 mm IOL implantation depth and a 4 mm pupil diameter. FIG. 60E illustrates the relative whole-field retinal irradiance for 0.3 mm IOL implantation depth and a 4.5 mm pupil diameter. FIG. 60F illustrates the relative whole-field retinal irradiance for 0.3 mm IOL implantation depth and a 5 mm pupil diameter. As illustrated, at this implantation depth, no dark band retinal region occurs for the illustrated pupil sizes.

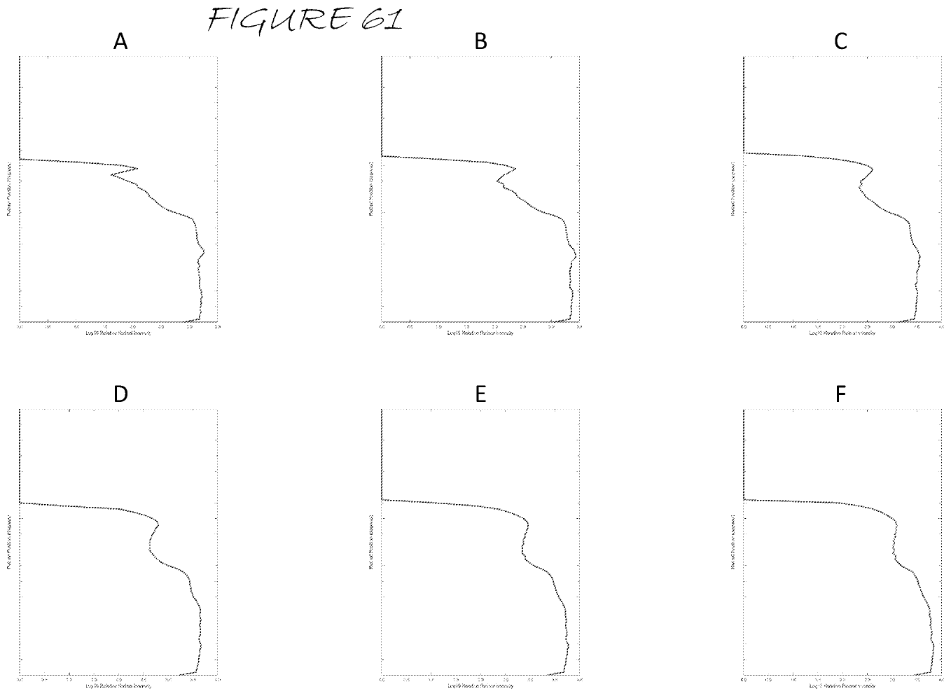

FIGS. 61A-F are relative retina irradiance plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.4 mm in accordance with certain embodiments. FIG. 61A illustrates the relative whole-field retinal irradiance for 0.4 mm IOL implantation depth and a 2.5 mm pupil diameter. FIG. 61B illustrates the relative whole-field retinal irradiance for 0.4 mm IOL implantation depth and a 3 mm pupil diameter. FIG. 61C illustrates the relative whole-field retinal irradiance for 0.4 mm IOL implantation depth and a 3.5 mm pupil diameter. FIG. 61D illustrates the relative whole-field retinal irradiance for 0.4 mm IOL implantation depth and a 4 mm pupil diameter. FIG. 61E illustrates the relative whole-field retinal irradiance for 0.4 mm IOL implantation depth and a 4.5 mm pupil diameter. FIG. 61F illustrates the relative whole-field retinal irradiance for 0.4 mm IOL implantation depth and a 5 mm pupil diameter. As illustrated, at this implantation depth, no dark band retinal region occurs for the illustrated pupil sizes.

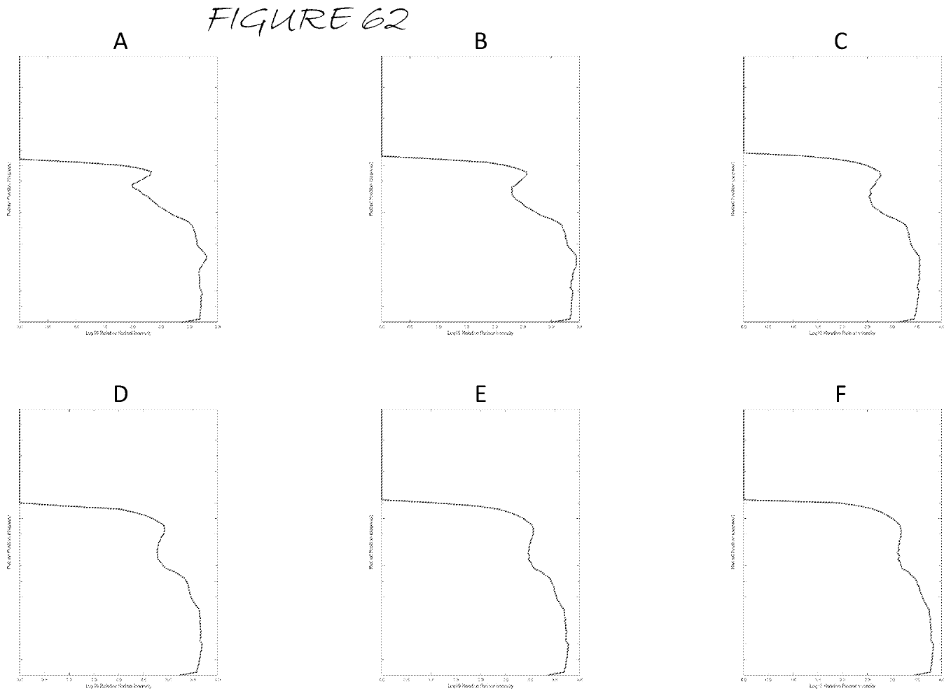

FIGS. 62A-F are relative retina irradiance plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.5 mm in accordance with certain embodiments. FIG. 62A illustrates the relative whole-field retinal irradiance for 0.5 mm IOL implantation depth and a 2.5 mm pupil diameter. FIG. 62B illustrates the relative whole-field retinal irradiance for 0.5 mm IOL implantation depth and a 3 mm pupil diameter. FIG. 62C illustrates the relative whole-field retinal irradiance for 0.5 mm IOL implantation depth and a 3.5 mm pupil diameter. FIG. 62D illustrates the relative whole-field retinal irradiance for 0.5 mm IOL implantation depth and a 4 mm pupil diameter. FIG. 62E illustrates the relative whole-field retinal irradiance for 0.5 mm IOL implantation depth and a 4.5 mm pupil diameter. FIG. 62F illustrates the relative whole-field retinal irradiance for 0.5 mm IOL implantation depth and a 5 mm pupil diameter. As illustrated, at this implantation depth, no dark band retinal region occurs for the illustrated pupil sizes.

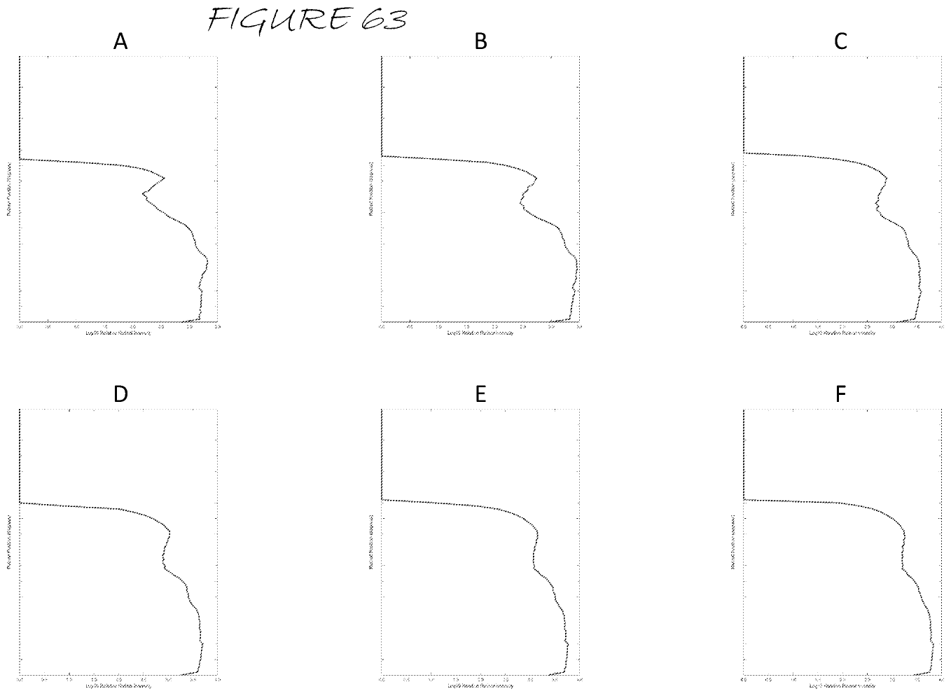

FIGS. 63A-F are relative retina irradiance plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.6 mm in accordance with certain embodiments. FIG. 63A illustrates the relative whole-field retinal irradiance for 0.6 mm IOL implantation depth and a 2.5 mm pupil diameter. FIG. 63B illustrates the relative whole-field retinal irradiance for 0.6 mm IOL implantation depth and a 3 mm pupil diameter. FIG. 63C illustrates the relative whole-field retinal irradiance for 0.6 mm IOL implantation depth and a 3.5 mm pupil diameter. FIG. 63D illustrates the relative whole-field retinal irradiance for 0.6 mm IOL implantation depth and a 4 mm pupil diameter. FIG. 63E illustrates the relative whole-field retinal irradiance for 0.6 mm IOL implantation depth and a 4.5 mm pupil diameter. FIG. 63F illustrates the relative whole-field retinal irradiance for 0.6 mm IOL implantation depth and a 5 mm pupil diameter. As illustrated, at this implantation depth, no dark band retinal region occurs for the illustrated pupil sizes.

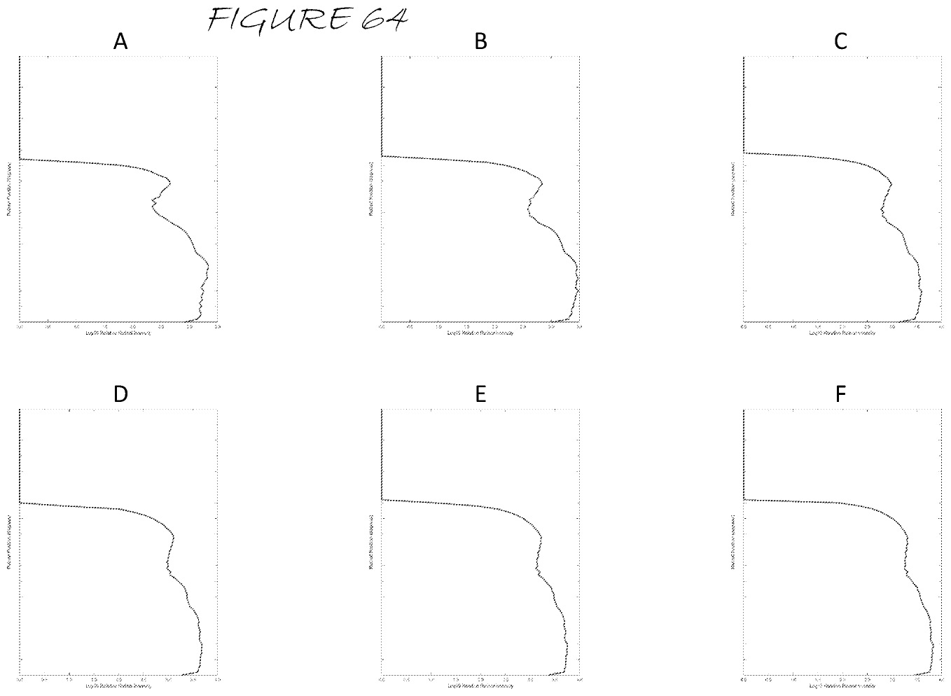

FIGS. 64A-F are relative retina irradiance plots showing the intensity (e.g., relative whole field retinal irradiance) of light distribution across the retina for the intraocular lens modelled in FIG. 36 for pupil sizes from 2.5 mm to 5 mm in 0.5 mm steps at an implantation depth of 0.7 mm in accordance with certain embodiments. FIG. 64A illustrates the relative whole-field retinal irradiance for 0.7 mm IOL implantation depth and a 2.5 mm pupil diameter. FIG. 64B illustrates the relative whole-field retinal irradiance for 0.7 mm IOL implantation depth and a 3 mm pupil diameter. FIG. 64C illustrates the relative whole-field retinal irradiance for 0.7 mm IOL implantation depth and a 3.5 mm pupil diameter. FIG. 64D illustrates the relative whole-field retinal irradiance for 0.7 mm IOL implantation depth and a 4 mm pupil diameter. FIG. 64E illustrates the relative whole-field retinal irradiance for 0.7 mm IOL implantation depth and a 4.5 mm pupil diameter. FIG. 64F illustrates the relative whole-field retinal irradiance for 0.7 mm IOL implantation depth and a 5 mm pupil diameter. As illustrated, at this implantation depth, no dark band retinal region occurs for the illustrated pupil sizes.

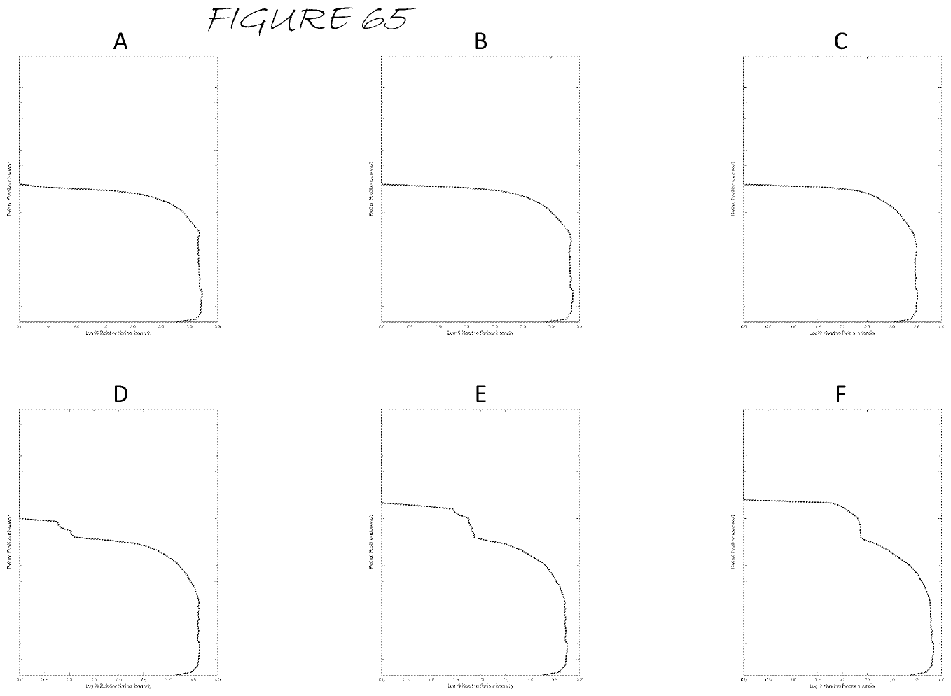

FIGS. 65-72 illustrate the relative whole-field retinal intensity of another exemplary IOL while varying pupil size from 2.5 mm to 5 mm diameter in 0.5 mm steps and an implantation depth from 0 mm to 0.7 mm in 0.1 mm steps. The IOL modelled is another example of an IOL (see FIG. 38 and Table 10) with a control zone for distributing light rays to irradiate the otherwise dark band on the retina. For FIGS. 65-72, about 10,000 rays are traced non-sequentially through the eye model for each field angle. A ray-density plot is generated integrating over the field angles (e.g., 58° to 98° in 0.5° steps). Then, the ray-density results are convolved around azimuthal angles to produce a relative whole-field retinal irradiance plot. The plot axes and scales, and intensity grey-scale, of all plots shown in FIGS. 65-72 are the same as those used in FIGS. 8-15 and FIGS. 19-26 with the exception that the horizontal axis indicating relative whole-field retinal irradiance values, while still using a logarithmic scale (base 10) has a full-scale range of 4 log units (versus the 4.5 log units range for FIGS. 8-15 and FIGS. 19-26). This is due to the fewer rays (10,000 versus 100,000) traced for the model, resulting in lower values in relative retinal irradiance.

FIGS. 65A-F are relative retina irradiance plots showing the intensity (e.g., relative whole field retinal intensity) of light distribution across the retina for the intraocular lens modelled in FIG. 38 for pupil sizes from 2.5 mm to 5 mm in 0.5 mm steps at an implantation depth of 0 mm in accordance with certain embodiments. FIG. 65A illustrates the relative whole-field retinal irradiance for 0 mm IOL implantation depth and a 2.5 mm pupil diameter. FIG. 65B illustrates the relative whole-field retinal irradiance for 0 mm IOL implantation depth and a 3 mm pupil diameter. FIG. 65C illustrates the relative whole-field retinal irradiance for 0 mm IOL implantation depth and a 3.5 mm pupil diameter. FIG. 65D illustrates the relative whole-field retinal irradiance for 0 mm IOL implantation depth and a 4 mm pupil diameter. FIG. 65E illustrates the relative whole-field retinal irradiance for 0 mm IOL implantation depth and a 4.5 mm pupil diameter. FIG. 65F illustrates the relative whole-field retinal irradiance for 0 mm IOL implantation depth and a 5 mm pupil diameter. As illustrated, at this implantation depth, no dark band retinal region occurs for the illustrated pupil sizes.

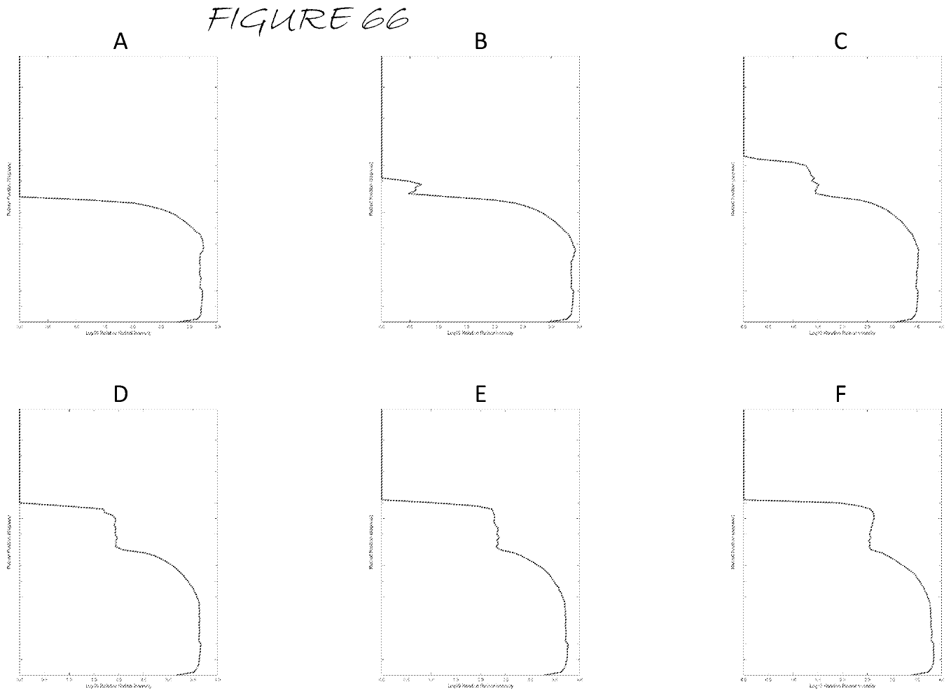

FIGS. 66A-F are relative retina irradiance plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.1 mm in accordance with certain embodiments. FIG. 66A illustrates the relative whole-field retinal irradiance for 0.1 mm IOL implantation depth and a 2.5 mm pupil diameter. FIG. 66B illustrates the relative whole-field retinal irradiance for 0.1 mm IOL implantation depth and a 3 mm pupil diameter. FIG. 66C illustrates the relative whole-field retinal irradiance for 0.1 mm IOL implantation depth and a 3.5 mm pupil diameter. FIG. 66D illustrates the relative whole-field retinal irradiance for 0.1 mm IOL implantation depth and a 4 mm pupil diameter. FIG. 66E illustrates the relative whole-field retinal irradiance for 0.1 mm IOL implantation depth and a 4.5 mm pupil diameter. FIG. 66F illustrates the relative whole-field retinal irradiance for 0.1 mm IOL implantation depth and a 5 mm pupil diameter. As illustrated, at this implantation depth, no dark band retinal region occurs for the illustrated pupil sizes.

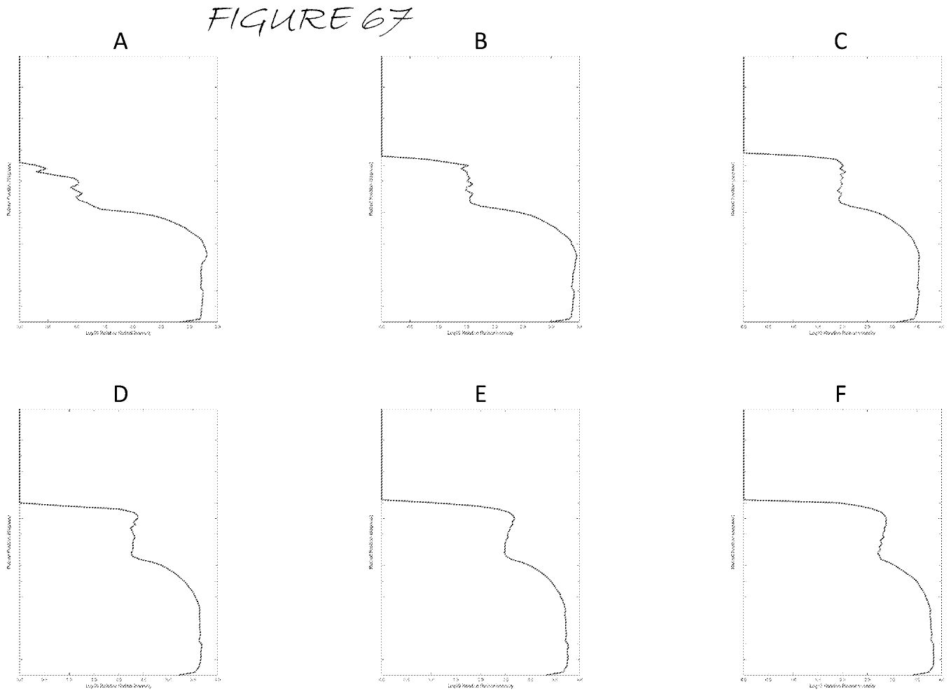

FIGS. 67A-F are relative retina irradiance plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.2 mm in accordance with certain embodiments. FIG. 67A illustrates the relative whole-field retinal irradiance for 0.2 mm IOL implantation depth and a 2.5 mm pupil diameter. FIG. 67B illustrates the relative whole-field retinal irradiance for 0.2 mm IOL implantation depth and a 3 mm pupil diameter. FIG. 67C illustrates the relative whole-field retinal irradiance for 0.2 mm IOL implantation depth and a 3.5 mm pupil diameter. FIG. 67D illustrates the relative whole-field retinal irradiance for 0.2 mm IOL implantation depth and a 4 mm pupil diameter. FIG. 67E illustrates the relative whole-field retinal irradiance for 0.2 mm IOL implantation depth and a 4.5 mm pupil diameter. FIG. 67F illustrates the relative whole-field retinal irradiance for 0.2 mm IOL implantation depth and a 5 mm pupil diameter. As illustrated, at this implantation depth, no dark band retinal region occurs for the illustrated pupil sizes.

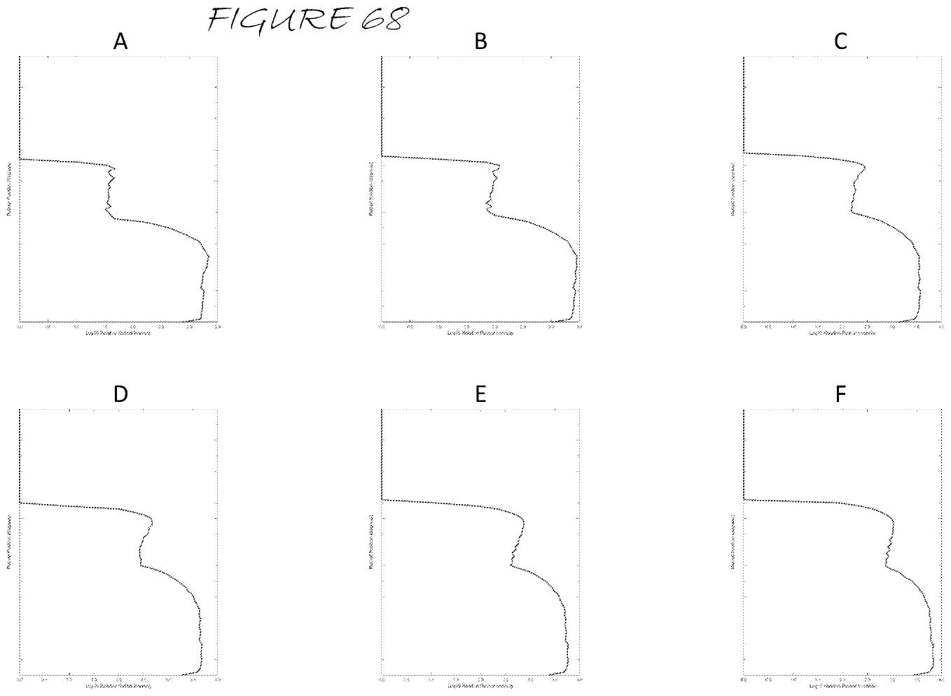

FIGS. 68A-F are relative retina irradiance plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.3 mm in accordance with certain embodiments. FIG. 68A illustrates the relative whole-field retinal irradiance for 0.3 mm IOL implantation depth and a 2.5 mm pupil diameter. FIG. 68B illustrates the relative whole-field retinal irradiance for 0.3 mm IOL implantation depth and a 3 mm pupil diameter. FIG. 68C illustrates the relative whole-field retinal irradiance for 0.3 mm IOL implantation depth and a 3.5 mm pupil diameter. FIG. 68D illustrates the relative whole-field retinal irradiance for 0.3 mm IOL implantation depth and a 4 mm pupil diameter. FIG. 68E illustrates the relative whole-field retinal irradiance for 0.3 mm IOL implantation depth and a 4.5 mm pupil diameter. FIG. 68F illustrates the relative whole-field retinal irradiance for 0.3 mm IOL implantation depth and a 5 mm pupil diameter. As illustrated, at this implantation depth, no dark band retinal region occurs for the illustrated pupil sizes.

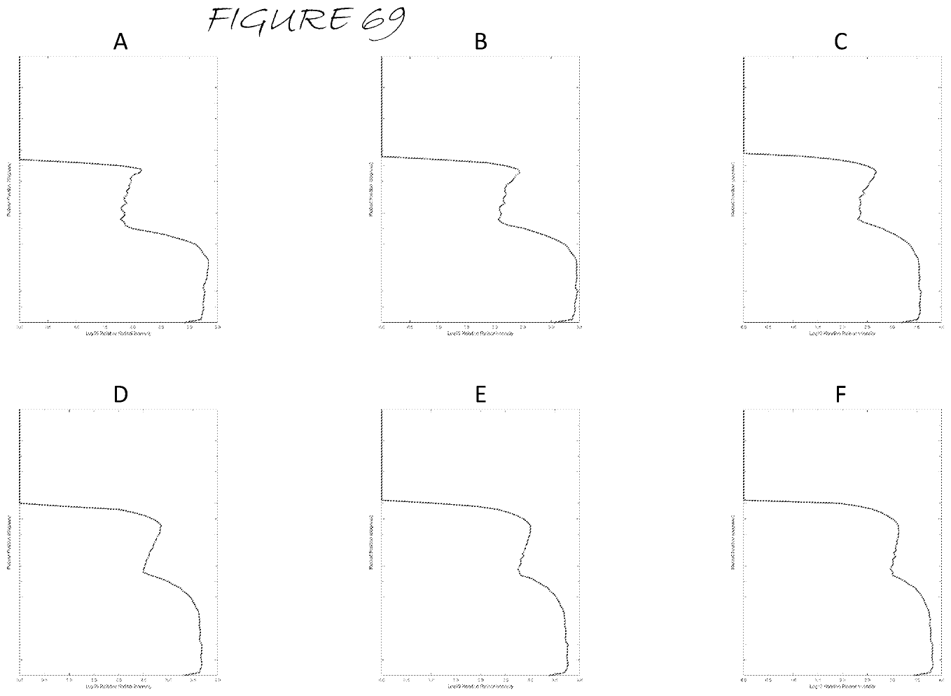

FIGS. 69A-F are relative retina irradiance plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.4 mm in accordance with certain embodiments. FIG. 69A illustrates the relative whole-field retinal irradiance for 0.4 mm IOL implantation depth and a 2.5 mm pupil diameter. FIG. 69B illustrates the relative whole-field retinal irradiance for 0.4 mm IOL implantation depth and a 3 mm pupil diameter. FIG. 69C illustrates the relative whole-field retinal irradiance for 0.4 mm IOL implantation depth and a 3.5 mm pupil diameter. FIG. 69D illustrates the relative whole-field retinal irradiance for 0.4 mm IOL implantation depth and a 4 mm pupil diameter. FIG. 69E illustrates the relative whole-field retinal irradiance for 0.4 mm IOL implantation depth and a 4.5 mm pupil diameter. FIG. 69F illustrates the relative whole-field retinal irradiance for 0.4 mm IOL implantation depth and a 5 mm pupil diameter. As illustrated, at this implantation depth, no dark band retinal region occurs for the illustrated pupil sizes.

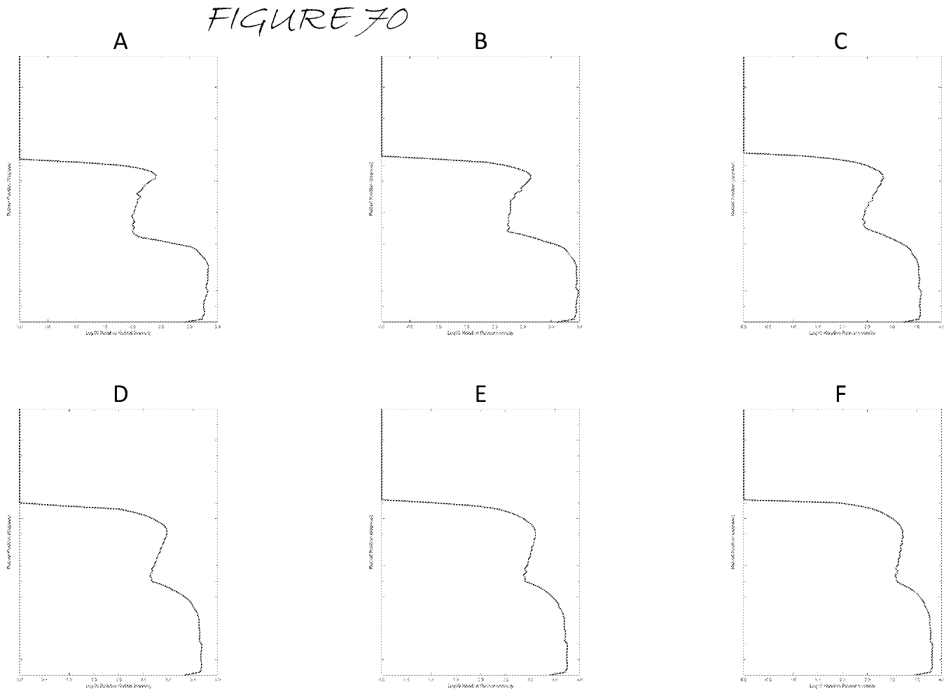

FIGS. 70A-F are relative retina irradiance plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.5 mm in accordance with certain embodiments. FIG. 70A illustrates the relative whole-field retinal irradiance for 0.5 mm IOL implantation depth and a 2.5 mm pupil diameter. FIG. 70B illustrates the relative whole-field retinal irradiance for 0.5 mm IOL implantation depth and a 3 mm pupil diameter. FIG. 70C illustrates the relative whole-field retinal irradiance for 0.5 mm IOL implantation depth and a 3.5 mm pupil diameter. FIG. 70D illustrates the relative whole-field retinal irradiance for 0.5 mm IOL implantation depth and a 4 mm pupil diameter. FIG. 70E illustrates the relative whole-field retinal irradiance for 0.5 mm IOL implantation depth and a 4.5 mm pupil diameter. FIG. 70F illustrates the relative whole-field retinal irradiance for 0.5 mm IOL implantation depth and a 5 mm pupil diameter. As illustrated, at this implantation depth, no dark band retinal region occurs for the illustrated pupil sizes.

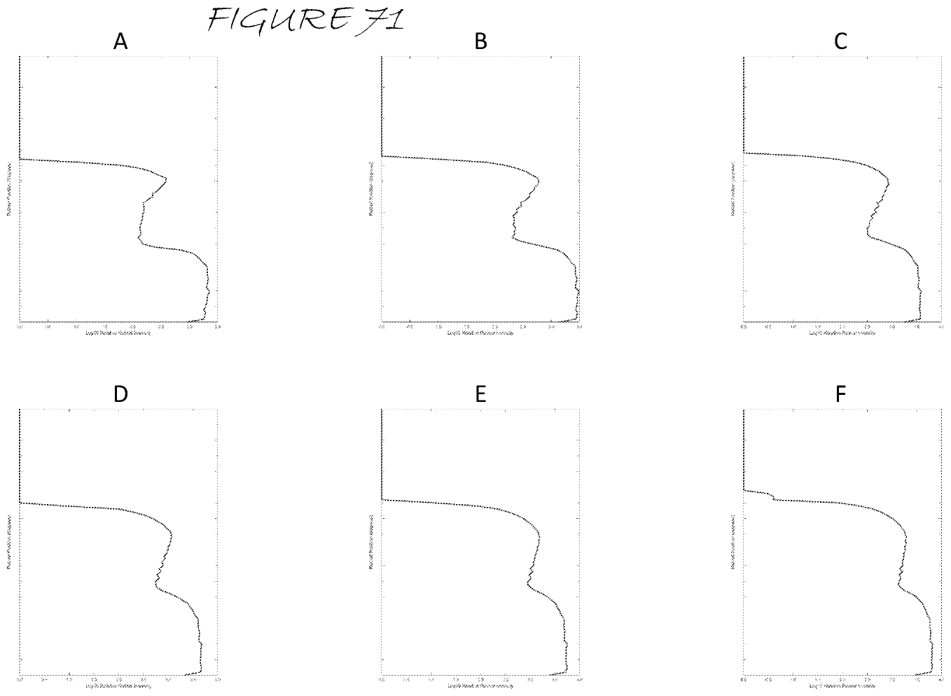

FIGS. 71A-F are relative retina irradiance plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.6 mm in accordance with certain embodiments. FIG. 71A illustrates the relative whole-field retinal irradiance for 0.6 mm IOL implantation depth and a 2.5 mm pupil diameter. FIG. 71B illustrates the relative whole-field retinal irradiance for 0.6 mm IOL implantation depth and a 3 mm pupil diameter. FIG. 71C illustrates the relative whole-field retinal irradiance for 0.6 mm IOL implantation depth and a 3.5 mm pupil diameter. FIG. 71D illustrates the relative whole-field retinal irradiance for 0.6 mm IOL implantation depth and a 4 mm pupil diameter. FIG. 71E illustrates the relative whole-field retinal irradiance for 0.6 mm IOL implantation depth and a 4.5 mm pupil diameter. FIG. 71F illustrates the relative whole-field retinal irradiance for 0.6 mm IOL implantation depth and a 5 mm pupil diameter. As illustrated, at this implantation depth, no dark band retinal region occurs for the illustrated pupil sizes.

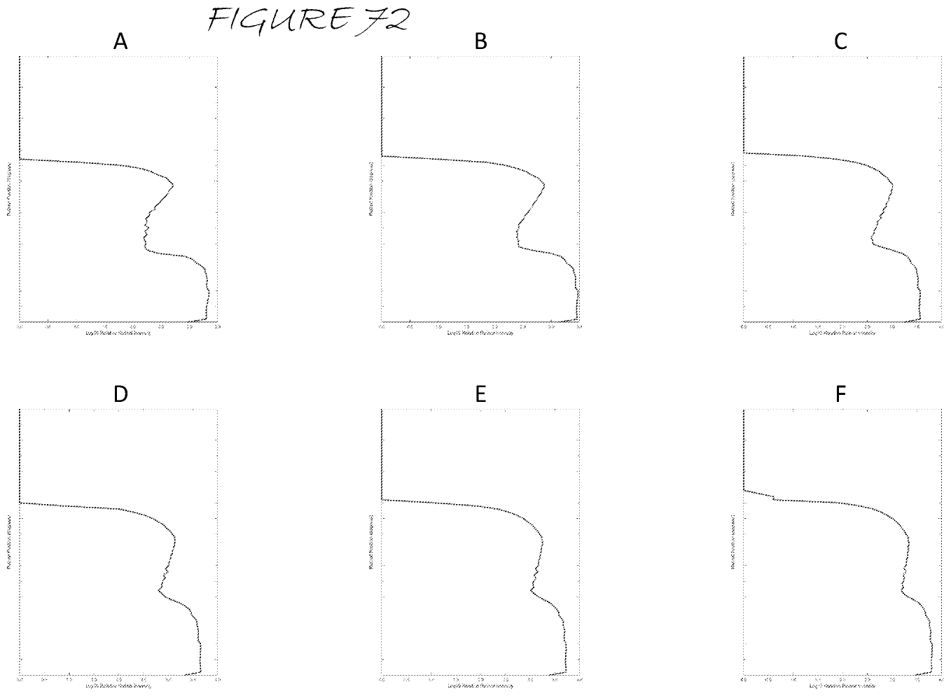

FIGS. 72A-F are relative retina irradiance plots showing the intensity (e.g., relative whole field retinal irradiance) of light distribution across the retina for the intraocular lens modelled in FIG. 38 for pupil sizes from 2.5 mm to 5 mm in 0.5 mm steps at an implantation depth of 0.7 mm in accordance with certain embodiments. FIG. 72A illustrates the relative whole-field retinal irradiance for 0.7 mm IOL implantation depth and a 2.5 mm pupil diameter. FIG. 72B illustrates the relative whole-field retinal irradiance for 0.7 mm IOL implantation depth and a 3 mm pupil diameter. FIG. 72C illustrates the relative whole-field retinal irradiance for 0.7 mm IOL implantation depth and a 3.5 mm pupil diameter. FIG. 72D illustrates the relative whole-field retinal irradiance for 0.7 mm IOL implantation depth and a 4 mm pupil diameter. FIG. 72E illustrates the relative whole-field retinal irradiance for 0.7 mm IOL implantation depth and a 4.5 mm pupil diameter. FIG. 72F illustrates the relative whole-field retinal irradiance for 0.7 mm IOL implantation depth and a 5 mm pupil diameter. As illustrated, at this implantation depth, no dark band retinal region occurs for the illustrated pupil sizes.

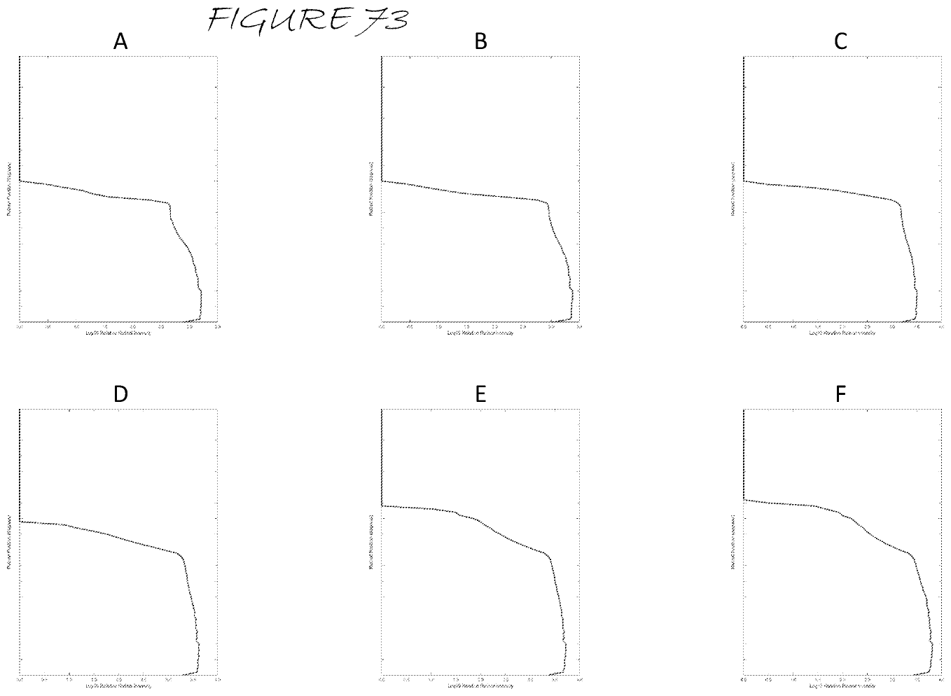

FIGS. 73-80 illustrate the relative whole-field retinal intensity of another exemplary IOL while varying pupil size from 2.5 mm to 5 mm diameter in 0.5 mm steps and an implantation depth from 0 mm to 0.7 mm in 0.1 mm steps. The IOL modelled is another example of an IOL (see FIG. 40 and Table 12) with a control zone for distributing light rays to irradiate the otherwise dark band on the retina. For FIGS. 73-80, about 10,000 rays are traced non-sequentially through the eye model for each field angle. A ray-density plot is generated integrating over the field angles (e.g., 58° to 98° in 0.5° steps). Then, the ray-density results are convolved around azimuthal angles to produce a relative whole-field retinal irradiance plot. The plot axes and scales, and intensity grey-scale, of all plots shown in FIGS. 73-80 are the same as those used in FIGS. 8-15 and FIGS. 19-26 with the exception that the horizontal axis indicating relative whole-field retinal irradiance values, while still using a logarithmic scale (base 10) has a full-scale range of 4 log units (versus the 4.5 log units range for FIGS. 8-15 and FIGS. 19-26). This is due to the fewer rays (10,000 versus 100,000) traced for the model, resulting in lower values in relative retinal irradiance.

FIGS. 73A-F are relative retina irradiance plots showing the intensity (e.g., relative whole field retinal intensity) of light distribution across the retina for the intraocular lens modelled in FIG. 40 for pupil sizes from 2.5 mm to 5 mm in 0.5 mm steps at an implantation depth of 0 mm in accordance with certain embodiments. FIG. 73A illustrates the relative whole-field retinal irradiance for 0 mm IOL implantation depth and a 2.5 mm pupil diameter. FIG. 73B illustrates the relative whole-field retinal irradiance for 0 mm IOL implantation depth and a 3 mm pupil diameter. FIG. 73C illustrates the relative whole-field retinal irradiance for 0 mm IOL implantation depth and a 3.5 mm pupil diameter. FIG. 73D illustrates the relative whole-field retinal irradiance for 0 mm IOL implantation depth and a 4 mm pupil diameter. FIG. 73E illustrates the relative whole-field retinal irradiance for 0 mm IOL implantation depth and a 4.5 mm pupil diameter. FIG. 73F illustrates the relative whole-field retinal irradiance for 0 mm IOL implantation depth and a 5 mm pupil diameter. As illustrated, at this implantation depth, no dark band retinal region occurs for the illustrated pupil sizes.

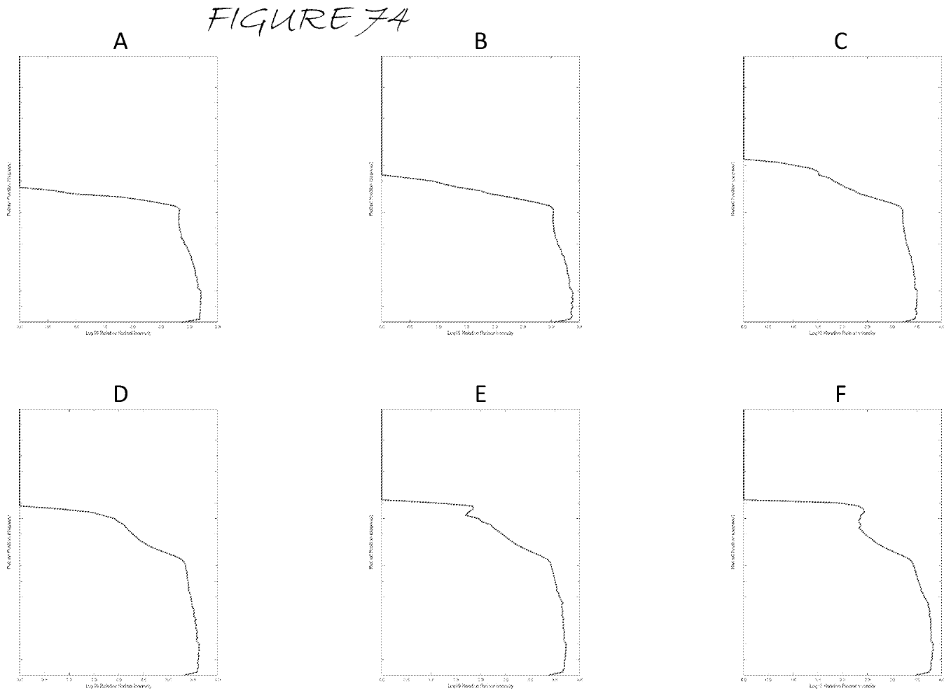

FIGS. 74A-F are relative retina irradiance plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.1 mm in accordance with certain embodiments. FIG. 74A illustrates the relative whole-field retinal irradiance for 0.1 mm IOL implantation depth and a 2.5 mm pupil diameter. FIG. 74B illustrates the relative whole-field retinal irradiance for 0.1 mm IOL implantation depth and a 3 mm pupil diameter. FIG. 74C illustrates the relative whole-field retinal irradiance for 0.1 mm IOL implantation depth and a 3.5 mm pupil diameter. FIG. 74D illustrates the relative whole-field retinal irradiance for 0.1 mm IOL implantation depth and a 4 mm pupil diameter. FIG. 74E illustrates the relative whole-field retinal irradiance for 0.1 mm IOL implantation depth and a 4.5 mm pupil diameter. FIG. 74F illustrates the relative whole-field retinal irradiance for 0.1 mm IOL implantation depth and a 5 mm pupil diameter. As illustrated, at this implantation depth, no dark band retinal region occurs for the illustrated pupil sizes.

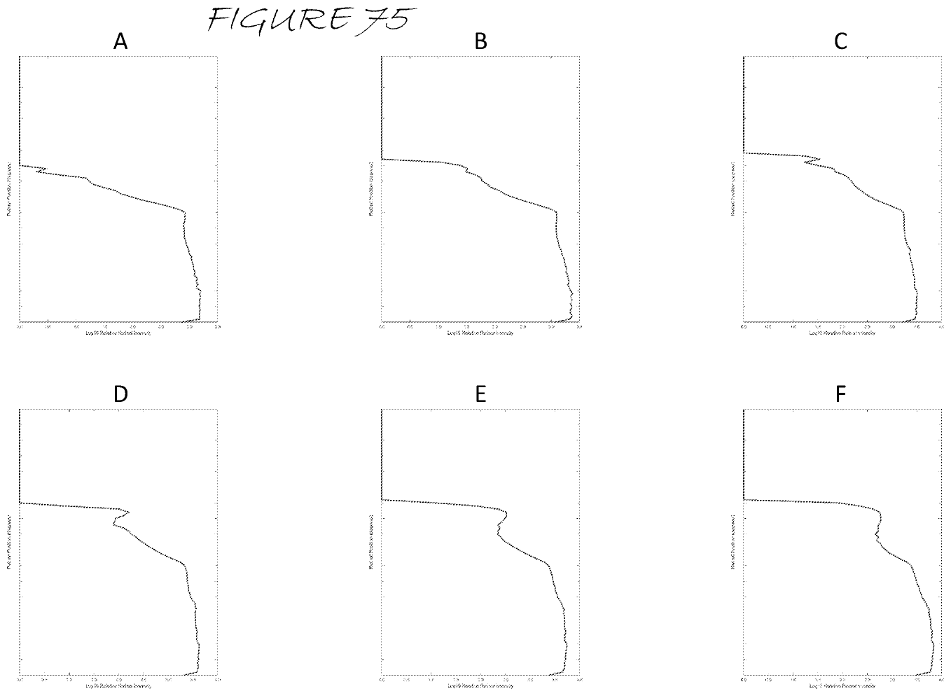

FIGS. 75A-F are relative retina irradiance plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.2 mm in accordance with certain embodiments. FIG. 75A illustrates the relative whole-field retinal irradiance for 0.2 mm IOL implantation depth and a 2.5 mm pupil diameter. FIG. 75B illustrates the relative whole-field retinal irradiance for 0.2 mm IOL implantation depth and a 3 mm pupil diameter. FIG. 75C illustrates the relative whole-field retinal irradiance for 0.2 mm IOL implantation depth and a 3.5 mm pupil diameter. FIG. 75D illustrates the relative whole-field retinal irradiance for 0.2 mm IOL implantation depth and a 4 mm pupil diameter. FIG. 75E illustrates the relative whole-field retinal irradiance for 0.2 mm IOL implantation depth and a 4.5 mm pupil diameter. FIG. 75F illustrates the relative whole-field retinal irradiance for 0.2 mm IOL implantation depth and a 5 mm pupil diameter. As illustrated, at this implantation depth, no dark band retinal region occurs for the illustrated pupil sizes.

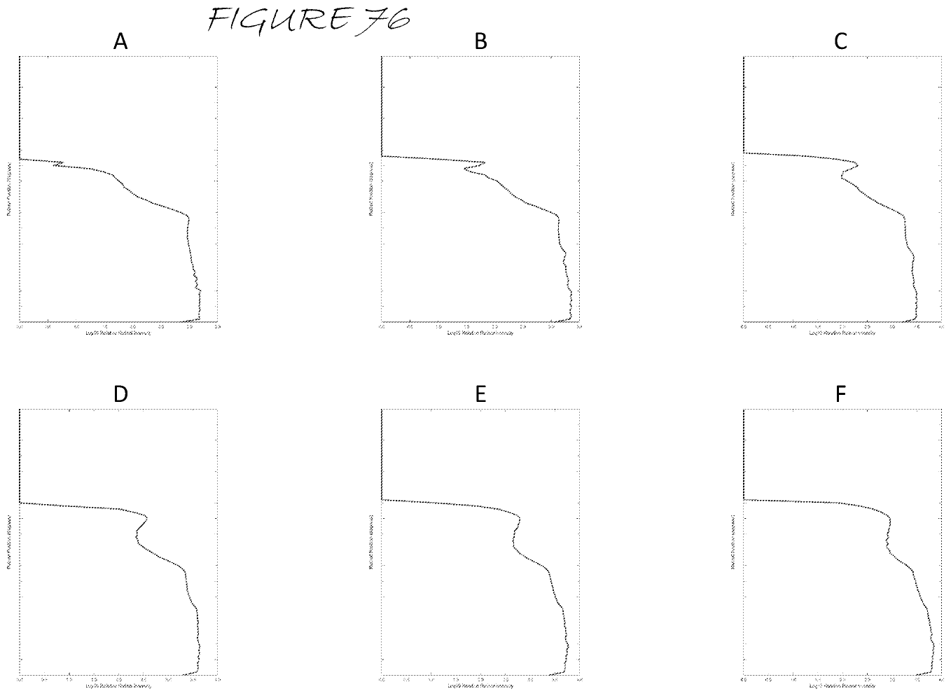

FIGS. 76A-F are relative retina irradiance plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.3 mm in accordance with certain embodiments. FIG. 76A illustrates the relative whole-field retinal irradiance for 0.3 mm IOL implantation depth and a 2.5 mm pupil diameter. FIG. 76B illustrates the relative whole-field retinal irradiance for 0.3 mm IOL implantation depth and a 3 mm pupil diameter. FIG. 76C illustrates the relative whole-field retinal irradiance for 0.3 mm IOL implantation depth and a 3.5 mm pupil diameter. FIG. 76D illustrates the relative whole-field retinal irradiance for 0.3 mm IOL implantation depth and a 4 mm pupil diameter. FIG. 76E illustrates the relative whole-field retinal irradiance for 0.3 mm IOL implantation depth and a 4.5 mm pupil diameter. FIG. 76F illustrates the relative whole-field retinal irradiance for 0.3 mm IOL implantation depth and a 5 mm pupil diameter. As illustrated, at this implantation depth, no dark band retinal region occurs for the illustrated pupil sizes.

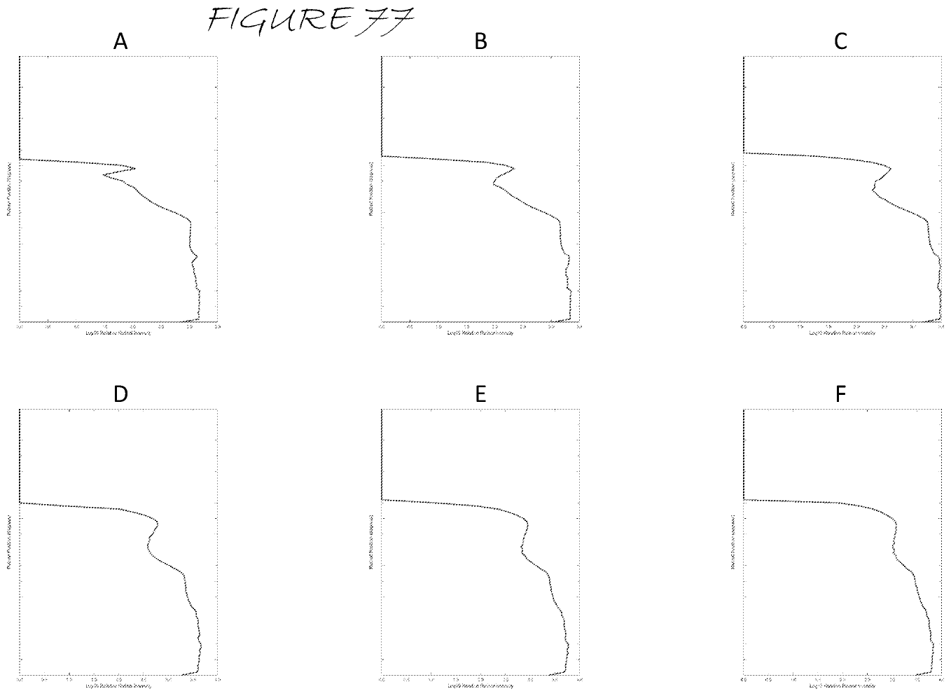

FIGS. 77A-F are relative retina irradiance plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.4 mm in accordance with certain embodiments. FIG. 77A illustrates the relative whole-field retinal irradiance for 0.4 mm IOL implantation depth and a 2.5 mm pupil diameter. FIG. 77B illustrates the relative whole-field retinal irradiance for 0.4 mm IOL implantation depth and a 3 mm pupil diameter. FIG. 77C illustrates the relative whole-field retinal irradiance for 0.4 mm IOL implantation depth and a 3.5 mm pupil diameter. FIG. 77D illustrates the relative whole-field retinal irradiance for 0.4 mm IOL implantation depth and a 4 mm pupil diameter. FIG. 77E illustrates the relative whole-field retinal irradiance for 0.4 mm IOL implantation depth and a 4.5 mm pupil diameter. FIG. 77F illustrates the relative whole-field retinal irradiance for 0.4 mm IOL implantation depth and a 5 mm pupil diameter. As illustrated, at this implantation depth, no dark band retinal region occurs for the illustrated pupil sizes.

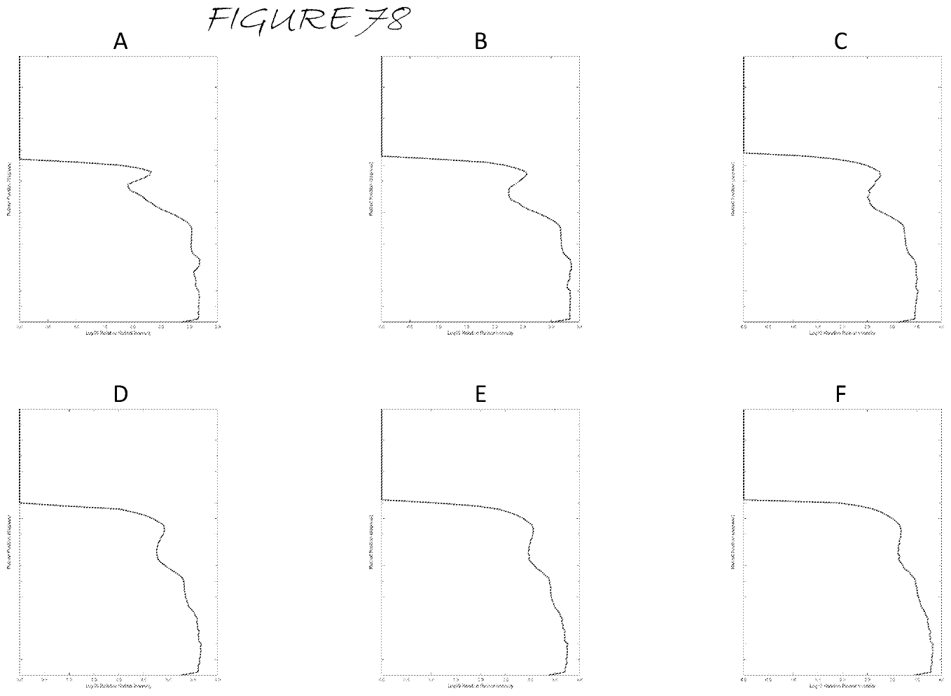

FIGS. 78A-F are relative retina irradiance plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.5 mm in accordance with certain embodiments. FIG. 78A illustrates the relative whole-field retinal irradiance for 0.5 mm IOL implantation depth and a 2.5 mm pupil diameter. FIG. 78B illustrates the relative whole-field retinal irradiance for 0.5 mm IOL implantation depth and a 3 mm pupil diameter. FIG. 78C illustrates the relative whole-field retinal irradiance for 0.5 mm IOL implantation depth and a 3.5 mm pupil diameter. FIG. 78D illustrates the relative whole-field retinal irradiance for 0.5 mm IOL implantation depth and a 4 mm pupil diameter. FIG. 78E illustrates the relative whole-field retinal irradiance for 0.5 mm IOL implantation depth and a 4.5 mm pupil diameter. FIG. 78F illustrates the relative whole-field retinal irradiance for 0.5 mm IOL implantation depth and a 5 mm pupil diameter. As illustrated, at this implantation depth, no dark band retinal region occurs for the illustrated pupil sizes.

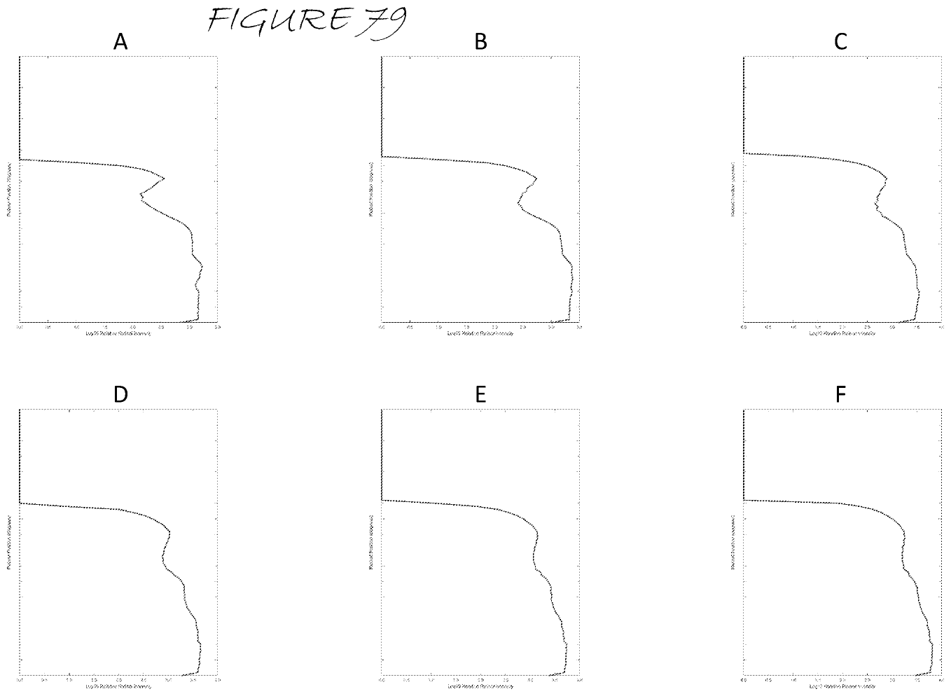

FIGS. 79A-F are relative retina irradiance plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.6 mm in accordance with certain embodiments. FIG. 79A illustrates the relative whole-field retinal irradiance for 0.6 mm IOL implantation depth and a 2.5 mm pupil diameter. FIG. 79B illustrates the relative whole-field retinal irradiance for 0.6 mm IOL implantation depth and a 3 mm pupil diameter. FIG. 79C illustrates the relative whole-field retinal irradiance for 0.6 mm IOL implantation depth and a 3.5 mm pupil diameter. FIG. 79D illustrates the relative whole-field retinal irradiance for 0.6 mm IOL implantation depth and a 4 mm pupil diameter. FIG. 79E illustrates the relative whole-field retinal irradiance for 0.6 mm IOL implantation depth and a 4.5 mm pupil diameter. FIG. 79F illustrates the relative whole-field retinal irradiance for 0.6 mm IOL implantation depth and a 5 mm pupil diameter. As illustrated, at this implantation depth, no dark band retinal region occurs for the illustrated pupil sizes.

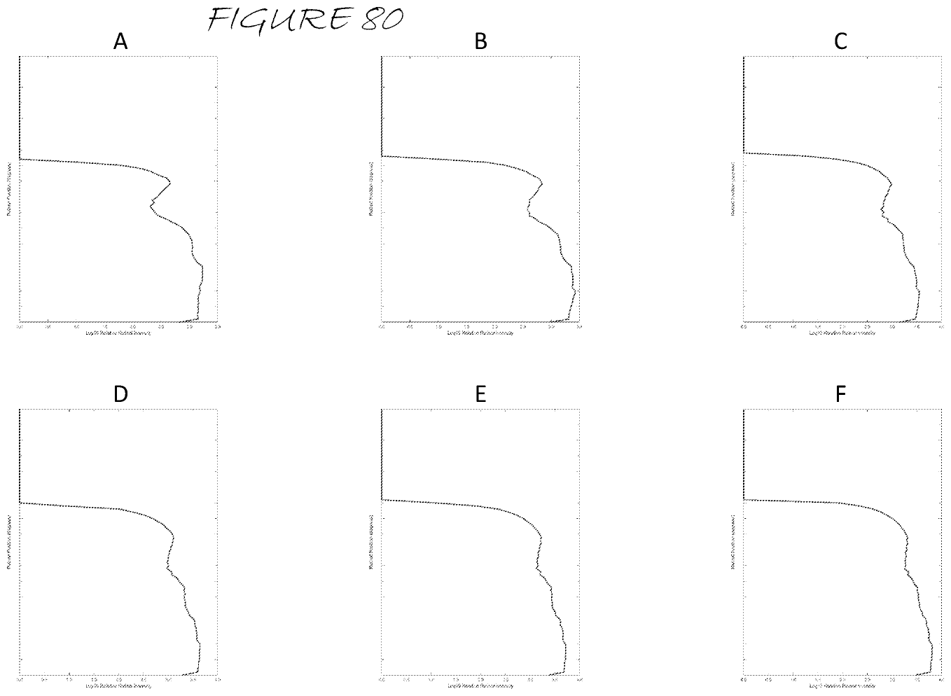

FIGS. 80A-F are relative retina irradiance plots showing the intensity (e.g., relative whole field retinal irradiance) of light distribution across the retina for the intraocular lens modelled in FIG. 40 for pupil sizes from 2.5 mm to 5 mm in 0.5 mm steps at an implantation depth of 0.7 mm in accordance with certain embodiments. FIG. 80A illustrates the relative whole-field retinal irradiance for 0.7 mm IOL implantation depth and a 2.5 mm pupil diameter. FIG. 80B illustrates the relative whole-field retinal irradiance for 0.7 mm IOL implantation depth and a 3 mm pupil diameter. FIG. 80C illustrates the relative whole-field retinal irradiance for 0.7 mm IOL implantation depth and a 3.5 mm pupil diameter. FIG. 80D illustrates the relative whole-field retinal irradiance for 0.7 mm IOL implantation depth and a 4 mm pupil diameter. FIG. 80E illustrates the relative whole-field retinal irradiance for 0.7 mm IOL implantation depth and a 4.5 mm pupil diameter. FIG. 80F illustrates the relative whole-field retinal irradiance for 0.7 mm IOL implantation depth and a 5 mm pupil diameter. As illustrated, at this implantation depth, no dark band retinal region occurs for the illustrated pupil sizes.

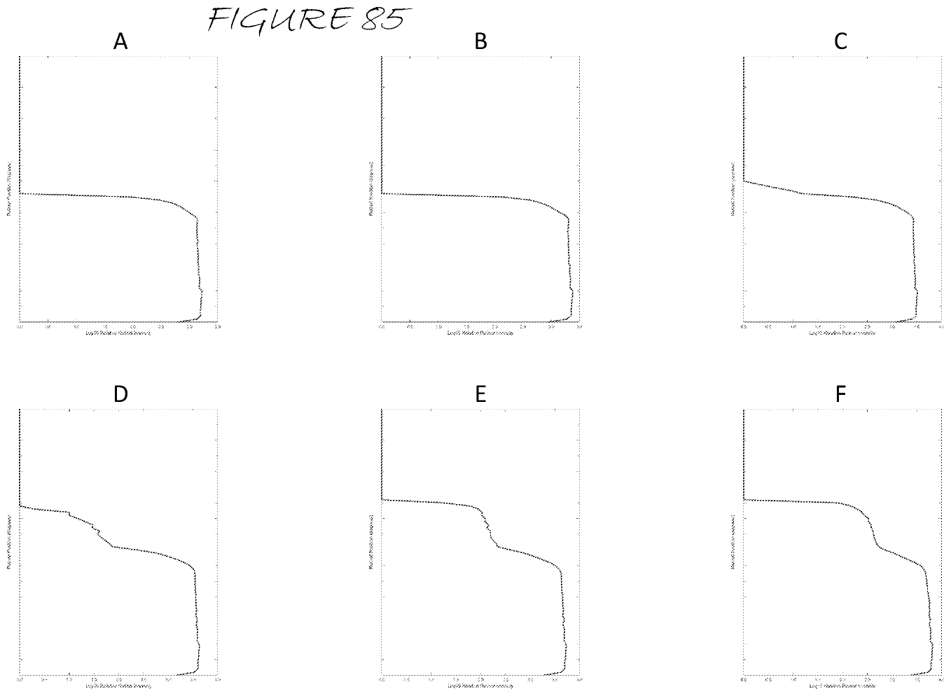

FIGS. 85-92 illustrate the relative whole-field retinal intensity of another exemplary IOL while varying pupil size from 2.5 mm to 5 mm diameter in 0.5 mm steps and an implantation depth from 0 mm to 0.7 mm in 0.1 mm steps. The IOL modelled is another example of an IOL (see FIG. 82 and Table 14) with a control zone for distributing light rays to irradiate the otherwise dark band on the retina. For FIGS. 85-92, about 10,000 rays are traced non-sequentially through the eye model for each field angle. A ray-density plot is generated integrating over the field angles (e.g., 58° to 98° in 0.5° steps). Then, the ray-density results are convolved around azimuthal angles to produce a relative whole-field retinal irradiance plot. The plot axes and scales, and intensity grey-scale, of all plots shown in FIGS. 85-92 are the same as those used in FIGS. 8-15 and FIGS. 19-26 with the exception that the horizontal axis indicating relative whole-field retinal irradiance values, while still using a logarithmic scale (base 10) has a full-scale range of 4 log units (versus the 4.5 log units range for FIGS. 8-15 and FIGS. 19-26). This is due to the fewer rays (10,000 versus 100,000) traced for the model, resulting in lower values in relative retinal irradiance.

FIGS. 85A-F are relative retina irradiance plots showing the intensity (e.g., relative whole field retinal intensity) of light distribution across the retina for the intraocular lens modelled in FIG. 82 for pupil sizes from 2.5 mm to 5 mm in 0.5 mm steps at an implantation depth of 0 mm in accordance with certain embodiments. FIG. 85A illustrates the relative whole-field retinal irradiance for 0 mm IOL implantation depth and a 2.5 mm pupil diameter. FIG. 85B illustrates the relative whole-field retinal irradiance for 0 mm IOL implantation depth and a 3 mm pupil diameter. FIG. 85C illustrates the relative whole-field retinal irradiance for 0 mm IOL implantation depth and a 3.5 mm pupil diameter. FIG. 85D illustrates the relative whole-field retinal irradiance for 0 mm IOL implantation depth and a 4 mm pupil diameter. FIG. 85E illustrates the relative whole-field retinal irradiance for 0 mm IOL implantation depth and a 4.5 mm pupil diameter. FIG. 85F illustrates the relative whole-field retinal irradiance for 0 mm IOL implantation depth and a 5 mm pupil diameter. As illustrated, at this implantation depth, no dark band retinal region occurs for the illustrated pupil sizes.

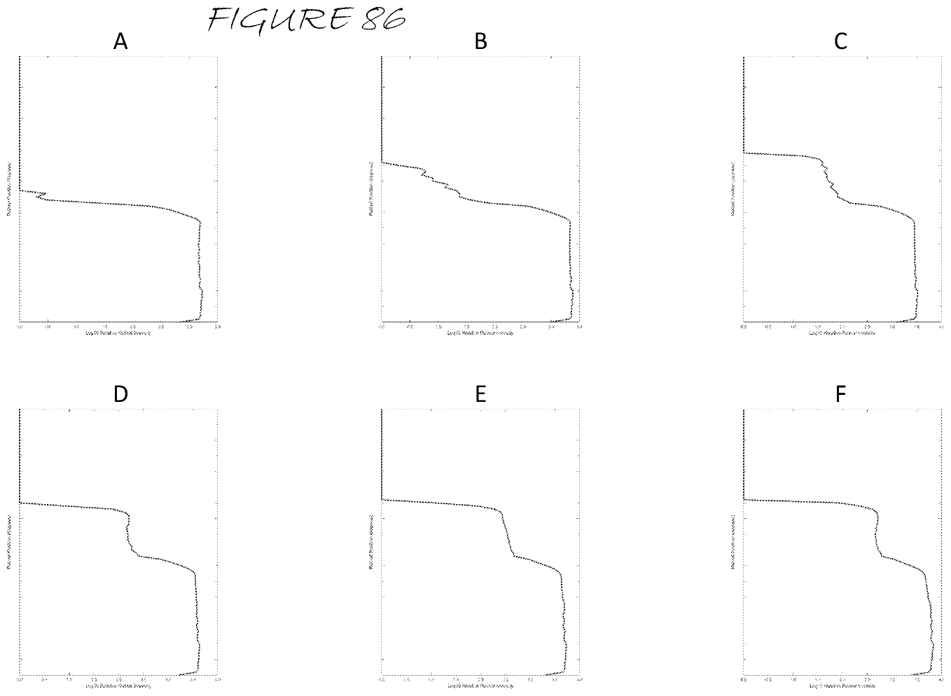

FIGS. 86A-F are relative retina irradiance plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.1 mm in accordance with certain embodiments. FIG. 86A illustrates the relative whole-field retinal irradiance for 0.1 mm IOL implantation depth and a 2.5 mm pupil diameter. FIG. 86B illustrates the relative whole-field retinal irradiance for 0.1 mm IOL implantation depth and a 3 mm pupil diameter. FIG. 86C illustrates the relative whole-field retinal irradiance for 0.1 mm IOL implantation depth and a 3.5 mm pupil diameter. FIG. 86D illustrates the relative whole-field retinal irradiance for 0.1 mm IOL implantation depth and a 4 mm pupil diameter. FIG. 86E illustrates the relative whole-field retinal irradiance for 0.1 mm IOL implantation depth and a 4.5 mm pupil diameter. FIG. 86F illustrates the relative whole-field retinal irradiance for 0.1 mm IOL implantation depth and a 5 mm pupil diameter. As illustrated, at this implantation depth, no dark band retinal region occurs for the illustrated pupil sizes.

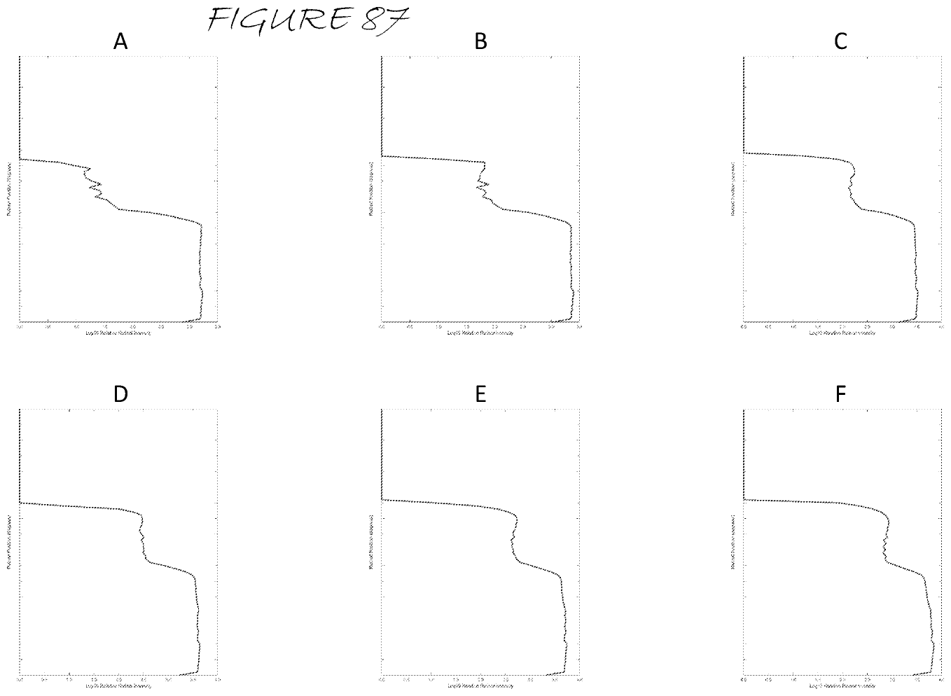

FIGS. 87A-F are relative retina irradiance plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.2 mm in accordance with certain embodiments. FIG. 87A illustrates the relative whole-field retinal irradiance for 0.2 mm IOL implantation depth and a 2.5 mm pupil diameter. FIG. 87B illustrates the relative whole-field retinal irradiance for 0.2 mm IOL implantation depth and a 3 mm pupil diameter. FIG. 87C illustrates the relative whole-field retinal irradiance for 0.2 mm IOL implantation depth and a 3.5 mm pupil diameter. FIG. 87D illustrates the relative whole-field retinal irradiance for 0.2 mm IOL implantation depth and a 4 mm pupil diameter. FIG. 87E illustrates the relative whole-field retinal irradiance for 0.2 mm IOL implantation depth and a 4.5 mm pupil diameter. FIG. 87F illustrates the relative whole-field retinal irradiance for 0.2 mm IOL implantation depth and a 5 mm pupil diameter. As illustrated, at this implantation depth, no dark band retinal region occurs for the illustrated pupil sizes.

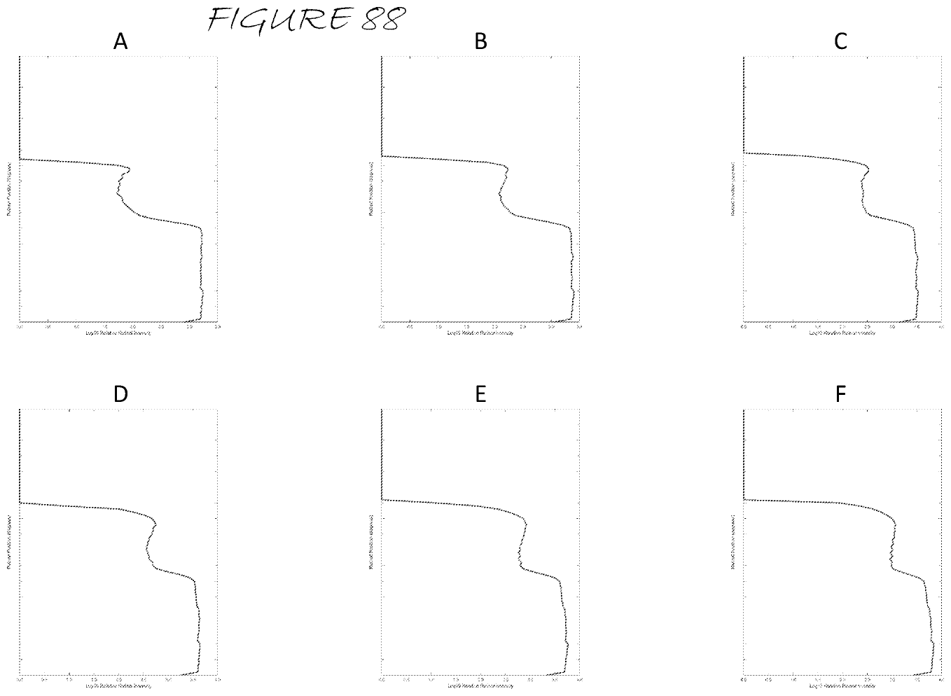

FIGS. 88A-F are relative retina irradiance plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.3 mm in accordance with certain embodiments. FIG. 88A illustrates the relative whole-field retinal irradiance for 0.3 mm IOL implantation depth and a 2.5 mm pupil diameter. FIG. 88B illustrates the relative whole-field retinal irradiance for 0.3 mm IOL implantation depth and a 3 mm pupil diameter. FIG. 88C illustrates the relative whole-field retinal irradiance for 0.3 mm IOL implantation depth and a 3.5 mm pupil diameter. FIG. 88D illustrates the relative whole-field retinal irradiance for 0.3 mm IOL implantation depth and a 4 mm pupil diameter. FIG. 88E illustrates the relative whole-field retinal irradiance for 0.3 mm IOL implantation depth and a 4.5 mm pupil diameter. FIG. 88F illustrates the relative whole-field retinal irradiance for 0.3 mm IOL implantation depth and a 5 mm pupil diameter. As illustrated, at this implantation depth, no dark band retinal region occurs for the illustrated pupil sizes.

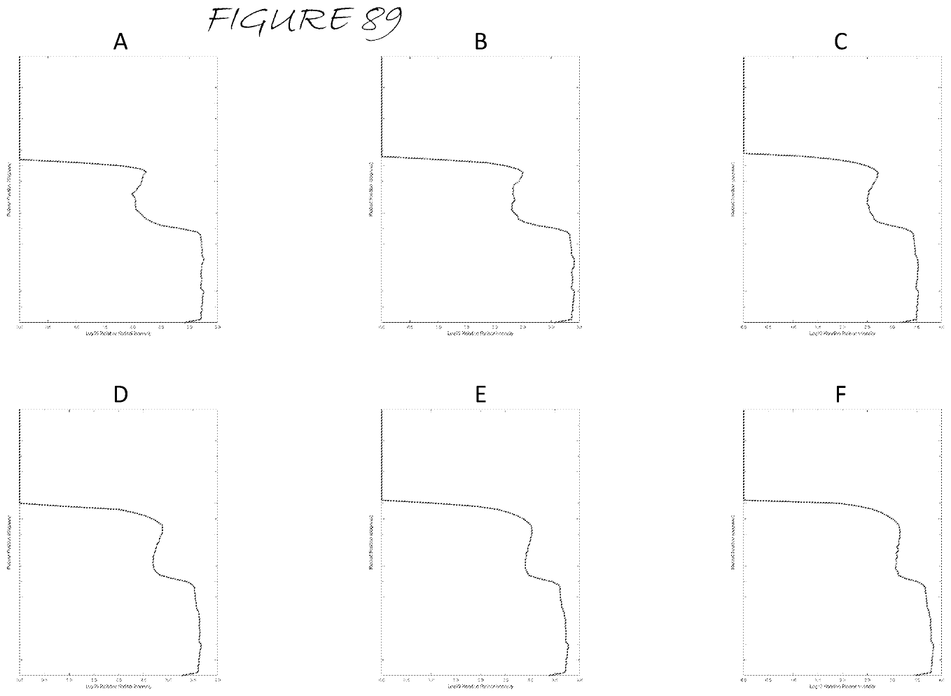

FIGS. 89A-F are relative retina irradiance plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.4 mm in accordance with certain embodiments. FIG. 89A illustrates the relative whole-field retinal irradiance for 0.4 mm IOL implantation depth and a 2.5 mm pupil diameter. FIG. 89B illustrates the relative whole-field retinal irradiance for 0.4 mm IOL implantation depth and a 3 mm pupil diameter. FIG. 89C illustrates the relative whole-field retinal irradiance for 0.4 mm IOL implantation depth and a 3.5 mm pupil diameter. FIG. 89D illustrates the relative whole-field retinal irradiance for 0.4 mm IOL implantation depth and a 4 mm pupil diameter. FIG. 89E illustrates the relative whole-field retinal irradiance for 0.4 mm IOL implantation depth and a 4.5 mm pupil diameter. FIG. 89F illustrates the relative whole-field retinal irradiance for 0.4 mm IOL implantation depth and a 5 mm pupil diameter. As illustrated, at this implantation depth, no dark band retinal region occurs for the illustrated pupil sizes.

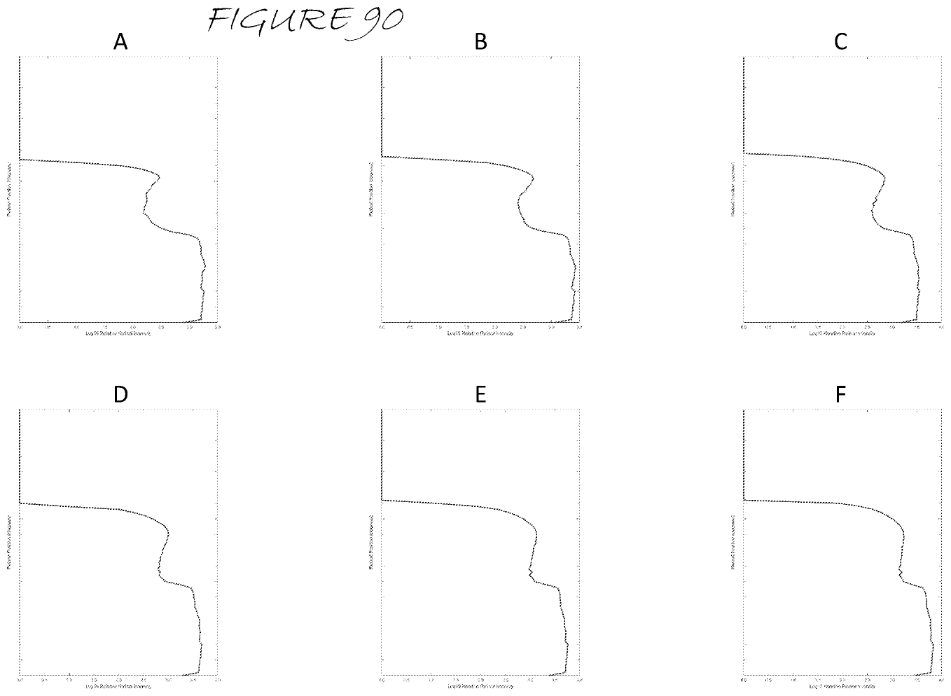

FIGS. 90A-F are relative retina irradiance plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.5 mm in accordance with certain embodiments. FIG. 90A illustrates the relative whole-field retinal irradiance for 0.5 mm IOL implantation depth and a 2.5 mm pupil diameter. FIG. 90B illustrates the relative whole-field retinal irradiance for 0.5 mm IOL implantation depth and a 3 mm pupil diameter. FIG. 90C illustrates the relative whole-field retinal irradiance for 0.5 mm IOL implantation depth and a 3.5 mm pupil diameter. FIG. 90D illustrates the relative whole-field retinal irradiance for 0.5 mm IOL implantation depth and a 4 mm pupil diameter. FIG. 90E illustrates the relative whole-field retinal irradiance for 0.5 mm IOL implantation depth and a 4.5 mm pupil diameter. FIG. 90F illustrates the relative whole-field retinal irradiance for 0.5 mm IOL implantation depth and a 5 mm pupil diameter. As illustrated, at this implantation depth, no dark band retinal region occurs for the illustrated pupil sizes.

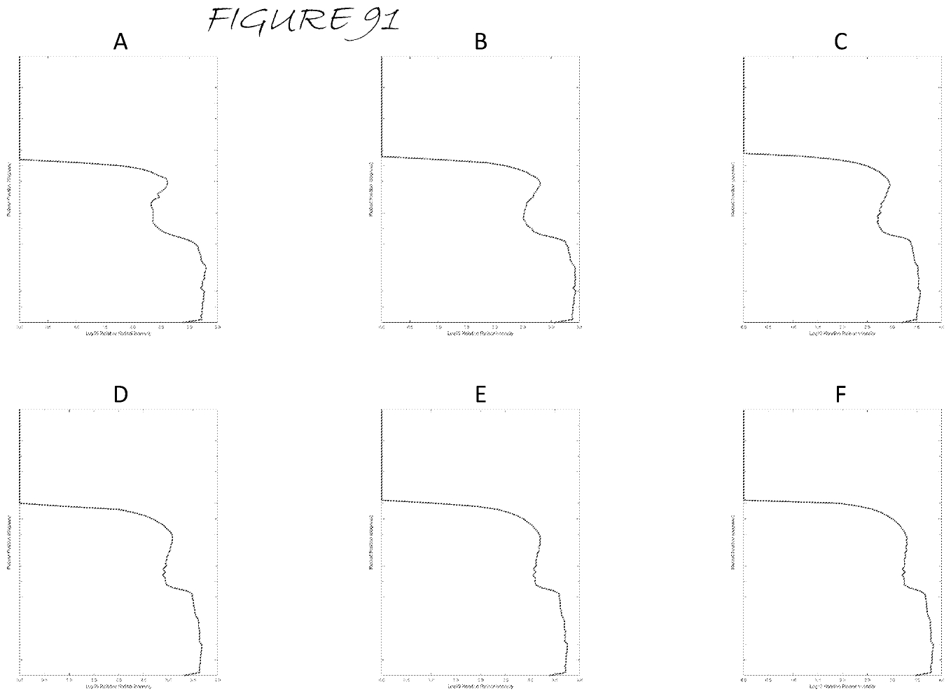

FIGS. 91A-F are relative retina irradiance plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.6 mm in accordance with certain embodiments. FIG. 91A illustrates the relative whole-field retinal irradiance for 0.6 mm IOL implantation depth and a 2.5 mm pupil diameter. FIG. 91B illustrates the relative whole-field retinal irradiance for 0.6 mm IOL implantation depth and a 3 mm pupil diameter. FIG. 91C illustrates the relative whole-field retinal irradiance for 0.6 mm IOL implantation depth and a 3.5 mm pupil diameter. FIG. 91D illustrates the relative whole-field retinal irradiance for 0.6 mm IOL implantation depth and a 4 mm pupil diameter. FIG. 91E illustrates the relative whole-field retinal irradiance for 0.6 mm IOL implantation depth and a 4.5 mm pupil diameter. FIG. 91F illustrates the relative whole-field retinal irradiance for 0.6 mm IOL implantation depth and a 5 mm pupil diameter. As illustrated, at this implantation depth, no dark band retinal region occurs for the illustrated pupil sizes.

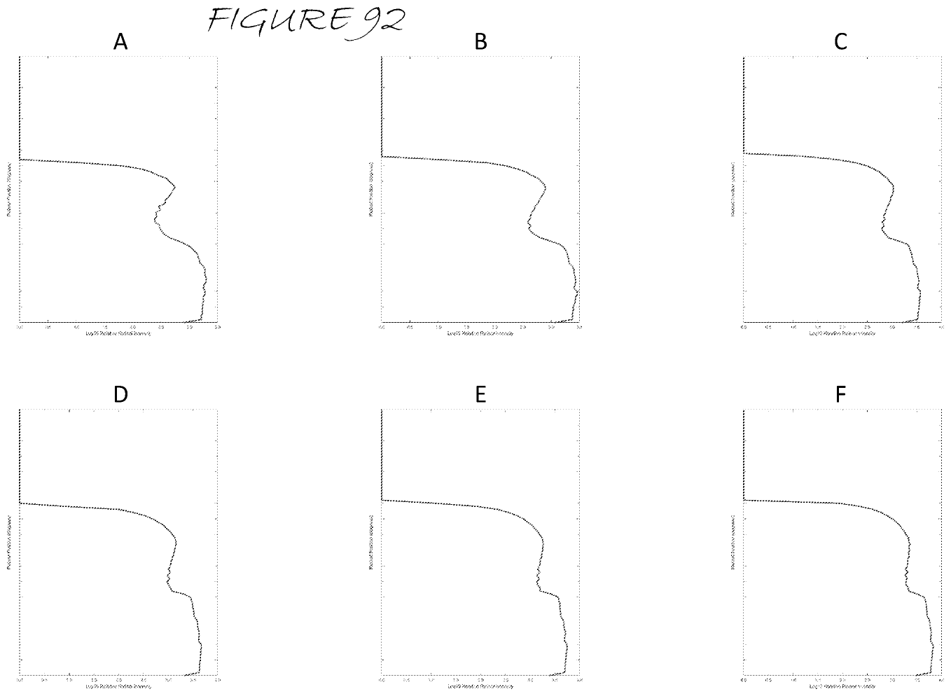

FIGS. 92A-F are relative retina irradiance plots showing the intensity (e.g., relative whole field retinal irradiance) of light distribution across the retina for the intraocular lens modelled in FIG. 82 for pupil sizes from 2.5 mm to 5 mm in 0.5 mm steps at an implantation depth of 0.7 mm in accordance with certain embodiments. FIG. 92A illustrates the relative whole-field retinal irradiance for 0.7 mm IOL implantation depth and a 2.5 mm pupil diameter. FIG. 92B illustrates the relative whole-field retinal irradiance for 0.7 mm IOL implantation depth and a 3 mm pupil diameter. FIG. 92C illustrates the relative whole-field retinal irradiance for 0.7 mm IOL implantation depth and a 3.5 mm pupil diameter. FIG. 92D illustrates the relative whole-field retinal irradiance for 0.7 mm IOL implantation depth and a 4 mm pupil diameter. FIG. 92E illustrates the relative whole-field retinal irradiance for 0.7 mm IOL implantation depth and a 4.5 mm pupil diameter. FIG. 92F illustrates the relative whole-field retinal irradiance for 0.7 mm IOL implantation depth and a 5 mm pupil diameter. As illustrated, at this implantation depth, no dark band retinal region occurs for the illustrated pupil sizes.

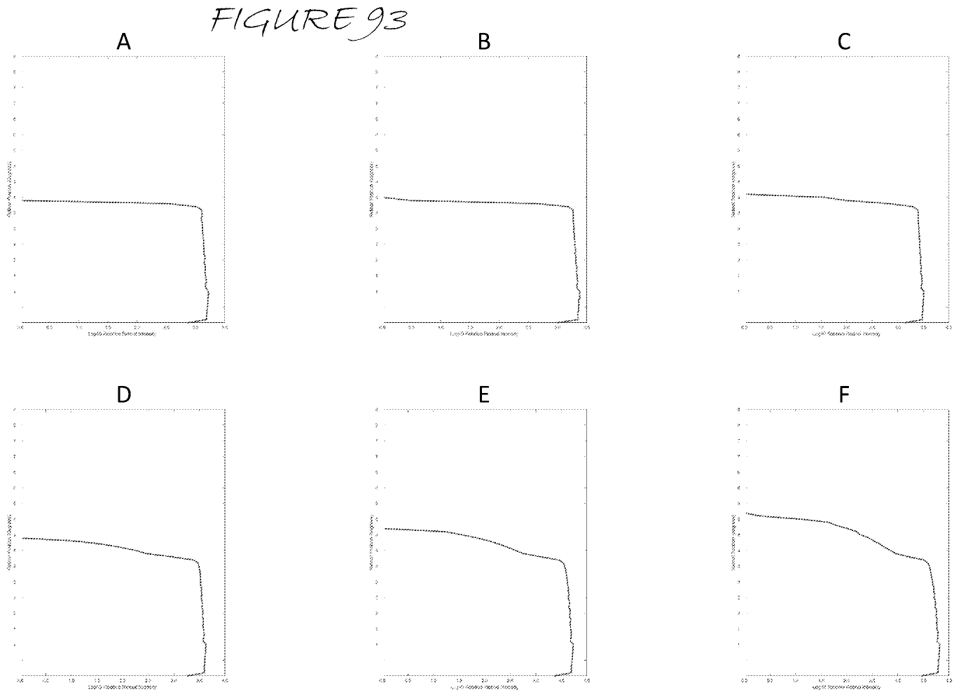

FIGS. 93-100 illustrate the relative whole-field retinal intensity of another exemplary IOL while varying pupil size from 2.5 mm to 5 mm diameter in 0.5 mm steps and an implantation depth from 0 mm to 0.7 mm in 0.1 mm steps. The IOL modelled is another example of an IOL (see FIG. 84 and Table 16) with a control zone for distributing light rays to irradiate the otherwise dark band on the retina. For FIGS. 93-100, about 10,000 rays are traced non-sequentially through the eye model for each field angle. A ray-density plot is generated integrating over the field angles (e.g., 58° to 98° in 0.5° steps). Then, the ray-density results are convolved around azimuthal angles to produce a relative whole-field retinal irradiance plot. The plot axes and scales, and intensity grey-scale, of all plots shown in FIGS. 93-100 are the same as those used in FIGS. 8-15 and FIGS. 19-26 with the exception that the horizontal axis indicating relative whole-field retinal irradiance values, while still using a logarithmic scale (base 10) has a full-scale range of 4 log units (versus the 4.5 log units range for FIGS. 8-15 and FIGS. 19-26). This is due to the fewer rays (10,000 versus 100,000) traced for the model, resulting in lower values in relative retinal irradiance.

FIGS. 93A-F are relative retina irradiance plots showing the intensity (e.g., relative whole field retinal intensity) of light distribution across the retina for the intraocular lens modelled in FIG. 84 for pupil sizes from 2.5 mm to 5 mm in 0.5 mm steps at an implantation depth of 0 mm in accordance with certain embodiments. FIG. 93A illustrates the relative whole-field retinal irradiance for 0 mm IOL implantation depth and a 2.5 mm pupil diameter. FIG. 93B illustrates the relative whole-field retinal irradiance for 0 mm IOL implantation depth and a 3 mm pupil diameter. FIG. 93C illustrates the relative whole-field retinal irradiance for 0 mm IOL implantation depth and a 3.5 mm pupil diameter. FIG. 93D illustrates the relative whole-field retinal irradiance for 0 mm IOL implantation depth and a 4 mm pupil diameter. FIG. 93E illustrates the relative whole-field retinal irradiance for 0 mm IOL implantation depth and a 4.5 mm pupil diameter. FIG. 93F illustrates the relative whole-field retinal irradiance for 0 mm IOL implantation depth and a 5 mm pupil diameter. As illustrated, at this implantation depth, no dark band retinal region occurs for the illustrated pupil sizes.

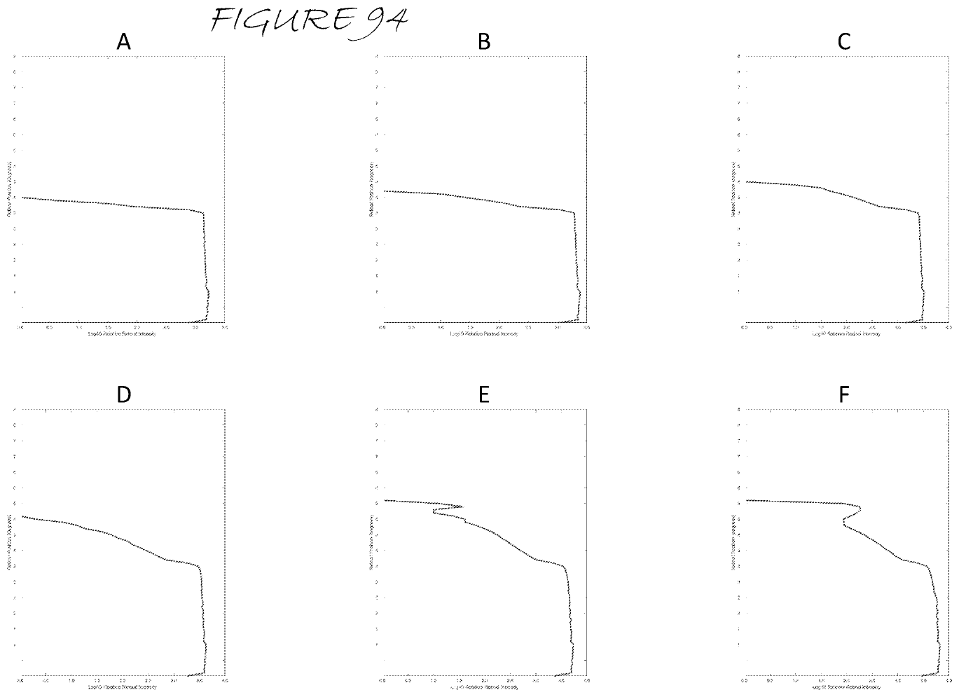

FIGS. 94A-F are relative retina irradiance plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.1 mm in accordance with certain embodiments. FIG. 94A illustrates the relative whole-field retinal irradiance for 0.1 mm IOL implantation depth and a 2.5 mm pupil diameter. FIG. 94B illustrates the relative whole-field retinal irradiance for 0.1 mm IOL implantation depth and a 3 mm pupil diameter. FIG. 94C illustrates the relative whole-field retinal irradiance for 0.1 mm IOL implantation depth and a 3.5 mm pupil diameter. FIG. 94D illustrates the relative whole-field retinal irradiance for 0.1 mm IOL implantation depth and a 4 mm pupil diameter. FIG. 94E illustrates the relative whole-field retinal irradiance for 0.1 mm IOL implantation depth and a 4.5 mm pupil diameter. FIG. 94F illustrates the relative whole-field retinal irradiance for 0.1 mm IOL implantation depth and a 5 mm pupil diameter. As illustrated, at this implantation depth, no dark band retinal region occurs for the illustrated pupil sizes.

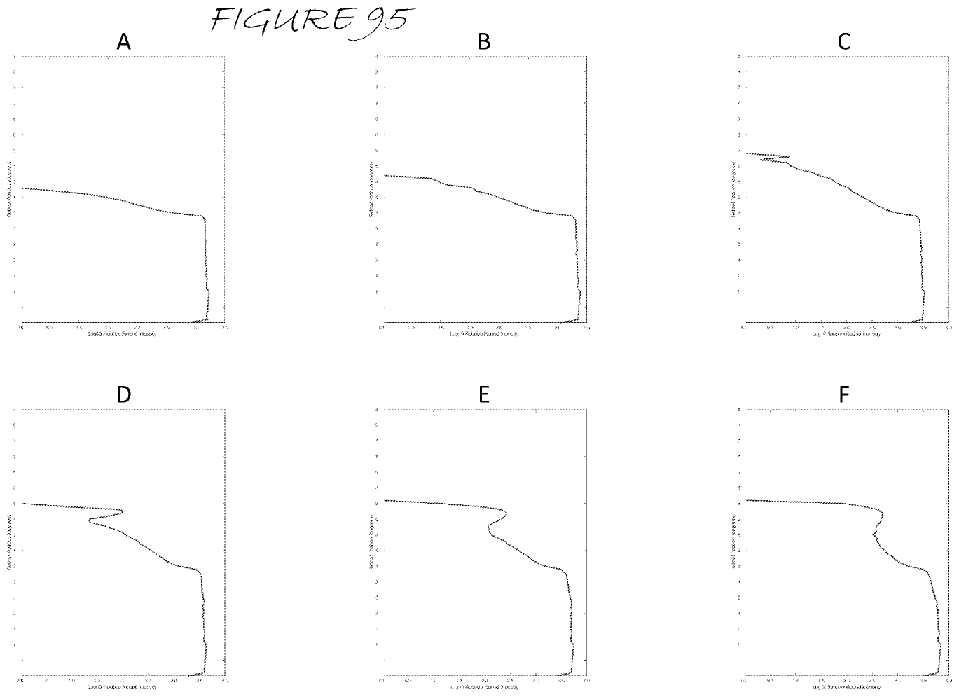

FIGS. 95A-F are relative retina irradiance plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.2 mm in accordance with certain embodiments. FIG. 95A illustrates the relative whole-field retinal irradiance for 0.2 mm IOL implantation depth and a 2.5 mm pupil diameter. FIG. 95B illustrates the relative whole-field retinal irradiance for 0.2 mm IOL implantation depth and a 3 mm pupil diameter. FIG. 95C illustrates the relative whole-field retinal irradiance for 0.2 mm IOL implantation depth and a 3.5 mm pupil diameter. FIG. 95D illustrates the relative whole-field retinal irradiance for 0.2 mm IOL implantation depth and a 4 mm pupil diameter. FIG. 95E illustrates the relative whole-field retinal irradiance for 0.2 mm IOL implantation depth and a 4.5 mm pupil diameter. FIG. 95F illustrates the relative whole-field retinal irradiance for 0.2 mm IOL implantation depth and a 5 mm pupil diameter. As illustrated, at this implantation depth, no dark band retinal region occurs for the illustrated pupil sizes.

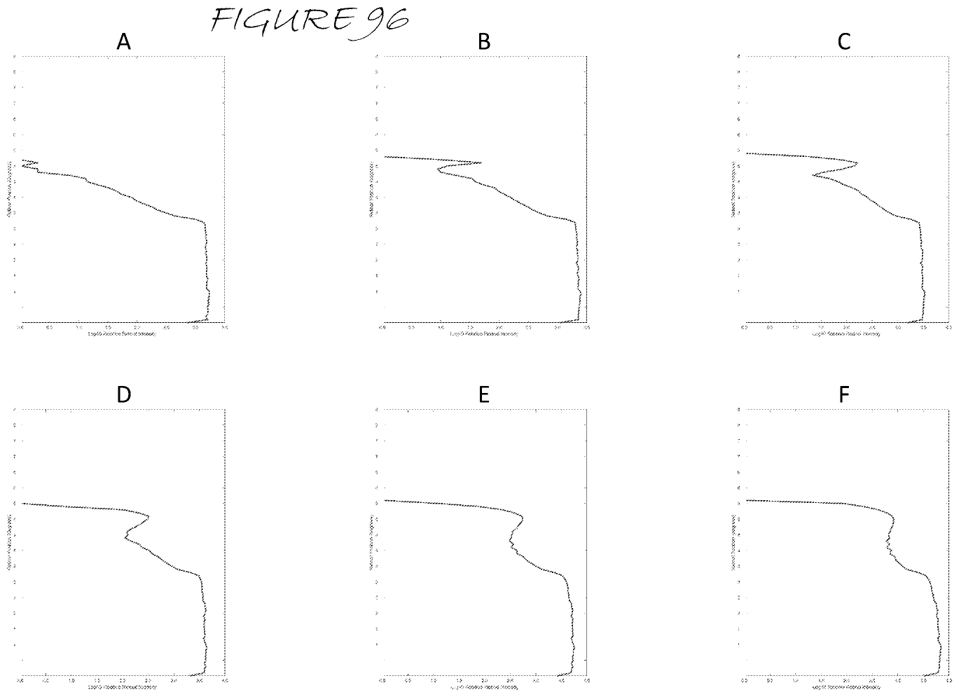

FIGS. 96A-F are relative retina irradiance plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.3 mm in accordance with certain embodiments. FIG. 96A illustrates the relative whole-field retinal irradiance for 0.3 mm IOL implantation depth and a 2.5 mm pupil diameter. FIG. 96B illustrates the relative whole-field retinal irradiance for 0.3 mm IOL implantation depth and a 3 mm pupil diameter. FIG. 96C illustrates the relative whole-field retinal irradiance for 0.3 mm IOL implantation depth and a 3.5 mm pupil diameter. FIG. 96D illustrates the relative whole-field retinal irradiance for 0.3 mm IOL implantation depth and a 4 mm pupil diameter. FIG. 96E illustrates the relative whole-field retinal irradiance for 0.3 mm IOL implantation depth and a 4.5 mm pupil diameter. FIG. 96F illustrates the relative whole-field retinal irradiance for 0.3 mm IOL implantation depth and a 5 mm pupil diameter. As illustrated, at this implantation depth, no dark band retinal region occurs for the illustrated pupil sizes. Although FIG. 96A illustrates a very small irregularity in the retinal intensity, it is believed that this irregularity is due to the number of rays traced and in any event is so small in terms of both spatial width and its intensity relative to neighboring points, that it is unlikely to be detected by the eye.

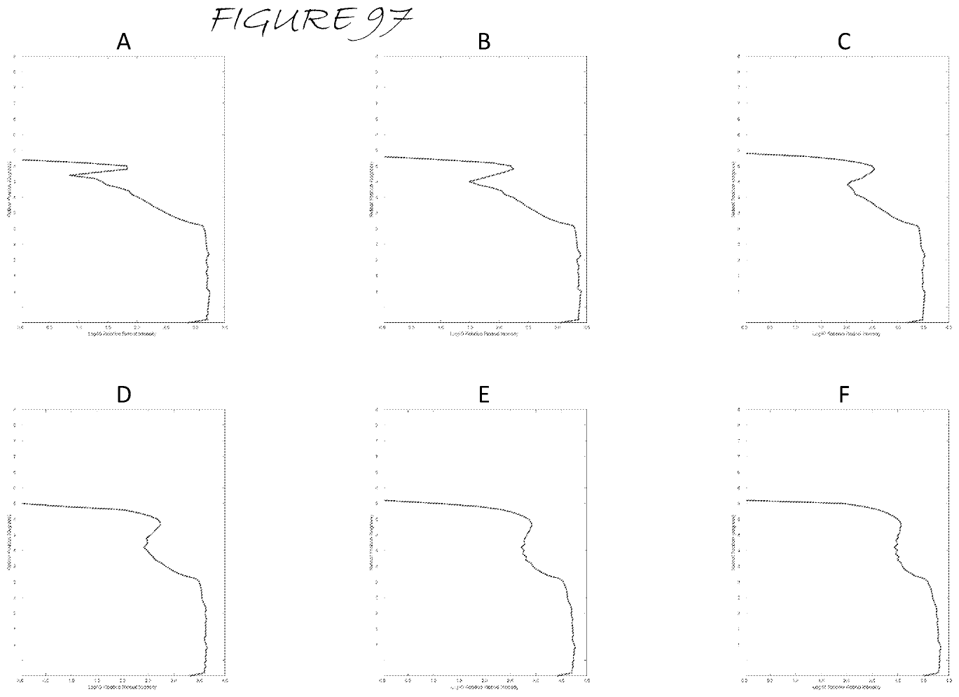

FIGS. 97A-F are relative retina irradiance plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.4 mm in accordance with certain embodiments. FIG. 97A illustrates the relative whole-field retinal irradiance for 0.4 mm IOL implantation depth and a 2.5 mm pupil diameter. FIG. 97B illustrates the relative whole-field retinal irradiance for 0.4 mm IOL implantation depth and a 3 mm pupil diameter. FIG. 97C illustrates the relative whole-field retinal irradiance for 0.4 mm IOL implantation depth and a 3.5 mm pupil diameter. FIG. 97D illustrates the relative whole-field retinal irradiance for 0.4 mm IOL implantation depth and a 4 mm pupil diameter. FIG. 97E illustrates the relative whole-field retinal irradiance for 0.4 mm IOL implantation depth and a 4.5 mm pupil diameter. FIG. 97F illustrates the relative whole-field retinal irradiance for 0.4 mm IOL implantation depth and a 5 mm pupil diameter. As illustrated, at this implantation depth, no dark band retinal region occurs for the illustrated pupil sizes.

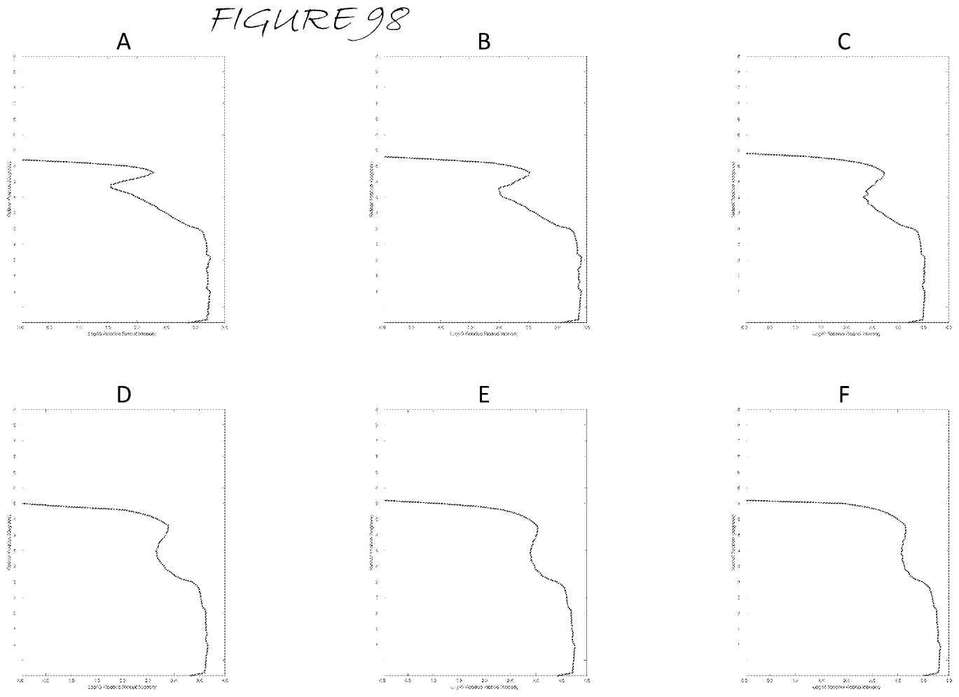

FIGS. 98A-F are relative retina irradiance plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.5 mm in accordance with certain embodiments. FIG. 98A illustrates the relative whole-field retinal irradiance for 0.5 mm IOL implantation depth and a 2.5 mm pupil diameter. FIG. 98B illustrates the relative whole-field retinal irradiance for 0.5 mm IOL implantation depth and a 3 mm pupil diameter. FIG. 98C illustrates the relative whole-field retinal irradiance for 0.5 mm IOL implantation depth and a 3.5 mm pupil diameter. FIG. 98D illustrates the relative whole-field retinal irradiance for 0.5 mm IOL implantation depth and a 4 mm pupil diameter. FIG. 98E illustrates the relative whole-field retinal irradiance for 0.5 mm IOL implantation depth and a 4.5 mm pupil diameter. FIG. 98F illustrates the relative whole-field retinal irradiance for 0.5 mm IOL implantation depth and a 5 mm pupil diameter. As illustrated, at this implantation depth, no dark band retinal region occurs for the illustrated pupil sizes.

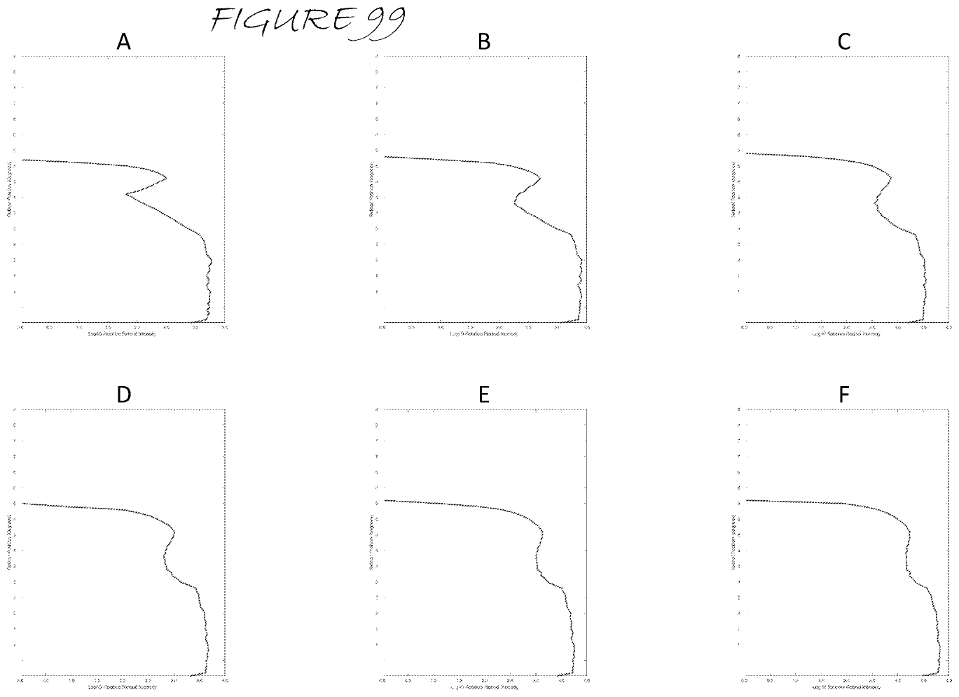

FIGS. 99A-F are relative retina irradiance plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.6 mm in accordance with certain embodiments. FIG. 99A illustrates the relative whole-field retinal irradiance for 0.6 mm IOL implantation depth and a 2.5 mm pupil diameter. FIG. 99B illustrates the relative whole-field retinal irradiance for 0.6 mm IOL implantation depth and a 3 mm pupil diameter. FIG. 99C illustrates the relative whole-field retinal irradiance for 0.6 mm IOL implantation depth and a 3.5 mm pupil diameter. FIG. 99D illustrates the relative whole-field retinal irradiance for 0.6 mm IOL implantation depth and a 4 mm pupil diameter. FIG. 99E illustrates the relative whole-field retinal irradiance for 0.6 mm IOL implantation depth and a 4.5 mm pupil diameter. FIG. 99F illustrates the relative whole-field retinal irradiance for 0.6 mm IOL implantation depth and a 5 mm pupil diameter. As illustrated, at this implantation depth, no dark band retinal region occurs for the illustrated pupil sizes.

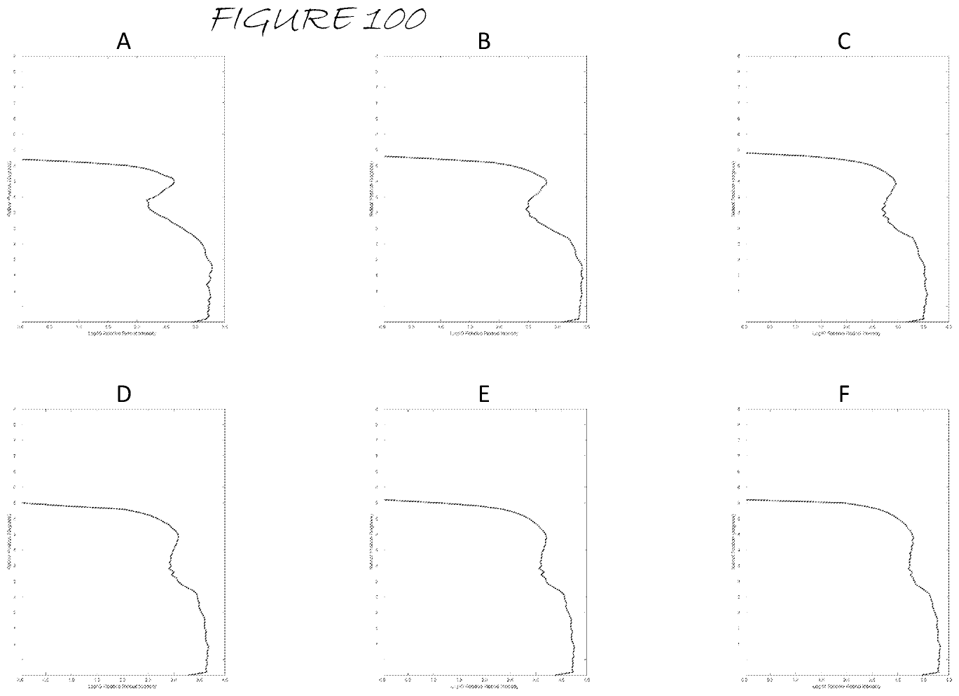

FIGS. 100A-F are relative retina irradiance plots showing the intensity (e.g., relative whole field retinal irradiance) of light distribution across the retina for the intraocular lens modelled in FIG. 84 for pupil sizes from 2.5 mm to 5 mm in 0.5 mm steps at an implantation depth of 0.7 mm in accordance with certain embodiments. FIG. 100A illustrates the relative whole-field retinal irradiance for 0.7 mm IOL implantation depth and a 2.5 mm pupil diameter. FIG. 100B illustrates the relative whole-field retinal irradiance for 0.7 mm IOL implantation depth and a 3 mm pupil diameter. FIG. 100C illustrates the relative whole-field retinal irradiance for 0.7 mm IOL implantation depth and a 3.5 mm pupil diameter. FIG. 100D illustrates the relative whole-field retinal irradiance for 0.7 mm IOL implantation depth and a 4 mm pupil diameter. FIG. 100E illustrates the relative whole-field retinal irradiance for 0.7 mm IOL implantation depth and a 4.5 mm pupil diameter. FIG. 100F illustrates the relative whole-field retinal irradiance for 0.7 mm IOL implantation depth and a 5 mm pupil diameter. As illustrated, at this implantation depth, no dark band retinal region occurs for the illustrated pupil sizes.

In certain situations when a pseudophakic individual experiences peripheral pseudophakic dysphotopsia, it may be inadvisable to remove the existing intraocular lens (for example due to surgical risk, etc.). In such applications, an intraocular lens for reducing, minimizing, and/or eliminating peripheral pseudophakic dysphotopsia may be implanted as a supplementary intraocular lens to operate in combination with (e.g., in conjunction with, together with, or in unison with) the existing intraocular lens.

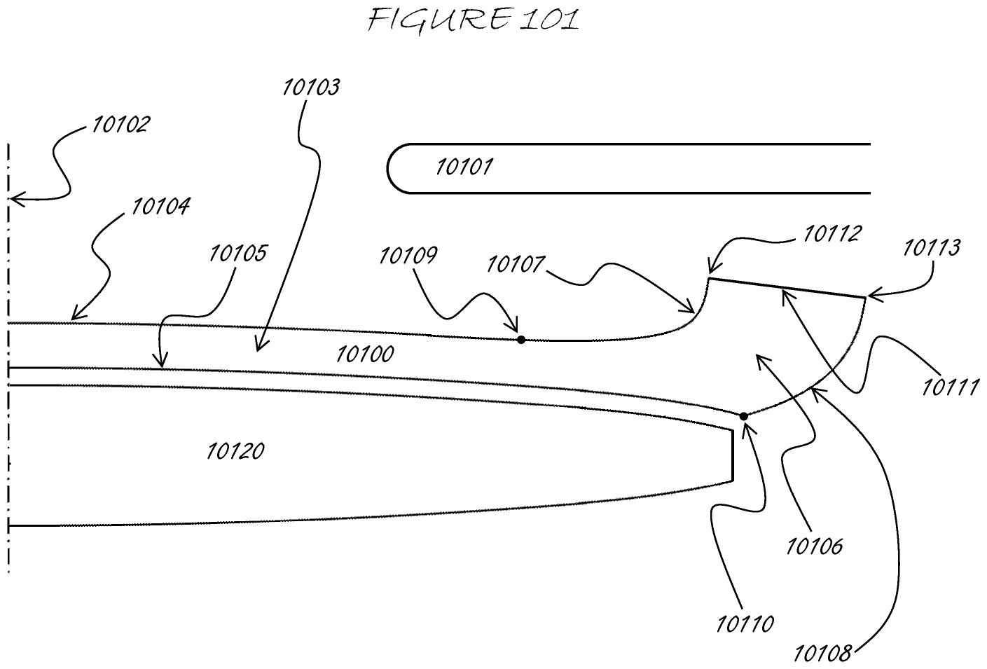

FIG. 101 is a schematic illustration of a half-meridian section of a supplementary intraocular lens reducing, minimizing, and/or eliminating peripheral pseudophakic dysphotopsia in accordance with certain embodiments. For orientation, the coordinate system of FIG. 101 is the same as that for FIG. 16. An axis of the eye/supplementary IOL 10102 is positioned at the left of FIG. 101.

As illustrated, FIG. 101 shows a half-meridional cross section of a supplementary IOL 10100 about the axis of the optical system 10102.

The meridional cross section of FIG. 101 is for purpose of illustration only and may not be isometric (e.g., anisometric); that is, the distance (or dimensions or scaling) in the horizontal direction may not be the same as that in the vertical direction.

The supplementary IOL 10100 is implanted to operate with an existing IOL 10120. The cross section of the supplementary IOL 10100 is illustrated as being implanted posteriorly to the iris 10101 of and eye. The supplementary IOL 10100 may be implanted to be in contact with the existing IOL 10120 or may be implanted to be spaced apart (as illustrated in FIG. 101) from the existing IOL 10120.

The supplementary IOL 10100 comprises an optic zone 10103 and a control zone 10106. In some embodiments, prescribed optical power of the supplementary IOL 10100 may be provided by the optic zone 10103. The combined optical power of the supplementary IOL 10100 and existing IOL 10120 may provide the requisite power for the eye.

The optic zone of the supplementary IOL may be characterized by any combination of one or more of a front (anterior) optic surface 10104, a back (posterior) optic surface 10105, a thickness (between front and back optic surfaces e.g., along axis 10102), and a refractive index of the supplementary IOL material.

In some embodiments, the back (or posterior) optic surface 10105 may have a surface profile (e.g., curvature, shape, asphericity) to facilitate spaced-apart alignment or substantially spaced-apart alignment of the back optic surface 10105 of supplementary IOL 10100 to the front surface of the existing IOL 10120.

In some embodiments, the back (or posterior) optic surface 10105 may have a surface profile (e.g., curvature, shape, asphericity) to facilitate contact alignment or substantially contact alignment (e.g. apposition) of the back optic surface 10105 of supplementary IOL 10100 to the front surface of the existing IOL 10120.

In some embodiments, the control zone 10106 may be configured to control PPD (e.g., negative and/or positive PPD). As illustrated, the control zone 10106 may comprise a front (anterior) control surface 10107, a back (posterior) control surface 10108 and an edge 10111. A boundary between the optic zone 10103 and the control zone 10106 forms an optic-control junction. The front optic-control junction 10109 marks the boundary or transition from the front optic surface 10104 to the front control surface 10107. The back optic-control junction 10110 marks the boundary or transition from the back optic surface 10105 to the back control surface 10108.

As the control zone 10106 is positioned towards the periphery of the supplementary IOL 10100, the optic zone 10103 (which is more centrally located) of the supplementary IOL 10100 may, in some embodiments, function in the same way as conventional IOLs, or in combination with the existing IOL function in the same way as conventional IOLs. For example, the optic zone 10103 may be configured to deliver an optical power within a large range. The optic zone 10103 may incorporate any combination of one or more of a range of conventional IOL optics including multifocal optics or extended depth of focus optics for supporting near vision, diffractive optics, toric optics for correcting astigmatism, etc. The combined optical power of the supplementary IOL 10100 and the existing IOL 10120 may provide the requisite power for the eye.

In some embodiments, the intersection of the control surfaces 10107, 10108 and the edge 10111 may form junctions 10112, 10113 between the control surfaces and the edge. For example, the front control surface 10107 may meet the edge 10111 at the front control-edge junction 10112 and the back control surface 10108 may meet the edge 10111 at the back control-edge junction 10113.

In some embodiments, the front and/or back control surfaces 10107, 10108 of control zone 10106 may be configured to have particular surface curvatures and/or profiles to redirect and/or distribute light to otherwise dark band regions of the retina. By configuring the control zone 10106 to fill-in (e.g., refract light to) the dark band region, the supplementary IOL 10100 may reduce, significantly reduce, and/or eliminate the occurrence/perception of PPD.

In some embodiments, the eye (either with natural crystalline lens, or with IOL) may be approximal to a rotationally symmetric optical system and an axis 10102 may be used to reference directions and radial or transverse distances.

In some embodiments, the optic zone 10103 may be located in the central portion of the supplementary IOL and provide the optical power (for example, in combination with the optical power of the existing IOL) for supporting vision of the patient. Optical characteristics (e.g., power, aberrations, depth of focus, etc.) of the optic zone may be determined by the curvature or profile of the front and back optic surfaces 10104, 10105, the supplementary IOL thickness, as well as the refractive index of the supplementary IOL material.

The control zone 10106 may be configured to control negative PPD. In some embodiments, the control zone, or front control surface, or back control surface configuration, may be based on configurations and/or design approaches disclosed throughout this specification (e.g., in the exemplary embodiments shown in any of FIGS. 27, 29, 31 to 40, and 81 to 84, or Tables 1 to 16).

In some embodiments, the control zone may be configured to intercept a portion of oblique light rays (e.g., from light incident on the eye from peripheral field angles) passing through the pupil and redirect and/or distribute the rays to a region on the retina that would otherwise be a dark band. In some embodiments, this may be achieved by appropriate configurations of a back control surface 10108, a front control surface 10107, the thickness or thickness profile of the supplementary IOL at the control zone 10106 and/or the width (or length, e.g., distance between the control-edge junction points 10112, 10113) of the edge 10111.

In some embodiments, the control zone 10106 may be positioned towards the periphery of the supplementary IOL but may not necessarily extend to the very edge of the supplementary IOL. In some embodiments, the control zone 10106 may extend to the edge of the supplementary IOL.

In some embodiments, the back (posterior) control surface 10108, together with the curvature/surface profile of the front (anterior) control surface 10107, and/or the thickness or thickness profile of the supplementary IOL at the control zone 10106, and/or the width (or length, e.g. distance between the control-edge junction points 10112 and 10113) of the edge 10111, may redirect and/or distribute light to a region on the retina that would otherwise be a dark band.

In some embodiments, the back control surface 10108 may be convex (e.g., substantially convex, or generally convex, i.e. convex when considered across the expanse of the back control surface 10108) towards the back of the eye (e.g., concave towards the front of the eye) as illustrated in FIG. 101. In some embodiments, the back control surface 10108 may have a steeper curvature (e.g., shorter radius of curvature) than the back optic surface 10105. In some embodiments, an absolute value for the radius of curvature of the back control surface 10108 may be smaller (e.g. lesser in value) than an absolute value of the radius of curvature of the back optic surface 10105. For example, the back optic surface 10105 may be a negative refracting surface which is concave and the absolute value of curvature for the back optic surface may be lower (e.g., has a greater absolute radius of curvature) than that for the back control surface 10108 which has greater curvature (e.g., has a lesser absolute radius of curvature) along the meridional cross section.

In some embodiments, the back control surface 10108 may have a curvature opposite in sign to the curvature of the back optic surface 10105. For example, the back optic surface 10105 may be a negative refracting surface (as the example illustrated in FIG. 101) which is convex towards the front of the eye while the back control surface 10108 may be concave towards the front of the eye; that is, the two surfaces have opposite signs in curvature.

In some embodiments, the back control surface 10108 may vary in curvature (e.g., local curvature or instantaneous curvature) along its profile.

In some embodiments, the back control surface 10108 profile may be increasing in curvature (e.g., radius of curvature becomes shorter) towards the edge 10111 of the supplementary IOL 10100. In some embodiments, the back control surface 10108 profile may be decreasing in curvature (e.g., radius of curvature becomes longer) towards the edge of the supplementary IOL 10100. In some embodiments, the back control surface 10108 profile may decrease in curvature (e.g., radius of curvature becomes longer) then increase in curvature (e.g., radius of curvature becomes shorter) towards the edge 10111 of the supplementary IOL 10100. In some embodiments, the back control surface 10108 profile may increase in curvature (e.g., radius of curvature becomes shorter) then decrease in curvature (e.g., radius of curvature becomes longer) towards the edge 10111 of the supplementary IOL 10100.

In some embodiments, the slope relative to (e.g., referenced to, or measured from) a frontal plane along the back control surface 10108 near to the back control-edge junction 10113 is such that as the back control surface 10108 progresses radially outwards (e.g., from axis of the supplementary IOL towards the peripheral retina), points on the back control surface 10108 become positioned more anteriorly (e.g., towards the iris).

In some embodiments, the absolute value of the angle of a slope relative to a frontal plane of the back control surface 10108 at or near to the back control-edge junction 10113 is greater than the absolute value of the angle of a slope relative to a frontal plane of the back control surface 10108 at, or near to, the back optic-control junction 10110.

In some embodiments, a slope of the back control surface 10108, relative to (e.g., referenced to, or measured from) a frontal plane, along the back control surface 10108 in a point or region not coincident with (e.g. not on, not co-located), but is proximal (e.g. near to, in the vicinity of) the back control-edge junction 10113, is such that as the back control surface 10108 progresses radially outwards (e.g., in a direction from axis of the supplementary IOL towards the peripheral retina), points on the back control surface 10108 close to (e.g., near to or at) the back control-edge junction 10113 become positioned more anteriorly (e.g., towards the cornea of the eye), and the absolute value of an angle of a slope, relative to a frontal plane, of the back control surface 10108 at the back control-edge junction 10113, is greater than the absolute value of a slope, relative to a frontal plane, of a point or region of the back control surface 10108 not on (e.g. not coincident with, not co-located with) but near to (e.g. proximal to) the back control-edge junction 10113.

In some embodiments, an angle of a slope of the back control surface 10108, relative to a frontal plane, at (e.g., co-located with, coincident with), or near to (e.g., proximal to, in the vicinity of), the back control-edge junction 10113 is more negative in value than an angle of a slope of the back control surface 10108, relative to a frontal plane at, or near to, the back optic-control junction 10110. For the angle of a slope of a supplementary IOL surface (e.g. optic surface, control surface, edge) relative to a frontal plane, the sign of the angle is considered to be positive when a point on a tangent to the surface of the slope becomes more posterior in position (e.g., nearer the back of the eye, or towards the fovea or retina) as the point progresses radially outwards (e.g., away from an axis of the supplementary IOL) along a tangent to the surface of the slope. Conversely, the sign of the angle of a slope relative to a frontal plane is considered to be negative when a point on the surface of the slope becomes more anterior in position (i.e. nearer the front of the eye, or towards the cornea or the incoming light source) as the point progresses radially outwards (e.g., away from an axis of the supplementary IOL) along a tangent of the surface of the slope. Note that the value of one or both of the angles may be positive or negative in sign in this comparison of such some embodiments. For example, the angle of the slope of the back control surface 10108 at the back optic-junction point 10110 as illustrated in FIG. 101 is negative in value, while the angle of the slope on the back control surface 10108 near to the control-edge junction 10113 as illustrated in FIG. 101 is more negative in value.

In some embodiments, the back control surface 10108 profile may be defined by an aspheric curve: definable by mathematical functions including conics, polynomials, Bezier curves, spline curves, Fourier series, wavelets, or combinations of two or more of such functions.

In some embodiments, the back control surface 10108 may be C0-continuous with the back optic surface 10105. For example, the back control surface 10108 may meet the back optic surface 10105 without a ledge or ‘jump’. In some embodiments, the back control surface 10108 may be C1-continuous with the back optic surface 10105. For example, the back control surface 10108 may have a common tangent with the back optic surface 10105 where they meet.

The front (anterior) control surface 10107 is the surface on the front surface of the supplementary IOL 10100 that lies within the control zone 10106. Together with the curvature/surface profile of the back control surface 10108, the thickness or thickness profile of the supplementary IOL at the control zone 10106 and/or the width (or length, e.g., distance between the control-edge junction points 10112, 10113) of the edge 10111, the curvature/surface profile of the front control surface 10107 may redirect and/or distribute light to a region on the retina that would otherwise be a dark band.

In some embodiments, the front control surface 10107 may be convex (e.g., substantially convex, or generally convex, e.g., convex when considered across the expanse of the front control surface 10107) towards the back of the eye (e.g., concave towards the front of the eye) as illustrated in FIG. 101. In some embodiments, the front control surface 10107 may have a steeper curvature (e.g., shorter radius of curvature) than the front optic surface 10104. In some embodiments, an absolute value for the radius of curvature of the front control surface 10107 may be smaller (e.g. lesser in value) than an absolute value of the radius of curvature of the front optic surface 10104. For example, the front optic surface 10104 may be a positive refracting surface which is convex and the absolute value of curvature for the front optic surface is lower (i.e. has a greater absolute radius of curvature) than that for the front control surface 10107 which has greater curvature (i.e. has a lesser absolute radius of curvature) along the meridional cross section.

In some embodiments, the front control surface 10107 may have a curvature opposite in sign to the curvature of the front optic surface 10104. For example, the front optic surface 10104 may be a positive refracting surface which is convex towards the front of the eye while the front control surface 10107 may be concave towards the front of the eye; that is, the two surfaces may have opposite signs in curvature.

In some embodiments, the front control surface 10107 may vary in curvature (e.g., local curvature or instantaneous curvature) along its profile.

In some embodiments, the front control surface 10107 profile may be increasing in curvature (e.g., the radius of curvature becomes shorter) towards the edge 10111 of the supplementary IOL 10100.

In some embodiments, the front control surface 10107 profile may be decreasing in curvature (e.g., radius of curvature becomes longer) towards the edge of the supplementary IOL 10100. In some embodiments, the front control surface 10107 profile may decrease in curvature (e.g., radius of curvature becomes longer) then increase in curvature (e.g., radius of curvature becomes shorter) towards the edge 10111 of the supplementary IOL 10100. In some embodiments, the front control surface 10107 profile may increase in curvature (e.g., radius of curvature becomes shorter) then decrease in curvature (e.g., radius of curvature becomes longer) towards the edge 10111 of the supplementary IOL 10100.

In some embodiments, the slope relative to (e.g., referenced to, or measured from) a frontal plane along the front control surface 10107 near to (e.g. proximal to, or in the vicinity of) the front control-edge junction 10112 is such that as the front control surface 10107 progresses radially outwards (e.g., from axis of the supplementary IOL towards the peripheral retina), points on the front control surface 10107 become positioned more anteriorly (e.g., towards the iris).

In some embodiments, the absolute value of a slope relative to a frontal plane of the front control surface 10107 at or near to the front control-edge junction 10112 is greater than the absolute value of the angle of a slope relative to a frontal plane of the front control surface 10107 at the front optic-control junction 10109.

In some embodiments, a slope of the front control surface 10107, relative to (e.g., referenced to, or measured from) a frontal plane, along the front control surface 10107 in a point or region not coincident with (e.g. not co-located, not on), but is proximal to (e.g. near to, in the vicinity of) the front control-edge junction 10112, is such that as the front control surface 10107 progresses radially outwards (e.g., in a direction from axis of the supplementary IOL towards the peripheral retina), points on the front control surface 10107 close to (e.g., near to or at) the front control-edge junction 10112 become positioned more anteriorly (e.g., towards the iris), and the absolute value of an angle of a slope, relative to a frontal plane, of the front control surface 10107 at the front control-edge junction 10112, is greater than the absolute value of a slope, relative to a frontal plane, of a point or region of the front control surface 10107 not on (e.g. not coincident with, not co-located with) but proximal to (e.g. near to) the front optic-control junction 10109.

In some embodiments, an angle of a slope of the front control surface 10107, relative to a frontal plane, at (e.g. co-located with, coincident with), or at near to (e.g. proximal to, in the vicinity of), the front control-edge junction 10112 is more negative in value than an angle of a slope of the front control surface 10107, relative to a frontal plane at, or at near to, the front optic-control junction 10109.

In some embodiments, the front control surface 10107 profile may be defined by an aspheric curve, definable by mathematical functions including conics, polynomials, Bezier curves, spline curves, Fourier series, wavelets, or combinations of two or more of such functions. In some embodiments, the front control surface 10107 may be C0-continuous with the front optic surface 10104. For example, the front control surface 10107 may meet the front optic surface at a common point, without a ledge or jump.

In some embodiments, the front control surface 10107 may be C1-continuous with the front optic surface 10104. For example, the front control surface 10107 may have a common tangent with the front optic surface 10104 where they meet.

The front optic-control junction 10109, also referred to as the front optic-control boundary, is the location or region on the front surface of the supplementary IOL 10100 where the front optic surface 10104 meets the front control surface 10107. In some embodiments, the radial/transverse position of the front optic-control junction 10109 may impose a limit on the size of the optic zone 10103. In some embodiments, the front optic-control junction 10109 may be easily definable as an individual point and in some embodiments, the front optic control junction may be a less definable region between the optic zone 10103 and the control zone 10106. In some embodiments, the front optic-control junction 10109 may be a ‘point’ (when viewed as a meridional cross-section) at which the front optic 10104 and control 10107 surfaces directly meet, or may be a region (e.g., annulus for a circular supplementary IOL) over which the front optic surface 10104 transitions (or is blended) to the front control surface 10107.

In some embodiments, the position of the front optic-control junction 10109 may be set such that the size of the optic zone 10103 matches (or closely matches) the size of the patient's pupil. In some embodiments, (e.g., due to the Stiles-Crawford Effect), light-rays passing the periphery of the pupil may produce a lesser response by the photo-receptors (e.g., rods and cones) of the retina. Accordingly, matching (or substantially matching) the size of the patient's pupil may not require the front optic-control junction 10109 to be positioned such that size of the optic zone 10103 is the same as the pupil size, but that it can be smaller (or larger) and still not significantly disturb vision.

The back optic-control junction 10110, also referred to as the back optic-control boundary, is the location or region on the back surface where the back optic surface 10105 meets the back control surface 10108. In some embodiments, the radial/transverse position of the back optic-control junction may impose a limit on the size of the optic zone 10103. In some embodiments, the back optic-control junction 10110 may be easily definable as an individual point and in some embodiments, the back optic control junction 10110 may be a less definable region between the optic zone 10103 and the control zone 10106. In some embodiments, the back optic-control junction may be a ‘point’ (when viewed as a meridional cross-section) at which the back optic 10105 and control 10108 surfaces directly meet, or may be a region (e.g., annulus for a circular supplementary IOL) over which the back optic surface 10105 transitions (or is blended) to the back control surface 10108.

In some embodiments, the position of the back optic-control junction may be set such that the size of the optic zone 10103 matches (or closely matches) the size of the patient's pupil. In some embodiments, the back optic-control junction 10110 position may be more peripheral (e.g. further from the axis, closer to the edge) than that of the front optic-control junction 10109.

The edge 10111 of the supplementary IOL 10100 is defined as a surface (e.g., substantially cylindrical or conical if the supplementary IOL is circular) between and joining the front 10107 and back 10108 surfaces of the supplementary IOL 10100. In some embodiments the edge 10111 may be substantially straight, at least partially curved, and/or undulating or otherwise varying between the front surface 10107 and the back (also more peripheral) surface 10108. In some embodiments, where the control zone 10106 extends to the limit of the lens size, the edge may be formed by the surface between and joining the front and back control surfaces 10107 and 10108 respectively. In some embodiments, the edge 10111 may be sloped so it faces anteriorly such that a normal to the edge surface 10111 and an axis 10102 of the supplementary IOL form an angle of less than 40°, 35°, 30°, or 200 (where 0° means the edge 10111 surface is facing directly forward (i.e., the normal to the edge surface 10111 is parallel to the axis 10102 and the edge surface lies in a frontal plane and faces anteriorly towards the iris), and 900 means the edge surface faces directly outwards, parallel to a meridional plane of the eye). For an angle between a normal of an edge and an axis of a supplementary IOL, the sign convention is such that a positive angle indicates a normal to an edge such that, at the edge, points that are more anterior along the normal of the edge (e.g. towards the front of the eye) are positioned further radially (e.g. towards the periphery) from the axis. In some embodiments, the angle between a normal of the edge and an axis of the supplementary IOL may be about 45°, 40°, 35°, 30°, 25°, 20°, 15°, 10°, or 5°. In some embodiments, the angle may be less than about 45°, 40°, 35°, 30°, 25°, 20°, 15°, or 10°. In some embodiments, the angle may be between about 35-45°, 35-40°, 25-35°, 25-30°, 15-25°, 10-20°, 10-15°, 5-10°, 0-15°, 0-20°, 0-30°, 0-40° or 10-40°.

In some embodiments, the edge surface 10111 may be sloped so the angle of the slope is substantially the same as a by-pass ray. That is, the direction of a by-pass ray is substantially parallel to the surface of the edge 10111.

In some embodiments, a slope of the back control surface 10108 at or near the back control-edge junction 10113 forms an angle of about 90° (e.g., is perpendicular to) with a slope of the edge surface 10111 at or near the back control-edge junction 10113.

In some embodiments, a slope of the back control surface 10108 at or near the back control-edge junction 10113 forms an angle equal to or less than about 90° with a slope of the edge surface 10111 at or near the back control-edge junction 10113 where the angle is subtended within the material of the lens (e.g., according to FIG. 101, the angle is formed clockwise from the back control surface 10108 to the edge surface 10111).

In some embodiments, a slope of the back control surface 10108 at or near the back control-edge junction 10113 forms an angle equal to or greater than about 90° with a slope of the edge surface 10111 at or near the back control-edge junction 10113 where the angle is subtended within the material of the lens.

In some embodiments, a slope of the back control surface 10108 at or near the back control-edge junction 10113 forms an angle between about 750 and about 1050 with a slope of the edge surface 10111 at or near the back control-edge junction 10113 where the angle is subtended within the material of the lens.

In some embodiments, a slope of the front control surface 10107 at or near the front control-edge junction 10112 forms an angle of about 90° (e.g., is perpendicular to) with a slope of the edge surface 10111 at or near the front control-edge junction 10112.

In some embodiments, a slope of the front control surface 10107 at or near the front control-edge junction 10112 forms an angle equal to or less than about 90° with a slope of the edge surface 10111 at or near the front control-edge junction 10112 where the angle is subtended within the material of the lens (e.g., according to FIG. 101, the angle is formed anti-clockwise from the front control surface 10107 to the edge surface 10111).

In some embodiments, a slope of the front control surface 10107 at or near the front control-edge junction 10112 forms an angle equal to or greater than about 90° with a slope of the edge surface 10111 at or near the front control-edge junction 10112 where the angle is subtended within the material of the lens.

In some embodiments, a slope of the front control surface 10107 at or near the front control-edge junction 10112 forms an angle between about 750 and about 1050 with a slope of the edge surface 10111 at or near the front control-edge junction 10112 where the angle is subtended within the material of the lens.

In some embodiments, the front control-edge junction 10112 and the back control-edge junction 10113 may be coincident (e.g., substantially coincident, in very close proximity) so the edge surface 10111 may be very narrow in width, or substantially a ‘knife edge’ (e.g. a wedge shape, a taper).

In some embodiments, the edge surface may be treated to alter its optical characteristics such as one or more of transmission/opacity, scattering/diffusing, spectral transmission, reflectance, etc. The treatment may eliminate or reduce the propagation of light rays (e.g. ‘edge’ rays) that may refract or reflect off the edge either from aqueous to lens (from outside inwards), or from lens to aqueous/vitreous (from inside outwards), or from lens to lens (internal reflection), or from aqueous/vitreous to aqueous/vitreous (external reflection).

In some embodiments, the edge surface 10111 may be a smooth refracting or reflecting surface, or may possess optical features such as diffraction gratings, meta-surfaces (e.g., nano-optics pillars), frosting (e.g., similar to shower screens to render the surface scattering/diffusing), etc.

The front control-edge junction 10112 is the location where the front control surface 10107, or a region or zone more peripheral than the front control surface, and the edge 10111 of the supplementary IOL meet. When regarded as a meridional cross-section, the front control-edge junction 10112 may be a sharp corner, a radiused/rounded corner, a chamfered corner, a beveled corner, a filleted corner, or a profile that joins the front control surface 10107 to the edge 10111.

In certain embodiments, the front control curve 10107 may be separated from the front control-edge junction 10112 or the edge 10111 in which case, the front control surface 10107 may appear as a ring or annulus shape that does not continue to the lens edge 10111 when seen from front-on to the supplementary IOL 10100.

The back control-edge junction 10113 is the location where the back control surface 10108, or a region or zone more peripheral than the front control surface, and the edge 10111 of the supplementary IOL meet. When regarded as a meridional cross-section, the back control-edge junction 10113 may be a sharp corner, a radiused/rounded corner, a chamfered corner, a beveled corner, a filleted corner, or a profile that joins the back control surface 10108 to the edge 10111.

In certain embodiments, the back control curve 10108 may be separated from the back control-edge junction 10113 or the supplementary IOL edge 10111 in which case, the back control surface 10108 may be seen as a ring or annulus shape that does not continue to the lens edge 10111 as seen from front-on to the supplementary IOL 10100.

Further advantages of the claimed subject matter will become apparent from the following examples describing certain embodiments of the claimed subject matter. In certain embodiments, one or more than one (including for instance all) of the following further embodiments may comprise each of the other embodiments or parts thereof.

### Examples

A1. An intraocular lens (IOL) comprising: an optic zone; and a control zone positioned peripherally relative to the optic zone and configured to reduce, minimize, and/or eliminate negative peripheral pseudophakic dysphotopsia (PPD).

A2. The intraocular lens of any of the A examples, wherein the optic zone comprises a front (anterior) optic surface, a back (posterior) optic surface, a thickness (between front and back optic surfaces which may be constant or vary radially and/or vary circumferentially and/or vary transversely across at least a portion of the optic zone), and a refractive index.

A3. The intraocular lens of any of the A examples, wherein the control zone comprises a front (anterior) control surface, a back (posterior) control surface, and an edge.

A4. The intraocular lens of any of the A examples, wherein the optic zone comprises a prescribed optical power.

A5. The intraocular lens of any of the A examples, wherein the optic zone is configured to deliver an optical power within a large range.

A6. The intraocular lens of any of the A examples, wherein the optic zone incorporates any combination of one or more of multifocal optics, which may be refractive and/or diffractive or combinations thereof, for supporting near vision, extended depth of focus optics for supporting near vision, and toric optics for correcting astigmatism.

A7. The intraocular lens of any of the A examples, wherein the optic zone is located in a central portion of the IOL and provides an optical power for supporting vision of the patient.

A8. The intraocular lens of any of the A examples, wherein the control zone is positioned towards the periphery of the IOL but does not extend to the very edge of the IOL.

A9. The intraocular lens of any of the A examples, wherein the control zone is positioned towards the periphery of the IOL and extends to the very edge of the IOL.

A10. The intraocular lens of any of the A examples, wherein the control zone is configured to control PPD.

A1 l. The intraocular lens of any of the A examples, wherein the control zone is configured to refract light to the dark band region to reduce, significantly reduce, and/or eliminate the occurrence/perception of PPD.

A12. The intraocular lens of any of the A examples, wherein the control zone is configured to intercept a portion of oblique light rays (e.g., from light incident on the eye from peripheral field angles) passing through the pupil and redirect and/or distribute the rays to a region on the retina that would otherwise be a dark band.

A13. The intraocular lens of any of the A examples, wherein the location where the redirected and/or redistributed light hitting the retina is achieved by appropriate configurations of a back control surface, a front control surface, width of the edge, and/or the thickness or thickness profile (e.g., a thickness profile that increases or decreases towards the periphery of the intraocular lens) of the IOL at the control zone.

A14. The intraocular lens of any of the A examples, wherein a boundary between the optic zone and the control zone forms an optic-control junction comprising a front optic-control junction that marks the boundary or transition from the front optic surface to the front control surface and a back optic-control junction that marks the boundary or transition from the back optic surface to the front control surface.

A15. The intraocular lens of any of the A examples, wherein the size (diameter if circular) of the optic zone is determined by the position of the front optic-control junction and/or the back optic control junction.

A16. The intraocular lens of any of the A examples, wherein the front optic-control junction is a point (when viewed as a meridional cross-section) at which the front optic and control surfaces meet.

A17. The intraocular lens of any of the A examples, wherein the front optic-control junction is a region (e.g., annulus for a circular IOL) over which the front optic surface transitions (or is blended) to the front control surface.

A18. The intraocular lens of any of the A examples, wherein the back optic-control junction is a point (when viewed as a meridional cross-section) at which the back optic and control surfaces meet.

A19. The intraocular lens of any of the A examples, wherein the back optic-control junction is a region (e.g., annulus for a circular IOL) over which the back optic surface transitions (or is blended) to the back control surface.

A20. The intraocular lens of any of the A examples, wherein the position of the front optic-control junction is set such that the size of the optic zone matches (or closely matches) the size of the patient's pupil.

A21. The intraocular lens of any of the A examples, wherein the position of the back optic-control junction is set such that the size of the optic zone matches (or closely matches) the size of the patient's pupil.

A22. The intraocular lens of any of the A examples, wherein the size of the optic zone is slightly smaller or larger than the size of the patients pupil and does not significantly disturb vision.

A23. The intraocular lens of any of the A examples, wherein the back optic-control junction position is more peripheral than that of the front optic-control junction.

A24. The intraocular lens of any of the A examples, wherein the front and/or back control surfaces of the control zone are configured to have particular surface curvatures and/or profiles to redirect and/or distribute light to otherwise dark band regions of the retina.

A25. The intraocular lens of any of the A examples, wherein the width of the control zone is as wide as possible to redirect as much light as possible to redirect light to the otherwise dark band region of the retina without significantly impacting vision.

A26. The intraocular lens of any of the A examples, wherein the back (posterior) control surface, together with the curvature/surface profile of the front (anterior) control surface redirects and/or distributes light to a region on the retina that would otherwise be a dark band.

A27. The intraocular lens of any of the A examples, wherein the back control surface is convex towards the back of the eye (e.g., concave towards the front of the eye).

A28. The intraocular lens of any of the A examples, wherein the back control surface has a steeper curvature (e.g., shorter radius of curvature) than the back optic surface.

A29. The intraocular lens of any of the A examples, wherein the back control surface profile varies in curvature (e.g., radius of curvature changes) between back optic-control junction and the edge of the IOL.

A30. The intraocular lens of any of the A examples, wherein the back control surface profile is gradually increasing in curvature (e.g., radius of curvature becomes shorter) towards the edge of the IOL.

A31. The intraocular lens of any of the A examples, wherein the back control surface profile is gradually decreasing in curvature (e.g., radius of curvature becomes longer) towards the edge of the IOL.

A32. The intraocular lens of any of the A examples, wherein the back control surface profile is gradually decreasing and then gradually increasing in curvature (e.g., radius of curvature becomes longer and then shorter) towards the edge of the IOL.

A33. The intraocular lens of any of the A examples, wherein the back control surface profile is gradually increasing and then gradually decreasing in curvature (e.g., radius of curvature becomes shorter and then longer) towards the edge of the IOL.

A34. The intraocular lens of any of the A examples, wherein the back control surface profile is defined by an aspheric curve; definable by mathematical functions including conics, polynomials, Bezier curves, spline curves, Fourier series, wavelets, or combinations of two or more of such functions.

A35. The intraocular lens of any of the A examples, wherein a slope of the back control surface proximal to the edge of the IOL is such that as the back control surface progresses radially outwards (e.g., from axis of the IOL towards the peripheral retina), points on the back control surface become positioned more anteriorly (e.g., towards the iris).

A36. The intraocular lens of any of the A examples, wherein the absolute value of the angle of a slope relative to the back control surface proximal to the edge of the IOL is greater than the absolute value of the angle of a slope relative to the back control surface at the back optic-control junction.

A37. The intraocular lens of any of the A examples, wherein a slope of the back control surface proximal to the edge of the IOL and the edge surface form an angle of less than 90 degrees, about 90 degrees, and/or greater than 90 degrees.

A38. The intraocular lens of any of the A examples, wherein the back control surface is C0-continuous with the back optic surface (e.g., the back control surface meets the back optic surface without a ledge or jump).

A39. The intraocular lens of any of the A examples, wherein the back control surface is C1-continuous with the back optic surface (e.g., the back control surface has a common tangent with the back optic surface where they meet).

A40. The intraocular lens of any of the A examples, wherein the back control surface is C2-continuous with the back optic surface (e.g., the back control surface has the same instantaneous curvature as the back optic surface at the point where they meet).

A41. The intraocular lens of any of the A examples, wherein the front control surface is convex towards the back of the eye (e.g., concave towards the front of the eye).

A42. The intraocular lens of any of the A examples, wherein the front control surface has a steeper curvature (e.g., shorter radius of curvature) than the back optic surface.

A43. The intraocular lens of any of the A examples, wherein the front optic surface is a positive refracting surface which is convex towards the front of the eye.

A44. The intraocular lens of any of the A examples, wherein the front control surface profile varies in curvature (e.g., radius of curvature changes) towards the edge of the IOL.

A45. The intraocular lens of any of the A examples, wherein the front control surface profile is gradually increasing in curvature (e.g., radius of curvature becomes shorter) between front optic-control junction and the edge of the IOL.

A46. The intraocular lens of any of the A examples, wherein the front control surface profile is gradually decreasing in curvature (e.g., radius of curvature becomes longer) towards the edge of the IOL.

A47. The intraocular lens of any of the A examples, wherein the front control surface profile is gradually decreasing and then gradually increasing in curvature (e.g., radius of curvature becomes longer and then shorter) towards the edge of the IOL.

A48. The intraocular lens of any of the A examples, wherein the front control surface profile is gradually increasing and then gradually decreasing in curvature (e.g., radius of curvature becomes shorter and then longer) towards the edge of the IOL.

A49. The intraocular lens of any of the A examples, wherein the front control surface profile is defined by an aspheric curve; definable by mathematical functions including conics, polynomials, Bezier curves, spline curves, Fourier series, wavelets, or combinations of two or more of such functions.

A50. The intraocular lens of any of the A examples, wherein a slope of the front control surface proximal to the edge of the IOL is such that as the front control surface progresses radially outwards (e.g., from axis of the IOL towards the peripheral retina), points on the front control surface become positioned more anteriorly (e.g., towards the iris).

A51. The intraocular lens of any of the A examples, wherein the absolute value of the angle of a slope relative to the front control surface proximal to the edge of the IOL is greater than the absolute value of the angle of a slope relative to the front control surface at the front optic-control junction.

A52. The intraocular lens of any of the A examples, wherein a slope of the front control surface proximal to the edge of the IOL and the edge surface form an angle of less than 90 degrees, about 90 degrees, and/or greater than 90 degrees.

A53. The intraocular lens of any of the A examples, wherein the front control surface is C0-continuous with the front optic surface (e.g., the front control surface meets the front optic surface without a ledge or jump).

A54. The intraocular lens of any of the A examples, wherein the front control surface is C1-continuous with the front optic surface (e.g., the front control surface has a common tangent with the front optic surface where they meet).

A55. The intraocular lens of any of the A examples, wherein the front control surface is C2-continuous with the front optic surface (e.g., the front control surface has the same instantaneous curvature as the front optic surface at the point where they meet).

A56. The intraocular lens of any of the A examples, wherein the back optic surface and the back control surface meet to create a gradual transition of ray refraction/deflection angles at the back surface for rays within the optic and control zones in the vicinity of the back optic junction.

A57. The intraocular lens of any of the A examples, wherein the front optic surface and the front control surface meet to create a gradual transition of ray refraction/deflection angles at the front surface for rays within the optic and control zones in the vicinity of (e.g. proximal to or near to) the front optic junction.

A58. The intraocular lens of any of the A examples, wherein the curvature/surface profile of the back control surface and/or the curvature/surface profile of the front control surface redirect and/or distribute light to a region on the retina that would otherwise be a dark band.

A59. The intraocular lens of any of the A examples, wherein the edge is formed by the surface between and joining the front and back control surfaces.

A60. The intraocular lens of any of the A examples, wherein the edge is sloped so it faces anteriorly such that a normal to the edge surface and an axis of the IOL form an angle of less than 45°, 40°, 35°, 30°, or 25°.

A61. The intraocular lens of any of the A examples, wherein the edge is sloped so it faces anteriorly such that a normal to the edge surface and an axis of the IOL form an angle of less than about 45°, 40°, 35°, 30°, 25°, 20°, 15°, or 10°.

A62. The intraocular lens of any of the A examples, wherein the edge is sloped so it faces anteriorly such that a normal to the edge surface and an axis of the IOL form an angle of about 45°, 40°, 35°, 30°, 25°, 20°, 15°, 10°, 5°, or 2.5°.

A63. The intraocular lens of any of the A examples, wherein the edge is sloped so it faces anteriorly such that a normal to the edge surface and an axis of the IOL form an angle of between about 35-45°, 35-40°, 25-35°, 25-30°, 15-25°, 10-20°, 10-15°, 5-15°, 0-15°, 5-10°, 0-10°, or 10-40°.

A64. The intraocular lens of any of the A examples, wherein the edge surface is sloped so the angle of the slope is substantially the same as a by-pass ray (e.g., the direction of a by-pass ray is substantially parallel to the surface of the edge).

A65. The intraocular lens of any of the A examples, wherein the edge surface is sloped so the angle of the slope is within about plus or minus 5° to a by-pass ray (e.g., the direction of a by-pass ray is less than about 5° in either directions relative to the slope of the surface of the edge).

A66. The intraocular lens of any of the A examples, wherein a width of the edge surface is about 2.5 mm, 2 mm, 1.5 mm, 1 mm, 0.5 mm, 0.25 mm or 0.1 mm.

A67. The intraocular lens of any of the A examples, wherein a width of the edge surface is less than about 2.5 mm, 2 mm, 1.5 mm, 1 mm, 0.5 mm or 0.25 mm.

A68. The intraocular lens of any of the A examples, wherein the edge surface may be treated to alter its optical characteristics (e.g., one or more of transmission/opacity, scattering/diffusing, spectral transmission, reflectance, etc.).

A69. The intraocular lens of any of the A examples, wherein the treatment eliminates or reduces the propagation of light rays that may refract or reflect off the edge either from aqueous to lens (from outside inwards) or from lens to aqueous or vitreous (from inside outwards), or from lens to lens (internal reflection), or from aqueous to aqueous (external reflection).

A70. The intraocular lens of any of the A examples, wherein, the edge surface is a smooth refracting or reflecting surface, or possesses optical features such as diffraction gratings, meta-surfaces (e.g., nano-optics pillars), frosting (e.g., similar to shower screens to render the surface scattering/diffusing).

A71. The intraocular lens of any of the A examples, wherein a front control-edge junction is the location where the front control surface, or a region or zone more peripheral than the front control surface, and the edge of the IOL meet.

A72. The intraocular lens of any of the A examples, wherein a front control-edge region is the region on the front surface where the front control surface, or a region or zone more peripheral than the front control surface, joins to the edge of the IOL.

A73. The intraocular lens of any of the A examples, wherein, when regarded as a meridional cross-section, the front control-edge junction may be a sharp corner, a radiused/rounded corner, a chamfered corner, a filleted corner, or a profile that joins the front control surface to the edge.

A74. The intraocular lens of any of the A examples, wherein a back control-edge junction is the location where the back control surface, or a region or zone more peripheral than the back control surface, and the edge of the IOL meet.

A75. The intraocular lens of any of the A examples, wherein, when regarded as a meridional cross-section, the back control-edge junction may be a sharp corner, a radiused/rounded corner, a chamfered corner, a filleted corner, or a profile that joins the back control surface to the edge.

A76. The intraocular lens of any of the A examples, wherein a back control-edge region is the region on the back surface where the back control surface, or a region or zone more peripheral than the back control surface, joins to the edge of the IOL.

B1. An intraocular lens (IOL) comprising: a front (anterior) surface comprising a front optic surface located in the central portion of the front surface and a front control surface located peripherally to the front optic surface; a back (posterior) surface comprising a back optic surface located in the central portion of the back surface and a back control surface located peripherally to the back optic surface; an optic zone defined by the front optic surface, the back optic surface, a thickness (which may be constant or vary radially or circumferentially) between front optic surface and the back optic surface, and a refractive index (e.g., one or more refractive index); and a control zone positioned peripherally relative to the optic zone and defined by the front control surface, the back control surface, and an edge; wherein the front optic surface has a first surface curvature and the front control surface has a second surface curvature different than the first surface curvature, and the back optic surface has a third surface curvature and the back control surface has a fourth surface curvature different than the third surface curvature; wherein the control zone is configured to reduce, minimize, and/or eliminate negative peripheral pseudophakic dysphotopsia (PPD) (e.g., negative PPD).

B2. An intraocular lens (IOL) comprising: an optic zone comprising a front (anterior) optic surface, a back (posterior) optic surface, a thickness (between front and back optic surfaces which may be constant or vary radially or circumferentially), and a refractive index (e.g., one or more refractive index); and a control zone positioned peripherally relative to the optic zone and comprising a front (anterior) control surface, a back (posterior) control surface, and an edge; wherein the front optic surface has a first surface curvature and the front control surface has a second surface curvature different than the first surface curvature, and the back optic surface has a third surface curvature and the back control surface has a fourth surface curvature different than the third surface curvature; wherein the control zone is configured to reduce, minimize, and/or eliminate negative peripheral pseudophakic dysphotopsia (PPD).

B3. The intraocular lens of any of the B examples, wherein the front control surface is convex (e.g., substantially convex, or generally convex, e.g., convex when considered across the expanse of the front control surface) towards the back of the eye (e.g., concave towards the front of the eye).

B4. The intraocular lens of any of the B examples, wherein the front control surface may have a curvature opposite in sign to the curvature of the front optic surface.

B5. The intraocular lens of any of the B examples, wherein an absolute value for the radius of curvature of the front control surface may be smaller (e.g. lesser in value) than an absolute value of the radius of curvature of the front optic surface.

B6. The intraocular lens of any of the B examples, wherein the front control surface has a steeper curvature (e.g., shorter radius of curvature) than the front optic surface.

B7. The intraocular lens of any of the B examples, wherein the front optic surface is a positive refracting surface which is convex and the absolute value of curvature for the front optic surface is lower (i.e. has a greater absolute radius of curvature) than that for the front control surface which has greater curvature (i.e. has a lesser absolute radius of curvature) along the meridional cross section.

B8. The intraocular lens of any of the B examples, wherein the front optic surface is a positive refracting surface which is convex towards the front of the eye while the front control surface is concave towards the front of the eye.

B9. The intraocular lens of any of the B examples, wherein the back control surface may be convex (e.g., substantially convex, or generally convex, i.e. convex when considered across the expanse of the back control surface) towards the back of the eye (e.g., concave towards the front of the eye).

B10. The intraocular lens of any of the B examples, wherein the back control surface has a curvature opposite in sign to the curvature of the back optic surface.

B11. The intraocular lens of any of the B examples, wherein an absolute value for the radius of curvature of the back control surface is smaller (e.g. lesser in value) than an absolute value of the radius of curvature of the back optic surface.

B12. The intraocular lens of any of the B examples, wherein the back control surface has a steeper curvature (e.g., shorter radius of curvature) than the back optic surface.

B13. The intraocular lens of any of the B examples, wherein the back optic surface is a positive refracting surface which is convex and the absolute value of curvature for the back optic surface is lower (e.g., has a greater absolute radius of curvature) than that for the back control surface which has greater curvature (e.g., has a lesser absolute radius of curvature) along the meridional cross section.

B14. The intraocular lens of any of the B examples, wherein the back optic surface is a negative refracting surface which is convex towards the front of the eye while the back control surface is concave towards the front of the eye.

B15. The intraocular lens of any of the B examples, wherein the optic zone comprises a front (anterior) optic surface, a back (posterior) optic surface, a thickness (between front and back optic surfaces which may be constant or vary radially or circumferentially), and a refractive index.

B16. The intraocular lens of any of the B examples, wherein the control zone comprises a front (anterior) control surface, a back (posterior) control surface, and an edge.

B17. The intraocular lens of any of the B examples, wherein the optic zone comprises a prescribed optical power.

B18. The intraocular lens of any of the B examples, wherein the optic zone is configured to deliver an optical power within a large range.

B19. The intraocular lens of any of the B examples, wherein the optic zone incorporates any combination of one or more of multifocal optics for supporting near vision, extended depth of focus optics for supporting near vision, diffractive optics, and toric optics for correcting astigmatism.

B20. The intraocular lens of any of the B examples, wherein the optic zone is located in a central portion of the IOL and provides an optical power for supporting vision of the patient.

B21. The intraocular lens of any of the B examples, wherein the control zone is positioned towards the periphery of the IOL but does not extend to the very edge of the IOL.

B22. The intraocular lens of any of the B examples, wherein the control zone is positioned towards the periphery of the IOL and extends to the very edge of the IOL.

B23. The intraocular lens of any of the B examples, wherein the control zone is configured to control negative PPD.

B24. The intraocular lens of any of the B examples, wherein the control zone is configured to refract light to the dark band region to reduce, significantly reduce, and/or eliminate the occurrence/perception of PPD.

B25. The intraocular lens of any of the B examples, wherein the control zone is configured to intercept a portion of oblique light rays (e.g., from light incident on the eye from peripheral field angles) passing through the pupil and redirect and/or distribute the rays to a region on the retina that would otherwise be a dark band.

B26. The intraocular lens of any of the B examples, wherein the location where the redirected and/or redistributed light hitting the retina is achieved by appropriate configurations of a back control surface, a front control surface, width of the edge, and/or the thickness or thickness profile (e.g., a thickness profile that increases or or decreases towards the periphery of the intraocular lens) of the IOL at the control zone.

B27. The intraocular lens of any of the B examples, wherein a boundary between an optic zone and a control zone forms an optic-control junction, said optic-control junction marks the boundary or transition from the optic surface to the control surface.

B28. The intraocular lens of any of the B examples, wherein the size (diameter if circular) of the optic zone is determined by the position of the front optic-control junction and/or the back optic control junction.

B29. The intraocular lens of any of the B examples, wherein the front optic-control junction is a point (when viewed as a meridional cross-section) at which the front optic and control surfaces meet.

B30. The intraocular lens of any of the B examples, wherein the front optic-control junction is a region (e.g., annulus for a circular IOL) over which the front optic surface transitions (or is blended) to the front control surface.

B31. The intraocular lens of any of the B examples, wherein the back optic-control junction is a point (when viewed as a meridional cross-section) at which the back optic and control surfaces meet.

B32. The intraocular lens of any of the B examples, wherein the optic-control junction is a region (e.g., annulus for a circular IOL) over which an optic surface transitions (or is blended) to a control surface.

B33. The intraocular lens of any of the B examples, wherein the position of the front optic-control junction is set such that the size of the optic zone matches (or closely matches) the size of the patient's pupil.

B34. The intraocular lens of any of the B examples, wherein the position of the back optic-control junction is set such that the size of the optic zone matches (or closely matches) the size of the patient's pupil.

B35. The intraocular lens of any of the B examples, wherein the size of the optic zone is slightly smaller or larger than the size of the patients pupil and does not significantly disturb vision.

B36. The intraocular lens of any of the B examples, wherein the back optic-control junction position is more peripheral than that of the front optic-control junction.

B37. The intraocular lens of any of the B examples, wherein the front and/or back control surfaces of the control zone are configured to have particular surface curvatures and/or profiles to redirect and/or distribute light to otherwise dark band regions of the retina.

B38. The intraocular lens of any of the B examples, wherein the back (posterior) control surface, together with the curvature/surface profile of the front (anterior) control surface redirects and/or distributes light to a region on the retina that would otherwise be a dark band.

B39. The intraocular lens of any of the B examples, wherein the back control surface is convex towards the back of the eye (e.g., concave towards the front of the eye).

B40. The intraocular lens of any of the B examples, wherein the back control surface has a steeper curvature (e.g., shorter radius of curvature) than the back optic surface.

B41. The intraocular lens of any of the B examples, wherein the back control surface profile varies in curvature (e.g., radius of curvature changes) towards the edge of the IOL.

B42. The intraocular lens of any of the B examples, wherein the back control surface profile is gradually increasing in curvature (e.g., radius of curvature becomes shorter) towards the edge of the IOL.

B43. The intraocular lens of any of the B examples, wherein the back control surface profile is gradually decreasing in curvature (e.g., radius of curvature becomes longer) towards the edge of the IOL.

B44. The intraocular lens of any of the B examples, wherein the back control surface profile is gradually decreasing and then gradually increasing in curvature (e.g., radius of curvature becomes longer and then shorter) towards the edge of the IOL.

B45. The intraocular lens of any of the B examples, wherein the back control surface profile is gradually increasing and then gradually decreasing in curvature (e.g., radius of curvature becomes shorter and then longer) towards the edge of the IOL.

B46. The intraocular lens of any of the B examples, wherein the back control surface profile is defined by an aspheric curve; definable by mathematical functions including conics, polynomials, Bezier curves, spline curves, Fourier series, wavelets, or combinations of two or more of such functions.

B47. The intraocular lens of any of the B examples, wherein a slope of the back control surface proximal to the edge of the IOL is such that as the back control surface progresses radially outwards (e.g., from axis of the IOL towards the peripheral retina), points on the back control surface become positioned more anteriorly (e.g., towards the iris).

B48. The intraocular lens of any of the B examples, wherein the absolute value of the angle of a slope relative to a frontal plane of the intraocular lens of the back control surface proximal to the edge of the IOL is greater than the absolute value of the angle of a slope relative to the frontal plane of the intraocular lens of the back control surface at the back optic-control junction.

B49. The intraocular lens of any of the B examples, wherein an angle of a slope of the back control surface relative to a frontal plane of the intraocular lens, at or proximal to the back control-edge junction is more negative in value than an angle of a slope of the back control surface relative to the frontal plane of the intraocular lens at or near to the back optic-control junction.

B50. The intraocular lens of any of the B examples, wherein an angle of a slope of the front control surface relative to a frontal plane of the intraocular lens, at or proximal to the front control-edge junction is more negative in value than an angle of a slope of the front control surface relative to the frontal plane of the intraocular lens at or near to the front optic-control junction.

B51. The intraocular lens of any of the B examples, wherein a slope of a control surface proximal to the edge of the IOL and the edge surface form an angle of between 70 degrees and 110 degrees, or between 75 degrees and 105 degrees, or between 80 degrees and 100 degrees.

B52. The intraocular lens of any of the B examples, wherein a control surface is C0-continuous with an optic surface (e.g., the back control surface meets the back optic surface without a ledge or jump).

B53. The intraocular lens of any of the B examples, wherein a control surface is C1-continuous with an optic surface (e.g., the back control surface has a common tangent with the back optic surface where they meet).

B54. The intraocular lens of any of the B examples, wherein the back control surface is C2-continuous with the back optic surface (e.g., the back control surface has the same instantaneous curvature as the back optic surface at the point where they meet).

B55. The intraocular lens of any of the B examples, wherein the front control surface is convex towards the back of the eye (e.g., concave towards the front of the eye).

B56. The intraocular lens of any of the B examples, wherein the front control surface has a steeper curvature (e.g., shorter radius of curvature) than the back optic surface.

B57. The intraocular lens of any of the B examples, wherein the front optic surface is a positive refracting surface which is convex towards the front of the eye.

B58. The intraocular lens of any of the B examples, wherein the front control surface profile varies in curvature (e.g., radius of curvature changes) towards the edge of the IOL.

B59. The intraocular lens of any of the B examples, wherein the front control surface profile is gradually increasing in curvature (e.g., radius of curvature becomes shorter) towards the edge of the IOL.

B60. The intraocular lens of any of the B examples, wherein the front control surface profile is gradually decreasing in curvature (e.g., radius of curvature becomes longer) towards the edge of the IOL.

B61. The intraocular lens of any of the B examples, wherein the front control surface profile is gradually decreasing and then gradually increasing in curvature (e.g., radius of curvature becomes longer and then shorter) towards the edge of the IOL.

B62. The intraocular lens of any of the B examples, wherein the front control surface profile is gradually increasing and then gradually decreasing in curvature (e.g., radius of curvature becomes shorter and then longer) towards the edge of the IOL.

B63. The intraocular lens of any of the B examples, wherein the front control surface profile is defined by an aspheric curve; definable by mathematical functions including conics, polynomials, Bezier curves, spline curves, Fourier series, wavelets, or combinations of two or more of such functions.

B64. The intraocular lens of any of the B examples, wherein a slope of the front control surface proximal to the edge of the IOL is such that as the front control surface progresses radially outwards (e.g., from axis of the IOL towards the peripheral retina), points on the front control surface become positioned more anteriorly (e.g., towards the iris).

B65. The intraocular lens of any of the B examples, wherein the absolute value of the angle of a slope relative to the front control surface proximal to the edge of the IOL is greater than the absolute value of the angle of a slope relative to the front control surface at the front optic-control junction.

B66. The intraocular lens of any of the B examples, wherein the front control surface is C2-continuous with the front optic surface (e.g., the front control surface has the same instantaneous curvature as the front optic surface at the point where they meet).

B67. The intraocular lens of any of the B examples, wherein the back optic surface and the back control surface meet to create a gradual transition of ray refraction/deflection angles at the back surface for rays within the optic and control zones in the vicinity of the back optic junction.

B68. The intraocular lens of any of the B examples, wherein the curvature/surface profile of the back control surface and/or the curvature/surface profile of the front control surface redirect and/or distribute light to a region on the retina that would otherwise be a dark band.

B69. The intraocular lens of any of the B examples, wherein the edge is formed by the surface between and joining the front and back control surfaces.

B70. The intraocular lens of any of the B examples, wherein the edge is sloped so it faces anteriorly such that a normal to the edge surface and an axis of the IOL form an angle of about 45°, 40°, 35°, 30°, 25°, 20°, 15°, 10°, or 5°.

B71. The intraocular lens of any of the B examples, wherein the edge is sloped so it faces anteriorly such that a normal to the edge surface and an axis of the IOL form an angle of less than about 45°, 40°, 35°, 30°, 25°, or 20°.

B72. The intraocular lens of any of the B examples, wherein the edge is sloped so it faces anteriorly such that a normal to the edge surface and an axis of the IOL form an angle of between about 35-45°, 35-40°, 25-35°, 25-30°, 15-25°, 10-20°, 10-15°, 5-10°, or 10-40°.

B73. The intraocular lens of any of the B examples, wherein the edge surface is sloped so the angle of the slope is substantially the same as a by-pass ray (e.g., the direction of a by-pass ray is parallel to the surface of the edge).

B74. The intraocular lens of any of the B examples, wherein a width of the edge surface is about 2.5 mm, 2 mm, 1.5 mm, 1 mm or 0.5 mm.

B75. The intraocular lens of any of the B examples, wherein a width of the edge surface is less than about 2.5 mm, 2 mm, 1.5 mm, 1 mm or 0.5 mm.

B76. The intraocular lens of any of the B examples, wherein the edge surface may be treated to alter its optical characteristics (e.g., one or more of transmission/opacity, scattering/diffusing, spectral transmission, reflectance, etc.).

B77. The intraocular lens of any of the B examples, wherein the treatment eliminates or reduces the propagation of light rays that may refract or reflect off the edge either from aqueous to lens (from outside inwards) or from lens to aqueous (from inside outwards), or from lens to lens (internal reflection).

B78. The intraocular lens of any of the B examples, wherein, the edge surface is a smooth refracting or reflecting surface, or possesses optical features such as diffraction gratings, meta-surfaces (e.g., nano-optics pillars), frosting (e.g., similar to shower screens to render the surface scattering/diffusing).

B79. The intraocular lens of ant of the B examples, wherein a front control-edge junction is the location where the front control surface, or a region or zone more peripheral than the front control surface, and the edge of the IOL meet.

B80. The intraocular lens of any of the B examples, wherein, when regarded as a meridional cross-section, the front control-edge junction may be a sharp corner, a radiused/rounded corner, a chamfered corner, a filleted corner, or a profile that joins the front control surface to the edge.

B81. The intraocular lens of any of the B examples, wherein a back control-edge junction is the location where the back control surface, or a region or zone more peripheral than the front control surface, and the edge of the IOL meet.

B82. The intraocular lens of any of the B examples, wherein, when regarded as a meridional cross-section, the back control-edge junction may be a sharp corner, a radiused/rounded corner, a chamfered corner, a filleted corner, or a profile that joins the back control surface to the edge.

B83. The intraocular lens of any of the B examples, wherein the intraocular lens is a supplementary intraocular lens that is implanted to function in conjunction with an existing intraocular lens.

It will be understood that the embodiments disclosed and defined in this specification extends to all alternative combinations of two or more of the individual features mentioned or evident from the text or drawings. All of these different combinations constitute various alternative aspects of the present disclosure.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. An intraocular lens (IOL) comprising:
a front (anterior) surface comprising a front optic surface located in the central portion of the front surface and a front control surface located peripherally to the front optic surface;
a back (posterior) surface comprising a back optic surface located in the central portion of the back surface and a back control surface located peripherally to the back optic surface;
an optic zone defined by the front optic surface, the back optic surface, a thickness (which may be constant or vary radially or circumferentially) between front optic surface and the back optic surface, and a refractive index (e.g., one or more refractive index); and
a control zone positioned peripherally relative to the optic zone and defined by the front control surface, the back control surface, and an edge;
wherein the front optic surface has a first surface curvature and the front control surface has a second surface curvature different than the first surface curvature, and the back optic surface has a third surface curvature and the back control surface has a fourth surface curvature different than the third surface curvature;
wherein the control zone is configured to reduce, minimize, and/or eliminate negative peripheral pseudophakic dysphotopsia (PPD) (e.g., negative PPD).

2. An intraocular lens (IOL) comprising:
an optic zone comprising a front (anterior) optic surface, a back (posterior) optic surface, a thickness (between front and back optic surfaces which may be constant or vary radially or circumferentially), and a refractive index (e.g., one or more refractive index); and
a control zone positioned peripherally relative to the optic zone and comprising a front (anterior) control surface, a back (posterior) control surface, and an edge;
wherein the front optic surface has a first surface curvature and the front control surface has a second surface curvature different than the first surface curvature, and the back optic surface has a third surface curvature and the back control surface has a fourth surface curvature different than the third surface curvature;
wherein the control zone is configured to reduce, minimize, and/or eliminate negative peripheral pseudophakic dysphotopsia (PPD).

3. The intraocular lens of claim 1, wherein the front control surface is convex (e.g., substantially convex, or generally convex, e.g., convex when considered across the expanse of the front control surface) towards the back of the eye (e.g., concave towards the front of the eye).

4. The intraocular lens of claim 1, wherein the front control surface may have a curvature opposite in sign to the curvature of the front optic surface.

5. The intraocular lens of claim 1, wherein an absolute value for the radius of curvature of the front control surface may be smaller (e.g., lesser in value) than an absolute value of the radius of curvature of the front optic surface.

6. The intraocular lens of claim 1, wherein the front control surface has a steeper curvature (e.g., shorter radius of curvature) than the front optic surface.

7. The intraocular lens of claim 1, wherein the front optic surface is a positive refracting surface which is convex and the absolute value of curvature for the front optic surface is lower (i.e., has a greater absolute radius of curvature) than that for the front control surface which has greater curvature (i.e. has a lesser absolute radius of curvature) along the meridional cross section.

8. The intraocular lens of claim 1, wherein the front optic surface is a positive refracting surface which is convex towards the front of the eye while the front control surface is concave towards the front of the eye.

9. The intraocular lens of claim 1, wherein the back control surface may be convex (e.g., substantially convex, or generally convex, i.e., convex when considered across the expanse of the back control surface) towards the back of the eye (e.g., concave towards the front of the eye).

10. The intraocular lens of claim 1, wherein the back control surface has a curvature opposite in sign to the curvature of the back optic surface.

11. The intraocular lens of claim 1, wherein an absolute value for the radius of curvature of the back control surface is smaller (e.g., lesser in value) than an absolute value of the radius of curvature of the back optic surface.

12. The intraocular lens of claim 1, wherein the back control surface has a steeper curvature (e.g., shorter radius of curvature) than the back optic surface.

13. The intraocular lens of claim 1, wherein the back optic surface is a positive refracting surface which is convex and the absolute value of curvature for the back optic surface is lower (e.g., has a greater absolute radius of curvature) than that for the back control surface which has greater curvature (e.g., has a lesser absolute radius of curvature) along the meridional cross section.

14. The intraocular lens of claim 1, wherein the optic zone comprises a prescribed optical power.

15. The intraocular lens of claim 1, wherein the optic zone incorporates any combination of one or more of multifocal optics for supporting near vision, extended depth of focus optics for supporting near vision, diffractive optics, and toric optics for correcting astigmatism.

16. The intraocular lens of claim 1, wherein the control zone is positioned towards the periphery of the IOL but does not extend to the very edge of the IOL.

17. The intraocular lens of claim 1, wherein the control zone is positioned towards the periphery of the IOL and extends to the very edge of the IOL.

18. The intraocular lens of claim 1, wherein the control zone is configured to intercept a portion of oblique light rays (e.g., from light incident on the eye from peripheral field angles) passing through the pupil and redirect and/or distribute the rays to a region on the retina that would otherwise be a dark band.

19. The intraocular lens of claim 1, wherein a boundary between an optic zone and a control zone forms an optic-control junction, said optic-control junction marks the boundary or transition from the optic surface to the control surface.

20. The intraocular lens of claim 19, wherein the optic-control junction is a region (e.g., annulus for a circular IOL) over which an optic surface transitions (or is blended) to a control surface.

21. The intraocular lens of claim 1, wherein a slope of the back control surface proximal to the edge of the IOL is such that as the back control surface progresses radially outwards (e.g., from axis of the IOL towards the peripheral retina), points on the back control surface become positioned more anteriorly (e.g., towards the iris).

22. The intraocular lens of claim 1, wherein the absolute value of the angle of a slope relative to a frontal plane of the intraocular lens of the back control surface proximal to the edge of the IOL is greater than the absolute value of the angle of a slope relative to the frontal plane of the intraocular lens of the back control surface at the back optic-control junction.

23. The intraocular lens of claim 1, wherein an angle of a slope of the back control surface relative to a frontal plane of the intraocular lens, at or proximal to the back control-edge junction is more negative in value than an angle of a slope of the back control surface relative to the frontal plane of the intraocular lens at or near to the back optic-control junction.

24. The intraocular lens of claim 1, wherein an angle of a slope of the front control surface relative to a frontal plane of the intraocular lens, at or proximal to the front control-edge junction is more negative in value than an angle of a slope of the front control surface relative to the frontal plane of the intraocular lens at or near to the front optic-control junction.

25. The intraocular lens of claim 1, wherein a slope of a control surface proximal to the edge of the IOL and the edge surface form an angle of between 70 degrees and 110 degrees, or between 75 degrees and 105 degrees, or between 80 degrees and 100 degrees.

26. The intraocular lens of claim 1, wherein a control surface is C0-continuous with an optic surface (e.g., the back control surface meets the back optic surface without a ledge or jump).

27. The intraocular lens of claim 1, wherein a control surface is C1-continuous with an optic surface (e.g., the back control surface has a common tangent with the back optic surface where they meet).

28. The intraocular lens of claim 1, wherein the absolute value of the angle of a slope relative to the front control surface proximal to the edge of the IOL is greater than the absolute value of the angle of a slope relative to the front control surface at the front optic-control junction.

29. The intraocular lens of claim 1, wherein the edge is sloped so it faces anteriorly such that a normal to the edge surface and an axis of the IOL form an angle of less than about 45°, 40°, 35°, 30°, 25°, or 20°.

30. The intraocular lens of claim 1, wherein the intraocular lens is a supplementary intraocular lens that is implanted to function in conjunction with an existing intraocular lens.
